SEQUENCE LISTING (509 sequences)

SEQ ID NO: 1 - RNA, 98 nt, Homo sapiens
aacauguugucugugguacccuacucuggagagugacaaucauguauaauuaaauuugauugacacuucugugaguagaguaacgcaugacacguacg

SEQ ID NO: 2 - RNA, 88 nt, Homo sapiens
guugucugugguacccuacucuggagagugacaaucauguauaacuaaauuugauugacacuucugugaguagaguaacgcaugacac

SEQ ID NO: 3 - RNA, 88 nt, Homo sapiens; identical to SEQ ID NO: 2

SEQ ID NO: 4 - RNA, 80 nt, Homo sapiens
caugugguacucuucucaagagggaggcaaucauguguaauuagauaugauugacaccucugugaguggaguaacacaug

SEQ ID NO: 5 - RNA, 115 nt, Homo sapiens
ccaccuucagcugaguguagugcccuacuccagagggcgucacucauguaaacuaaaacaugauuguagccuuuuggaguagaguaauacacaucacguaacgcauauuuggugg

SEQ ID NO: 6 - RNA, 124 nt, Homo sapiens
gccaccaccaucagccauacuauguguagugccuuauucaggaagguguuacuuaauagauuaauauuuguaaggcacccuucugaguagaguaaugugcaacauggacaacauuugugguggc

SEQ ID NO: 7 - RNA, 94 nt, Homo sapiens
caugcugugugugguacccuacugcagacaguggcaaucauguauaauuaaaaaugauugguacgucuguggguagaguacugcaugacacaug

SEQ ID NO: 8 - RNA, 91 nt, Homo sapiens
caugcugugugugguacccuacugcagacaguggcaaucauguauaauuaaaaaugauugguacgucuguggguagaguacugcaugacac

SEQ ID NO: 9 - RNA, 75 nt, Homo sapiens
gugguacccuacugcagacguggcaaucauguauaauuaaaaaugauugguacgucuguggguagaguacugcau

SEQ ID NO: 10 - RNA, 74 nt, Homo sapiens
gugguguccuacucaggagaguggcaaucacauguaauuaggugugauugaaaccucuaagaguggaguaacac

SEQ ID NO: 11 - RNA, 21 nt, Homo sapiens
uuuugcaccuuuuggagugaa

SEQ ID NO: 12 - RNA, 99 nt, Homo sapiens
ccgauguguauccucagcuuugagaacugaauuccauggguugugucagugucagaccucugaaauucaguucuucagcugggauaucucugucaucgu

SEQ ID NO: 13 - RNA, 97 nt, Homo sapiens
cagaucucagacaucucggggaucaucaugucacgagauaccagugugcacuugugacagauugauaacugaaaggucugggagccacucaucuuca

SEQ ID NO: 14 - RNA, 110 nt, Homo sapiens
ucaccuggccaugugacuugugggcuucccuuugucauccuucgccuagggcucugagcagggcagggacagcaaaggggugcucaguugucacuucccacagcacggag

SEQ ID NO: 15 - RNA, 129 nt, Homo sapiens
gggaugccacauucagccauucagcguacagugccuuucacagggaggugucauuuaugugaacuaaaauauaaauuucaccuuucugagaaggguaauguacagcaugcacugcauaugugguguccc

SEQ ID NO: 16 - RNA, 127 nt, Homo sapiens
ggaugccacauucagccauucagugugcagugccuuucacagggaggugucauuuaugugaacuaaaauauaaauuucaccuuucugagaaggguaauguacagcaugcacugcauauguggugucc

SEQ ID NO: 17 - RNA, 84 nt, Homo sapiens
guguacagugccuuucacaaggaggugucauuuaugugaacuaaaauauaaaugucaccuuuuugagaggaguaauguacagca

SEQ ID NO: 18 - RNA, 84 nt, Homo sapiens
gcguacagugccuuucucaaggaggugucguuuaugugaacuaaaauauaaauuucaccuuucugagaagaguaauguacagca

SEQ ID NO: 19 - RNA, 71 nt, Homo sapiens
ggagaggaggcaagaugcuggcauagcuguugaacugggaaccugcuaugccaacauauugccaucuuucc

SEQ ID NO: 20 - RNA, 75 nt, Homo sapiens
uguugucggguggaucacgaugcaauuuugaugaguaucauaggagaaaaauugcacgguauccaucuguaaacc

SEQ ID NO: 21 - RNA, 89 nt, Homo sapiens
cgggguugguuguuaucuuugguuaucuagcuguaugagugguguggagucuucauaaagcuagauaaccgaaaguaaaaauaacccca

SEQ ID NO: 22 - RNA, 87 nt, Homo sapiens
ggaagcgaguuguuaucuuugguuaucuagcuguaugaguguauuggucuucauaaagcuagauaaccgaaaguaaaaacuccuuca

SEQ ID NO: 23 - RNA, 90 nt, Homo sapiens
ggaggcccguuucucucuuugguuaucuagcuguaugagugccacagagccgucauaaagcuagauaaccgaaaguagaaaugauucuca

SEQ ID NO: 24 - RNA, 84 nt, Homo sapiens
gugcucgguuuguaggcagugucauuagcugauuguacuguggugguuacaaucacuaacuccacugccaucaaaacaaggcac

SEQ ID NO: 25 - RNA, 98 nt, Homo sapiens
cgaggggauacagcagcaauucauguuuugaaguguucuaaaugguucaaaacgugaggcgcugcuauacccccucguggggaagguagaaggugggg

SEQ ID NO: 26 - RNA, 72 nt, Homo sapiens
ugucggguagcuuaucagacugauguugacuguugaaucucauggcaacaccagucgaugggcugucugaca

SEQ ID NO: 27 - RNA, 65 nt, Homo sapiens
cuguuaaugcuaaucgugauagggguuuuugccuccaacugacuccuacauauuagcauuaacag

SEQ ID NO: 28 - RNA, 97 nt, Homo sapiens
cacucugcuguggccuauggcuuuucauuccuaugugauugcugucccaaacucauguagggcuaaaagccaugggcuacagugaggggcgagcucc

SEQ ID NO: 29 - RNA, 110 nt, Homo sapiens
uuggauguuggccuaguucuguguggaagacuagugauuuuguuguuuuuagauaacuaaaucgacaacaaaucacagucugccauauggcacaggccaugccucuacag

SEQ ID NO: 30 - RNA, 110 nt, Homo sapiens
cuggauacagaguggaccggcuggccccaucuggaagacuagugauuuuguuguugucuuacugcgcucaacaacaaaucccagucuaccuaauggugccagccaucgca

SEQ ID NO: 31 - RNA, 110 nt, Homo sapiens
agauuagaguggcuguggucuagugcuguguggaagacuagugauuuuguuguucugauguacuacgacaacaagucacagccggccucauagcgcagacucccuucgac

SEQ ID NO: 32 - RNA, 91 nt, Homo sapiens
cugugugugaugagcuggcaguguauuguuagcugguugaauaugugaauggcaucggcuaacaugcaacugcugucuuauugcauauaca

SEQ ID NO: 33 - RNA, 97 nt, Homo sapiens
ugaccugaaucagguaggcaguguauuguuagcuggcugcuugggucaagucagcagccacaacuacccugccacuugcuucuggauaaauucuucu

SEQ ID NO: 34 - RNA, 92 nt, Homo sapiens
gcugggaugugucagguaggcaguguauugcuagcggcuguuaaugauuuuaacaguugcuaguugcacuccucucuguugcauucagaagc

SEQ ID NO: 35 - RNA, 110 nt, Homo sapiens
ggccagcugugaguguuucuuuggcagugucuuagcugguuguugugagcaauaguaaggaagcaaucagcaaguauacugcccuagaagugcugcacguuguggggccc

SEQ ID NO: 36 - RNA, 71 nt, Homo sapiens
ugcccuagcagcgggaacaguucugcagugagcgaucggugcucugggguauuguuuccgcugccagggua

SEQ ID NO: 37 - RNA, 77 nt, Homo sapiens
agucuaguuacuaggcaguguaguuagcugauugcuaauaguaccaaucacuaaccacacggccagguaaaaagauu

SEQ ID NO: 38 - RNA, 69 nt, Homo sapiens
aguaccaaagugcucauagugcagguaguuuuggcaugacucuacuguaguaugggcacuuccaguacu

SEQ ID NO: 39 - RNA, 97 nt, Homo sapiens; identical to SEQ ID NO: 33

SEQ ID NO: 40 - RNA, 84 nt, Homo sapiens
ccagucacguccccuuaucacuuuuccagcccagcuuugugacuguaaguguuggacggagaacugauaaggguaggugauuga

SEQ ID NO: 41 - RNA, 86 nt, Homo sapiens
acugcuaacgaaugcucugacuuuauugcacuacuguacuuuacagcuagcagugcaauaguauugucaaagcaucugaaagcagg

SEQ ID NO: 42 - RNA, 78 nt, Homo sapiens
gccgcaggugcucugacgagguugcacuacugugcucugagaagcagugcaaugauauugucaaagcaucugggacca

SEQ ID NO: 43 - RNA, 89 nt, Homo sapiens
cuggccuccagggcuuuguacaugguaggcuuucauucauucguuugcacauucggugaaggucuacugugugccaggcccugugccag

SEQ ID NO: 44 - RNA, 81 nt, Homo sapiens
ugugcaucguggucaaaugcucagacuccugugguggcugcucaugcaccacggauguuugagcaugugcuacggugucua

SEQ ID NO: 45 - RNA, 81 nt, Homo sapiens
ugugcaucguggucaaaugcucagacuccugugguggcugcuuaugcaccacggauguuugagcaugugcuauggugucua

SEQ ID NO: 46 - RNA, 137 nt, Homo sapiens
guccccuccccuaggccacagccgaggucacaaucaacauucauuguugucgguggguugugaggacugaggccagacccaccgggggaugaaugucacuguggcugggccagacacggcuuaaggggaauggggac

SEQ ID NO: 47 - RNA, 71 nt, Homo sapiens
uguucuaaggugcaucuagugcagauagugaaguagauuagcaucuacugcccuaagugcuccuucuggca

SEQ ID NO: 48 - RNA, 89 nt, Homo sapiens; identical to SEQ ID NO: 21

SEQ ID NO: 49 - RNA, 87 nt, Homo sapiens; identical to SEQ ID NO: 22

SEQ ID NO: 50 - RNA, 90 nt, Homo sapiens; identical to SEQ ID NO: 23

SEQ ID NO: 51 - RNA, 71 nt, Homo sapiens
uguguuaaggugcaucuagugcaguuagugaagcagcuuagaaucuacugcccuaaaugccccuucuggca

SEQ ID NO: 52 - RNA, 94 nt, Homo sapiens
cuuuggcgaucacugccucucugggccugugucuuaggcucugcaagaucaaccgagcaaagcacacggccugcagagaggcagcgcucugccc

SEQ ID NO: 53 - RNA, 110 nt, Homo sapiens
ccugugcagagauuauuuuuuaaaaggucacaaucaacauucauugcugucgguggguugaacuguguggacaagcucacugaacaaugaaugcaacuguggccccgcuu

SEQ ID NO: 54 - RNA, 89 nt, Homo sapiens
cugauggcugcacucaacauucauugcugucgguggguuugagucugaaucaacucacugaucaaugaaugcaaacugcggaccaaaca

SEQ ID NO: 55 - RNA, 69 nt, Homo sapiens
cuguggugcauuguaguugcauugcauguucuggugguacccaugcaauguuuccacagugcaucacag

SEQ ID NO: 56 - RNA, 96 nt, Homo sapiens
gcgggcggccccgcggugcauugcuguugcauugcacgugugugaggcgggugcagugccucggcagugcagcccggagccggccccuggcaccac

SEQ ID NO: 57 - RNA, 87 nt, Homo sapiens
gguugcuucagugaacauucaacgcugucggugaguuuggaauucaaauaaaaaccaucgaccguugauuguacccuauagcuaacc

SEQ ID NO: 58 - RNA, 87 nt, Homo sapiens
ucucagccugugacccucuagagggaagcgcuuucuguugucugaaagaaaagaaagugcaucuuuuuagaggauuacaguuugaga

SEQ ID NO: 59 - RNA, 84 nt, Homo sapiens
aggcugagguaguaguuuguacaguuugagggucuaugauaccacccgguacaggagauaacuguacaggccacugccuugcca

SEQ ID NO: 60 - RNA, 70 nt, Homo sapiens
uugaagggagaucgaccguguuauauucgcuuuauugacuucgaauaauacaugguugaucuuuucucag

SEQ ID NO: 61 - RNA, 63 nt, Homo sapiens
aagaaaugguuuaccgucccacauacauuuugaauauguaugugggaugguaaaccgcuucuu

SEQ ID NO: 62 - RNA, 79 nt, Homo sapiens
ugcaugaguucgucuuggucuaggauuguuggaggagucagaaaaacuaccccagggauccugaaguccuuugggugga

SEQ ID NO: 63 - RNA, 83 nt, Homo sapiens
ucaugcuguggcccuccagagggaagcgcuuucuguugucugaaagaaaacaaagcgcuccccuuuagagguuuacgguuuga

SEQ ID NO: 64 - RNA, 79 nt, Homo sapiens
ugguacucggggagagguuacccgagcaacuuugcaucuggacgacgaauguugcucggugaaccccuuuucgguauca

SEQ ID NO: 65 - RNA, 102 nt, Homo sapiens
cccaagucagguacucgaauggagguuguccaugguguguucauuuuauuuaugaugaguauuacauggccaaucuccuuucgguacucaauucuucuuggg

SEQ ID NO: 66 - RNA, 110 nt, Homo sapiens
gccgagaccgagugcacagggcucugaccuaugaauugacagccagugcucucgucuccccucuggcugccaauuccauaggucacagguauguucgccucaaugccagc

SEQ ID NO: 67 - RNA, 85 nt, Homo sapiens
ggcugagccgcaguaguucuucaguggcaagcuuuauguccugacccagcuaaagcugccaguugaagaacuguugcccucugcc

SEQ ID NO: 68 - RNA, 87 nt, Homo sapiens
ucagagugagguaguagauuguauaguugugggguagugauuuuacccuguucaggagauaacuauacaaucuauugccuucccuga

SEQ ID NO: 69 - RNA, 83 nt, Homo sapiens
ugugggaugagguaguagauuguauaguuuuagggucauaccccaucuuggagauaacuauacagucuacugucuuucccacg

SEQ ID NO: 70 - RNA, 85 nt, Homo sapiens
augguguuaucaaguguaacagcaacuccauguggacuguguaccaauuuccaguggagaugcuguuacuuuugaugguuaccaa

SEQ ID NO: 71 - RNA, 85 nt, Homo sapiens
ugguucccgcccccuguaacagcaacuccauguggaagugcccacugguuccaguggggcugcuguuaucuggggcgagggccag

SEQ ID NO: 72 - RNA, 95 nt, Homo sapiens
gucgaggccguggcccggaaguggucggggccgcugcgggcggaagggcgccugugcuucguccgcucggcgguggcccagccaggcccgcggga

SEQ ID NO: 73 - RNA, 94 nt, Homo sapiens
gaguuugguuuuguuuggguuuguucuagguauggucccagggaucccagaucaaaccaggccccugggccuauccuagaaccaaccuaagcuc

SEQ ID NO: 74 - RNA, 84 nt, Homo sapiens
guggcagcuugguggucguaugugugacgccauuuacuugaaccuuuaggagugacaucacauauacggcagcuaaacugcuac

SEQ ID NO: 75 - RNA, 94 nt, Homo sapiens
ugacuccuccaggucuuggaguaggucauuggguggauccucuauuuccuuacgugggccacuggauggcuccuccaugucuuggaguagauca

SEQ ID NO: 76 - RNA, 97 nt, Homo sapiens
ucccuuucccaggggaggggcuggguuuacguugggagaacuuuuacggugaaccaggagguucucccaacguaagcccagccccuccccucugccu

SEQ ID NO: 77 - RNA, 110 nt, Homo sapiens
cgccucagagccgcccgccguuccuuuuuccuaugcauauacuucuuugaggaucuggccuaaagagguauagggcaugggaaaacggggcggucggguccuccccagcg

SEQ ID NO: 78 - RNA, 68 nt, Homo sapiens
gaggcaaaguucugagacacuccgacucugaguaugauagaagucagugcacuacagaacuuugucuc

SEQ ID NO: 79 - RNA, 97 nt, Homo sapiens
uacuuauuacugguagugagucucuaagaaaagaggaggugguuguuuuccuccucuuuucuuugagacucacuaccaauaauaagaaauacuacua

SEQ ID NO: 80 - RNA, 87 nt, Homo sapiens
caauagacacccaucgugucuuuugcucugcagucaguaaauauuuuuuugugaauguguagcaaaagacagaauggugguccauug

SEQ ID NO: 81 - RNA, 87 nt, Homo sapiens; identical to SEQ ID NO: 80

SEQ ID NO: 82 - RNA, 68 nt, Homo sapiens
cuccggugccuacugagcugauaucaguucucauuuuacacacuggcucaguucagcaggaacaggag

SEQ ID NO: 83 - RNA, 73 nt, Homo sapiens
cucugccucccgugccuacugagcugaaacacaguugguuuguguacacuggcucaguucagcaggaacaggg

SEQ ID NO: 84 - RNA, 80 nt, Homo sapiens
ugggaugagguaguagguuguauaguuuuagggucacacccaccacugggagauaacuauacaaucuacugucuuuccua

SEQ ID NO: 85 - RNA, 72 nt, Homo sapiens
agguugagguaguagguuguauaguuuagaauuacaucaagggagauaacuguacagccuccuagcuuuccu

SEQ ID NO: 86 - RNA, 74 nt, Homo sapiens
gggugagguaguagguuguauaguuuggggcucugcccugcuaugggauaacuauacaaucuacugucuuuccu

SEQ ID NO: 87 - RNA, 110 nt, Homo sapiens
gaucugucugucuucuguauauacccuguagauccgaauuuguguaaggaauuuuguggucacaaauucguaucuaggggaauauguaguugacauaaacacuccgcucu

SEQ ID NO: 88 - RNA, 110 nt, Homo sapiens
acccggcagugccuccaggcgcagggcagccccugcccaccgcacacugcgcugccccagacccacugugcgugugacagcggcugaucugugccugggcagcgcgaccc

SEQ ID NO: 89 - RNA, 78 nt, Homo sapiens
cugaggagcagggcuuagcugcuugugagcaggguccacaccaagucguguucacaguggcuaaguuccgccccccag

SEQ ID NO: 90 - RNA, 84 nt, Homo sapiens
acuggucggugauuuagguaguuuccuguuguugggauccaccuuucucucgacagcacgacacugccuucauuacuucaguug

SEQ ID NO: 91 - RNA, 77 nt, Homo sapiens
ccgggacccaguucaaguaauucaggauagguugugugcuguccagccuguucuccauuacuuggcucggggaccgg

SEQ ID NO: 92 - RNA, 92 nt, Homo sapiens
gggcagucuuugcuacuguaaacauccuugacuggaagcuguaagguguucagaggagcuuucagucggauguuuacagcggcaggcugcca

SEQ ID NO: 93 - RNA, 66 nt, Homo sapiens
agggcuccugacuccagguccuguguguuaccuagaaauagcacuggacuuggagucagaaggccu

SEQ ID NO: 94 - RNA, 96 nt, Homo sapiens
cucgggaggggcgggagggggguccccggugcucggaucucgagggugcuuauuguucgguccgagccugggucucccucuuccccccaacccccc

SEQ ID NO: 95 - RNA, 87 nt, Homo sapiens
ugucccccccggcccagguucugugauacacuccgacucgggcucuggagcagucagugcaugacagaacuugggcccggaaggacc

SEQ ID NO: 96 - RNA, 73 nt, Homo sapiens
ggccggcugggguuccuggggaugggauuugcuuccugucacaaaucacauugccagggauuuccaaccgacc

SEQ ID NO: 97 - RNA, 79 nt, Homo sapiens
cccgggcugagguaggagguuguauaguugaggaggacacccaaggagaucacuauacggccuccuagcuuuccccagg

SEQ ID NO: 98 - RNA, 85 nt, Homo sapiens
ugcaggccucugugugauauguuugauauauuagguuguuauuuaauccaacuauauaucaaacauauuccuacagugucuugcc

SEQ ID NO: 99 - RNA, 70 nt, Homo sapiens; identical to SEQ ID NO: 60

SEQ ID NO: 100 - RNA, 92 nt, Homo sapiens; identical to SEQ ID NO: 92

SEQ ID NO: 101 - RNA, 83 nt, Homo sapiens
gcugcuguugggagacccuggucugcacucuaucuguauucuuacugaagggagugcagggcaggguuucccauacagagggc

SEQ ID NO: 102 - RNA, 88 nt, Homo sapiens
aucucuuacacaggcugaccgauuucuccugguguucagagucuguuuuugucuagcaccauuugaaaucgguuaugauguaggggga

SEQ ID NO: 103 - RNA, 77 nt, Homo sapiens
guggccucguucaaguaauccaggauaggcugugcaggucccaaugggccuauucuugguuacuugcacggggacgc

SEQ ID NO: 104 - RNA, 84 nt, Homo sapiens
ggcuguggcuggauucaaguaauccaggauaggcuguuuccaucugugaggccuauucuugauuacuuguuucuggaggcagcu

SEQ ID NO: 105 - RNA, 75 nt, Homo sapiens
ugcccuggcucaguuaucacagugcugaugcugucuauucuaaagguacaguacugugauaacugaaggauggca

SEQ ID NO: 106 - RNA, 79 nt, Homo sapiens
acuguccuuuuucgguuaucaugguaccgaugcuguauaucugaaagguacaguacugugauaacugaagaaugguggu

SEQ ID NO: 107 - RNA, 76 nt, Homo sapiens
uacuugaagagaaguuguucgugguggauucgcuuuacuuaugacgaaucauucacggacaacacuuuuuucagua

SEQ ID NO: 108 - RNA, 75 nt, Homo sapiens
uggagugggggggcaggaggggcucagggagaaagugcauacagccccuggcccucucugcccuuccguccccug

SEQ ID NO: 109 - RNA, 110 nt, Homo sapiens
ggcuacagucuuucuucaugugacucguggacuucccuuugucauccuaugccugagaauauaugaaggaggcugggaaggcaaagggacguucaauugucaucacuggc

SEQ ID NO: 110 - RNA, 97 nt, Homo sapiens
ugugaucacugucuccagccugcugaagcucagagggcucugauucagaaagaucaucggauccgucugagcuuggcuggucggaagucucaucauc

SEQ ID NO: 111 - RNA, 71 nt, Homo sapiens
gcgacuguaaacauccucgacuggaagcugugaagccacagaugggcuuucagucggauguuugcagcugc

SEQ ID NO: 112 - RNA, 96 nt, Homo sapiens
agaugugcucuccuggcccaugaaaucaagcgugggugagaccuggugcagaacgggaaggcgacccauacuugguuucagaggcugugagaauaa

SEQ ID NO: 113 - RNA, 68 nt, Homo sapiens; identical to SEQ ID NO: 82

SEQ ID NO: 114 - RNA, 88 nt, Homo sapiens
cgaggaugggagcugagggcugggucuuugcgggcgagaugagggugucggaucaacuggccuacaaagucccaguucucggcccccg

SEQ ID NO: 115 - RNA, 66 nt, Homo sapiens; identical to SEQ ID NO: 93

SEQ ID NO: 116 - RNA, 110 nt, Homo sapiens
ccagagguuguaacguugucuauauauacccuguagaaccgaauuugugugguauccguauagucacagauucgauucuaggggaauauauggucgaugcaaaaacuuca

SEQ ID NO: 117 - RNA, 73 nt, Homo sapiens
acuuggagagaggcuggccgugaugaauucgauucaucaaagcgagucauacacggcucuccucucuuuuagu

SEQ ID NO: 118 - RNA, 80 nt, Homo sapiens
gguaccugagaagagguugucugugaugaguucgcuuuuauuaaugacgaauauaacacagauggccuguuuucaguacc

SEQ ID NO: 119 - RNA, 70 nt, Homo sapiens
gugaauuagguaguuucauguuguugggccuggguuucugaacacaacaacauuaaaccacccgauucac

SEQ ID NO: 120 - RNA, 110 nt, Homo sapiens
ugcucgcucagcugaucuguggcuuagguaguuucauguuguugggauugaguuuugaacucggcaacaagaaacugccugaguuacaucagucgguuuucgucgagggc

SEQ ID NO: 121 - RNA, 83 nt, Homo sapiens
cggggugagguaguagguugugugguuucagggcagugauguugccccucggaagauaacuauacaaccuacugccuucccug

SEQ ID NO: 122 - RNA, 84 nt, Homo sapiens
uugguacuuggagagugguuaucccuguccuguucguuuugcucaugucgaaucguacagggucauccacuuuuucaguaucaa

SEQ ID NO: 123 - RNA, 87 nt, Homo sapiens
accgcagggaaaaugagggacuuuugggggcagauguguuuccauuccacuaucauaaugccccuaaaaauccuuauugcucuugca

SEQ ID NO: 124 - RNA, 111 nt, Homo sapiens
agaguguucaaggacagcaagaaaaaugagggacuuucaggggcagcuguguuuucugacucagucauaaugccccuaaaaauccuuauuguucuugcagugugcaucggg

SEQ ID NO: 125 - RNA, 85 nt, Homo sapiens
cgcuggcgacgggacauuauuacuuuugguacgcgcugugacacuucaaacucguaccgugaguaauaaugcgccguccacggca

SEQ ID NO: 126 - RNA, 73 nt, Homo sapiens
cagggugugugacugguugaccagaggggcaugcacuguguucacccugugggccaccuagucaccaacccuc

SEQ ID NO: 127 - RNA, 85 nt, Homo sapiens; identical to SEQ ID NO: 125

SEQ ID NO: 128 - RNA, 73 nt, Homo sapiens; identical to SEQ ID NO: 117

SEQ ID NO: 129 - RNA, 97 nt, Homo sapiens
accucucuaacaaggugcagagcuuagcugauuggugaacagugauugguuuccgcuuuguucacaguggcuaaguucugcaccugaagagaaggug

SEQ ID NO: 130 - RNA, 93 nt, Homo sapiens
ccggggagaaguacggugagccugucauuauucagagaggcuagauccucuguguugagaaggaucaugaugggcuccucgguguucuccagg

SEQ ID NO: 131 - RNA, 96 nt, Homo sapiens
ugguacuuggagagauaguagaccguauagcguacgcuuuaucugugacguauguaacacgguccacuaacccucaguaucaaauccauccccgag

SEQ ID NO: 132 - RNA, 68 nt, Homo sapiens
uaaaagguagauucuccuucuaugaguacauuauuuaugauuaaucauagaggaaaauccacguuuuc

SEQ ID NO: 133 - RNA, 80 nt, Homo sapiens
gguauuuaaaagguagauuuuccuucuaugguuacguguuugaugguuaaucauagaggaaaauccacguuuucaguauc

SEQ ID NO: 134 - RNA, 71 nt, Homo sapiens
gccaacccaguguucagacuaccuguucaggaggcucucaauguguacaguagucugcacauugguuaggc

SEQ ID NO: 135 - RNA, 110 nt, Homo sapiens
aggaagcuucuggagauccugcuccgucgccccaguguucagacuaccuguucaggacaaugccguuguacaguagucugcacauugguuagacugggcaagggagagca

SEQ ID NO: 136 - RNA, 88 nt, Homo sapiens
ugcgcuccucucagucccugagacccuaacuugugauguuuaccguuuaaauccacggguuaggcucuugggagcugcgagucgugcu

SEQ ID NO: 137 - RNA, 89 nt, Homo sapiens
accagacuuuuccuagucccugagacccuaacuugugagguauuuuaguaacaucacaagucaggcucuugggaccuaggcggagggga

SEQ ID NO: 138 - RNA, 67 nt, Homo sapiens
agagaugguagacuauggaacguaggcguuaugauuucugaccuauguaacaugguccacuaacucu

SEQ ID NO: 139 - RNA, 80 nt, Homo sapiens
ccuguugccacaaacccguagauccgaacuugugguauuaguccgcacaagcuuguaucuauagguaugugucuguuagg

SEQ ID NO: 140 - RNA, 110 nt, Homo sapiens
aucauucagaaaugguauacaggaaaaugaccuaugaauugacagacaauauagcugaguuugucugucauuucuuuaggccaauauucuguaugacugugcuacuucaa

SEQ ID NO: 141 - RNA, 110 nt, Homo sapiens
gugauaauguagcgagauuuucuguugugcuugaucuaaccaugugguugcgagguaugaguaaaacaugguuccgucaagcaccauggaacgucacgcagcuuucuaca

SEQ ID NO: 142 - RNA, 110 nt, Homo sapiens
gaccagucgcugcggggcuuuccuuugugcuugaucuaaccaugugguggaacgauggaaacggaacaugguucugucaagcaccgcggaaagcaccgugcucuccugca

SEQ ID NO: 143 - RNA, 97 nt, Homo sapiens
aucugaguugggagggucccucuccaaaugugucuuggggugggggaucaagacacauuuggagagggaaccucccaacucggccucugccaucauu

SEQ ID NO: 144 - RNA, 90 nt, Homo sapiens
gagagaagcacuggacuuagggucagaaggccugagucucucugcugcagaugggcucucugucccugagccaagcuuuguccucccugg

SEQ ID NO: 145 - RNA, 68 nt, Homo sapiens; identical to SEQ ID NO: 132

SEQ ID NO: 146 - RNA, 80 nt, Homo sapiens; identical to SEQ ID NO: 133

SEQ ID NO: 147 - RNA, 110 nt, Homo sapiens
ggccuggcuggacagaguugucaugugucugccugucuacacuugcugugcagaacauccgcucaccuguacagcaggcacagacaggcagucacaugacaacccagccu

SEQ ID NO: 148 - RNA, 81 nt, Homo sapiens
gggcuuucaagucacuagugguuccguuuaguagaugauugugcauuguuucaaaauggugcccuagugacuacaaagccc

SEQ ID NO: 149 - RNA, 87 nt, Homo sapiens
agcuucccuggcucuagcagcacagaaauauuggcacagggaagcgagucugccaauauuggcugugcugcuccaggcaggguggug

SEQ ID NO: 150 - RNA, 78 nt, Homo sapiens
uggccgauuuuggcacuagcacauuuuugcuugugucucuccgcucugagcaaucaugugcagugccaauaugggaaa

SEQ ID NO: 151 - RNA, 81 nt, Homo sapiens
aacacagugggcacucaauaaaugucuguugaauugaaaugcguuacauucaacggguauuuauugagcacccacucugug

SEQ ID NO: 152 - RNA, 97 nt, Homo sapiens
cucaggugcucuggcugcuuggguuccuggcaugcugauuugugacuuaagauuaaaaucacauugccagggauuaccacgcaaccacgaccuuggc

SEQ ID NO: 153 - RNA, 78 nt, Homo sapiens
cugaaauagguugccugugagguguucacuuucuauaugaugaauauuauacagucaaccucuuuccgauaucgaauc

SEQ ID NO: 154 - RNA, 72 nt, Homo sapiens
cuugggaauggcaaggaaaccguuaccauuacugaguuuaguaaugguaaugguucucuugcuauacccaga

SEQ ID NO: 155 - RNA, 110 nt, Homo sapiens
gagcugcuugccuccccccguuuuuggcaaugguagaacucacacuggugagguaacaggauccggugguucuagacuugccaacuauggggcgaggacucagccggcac

SEQ ID NO: 156 - RNA, 110 nt, Homo sapiens
ccagaggacaccuccacuccgucuacccaguguuuagacuaucuguucaggacucccaaauuguacaguagucugcacauugguuaggcugggcuggguuagacccucgg

SEQ ID NO: 157 - RNA, 71 nt, Homo sapiens; identical to SEQ ID NO: 134

SEQ ID NO: 158 - RNA, 110 nt, Homo sapiens; identical to SEQ ID NO: 135

SEQ ID NO: 159 - RNA, 81 nt, Homo sapiens
cccauuggcauaaacccguagauccgaucuuguggugaaguggaccgcacaagcucgcuucuaugggucugugucagugug

SEQ ID NO: 160 - RNA, 112 nt, Homo sapiens
ccaccccgguccugcucccgccccagcagcacacugugguuuguacggcacuguggccacguccaaaccacacugugguguuagagcgagggugggggaggcaccgccgagg

SEQ ID NO: 161 - RNA, 68 nt, Homo sapiens
gcauccuguacugagcugccccgaggcccuucaugcugcccagcucggggcagcucaguacaggauac

SEQ ID NO: 162 - RNA, 63 nt, Homo sapiens; identical to SEQ ID NO: 61

SEQ ID NO: 163 - RNA, 88 nt, Homo sapiens
caccuuguccucacgguccaguuuucccaggaaucccuuagaugcuaagauggggauuccuggaaauacuguucuugaggucaugguu

SEQ ID NO: 164 - RNA, 85 nt, Homo sapiens
gcuaagcacuuacaacuguuugcagaggaaacugagacuuuguaacuaugucucagucucaucugcaaagaaguaagugcuuugc

SEQ ID NO: 165 - RNA, 110 nt, Homo sapiens
ccgcagagugugacuccuguucuguguauggcacugguagaauucacugugaacagucucagucagugaauuaccgaagggccauaaacagagcagagacagauccacga

SEQ ID NO: 166 - RNA, 84 nt, Homo sapiens
gcauccggguugagguaguagguuguaugguuuagaguuacacccugggaguuaacuguacaaccuucuagcuuuccuuggagc

SEQ ID NO: 167 - RNA, 106 nt, Homo sapiens
gcgcagcgcccugucucccagccugaggugcagugcugcaucucuggucaguugggagucugagaugaagcacuguagcucaggaagagagaaguuguucugcagc

SEQ ID NO: 168 - RNA, 94 nt, Homo sapiens
uguuuugagcgggggucaagagcaauaacgaaaaauguuugucauaaaccguuuuucauuauugcuccugaccuccucucauuugcuauauuca

SEQ ID NO: 169 - RNA, 62 nt, Homo sapiens
ucauugguccagaggggagauagguuccugugauuuuuccuucuucucuauagaauaaauga

SEQ ID NO: 170 - RNA, 83 nt, Homo sapiens
guggucucagaaucgggguuuugagggcgagaugaguuuauguuuuauccaacuggcccucaaagucccgcuuuuggggucau

SEQ ID NO: 171 - RNA, 68 nt, Homo sapiens
guguauucuacagugcacgugucuccaguguggcucggaggcuggagacgcggcccuguuggaguaac

SEQ ID NO: 172 - RNA, 85 nt, Homo sapiens; identical to SEQ ID NO: 164

SEQ ID NO: 173 - RNA, 90 nt, Homo sapiens
ccgggccccugugagcaucuuaccggacagugcuggauuucccagcuugacucuaacacugucugguaacgauguucaaaggugacccgc

SEQ ID NO: 174 - RNA, 89 nt, Homo sapiens
gccggcgcccgagcucuggcuccgugucuucacucccgugcuuguccgaggagggagggagggacgggggcugugcuggggcagcugga

SEQ ID NO: 175 - RNA, 95 nt, Homo sapiens
ccagcucgggcagccguggccaucuuacugggcagcauuggauggagucaggucucuaauacugccugguaaugaugacggcggagcccugcacg

SEQ ID NO: 176 - RNA, 68 nt, Homo sapiens
cccucgucuuacccagcaguguuugggugcgguugggagucucuaauacugccggguaaugauggagg

SEQ ID NO: 177 - RNA, 64 nt, Homo sapiens
ccccgcgacgagccccucgcacaaaccggaccugagcguuuuguucguucggcucgcgugaggc

SEQ ID NO: 178 - RNA, 90 nt, Homo sapiens; identical to SEQ ID NO: 173

SEQ ID NO: 179 - RNA, 83 nt, Homo sapiens
cgccggccgaugggcgucuuaccagacaugguuagaccuggcccucugucuaauacugucugguaaaaccguccauccgcugc

SEQ ID NO: 180 - RNA, 95 nt, Homo sapiens
cggccggcccuggguccaucuuccaguacaguguuggauggucuaauugugaagcuccuaacacugucugguaaagauggcucccggguggguuc

SEQ ID NO: 181 - RNA, 119 nt, Homo sapiens
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 182 - RNA, 73 nt, Homo sapiens
cuccucagaucagaaggugauuguggcuuuggguggauauuaaucagccacagcacugccuggucagaaagag

SEQ ID NO: 183 - RNA, 86 nt, Homo sapiens
ugcuucccgaggccacaugcuucuuuauauccccauauggauuacuuugcuauggaauguaaggaagugugugguuucggcaagug

SEQ ID NO: 184 - RNA, 71 nt, Homo sapiens
ugggaaacauacuucuuuauaugcccauauggaccugcuaagcuauggaauguaaagaaguauguaucuca

SEQ ID NO: 185 - RNA, 85 nt, Homo sapiens
accuacucagaguacauacuucuuuauguacccauaugaacauacaaugcuauggaauguaaagaaguauguauuuuugguaggc

SEQ ID NO: 186 - RNA, 88 nt, Homo sapiens
acaaugcuuugcuagagcugguaaaauggaaccaaaucgccucuucaauggauuugguccccuucaaccagcuguagcuaugcauuga

SEQ ID NO: 187 - RNA, 102 nt, Homo sapiens
gggagccaaaugcuuugcuagagcugguaaaauggaaccaaaucgacuguccaauggauuugguccccuucaaccagcuguagcugugcauugauggcgccg

SEQ ID NO: 188 - RNA, 110 nt, Homo sapiens
guguuggggacucgcgcgcuggguccagugguucuuaacaguucaacaguucuguagcgcaauugugaaauguuuaggaccacuagacccggcgggcgcggcgacagcga

SEQ ID NO: 189 - RNA, 110 nt, Homo sapiens
aaagauccucagacaauccaugugcuucucuuguccuucauuccaccggagucugucucauacccaaccagauuucaguggagugaaguucaggaggcauggagcugaca

SEQ ID NO: 190 - RNA, 21 nt, Homo sapiens
auugacacuucugugaguaga

SEQ ID NO: 191 - RNA, 21 nt, Homo sapiens
auugacacuucugugaguaga

SEQ ID NO: 192 - RNA, 21 nt, Homo sapiens
auugacacuucugugaguaga

SEQ ID NO: 193 - RNA, 22 nt, Homo sapiens
uucucaagagggaggcaaucau

SEQ ID NO: 194 - RNA, 23 nt, Homo sapiens
uacuccagagggcgucacucaug

SEQ ID NO: 195 - RNA, 21 nt, Homo sapiens
uaaggcacccuucugaguaga

SEQ ID NO: 196 - RNA, 21 nt, Homo sapiens
uacugcagacaguggcaauca

SEQ ID NO: 197 - RNA, 21 nt, Homo sapiens
uacugcagacaguggcaauca

SEQ ID NO: 198 - RNA, 22 nt, Homo sapiens
uacugcagacguggcaaucaug

SEQ ID NO: 199 - RNA, 22 nt, Homo sapiens
uacucaggagaguggcaaucac

SEQ ID NO: 200 - RNA, 94 nt, Homo sapiens
gugcuguguguagugcuucacuucaagaagugccaugcaugugucuagaaauauguuuugcaccuuuuggagugaaauaaugcacaacagauac

SEQ ID NO: 201 - RNA, 22 nt, Homo sapiens
ugagaacugaauuccauggguu

SEQ ID NO: 202 - RNA, 23 nt, Homo sapiens
ucggggaucaucaugucacgaga

SEQ ID NO: 203 - RNA, 22 nt, Homo sapiens
uucccuuugucauccuucgccu

SEQ ID NO: 204 - RNA, 18 nt, Homo sapiens
uucacagggaggugucau

SEQ ID NO: 205 - RNA, 18 nt, Homo sapiens
uucacagggaggugucau

SEQ ID NO: 206 - RNA, 22 nt, Homo sapiens
uucacaaggaggugucauuuau

SEQ ID NO: 207 - RNA, 22 nt, Homo sapiens
uucucaaggaggugucguuuau

SEQ ID NO: 208 - RNA, 21 nt, Homo sapiens
aggcaagaugcuggcauagcu

SEQ ID NO: 209 - RNA, 22 nt, Homo sapiens
aauugcacgguauccaucugua

SEQ ID NO: 210 - RNA, 23 nt, Homo sapiens
ucuuugguuaucuagcuguauga

SEQ ID NO: 211 - RNA, 23 nt, Homo sapiens
ucuuugguuaucuagcuguauga

SEQ ID NO: 212 - RNA, 23 nt, Homo sapiens
ucuuugguuaucuagcuguauga

SEQ ID NO: 213 - RNA, 22 nt, Homo sapiens
caaucacuaacuccacugccau

SEQ ID NO: 214 - RNA, 22 nt, Homo sapiens
cagcagcaauucauguuuugaa

SEQ ID NO: 215 - RNA, 22 nt, Homo sapiens
uagcuuaucagacugauguuga

SEQ ID NO: 216 - RNA, 23 nt, Homo sapiens
uuaaugcuaaucgugauaggggu

SEQ ID NO: 217 - RNA, 23 nt, Homo sapiens
uauggcuuuucauuccuauguga

SEQ ID NO: 218 - RNA, 23 nt, Homo sapiens
uggaagacuagugauuuuguugu

SEQ ID NO: 219 - RNA, 23 nt, Homo sapiens
uggaagacuagugauuuuguugu

SEQ ID NO: 220 - RNA, 23 nt, Homo sapiens
uggaagacuagugauuuuguugu

SEQ ID NO: 221 - RNA, 22 nt, Homo sapiens
uggcaguguauuguuagcuggu

SEQ ID NO: 222 - RNA, 22 nt, Homo sapiens
aggcaguguauuguuagcuggc

SEQ ID NO: 223 - RNA, 25 nt, Homo sapiens
uaggcaguguauugcuagcggcugu

SEQ ID NO: 224 - RNA, 22 nt, Homo sapiens
uggcagugucuuagcugguugu

SEQ ID NO: 225 - RNA, 23 nt, Homo sapiens
uagcagcgggaacaguucugcag

SEQ ID NO: 226 - RNA, 23 nt, Homo sapiens
aggcaguguaguuagcugauugc

SEQ ID NO: 227 - RNA, 23 nt, Homo sapiens
caaagugcucauagugcagguag

SEQ ID NO: 228 - RNA, 22 nt, Homo sapiens
aggcaguguauuguuagcuggc

SEQ ID NO: 229 - RNA, 22 nt, Homo sapiens
uggacggagaacugauaagggu

SEQ ID NO: 230 - RNA, 23 nt, Homo sapiens
cagugcaauaguauugucaaagc

SEQ ID NO: 231 - RNA, 23 nt, Homo sapiens
cagugcaaugauauugucaaagc

SEQ ID NO: 232 - RNA, 22 nt, Homo sapiens
ugaaggucuacugugugccagg

SEQ ID NO: 233 - RNA, 23 nt, Homo sapiens
ucaaaugcucagacuccuguggu

SEQ ID NO: 234 - RNA, 23 nt, Homo sapiens
ucaaaugcucagacuccuguggu

SEQ ID NO: 235 - RNA, 23 nt, Homo sapiens
aacauucauuguugucggugggu

SEQ ID NO: 236 - RNA, 23 nt, Homo sapiens
uaaggugcaucuagugcagauag

SEQ ID NO: 237 - RNA, 23 nt, Homo sapiens
ucuuugguuaucuagcuguauga

SEQ ID NO: 238 - RNA, 23 nt, Homo sapiens
ucuuugguuaucuagcuguauga

SEQ ID NO: 239 - RNA, 23 nt, Homo sapiens
ucuuugguuaucuagcuguauga

SEQ ID NO: 240 - RNA, 23 nt, Homo sapiens
uaaggugcaucuagugcaguuag

SEQ ID NO: 241 - RNA, 22 nt, Homo sapiens
ucucugggccugugucuuaggc

SEQ ID NO: 242 - RNA, 23 nt, Homo sapiens
aacauucauugcugucggugggu

SEQ ID NO: 243 - RNA, 23 nt, Homo sapiens
aacauucauugcugucggugggu

SEQ ID NO: 244 - RNA, 21 nt, Homo sapiens
gugcauuguaguugcauugca

SEQ ID NO: 245 - RNA, 20 nt, Homo sapiens
gugcauugcuguugcauugc

SEQ ID NO: 246 - RNA, 23 nt, Homo sapiens
aacauucaacgcugucggugagu

SEQ ID NO: 247 - RNA, 22 nt, Homo sapiens
cucuagagggaagcgcuuucug

SEQ ID NO: 248 - RNA, 22 nt, Homo sapiens
ugagguaguaguuuguacaguu

SEQ ID NO: 249 - RNA, 22 nt, Homo sapiens
agaucgaccguguuauauucgc

SEQ ID NO: 250 - RNA, 22 nt, Homo sapiens
ugguuuaccgucccacauacau

SEQ ID NO: 251 - RNA, 22 nt, Homo sapiens
uggucuaggauuguuggaggag

SEQ ID NO: 252 - RNA, 22 nt, Homo sapiens
caaagcgcuccccuuuagaggu

SEQ ID NO: 253 - RNA, 23 nt, Homo sapiens
agguuacccgagcaacuuugcau

SEQ ID NO: 254 - RNA, 22 nt, Homo sapiens
ugaguauuacauggccaaucuc

SEQ ID NO: 255 - RNA, 21 nt, Homo sapiens
cugaccuaugaauugacagcc

SEQ ID NO: 256 - RNA, 22 nt, Homo sapiens
aagcugccaguugaagaacugu

SEQ ID NO: 257 - RNA, 22 nt, Homo sapiens
ugagguaguagauuguauaguu

SEQ ID NO: 258 - RNA, 22 nt, Homo sapiens
ugagguaguagauuguauaguu

SEQ ID NO: 259 - RNA, 22 nt, Homo sapiens
uguaacagcaacuccaugugga

SEQ ID NO: 260 - RNA, 22 nt, Homo sapiens
uguaacagcaacuccaugugga

SEQ ID NO: 261 - RNA, 20 nt, Homo sapiens
guccgcucggcgguggccca

SEQ ID NO: 262 - RNA, 22 nt, Homo sapiens
cuagguauggucccagggaucc

SEQ ID NO: 263 - RNA, 22 nt, Homo sapiens
gugacaucacauauacggcagc

SEQ ID NO: 264 - RNA, 23 nt, Homo sapiens
ucuuggaguaggucauugggugg

SEQ ID NO: 265 - RNA, 21 nt, Homo sapiens
uggguuuacguugggagaacu

SEQ ID NO: 266 - RNA, 20 nt, Homo sapiens
agagguauagggcaugggaa

SEQ ID NO: 267 - RNA, 22 nt, Homo sapiens
ucagugcacuacagaacuuugu

SEQ ID NO: 268 - RNA, 22 nt, Homo sapiens
gugagucucuaagaaaagagga

SEQ ID NO: 269 - RNA, 21 nt, Homo sapiens
gugucuuuugcucugcaguca

SEQ ID NO: 270 - RNA, 21 nt, Homo sapiens
gugucuuuugcucugcaguca

SEQ ID NO: 271 - RNA, 22 nt, Homo sapiens
uggcucaguucagcaggaacag

SEQ ID NO: 272 - RNA, 22 nt, Homo sapiens
uggcucaguucagcaggaacag

SEQ ID NO: 273 - RNA, 22 nt, Homo sapiens
ugagguaguagguuguauaguu

SEQ ID NO: 274 - RNA, 22 nt, Homo sapiens
ugagguaguagguuguauaguu

SEQ ID NO: 275 - RNA, 22 nt, Homo sapiens
ugagguaguagguuguauaguu

SEQ ID NO: 276 - RNA, 23 nt, Homo sapiens
uacccuguagauccgaauuugug

SEQ ID NO: 277 - RNA, 22 nt, Homo sapiens
cugugcgugugacagcggcuga

SEQ ID NO: 278 - RNA, 21 nt, Homo sapiens
uucacaguggcuaaguuccgc

SEQ ID NO: 279 - RNA, 22 nt, Homo sapiens
uagguaguuuccuguuguuggg

SEQ ID NO: 280 - RNA, 21 nt, Homo sapiens
uucaaguaauucaggauaggu

SEQ ID NO: 281 - RNA, 22 nt, Homo sapiens
uguaaacauccuugacuggaag

SEQ ID NO: 282 - RNA, 21 nt, Homo sapiens
acuggacuuggagucagaagg

SEQ ID NO: 283 - RNA, 22 nt, Homo sapiens
ggggguccccggugcucggauc

SEQ ID NO: 284 - RNA, 21 nt, Homo sapiens
ucagugcaugacagaacuugg

SEQ ID NO: 285 - RNA, 21 nt, Homo sapiens
aucacauugccagggauuucc

SEQ ID NO: 286 - RNA, 22 nt, Homo sapiens
ugagguaggagguuguauaguu

SEQ ID NO: 287 - RNA, 22 nt, Homo sapiens
ugauauguuugauauauuaggu

SEQ ID NO: 288 - RNA, 22 nt, Homo sapiens
agaucgaccguguuauauucgc

SEQ ID NO: 289 - RNA, 22 nt, Homo sapiens
uguaaacauccuugacuggaag

SEQ ID NO: 290 - RNA, 22 nt, Homo sapiens
agacccuggucugcacucuauc

SEQ ID NO: 291 - RNA, 22 nt, Homo sapiens
uagcaccauuugaaaucgguua

SEQ ID NO: 292 - RNA, 22 nt, Homo sapiens
uucaaguaauccaggauaggcu

SEQ ID NO: 293 - RNA, 22 nt, Homo sapiens
uucaaguaauccaggauaggcu

SEQ ID NO: 294 - RNA, 21 nt, Homo sapiens
uacaguacugugauaacugaa

SEQ ID NO: 295 - RNA, 21 nt, Homo sapiens
uacaguacugugauaacugaa

SEQ ID NO: 296 - RNA, 22 nt, Homo sapiens
gaaguuguucgugguggauucg

SEQ ID NO: 297 - RNA, 22 nt, Homo sapiens
cuggcccucucugcccuuccgu

SEQ ID NO: 298 - RNA, 22 nt, Homo sapiens
uucccuuugucauccuaugccu

SEQ ID NO: 299 - RNA, 22 nt, Homo sapiens
cugaagcucagagggcucugau

SEQ ID NO: 300 - RNA, 22 nt, Homo sapiens
uguaaacauccucgacuggaag

SEQ ID NO: 301 - RNA, 21 nt, Homo sapiens
gcgacccauacuugguuucag

SEQ ID NO: 302 - RNA, 22 nt, Homo sapiens
uggcucaguucagcaggaacag

SEQ ID NO: 303 - RNA, 22 nt, Homo sapiens
ugggucuuugcgggcgagauga

SEQ ID NO: 304 - RNA, 21 nt, Homo sapiens
acuggacuuggagucagaagg

SEQ ID NO: 305 - RNA, 23 nt, Homo sapiens
uacccuguagaaccgaauuugug

SEQ ID NO: 306 - RNA, 22 nt, Homo sapiens
agaggcuggccgugaugaauuc

SEQ ID NO: 307 - RNA, 21 nt, Homo sapiens
aauauaacacagauggccugu

SEQ ID NO: 308 - RNA, 22 nt, Homo sapiens
uagguaguuucauguuguuggg

SEQ ID NO: 309 - RNA, 22 nt, Homo sapiens
uagguaguuucauguuguuggg

SEQ ID NO: 310 - RNA, 22 nt, Homo sapiens
ugagguaguagguugugugguu

SEQ ID NO: 311 - RNA, 22 nt, Homo sapiens
aaucguacagggucauccacuu

SEQ ID NO: 312 - RNA, 22 nt, Homo sapiens
uaaugccccuaaaaauccuuau

SEQ ID NO: 313 - RNA, 22 nt, Homo sapiens
uaaugccccuaaaaauccuuau

SEQ ID NO: 314 - RNA, 22 nt, Homo sapiens
ucguaccgugaguaauaaugcg

SEQ ID NO: 315 - RNA, 22 nt, Homo sapiens
ugugacugguugaccagagggg

SEQ ID NO: 316 - RNA, 22 nt, Homo sapiens
ucguaccgugaguaauaaugcg

SEQ ID NO: 317 - RNA, 22 nt, Homo sapiens
agaggcuggccgugaugaauuc

SEQ ID NO: 318 - RNA, 21 nt, Homo sapiens
uucacaguggcuaaguucugc

SEQ ID NO: 319 - RNA, 22 nt, Homo sapiens
aucaugaugggcuccucggugu

SEQ ID NO: 320 - RNA, 21 nt, Homo sapiens
uaguagaccguauagcguacg

SEQ ID NO: 321 - RNA, 21 nt, Homo sapiens
aucauagaggaaaauccacgu

SEQ ID NO: 322 - RNA, 21 nt, Homo sapiens
aucauagaggaaaauccacgu

SEQ ID NO: 323 - RNA, 23 nt, Homo sapiens
cccaguguucagacuaccuguuc

SEQ ID NO: 324 - RNA, 23 nt, Homo sapiens
cccaguguucagacuaccuguuc

SEQ ID NO: 325 - RNA, 22 nt, Homo sapiens
ucccugagacccuaacuuguga

SEQ ID NO: 326 - RNA, 22 nt, Homo sapiens
ucccugagacccuaacuuguga

SEQ ID NO: 327 - RNA, 21 nt, Homo sapiens
ugguagacuauggaacguagg

SEQ ID NO: 328 - RNA, 22 nt, Homo sapiens
aacccguagauccgaacuugug

SEQ ID NO: 329 - RNA, 21 nt, Homo sapiens
augaccuaugaauugacagac

SEQ ID NO: 330 - RNA, 21 nt, Homo sapiens
uugugcuugaucuaaccaugu

SEQ ID NO: 331 - RNA, 21 nt, Homo sapiens
uugugcuugaucuaaccaugu

SEQ ID NO: 332 - RNA, 22 nt, Homo sapiens
gucccucuccaaaugugucuug

SEQ ID NO: 333 - RNA, 22 nt, Homo sapiens
acuggacuuagggucagaaggc

SEQ ID NO: 334 - RNA, 21 nt, Homo sapiens
aucauagaggaaaauccacgu

SEQ ID NO: 335 - RNA, 21 nt, Homo sapiens
aucauagaggaaaauccacgu

SEQ ID NO: 336 - RNA, 22 nt, Homo sapiens
acagcaggcacagacaggcagu

SEQ ID NO: 337 - RNA, 21 nt, Homo sapiens
caagucacuagugguuccguu

SEQ ID NO: 338 - RNA, 21 nt, Homo sapiens
uagcagcacagaaauauuggc

SEQ ID NO: 339 - RNA, 23 nt, Homo sapiens
uuuggcacuagcacauuuuugcu

SEQ ID NO: 340 - RNA, 22 nt, Homo sapiens
uucaacggguauuuauugagca

SEQ ID NO: 341 - RNA, 21 nt, Homo sapiens
aucacauugccagggauuacc

SEQ ID NO: 342 - RNA, 21 nt, Homo sapiens
aauauuauacagucaaccucu

SEQ ID NO: 343 - RNA, 22 nt, Homo sapiens
aaaccguuaccauuacugaguu

SEQ ID NO: 344 - RNA, 24 nt, Homo sapiens
uuuggcaaugguagaacucacacu

SEQ ID NO: 345 - RNA, 23 nt, Homo sapiens
cccaguguuuagacuaucuguuc

SEQ ID NO: 346 - RNA, 23 nt, Homo sapiens
cccaguguucagacuaccuguuc

SEQ ID NO: 347 - RNA, 23 nt, Homo sapiens
cccaguguucagacuaccuguuc

SEQ ID NO: 348 - RNA, 22 nt, Homo sapiens
aacccguagauccgaucuugug

SEQ ID NO: 349 - RNA, 21 nt, Homo sapiens
cagcagcacacugugguuugu

SEQ ID NO: 350 - RNA, 22 nt, Homo sapiens
uccuguacugagcugccccgag

SEQ ID NO: 351 - RNA, 22 nt, Homo sapiens
ugguuuaccgucccacauacau

SEQ ID NO: 352 - RNA, 23 nt, Homo sapiens
guccaguuuucccaggaaucccu

SEQ ID NO: 353 - RNA, 22 nt, Homo sapiens
aacuguuugcagaggaaacuga

SEQ ID NO: 354 - RNA, 22 nt, Homo sapiens
uauggcacugguagaauucacu

SEQ ID NO: 355 - RNA, 22 nt, Homo sapiens
ugagguaguagguuguaugguu

SEQ ID NO: 356 - RNA, 21 nt, Homo sapiens
ugagaugaagcacuguagcuc

SEQ ID NO: 357 - RNA, 23 nt, Homo sapiens
ucaagagcaauaacgaaaaaugu

SEQ ID NO: 358 - RNA, 22 nt, Homo sapiens
gguccagaggggagauagguuc

SEQ ID NO: 359 - RNA, 22 nt, Homo sapiens
aacuggcccucaaagucccgcu

SEQ ID NO: 360 - RNA, 22 nt, Homo sapiens
ucuacagugcacgugucuccag

SEQ ID NO: 361 - RNA, 22 nt, Homo sapiens
aacuguuugcagaggaaacuga

SEQ ID NO: 362 - RNA, 22 nt, Homo sapiens
uaacacugucugguaacgaugu

SEQ ID NO: 363 - RNA, 23 nt, Homo sapiens
ucuggcuccgugucuucacuccc

SEQ ID NO: 364 - RNA, 22 nt, Homo sapiens
uaauacugccugguaaugauga

SEQ ID NO: 365 - RNA, 23 nt, Homo sapiens
uaauacugccggguaaugaugga

SEQ ID NO: 366 - RNA, 22 nt, Homo sapiens
uuuguucguucggcucgcguga

SEQ ID NO: 367 - RNA, 22 nt, Homo sapiens
uaacacugucugguaacgaugu

SEQ ID NO: 368 - RNA, 22 nt, Homo sapiens
uaauacugucugguaaaaccgu

SEQ ID NO: 369 - RNA, 22 nt, Homo sapiens
uaacacugucugguaaagaugg

SEQ ID NO: 370 - RNA, 22 nt, Homo sapiens
uuugguccccuucaaccagcua

SEQ ID NO: 371 - RNA, 22 nt, Homo sapiens
agaucagaaggugauuguggcu

SEQ ID NO: 372 - RNA, 22 nt, Homo sapiens
uggaauguaaggaagugugugg

SEQ ID NO: 373 - RNA, 22 nt, Homo sapiens
uggaauguaaagaaguauguau

SEQ ID NO: 374 - RNA, 22 nt, Homo sapiens
uggaauguaaagaaguauguau

SEQ ID NO: 375 - RNA, 22 nt, Homo sapiens
uuugguccccuucaaccagcug

SEQ ID NO: 376 - RNA, 22 nt, Homo sapiens
uuugguccccuucaaccagcug

SEQ ID NO: 377 - RNA, 22 nt, Homo sapiens
gugaaauguuuaggaccacuag

SEQ ID NO: 378 - RNA, 22 nt, Homo sapiens
uccuucauuccaccggagucug

SEQ ID NO: 379 - RNA, 21 nt, Homo sapiens
auugacaccucugugagugga

SEQ ID NO: 380 - RNA, 23 nt, Homo sapiens
ugauuguagccuuuuggaguaga

SEQ ID NO: 381 - RNA, 22 nt, Homo sapiens
ugauugguacgucuguggguag

SEQ ID NO: 382 - RNA, 22 nt, Homo sapiens
ugauugguacgucuguggguag

SEQ ID NO: 383 - RNA, 22 nt, Homo sapiens
ugauugguacgucuguggguag

SEQ ID NO: 384 - RNA, 22 nt, Homo sapiens
ccucugaaauucaguucuucag

SEQ ID NO: 385 - RNA, 22 nt, Homo sapiens
ugugacagauugauaacugaaa

SEQ ID NO: 386 - RNA, 23 nt, Homo sapiens
uaaauuucaccuuucugagaagg

SEQ ID NO: 387 - RNA, 23 nt, Homo sapiens
uaaauuucaccuuucugagaagg

SEQ ID NO: 388 - RNA, 22 nt, Homo sapiens
ugcuaugccaacauauugccau

SEQ ID NO: 389 - RNA, 22 nt, Homo sapiens
cggguggaucacgaugcaauuu

SEQ ID NO: 390 - RNA, 22 nt, Homo sapiens
auaaagcuagauaaccgaaagu

SEQ ID NO: 391 - RNA, 22 nt, Homo sapiens
auaaagcuagauaaccgaaagu

SEQ ID NO: 392 - RNA, 22 nt, Homo sapiens
auaaagcuagauaaccgaaagu

SEQ ID NO: 393 - RNA, 23 nt, Homo sapiens
uaggcagugucauuagcugauug

SEQ ID NO: 394 - RNA, 21 nt, Homo sapiens
caaaacgugaggcgcugcuau

SEQ ID NO: 395 - RNA, 21 nt, Homo sapiens
caacaccagucgaugggcugu

SEQ ID NO: 396 - RNA, 22 nt, Homo sapiens
cuccuacauauuagcauuaaca

SEQ ID NO: 397 - RNA, 22 nt, Homo sapiens
auguagggcuaaaagccauggg

SEQ ID NO: 398 - RNA, 22 nt, Homo sapiens
caacaaaucacagucugccaua

SEQ ID NO: 399 - RNA, 22 nt, Homo sapiens
caacaaaucccagucuaccuaa

SEQ ID NO: 400 - RNA, 22 nt, Homo sapiens
cagccacaacuacccugccacu

SEQ ID NO: 401 - RNA, 23 nt, Homo sapiens
uugcuaguugcacuccucucugu

SEQ ID NO: 402 - RNA, 22 nt, Homo sapiens
caaucagcaaguauacugcccu

SEQ ID NO: 403 - RNA, 22 nt, Homo sapiens
aaucacuaaccacacggccagg

SEQ ID NO: 404 - RNA, 22 nt, Homo sapiens
acuguaguaugggcacuuccag

SEQ ID NO: 405 - RNA, 22 nt, Homo sapiens
cagccacaacuacccugccacu

SEQ ID NO: 406 - RNA, 22 nt, Homo sapiens
uuguacaugguaggcuuucauu

SEQ ID NO: 407 - RNA, 22 nt, Homo sapiens
acggauguuugagcaugugcua

SEQ ID NO: 408 - RNA, 22 nt, Homo sapiens
acggauguuugagcaugugcua

SEQ ID NO: 409 - RNA, 23 nt, Homo sapiens
acugcccuaagugcuccuucugg

SEQ ID NO: 410 - RNA, 22 nt, Homo sapiens
auaaagcuagauaaccgaaagu

SEQ ID NO: 411 - RNA, 22 nt, Homo sapiens
auaaagcuagauaaccgaaagu

SEQ ID NO: 412 - RNA, 22 nt, Homo sapiens
auaaagcuagauaaccgaaagu

SEQ ID NO: 413 - RNA, 22 nt, Homo sapiens
ugcccuaaaugccccuucuggc

SEQ ID NO: 414 - RNA, 23 nt, Homo sapiens
gcaaagcacacggccugcagaga

SEQ ID NO: 415 - RNA, 22 nt, Homo sapiens
caauguuuccacagugcaucac

SEQ ID NO: 416 - RNA, 22 nt, Homo sapiens
cagugccucggcagugcagccc

SEQ ID NO: 417 - RNA, 22 nt, Homo sapiens
accaucgaccguugauuguacc

SEQ ID NO: 418 - RNA, 22 nt, Homo sapiens
aaagugcaucuuuuuagaggau

SEQ ID NO: 419 - RNA, 21 nt, Homo sapiens
cuguacaggccacugccuugc

SEQ ID NO: 420 - RNA, 21 nt, Homo sapiens
aauaauacaugguugaucuuu

SEQ ID NO: 421 - RNA, 22 nt, Homo sapiens
uaugugggaugguaaaccgcuu

SEQ ID NO: 422 - RNA, 22 nt, Homo sapiens
gaauguugcucggugaaccccu

SEQ ID NO: 423 - RNA, 22 nt, Homo sapiens
cugccaauuccauaggucacag

SEQ ID NO: 424 - RNA, 22 nt, Homo sapiens
aguucuucaguggcaagcuuua

SEQ ID NO: 425 - RNA, 22 nt, Homo sapiens
cuauacaaucuauugccuuccc

SEQ ID NO: 426 - RNA, 22 nt, Homo sapiens
cuauacagucuacugucuuucc

SEQ ID NO: 427 - RNA, 22 nt, Homo sapiens
ccaguggggcugcuguuaucug

SEQ ID NO: 428 - RNA, 21 nt, Homo sapiens
gccccugggccuauccuagaa

SEQ ID NO: 429 - RNA, 21 nt, Homo sapiens
cuggauggcuccuccaugucu

SEQ ID NO: 430 - RNA, 22 nt, Homo sapiens
guucucccaacguaagcccagc

SEQ ID NO: 431 - RNA, 22 nt, Homo sapiens
uuccuaugcauauacuucuuug

SEQ ID NO: 432 - RNA, 22 nt, Homo sapiens
aaaguucugagacacuccgacu

SEQ ID NO: 433 - RNA, 22 nt, Homo sapiens
ugccuacugagcugauaucagu

SEQ ID NO: 434 - RNA, 22 nt, Homo sapiens
ugccuacugagcugaaacacag

SEQ ID NO: 435 - RNA, 21 nt, Homo sapiens
cuauacaaucuacugucuuuc

SEQ ID NO: 436 - RNA, 22 nt, Homo sapiens
cuguacagccuccuagcuuucc

SEQ ID NO: 437 - RNA, 21 nt, Homo sapiens
cuauacaaucuacugucuuuc

SEQ ID NO: 438 - RNA, 22 nt, Homo sapiens
caaauucguaucuaggggaaua

SEQ ID NO: 439 - RNA, 22 nt, Homo sapiens
agggcuuagcugcuugugagca

SEQ ID NO: 440 - RNA, 22 nt, Homo sapiens
ucgacagcacgacacugccuuc

SEQ ID NO: 441 - RNA, 22 nt, Homo sapiens
ccuguucuccauuacuuggcuc

SEQ ID NO: 442 - RNA, 22 nt, Homo sapiens
cuuucagucggauguuuacagc

SEQ ID NO: 443 - RNA, 22 nt, Homo sapiens
cuccugacuccagguccugugu

SEQ ID NO: 444 - RNA, 22 nt, Homo sapiens
uccgagccugggucucccucuu

SEQ ID NO: 445 - RNA, 22 nt, Homo sapiens
gggguuccuggggaugggauuu

SEQ ID NO: 446 - RNA, 22 nt, Homo sapiens
cuauacggccuccuagcuuucc

SEQ ID NO: 447 - RNA, 21 nt, Homo sapiens
aauaauacaugguugaucuuu

SEQ ID NO: 448 - RNA, 22 nt, Homo sapiens
cuuucagucggauguuuacagc

SEQ ID NO: 449 - RNA, 22 nt, Homo sapiens
ugaccgauuucuccugguguuc

SEQ ID NO: 450 - RNA, 22 nt, Homo sapiens
ccuauucuugguuacuugcacg

SEQ ID NO: 451 - RNA, 22 nt, Homo sapiens
ccuauucuugauuacuuguuuc

SEQ ID NO: 452 - RNA, 22 nt, Homo sapiens
caguuaucacagugcugaugcu

SEQ ID NO: 453 - RNA, 22 nt, Homo sapiens
ucggauccgucugagcuuggcu

SEQ ID NO: 454 - RNA, 22 nt, Homo sapiens
cuuucagucggauguuugcagc

SEQ ID NO: 455 - RNA, 22 nt, Homo sapiens
gaaaucaagcgugggugagacc

SEQ ID NO: 456 - RNA, 22 nt, Homo sapiens
ugccuacugagcugauaucagu

SEQ ID NO: 457 - RNA, 22 nt, Homo sapiens
aacuggccuacaaagucccagu

SEQ ID NO: 458 - RNA, 22 nt, Homo sapiens
cuccugacuccagguccugugu

SEQ ID NO: 459 - RNA, 22 nt, Homo sapiens
acagauucgauucuaggggaau

SEQ ID NO: 460 - RNA, 22 nt, Homo sapiens
gucauacacggcucuccucucu

SEQ ID NO: 461 - RNA, 22 nt, Homo sapiens
cggcaacaagaaacugccugag

SEQ ID NO: 462 - RNA, 22 nt, Homo sapiens
cuauacaaccuacugccuuccc

SEQ ID NO: 463 - RNA, 22 nt, Homo sapiens
agggacuuucaggggcagcugu

SEQ ID NO: 464 - RNA, 21 nt, Homo sapiens
cauuauuacuuuugguacgcg

SEQ ID NO: 465 - RNA, 21 nt, Homo sapiens
cauuauuacuuuugguacgcg

SEQ ID NO: 466 - RNA, 22 nt, Homo sapiens
gucauacacggcucuccucucu

SEQ ID NO: 467 - RNA, 22 nt, Homo sapiens
agagcuuagcugauuggugaac

SEQ ID NO: 468 - RNA, 22 nt, Homo sapiens
uauguaacacgguccacuaacc

SEQ ID NO: 469 - RNA, 22 nt, Homo sapiens
guagauucuccuucuaugagua

SEQ ID NO: 470 - RNA, 22 nt, Homo sapiens
acaguagucugcacauugguua

SEQ ID NO: 471 - RNA, 22 nt, Homo sapiens
acaguagucugcacauugguua

SEQ ID NO: 472 - RNA, 22 nt, Homo sapiens
acggguuaggcucuugggagcu

SEQ ID NO: 473 - RNA, 22 nt, Homo sapiens
ucacaagucaggcucuugggac

SEQ ID NO: 474 - RNA, 22 nt, Homo sapiens
uauguaacaugguccacuaacu

SEQ ID NO: 475 - RNA, 22 nt, Homo sapiens
caagcuuguaucuauagguaug

SEQ ID NO: 476 - RNA, 22 nt, Homo sapiens
augguuccgucaagcaccaugg

SEQ ID NO: 477 - RNA, 22 nt, Homo sapiens
caugguucugucaagcaccgcg

SEQ ID NO: 478 - RNA, 22 nt, Homo sapiens
guagauucuccuucuaugagua

SEQ ID NO: 479 - RNA, 22 nt, Homo sapiens
ugccugucuacacuugcugugc

SEQ ID NO: 480 - RNA, 23 nt, Homo sapiens
aaaauggugcccuagugacuaca

SEQ ID NO: 481 - RNA, 22 nt, Homo sapiens
ccaauauuggcugugcugcucc

SEQ ID NO: 482 - RNA, 22 nt, Homo sapiens
aaucaugugcagugccaauaug

SEQ ID NO: 483 - RNA, 22 nt, Homo sapiens
uggguuccuggcaugcugauuu

SEQ ID NO: 484 - RNA, 21 nt, Homo sapiens
ugguucuagacuugccaacua

SEQ ID NO: 485 - RNA, 22 nt, Homo sapiens
acaguagucugcacauugguua

SEQ ID NO: 486 - RNA, 22 nt, Homo sapiens
acaguagucugcacauugguua

SEQ ID NO: 487 - RNA, 22 nt, Homo sapiens
acaguagucugcacauugguua

SEQ ID NO: 488 - RNA, 22 nt, Homo sapiens
caagcucgcuucuaugggucug

SEQ ID NO: 489 - RNA, 22 nt, Homo sapiens
caaaccacacugugguguuaga

SEQ ID NO: 490 - RNA, 21 nt, Homo sapiens
cggggcagcucaguacaggau

SEQ ID NO: 491 - RNA, 22 nt, Homo sapiens
uaugugggaugguaaaccgcuu

SEQ ID NO: 492 - RNA, 22 nt, Homo sapiens
ggauuccuggaaauacuguucu

SEQ ID NO: 493 - RNA, 22 nt, Homo sapiens
cucaucugcaaagaaguaagug

SEQ ID NO: 494 - RNA, 22 nt, Homo sapiens
gugaauuaccgaagggccauaa

SEQ ID NO: 495 - RNA, 22 nt, Homo sapiens
ggugcagugcugcaucucuggu

SEQ ID NO: 496 - RNA, 22 nt, Homo sapiens
uuuuucauuauugcuccugacc

SEQ ID NO: 497 - RNA, 22 nt, Homo sapiens
cgggguuuugagggcgagauga

SEQ ID NO: 498 - RNA, 22 nt, Homo sapiens
ggagacgcggcccuguuggagu

SEQ ID NO: 499 - RNA, 22 nt, Homo sapiens
cucaucugcaaagaaguaagug

SEQ ID NO: 500 - RNA, 22 nt, Homo sapiens
caucuuaccggacagugcugga

SEQ ID NO: 501 - RNA, 21 nt, Homo sapiens
agggagggacgggggcugugc

SEQ ID NO: 502 - RNA, 22 nt, Homo sapiens
caucuuacugggcagcauugga

SEQ ID NO: 503 - RNA, 22 nt, Homo sapiens
cgucuuacccagcaguguuugg

SEQ ID NO: 504 - RNA, 22 nt, Homo sapiens
caucuuaccggacagugcugga

SEQ ID NO: 505 - RNA, 22 nt, Homo sapiens
caucuuccaguacaguguugga

SEQ ID NO: 506 - RNA, 21 nt, Homo sapiens
gauuucaguggagugaaguuc

SEQ ID NO: 507 - RNA, 23 nt, Homo sapiens
gauggucaucuuggagggaccaa

SEQ ID NO: 508 - RNA, 22 nt, Homo sapiens
uugguccccuccaaccagcugg

SEQ ID NO: 509 - RNA, 21 nt, Homo sapiens
uugguccccuucaaccagcua

(19) United States
(12) Patent Application Publication
(10) Pub. No.: US 2014/0045924 A1
(43) Pub. Date: Feb. 13, 2014

(54) MELANOMA TREATMENTS

(71) Applicants: Yihong Yao, Boyds, MD (US); Katie Streicher, Bethesda, MD (US); Robert William Gaithersburg, III, Gaithersburg, MD (US); Wei Zhu, Boyds, MD (US); Koustubh Ranade, Potomac, MD (US)

(72) Inventors: Yihong Yao, Boyds, MD (US); Katie Streicher, Bethesda, MD (US); Robert William Gaithersburg, III, Gaithersburg, MD (US); Wei Zhu, Boyds, MD (US); Koustubh Ranade, Potomac, MD (US)

(73) Assignee: MEDIMMUNE, LLC, Gaithersburg, MD (US)

(21) Appl. No.: 13/994,291

(22) PCT Filed: Dec. 14, 2011
(86) PCT No.: PCT/US11/64778

§ 371 Date: Oct. 25, 2013

Related U.S. Application Data
(60) Provisional application No. 61/423,305, filed on Dec. 15, 2010.
(60) Provisional application No. 61/521,024, filed on Aug. 8, 2011.

(51) Int. Cl. C12N 15/113

(52) U.S. Cl. CPC C12N 15/1135
USPC 514/44 R; 536/23.1

(57) ABSTRACT

Provided herein, in some embodiments, are methods for treating melanoma, improving therapeutic effectiveness of drugs and reducing drug toxicity.

### FIELD

The technology relates in part to melanoma and the use of microRNAs (miRNAs) in melanoma diagnosis and treatment.

### BACKGROUND

Melanoma is one of the most aggressive forms of cancer, typically beginning in the skin and often metastasizing to vital organs and other tissues. A specific type of RNA, referred to as microRNA, plays a role in melanoma, with certain microRNAs consistently under-expressed or over-expressed in melanoma cells.

### SUMMARY

Featured herein are personalized treatments of cancer (e.g., melanoma) that optimize the therapeutic effect of a drug and provided, in some embodiments, are methods comprising: (a) administering an anti-cancer drug to a subject having melanoma; (b) identifying or determining the presence, absence or amount of at least one biomarker in the subject, where the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing and (c) maintaining a subsequent dosage of the drug or adjusting a subsequent dosage of the drug administered to the subject based on the presence, absence or amount of the biomarker identified in the subject. The “microRNA506-514 cluster” consists of the following microRNAs: microRNA-506, microRNA-507, microRNA-508, microRNA-509, microRNA-510, microRNA-513 and microRNA-514. The “microRNA506-513 cluster” consists of the following microRNAs: microRNA-506, microRNA-507, microRNA-508 and microRNA-513.

In some embodiments, methods comprise: (a) administering an anti-cancer drug to a subject having melanoma, (b) identifying or determining the presence, absence or amount of a biomarker in the subject, where the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (c) determining whether the dosage of the drug subsequently administered to the subject is adjusted based on the presence, absence or amount of the biomarker identified in the subject. In certain embodiments, methods comprise: (a) identifying the presence, absence or amount of a biomarker in a subject having melanoma to whom an anti-cancer drug has been administered, where the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (c) maintaining a subsequent dosage of the drug or adjusting a subsequent dosage of the drug administered to the subject based on the presence, absence or amount of the biomarker identified in the subject.

Methods comprise, in some embodiments, (a) identifying the presence, absence or amount of a biomarker in a subject having melanoma to whom an anti-cancer drug has been administered, where the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (c) determining whether the dosage of the drug subsequently administered to the subject is adjusted based on the presence, absence or amount of the biomarker identified in the subject.

In various embodiments, methods comprise: (a) receiving information comprising the presence, absence or amount of a biomarker in a subject having melanoma to whom an anti-cancer drug has been administered, where the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (b) maintaining a subsequent dosage of the drug or adjusting a subsequent dosage of the drug administered to the subject based on the presence, absence or amount of the biomarker identified in the subject. In certain embodiments, methods comprise: (a) identifying the presence, absence or amount of a biomarker in a subject having melanoma to whom an anti-cancer drug has been administered, where the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (b) transmitting the presence, absence or amount of the biomarker to a decision maker who maintains a subsequent dosage of the drug or adjusts a subsequent dosage of the drug administered to the subject based on the presence, absence or amount of the biomarker identified in the subject. In some embodiments, methods comprise: (a) identifying the presence, absence or amount of a biomarker in a subject having melanoma to whom an anti-cancer drug has been administered, where the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing and (b) transmitting an indication to maintain a subsequent dosage of the drug or adjust a subsequent dosage of the drug administered to the subject based on the presence, absence or amount of the biomarker identified in the subject.

Provided also, in some embodiments, are methods for optimizing therapeutic efficacy of a treatment of melanoma in a subject, comprising (a) identifying the presence, absence or amount of a biomarker in a subject having melanoma to whom an anti-cancer drug has been administered, where the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (b) maintaining a subsequent dosage of the drug or adjusting a subsequent dosage of the drug administered to the subject based on the presence, absence or amount of the biomarker identified in the subject.

Also provided, in some embodiments, are methods for reducing toxicity of a treatment of melanoma in a subject, comprising (a) identifying the presence, absence or amount of a biomarker in a subject having melanoma to whom an anti-cancer drug has been administered, where the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (b) maintaining a subsequent dosage of the drug or adjusting a subsequent dosage of the drug administered to the subject based on the presence, absence or amount of the biomarker identified in the subject.

Provided also, in some embodiments, are methods for treating melanoma in a subject that comprise administering a composition that delivers to a subject in need thereof a microRNA composition in an amount effective to treat the melanoma in the subject, where the microRNA composition comprises a microRNA selected from the group consisting of microRNA selected from the group consisting of microRNA-10, microRNA-126, microRNA-193, microRNA-203, microRNA-206, and combinations of the foregoing. Also provided in certain embodiments are methods that comprise contacting melanoma cells with a microRNA composition in an amount effective to inhibit proliferation of the melanoma cells, where the microRNA composition comprises a microRNA selected from the group consisting of microRNA-126, microRNA-193, microRNA-206, and combinations of the foregoing. Provided also in some embodiments are methods that comprise contacting melanoma cells with a microRNA composition in an amount effective to induce apoptosis of the melanoma cells, where the microRNA composition comprises a microRNA selected from the group consisting of microRNA-10, microRNA-126, microRNA-193, microRNA-203, and combinations of the foregoing.

Also provided in certain embodiments, are methods for treating melanoma in a subject that comprise administering a composition that delivers to a subject in need thereof a microRNA inhibitor composition in an amount effective to treat the melanoma in the subject, where the microRNA inhibitor composition comprises an inhibitor of a microRNA selected from the group consisting of microRNA-21, microRNA-146, microRNA-155, microRNA-506, microRNA-507, microRNA-508, microRNA-509, microRNA-510, microRNA-513, microRNA-514, microRNA-506-514 cluster, microRNA-506-513 cluster, and combinations of the foregoing. Provided also in some embodiments are methods that comprise contacting melanoma cells with a microRNA inhibitor composition in an amount effective to inhibit proliferation of the melanoma cells, where the microRNA inhibitor composition comprises an inhibitor of a microRNA, which microRNA is selected from the group consisting of microRNA-21, microRNA-146, microRNA-155, microRNA-506, microRNA-507, microRNA-508, microRNA-509, microRNA-510, microRNA-513, microRNA-514, microRNA-506-514 cluster, microRNA-506-513 cluster, and combinations of the foregoing. Also provided in certain embodiments are methods that comprise contacting melanoma cells with a microRNA inhibitor composition in an amount effective to induce apoptosis of the melanoma cells, where the microRNA inhibitor composition comprises an inhibitor of a microRNA, which microRNA is selected from the group consisting of microRNA-21, microRNA-146, microRNA-506, microRNA-507, microRNA-508, microRNA-509, microRNA-510, microRNA-513, microRNA-514, microRNA-506-514 cluster, microRNA-506-513 cluster, and combinations of the foregoing.

In various embodiments, the presence, absence or amount of a microRNA selected from the group consisting of microRNA-21, microRNA-146 and microRNA-155 is determined. In certain embodiments, a composition comprising one or more microRNA inhibitors selected from the group consisting of microRNA-21, microRNA-146 and microRNA-155 is utilized. In some embodiments, the microRNA-193 is microRNA-193b. In some embodiments, the microRNA-10 is microRNA-10a. In certain embodiments, the microRNA-146 is microRNA-146a. In some embodiments, the melanoma cells are in a tumor.

Also provided, in certain embodiments, are methods comprising administering a composition to a subject in need thereof in an amount effective to treat melanoma in the subject, where the composition comprises one or more components that deliver to the subject (i) imidazole carboxamide, and (ii) a microRNA composition that increases sensitivity of melanoma cells to imidazole carboxamide anti-cell proliferation activity. In certain embodiments, methods comprise contacting melanoma cells with a composition in an amount effective to inhibit proliferation of the melanoma cells, where the composition comprises one or more components that deliver (i) imidazole carboxamide, and (ii) a microRNA composition that increases sensitivity of melanoma cells to imidazole carboxamide anti-cell proliferation activity. In various embodiments, a method comprises contacting melanoma cells with a composition in an amount effective to induce apoptosis of the melanoma cells, where the composition comprises one or more components that deliver (i) imidazole carboxamide, and (ii) a microRNA composition that increases sensitivity of melanoma cells to imidazole carboxamide anti-cell proliferation activity.

A method, in some embodiments, comprises administering a composition that delivers an imidazole carboxamide drug to a subject having melanoma, identifying the presence, absence or amount of a biomarker in the subject, where the biomarker comprises a microRNA that increases sensitivity of melanoma cells to imidazole carboxamide anti-cell proliferation activity and maintaining a subsequent dosage of the drug or adjusting a subsequent dosage of the drug administered to the subject based on the presence, absence or amount of the biomarker identified in the subject.

In certain embodiments, a method comprises administering a composition that delivers an imidazole carboxamide drug to a subject having melanoma, identifying the presence, absence or amount of a biomarker in the subject, where the biomarker comprises a microRNA that increases sensitivity of melanoma cells to imidazole carboxamide anti-cell proliferation activity, and determining whether the dosage of the drug subsequently administered to the subject is adjusted based on the presence, absence or amount of the biomarker identified in the subject. In some embodiments, a method comprises identifying the presence, absence or amount of a biomarker in a subject having melanoma to whom a composition that delivers an imidazole carboxamide drug has been administered, where the biomarker comprises a microRNA that increases sensitivity of melanoma cells to imidazole carboxamide anti-cell proliferation activity, and maintaining a subsequent dosage of the drug or adjusting a subsequent dosage of the drug administered to the subject based on the presence, absence or amount of the biomarker identified in the subject. A method may sometimes comprise identifying the presence, absence or amount of a biomarker in a subject having melanoma to whom a composition that delivers an imidazole carboxamide drug has been administered, where the biomarker comprises a microRNA that increases sensitivity of melanoma cells to imidazole carboxamide anti-cell proliferation activity and determining whether the dosage of the drug subsequently administered to the subject is adjusted based on the presence, absence or amount of the biomarker identified in the subject.

Also provided, in some embodiments, are methods that comprise receiving information comprising the presence, absence or amount of a biomarker in a subject having melanoma to whom a composition that delivers an imidazole carboxamide drug has been administered, where the biomarker comprises a microRNA that increases sensitivity of melanoma cells to imidazole carboxamide anti-cell proliferation activity, and maintaining a subsequent dosage of the drug or adjusting a subsequent dosage of the drug administered to the subject based on the presence, absence or amount of the biomarker identified in the subject. In some embodiments, a method comprises identifying the presence, absence or amount of a biomarker in a subject having melanoma to whom a composition that delivers an imidazole carboxamide drug has been administered, where the biomarker comprises a microRNA that increases sensitivity of melanoma cells to imidazole carboxamide anti-cell proliferation activity and transmitting the presence, absence or amount of the biomarker to a decision maker who maintains a subsequent dosage of the drug or adjusts a subsequent dosage of the drug administered to the subject based on the presence, absence or amount of the biomarker identified in the subject. A method sometimes comprises identifying the presence, absence or amount of a biomarker in a subject having melanoma to whom a composition that delivers an imidazole carboxamide drug has been administered, where the biomarker comprises a microRNA that increases sensitivity of melanoma cells to imidazole carboxamide anti-cell proliferation activity and transmitting an indication to maintain a subsequent dosage of the drug or adjust a subsequent dosage of the drug administered to the subject based on the presence, absence or amount of the biomarker identified in the subject.

Provided also, in some embodiments, are methods for optimizing therapeutic efficacy of a treatment of melanoma in a subject, comprising identifying the presence, absence or amount of a biomarker in a subject having melanoma to whom a composition that delivers an imidazole carboxamide drug has been administered, where the biomarker comprises a microRNA that increases sensitivity of melanoma cells to imidazole carboxamide anti-cell proliferation activity and maintaining a subsequent dosage of the drug or adjusting a subsequent dosage of the drug administered to the subject based on the presence, absence or amount of the biomarker identified in the subject.

Also provided, in some embodiments, are methods for reducing toxicity of a treatment of melanoma in a subject, comprising identifying the presence, absence or amount of a biomarker in a subject having melanoma to whom a composition that delivers an imidazole carboxamide drug has been administered, where the biomarker comprises a microRNA that increases sensitivity of melanoma cells to imidazole carboxamide anti-cell proliferation activity, and maintaining a subsequent dosage of the drug or adjusting a subsequent dosage of the drug administered to the subject based on the presence, absence or amount of the biomarker identified in the subject. A method sometimes comprises identifying the presence, absence or amount of a biomarker in a subject having melanoma, where the biomarker comprises a microRNA that increases sensitivity of melanoma cells to imidazole carboxamide anti-cell proliferation activity and determining whether a composition that delivers an imidazole carboxamide drug is administered, or not administered, to the subject based on the presence, absence or amount of the biomarker.

Provided also, in some embodiments, are methods comprising receiving information comprising the presence, absence or amount of a biomarker in a subject having melanoma, where the biomarker comprises a microRNA that increases sensitivity of melanoma cells to imidazole carboxamide anti-cell proliferation activity and determining whether a composition that delivers an imidazole carboxamide drug is administered, or not administered, to the subject based on the presence, absence or amount of the biomarker. In certain embodiments, a method comprises identifying the presence, absence or amount of a biomarker in a subject having melanoma, where the biomarker comprises a microRNA that increases sensitivity of melanoma cells to imidazole carboxamide anti-cell proliferation activity, and transmitting the presence, absence or amount of the biomarker to a decision maker who determines whether a composition that delivers an imidazole carboxamide drug is administered to the subject based on the presence, absence or amount of the biomarker.

Also provided, in certain embodiments, are methods comprising identifying the presence, absence or amount of a biomarker in a subject having melanoma, where the biomarker comprises a microRNA that increases sensitivity of melanoma cells to imidazole carboxamide anti-cell proliferation activity, and providing an indication for administering, or not administering, a composition that delivers an imidazole carboxamide drug to the subject based on the presence, absence or amount of the biomarker. Methods may sometimes comprise administering, or not administering, the composition. In certain embodiments, methods comprise administering the composition, where the composition includes a microRNA composition that increases sensitivity of melanoma cells to imidazole carboxamide anti-cell proliferation activity. In some embodiments, methods comprise administering the composition and administering a composition that includes a microRNA composition that increases sensitivity of melanoma cells to imidazole carboxamide anti-cell proliferation activity. In various embodiments, a method comprises identifying the presence, absence or amount of a biomarker in a subject having melanoma, where the biomarker comprises a microRNA that increases sensitivity of melanoma cells to imidazole carboxamide anti-cell proliferation activity, and administering an imidazole carboxamide drug to the subject based on the presence or amount of the biomarker identified.

In certain embodiments, the decision maker administers, or does not administer, the composition based on the presence, absence or amount of the biomarker. In various embodiments, methods comprise administering a composition that includes one or more components that deliver to the subject a microRNA composition that increases sensitivity of melanoma cells to imidazole carboxamide anti-cell proliferation activity.

A method sometimes comprises identifying the presence, absence or amount of a biomarker in a subject having melanoma, where the biomarker comprises a microRNA that increases sensitivity of melanoma cells to imidazole carboxamide anti-cell proliferation activity, and not administering an imidazole carboxamide drug to the subject based on the absence or amount of the biomarker identified. In certain embodiments, a method comprises selecting a composition that does not deliver imidazole carboxamide for administration to the subject. In various embodiments, the composition does not deliver an alkylating agent. In some embodiments of a method herein, the composition is administered to the subject.

Also provided, in certain embodiments, are methods where the microRNA composition comprises a microRNA selected from the group consisting of a microRNA-27, a microRNA-143, microRNA-215, microRNA-335, and combinations of the foregoing. In various embodiments, the microRNA-27 is microRNA-27a or -27b. In certain embodiments, the microRNA-143 is microRNA-143a. The microRNA sometimes is present at decreased levels in melanoma cells relative to non-cancerous quiescent cells. In certain embodiments, the microRNA modulates expression of IL-6 receptor or an IL-6 receptor pathway member. In some embodiments, the melanoma cells are in a tumor.

Provided also, in some embodiments, are methods comprising (a) administering an anti-cancer drug to a subject having metastatic melanoma, (b) identifying the presence, absence or amount of a biomarker in the subject, where the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing and (c) maintaining a subsequent dosage of the drug or adjusting a subsequent dosage of the drug administered to the subject based on the presence, absence or amount of the biomarker identified in the subject. In certain embodiments, methods comprise (a) administering an anti-cancer drug to a subject having metastatic melanoma, (b) identifying the presence, absence or amount of a biomarker in the subject, where the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing and (c) determining whether the dosage of the drug subsequently administered to the subject is adjusted based on the presence, absence or amount of the biomarker identified in the subject.

Also provided, in certain embodiments, are methods comprising identifying the presence, absence or amount of a biomarker in a subject having metastatic melanoma to whom an anti-cancer drug has been administered, where the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and maintaining a subsequent dosage of the drug or adjusting a subsequent dosage of the drug administered to the subject based on the presence, absence or amount of the biomarker identified in the subject. In some embodiments, a method comprises identifying the presence, absence or amount of a biomarker in a subject having metastatic melanoma to whom an anti-cancer drug has been administered, where the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing and determining whether the dosage of the drug subsequently administered to the subject is adjusted based on the presence, absence or amount of the biomarker identified in the subject.

In certain embodiments, provided are methods that comprise (a) receiving information comprising the presence, absence or amount of a biomarker in a subject having metastatic melanoma to whom an anti-cancer drug has been administered, where the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (b) maintaining a subsequent dosage of the drug or adjusting a subsequent dosage of the drug administered to the subject based on the presence, absence or amount of the biomarker identified in the subject.

Provided also, in some embodiments, are methods comprising (a) identifying the presence, absence or amount of a biomarker in a subject having metastatic melanoma to whom an anti-cancer drug has been administered, where the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (b) transmitting the presence, absence or amount of the biomarker to a decision maker who maintains a subsequent dosage of the drug or adjusts a subsequent dosage of the drug administered to the subject based on the presence, absence or amount of the biomarker identified in the subject. Also provided in some embodiments are methods that comprise (a) identifying the presence, absence or amount of a biomarker in a subject having metastatic melanoma to whom an anti-cancer drug has been administered, where the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (b) transmitting an indication to maintain a subsequent dosage of the drug or adjust a subsequent dosage of the drug administered to the subject based on the presence, absence or amount of the biomarker identified in the subject.

Also provided, in certain embodiments, are methods for optimizing therapeutic efficacy of a treatment of metastatic melanoma in a subject, comprising (a) identifying the presence, absence or amount of a biomarker in a subject having metastatic melanoma to whom an anti-cancer drug has been administered, where the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (b) maintaining a subsequent dosage of the drug or adjusting a subsequent dosage of the drug administered to the subject based on the presence, absence or amount of the biomarker identified in the subject.

Provided also, in some embodiments, are methods for reducing toxicity of a treatment of metastatic melanoma in a subject, comprising (a) identifying the presence, absence or amount of a biomarker in a subject having metastatic melanoma to whom an anti-cancer drug has been administered, where the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (b) maintaining a subsequent dosage of the drug or adjusting a subsequent dosage of the drug administered to the subject based on the presence, absence or amount of the biomarker identified in the subject.

Also provided, in certain embodiments, are methods comprising (a) identifying the presence, absence or amount of a biomarker in a subject, where the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, or combinations thereof, and (b) determining whether the subject is at risk, or not at risk, of having metastatic melanoma based on the presence, absence or amount of the biomarker. In some embodiments, provided are methods that comprise (a) receiving information comprising the presence, absence or amount of a biomarker in a subject, where the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (b) determining whether the subject is at risk, or not at risk, of having metastatic melanoma based on the presence, absence or amount of the biomarker.

In certain embodiments, a provided are methods that comprise (a) identifying the presence, absence or amount of a biomarker in a subject having melanoma, where the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (b) transmitting the presence, absence or amount of the biomarker to a decision maker who determines whether the subject is at risk, or not at risk, of having metastatic melanoma based on the presence, absence or amount of the biomarker. Provided also, in some embodiments, are methods that comprise (a) identifying the presence, absence or amount of a biomarker in a subject having melanoma, where the biomarker comprises a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations of the foregoing, and (b) providing an indication that the subject is at risk, or not at risk, of having metastatic melanoma based on the presence, absence or amount of the biomarker.

Provided also, in some embodiments, are methods comprising administering a composition that treats melanoma to a subject at risk of metastatic melanoma. In certain embodiments, a method comprises not administering a composition that treats melanoma to a subject not at risk of metastatic melanoma. In some embodiments, the subject has melanoma. In certain embodiments, the subject has been diagnosed with melanoma.

Also provided, in certain embodiments, are methods for treating metastatic melanoma in a subject, comprising administering a composition that delivers to a subject in need thereof a microRNA composition in an amount effective to inhibit metastasis of the melanoma cells in the subject, where the microRNA composition comprises a microRNA selected from the group consisting of microRNA-let7, microRNA-193, microRNA-206, and combinations of the foregoing. In some embodiments, provided are methods that comprise contacting metastatic melanoma cells with a microRNA composition in an amount effective to inhibit metastasis of the melanoma cells, where the microRNA composition comprises a microRNA selected from the group consisting of a microRNA-let7, microRNA-193, microRNA-206, and combinations of the foregoing. Also provided are methods that comprise, in certain embodiments, contacting metastatic melanoma cells with a microRNA composition in an amount effective to inhibit metastasis of the melanoma cells, where the microRNA composition comprises a microRNA selected from the group consisting of a microRNA-let7, microRNA-193, microRNA-206, and combinations of the foregoing.

In various embodiments, methods for treating metastatic melanoma in a subject comprise administering a composition that delivers to a subject in need thereof a microRNA inhibitor composition in an amount effective to inhibit metastasis of the melanoma in the subject, where the microRNA inhibitor composition comprises an inhibitor of a microRNA selected from the group consisting of a microRNA-21, microRNA-146, microRNA-506, microRNA-507, microRNA-508, microRNA-509, microRNA-510, microRNA-513, microRNA-514, microRNA-506-514 cluster, microRNA-506-513 cluster, and combinations of the foregoing. In some embodiments, provided are methods that comprise contacting metastatic melanoma cells with a microRNA inhibitor composition in an amount effective to inhibit metastasis of the melanoma cells, where the microRNA inhibitor composition comprises an inhibitor of a microRNA selected from the group consisting of a microRNA-21, microRNA-146, microRNA-506, microRNA-507, microRNA-508, microRNA-509, microRNA-510, microRNA-513, microRNA-514, microRNA-506-514 cluster, microRNA-506-513 cluster, and combinations of the foregoing. In certain embodiments, the metastasis is invasion by melanoma cells of non-cancer tissue. In some embodiments, the tissue is not skin. In various embodiments, the metastasis is migration of melanoma cells. In some embodiments, the metastatic melanoma cells are in a tumor. In certain embodiments, the melanoma is metastatic melanoma. In some embodiments, the microRNA is a human microRNA. In certain embodiments, the subject is human.

The microRNA-let7 sometimes is a microRNA-let7c. In certain embodiments, the microRNA-10 is a microRNA-10a. In certain embodiments, the microRNA-193 is a microRNA-193b. In various embodiments, the microRNA-146 is a microRNA-146a. In some embodiments, the microRNA-509 is a microRNA-509-1, -2 or -3. In various embodiments, the presence, absence or amount of microRNA-506, microRNA-507, microRNA-508, microRNA-509, microRNA-510, microRNA-513 and microRNA-514 is determined, and sometimes the presence, absence or amount of microRNA-506, microRNA-507, microRNA-508 and microRNA-513 is determined. In certain embodiments, a composition comprising microRNA inhibitors of microRNA-506, microRNA-507, microRNA-508, microRNA-509, microRNA-510, microRNA-513 and microRNA-514 is utilized, and sometimes a composition comprising microRNA inhibitors of microRNA-506, microRNA-507, microRNA-508 and microRNA-513 is utilized.

Also provided, in some embodiments, are methods where the presence, absence or amount of the biomarker is determined from a biological sample from the subject. In certain embodiments, the sample contains blood or a blood fraction. In various embodiments, the sample contains a skin biopsy product.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 illustrates a heat map of differentially expressed miRNAs. Fifteen (15) miRs were over-expressed and 83 miRs were under-expressed in melanomas compared to normal skin biopsies. MicroRNAs were measured by ABI TLDA in 36 melanoma patient and 16 normal donor skin punch biopsies. MicroRNAs were considered differentially expressed only if they were >2-fold different in melanoma compared to normal samples, and had a Bonferroni-adjusted p-value <0.05.

FIG. 2 schematically illustrates the M²IP³ informatics method used to predict microRNAs with putative Tumor Suppressor or Oncogene function. MicroRNA expression was measured by TaqMan RT-PCR assays arrayed on TaqMan Low-Density Arrays (TLDA). Differentially expressed microRNAs were determined by standard statistical analysis. Differentially expressed miRs were then annotated in the M²IP³ for PubMed keywords associated with cancer. Experimental details and results are described in Example 3.

FIG. 3 shows a non-limiting example of some of the miRNAs that were over expressed in melanoma cells. The increase in expression over normal or control cells is shown in the column labeled “fold change.” Experimental details and results are described in Example 3.

FIG. 4 shows the high throughput screening (HTS) methods used to determine the effectiveness of the miRs. Experimental details are described in Example 3.

FIGS. 5-6 graphically illustrate the results of growth inhibition experiments using certain onco-microRNAs (onco-miRs) and tumor suppressor-microRNAs (TS-miRs) in various melanoma cell lines. Experimental details and results are described in Example 4.

FIG. 5 graphically illustrates the results for microRNAs that significantly decreased cell growth relative to non-targeting microRNA controls and normal melanocytes in multiple melanoma cell lines.

FIG. 6 graphically illustrates the results for miRNAs that did not significantly decrease cell growth in multiple melanoma cell lines.

FIGS. 7-8 graphically illustrate the results of apoptosis experiments using certain onco-miRs and TS-miRs in various melanoma cell lines. Experimental details and results are described in Example 5.

FIG. 7 graphically illustrates the results for miRNAs that significantly increased apoptosis, as measured by caspase 3/7 activation, relative to non-targeting microRNA controls and normal melanocytes in multiple melanoma cell lines.

FIG. 8 graphically illustrates the results for miRNAs that did not significantly increase apoptosis in multiple melanoma cell lines.

FIGS. 9-11 graphically illustrate the results of migration and invasion experiments. Experimental details and results are described in Example 6.

FIG. 9 illustrates the basal migration and invasion capabilities of various melanoma and primary melanocyte cell lines.

FIG. 10 graphically illustrates the significant inhibition of migration and/or invasion due to various onco-miRs and TS-miRs in MALME-3M cells.

FIG. 11 graphically illustrates the various onco-miRs and TS-miRs that did not have a significant effect on migration and/or invasion in MALME-3M cells.

FIGS. 12-14 show expression profiles of miRNAs in the miRNA-506-514 cluster. Experimental details and results are described in Example 7.

FIG. 12 shows a representative heat map of miRNA expression in various normal and melanoma skin biopsies, and highlights the expression pattern of miRNAs in the miRNA-506-514 cluster (see vertical bar and expanded region). The miRNAs in the miRNA-506-514 cluster were over expressed in melanoma tumor biopsies when compared to normal skin biopsies.

FIG. 13 illustrates the results of expression analysis of miRNAs that were over expressed between about 30-fold and about 100-fold in melanoma samples (e.g., miR-506, miR-508, miR-509, and miR-514), or miRNAs that were absent in normal tissue and present in tumor samples (e.g., miR-507, miR-510, and miR-513).

FIG. 14 illustrates the expression levels of the miRNA-506-514 cluster in melanoma cells lines compared to normal melanocytes.

FIGS. 15 and 16 illustrate results of experiments to determine the level of cell growth and invasion/migration reduction by inhibition of the miRNA-506-514 cluster. Experimental details and results are described in Example 8.

FIG. 15 illustrates results of cell growth inhibition studies.

FIG. 16 illustrates results of invasion/migration inhibition studies.

FIGS. 17 and 18 illustrate results of experiments to determine the level of apoptosis activation due to inhibition of the miRNA-506-514 cluster. Experimental details and results are described in Example 9.

FIG. 17 illustrates the results of Caspase 3/7 activation analysis.

FIG. 18 illustrates the results of Annexin V/Propidium iodide flow cytometry analysis.

FIGS. 19 and 20 illustrate results of experiments to determine alterations in anchorage-independent growth due to inhibition of the miRNA-506-514 cluster. Experimental details and results are described in Example 10.

FIG. 19 shows representative photographs of colonies photographed at 10× magnification.

FIG. 20 graphically represents the increase in number of colonies counted in 2 fields per well in triplicate using a Nikon TE2000 microscope.

FIG. 21 illustrates physical distance mapping and analysis of the phylogenetic relationships of the miRNA-506-514 cluster members.

FIGS. 22 and 23 illustrate the results of experiments to determine the level of cell growth and invasion/migration inhibition by inhibition of the miRNA-506-514 cluster and relevant sub-clusters. Experimental details and results are described in Example 11.

FIG. 22 illustrates the results of cell growth inhibition studies.

FIG. 23 illustrates the results of invasion/migration inhibition in studies.

FIGS. 24 and 25 illustrate the results of experiments to determine the level of apoptosis activation due to inhibition of the miRNA-506-514 cluster and relevant sub-clusters. Experimental details and results are described in Example 11.

FIG. 24 illustrates results of Caspase 3/7 activation analysis.

FIG. 25 illustrates results of Annexin V/Propidium iodide flow cytometry analysis.

FIGS. 26 and 27 illustrate the results of experiments to determine alterations in anchorage-independent growth due to inhibition of the miRNA-506-514 cluster and relevant sub-clusters. Experimental details and results are described in Example 11.

FIG. 26 shows representative photographs of colonies photographed at 10× magnification.

FIG. 27 graphically represents the increase in number of colonies counted in 2 fields per well in triplicate using a Nikon TE2000 microscope.

FIGS. 28 and 29 illustrate results of experiments performed to determine the oncogenic potential of over-expression of the miRNA-506-514 cluster and relevant sub-clusters. Experimental details and results are described in Example 12.

FIG. 28 shows representative photographs of colonies photographed at 10× magnification.

FIG. 29 graphically represents the increase in number of colonies counted in 2 fields per well in triplicate using a Nikon TE2000 microscope.

FIG. 30 depicts a heat map (similar to that shown in FIG. 1) illustrating that miRNAs in the miR506-514 cluster are differentially expressed. MicroRNAs were measured by ABI TLDA in melanoma patients with comparisons made to normal skin punches or normal melanocytes collected from healthy donors.

FIG. 31 illustrates the results of expression analysis of the miRNA-506-514 cluster that were over expressed between about 30-fold and about 100-fold in melanoma samples (described in Example 7).

FIG. 32 illustrates the expression levels of the miRNA-506-514 cluster in melanoma cell lines compared to normal melanocytes (described in Example 7).

FIG. 33 illustrates additional results of invasion/migration inhibition studies at 6 and 24 hours in MALME-3M and A375 cells as described in Example 8.

FIGS. 34 and 35 illustrate results of experiments in SKMEL-5 and A375 cells to determine alterations in anchorage-independent growth due to inhibition of the miRNA-506-514 cluster. Experimental details and results are described in Example 10.

FIG. 34 shows representative photographs of colonies photographed at 10× magnification.

FIG. 35 graphically represents the increase in number of colonies by using a measure of fluorescence level that represents numbers of viable cells.

FIG. 36 illustrates the results of invasion/migration inhibition at 6 and 24 hours in MALME-3M and A375 cells as described in Example 11.

FIGS. 37 and 38 illustrate the results of experiments to determine alterations in anchorage-independent growth due to inhibition of the miRNA-506-514 cluster and relevant sub-clusters in SKMEL-5 and A375 cells. Experimental details and results are described in Example 12.

FIG. 37 shows representative photographs of colonies photographed at 10× magnification.

FIG. 38 graphically represents the increase in number of colonies by a measure of fluorescence level representing numbers of viable cells.

FIG. 39 graphically represents the level of gene expression as measured by TaqMan RT-PCR (Biomark dynamic array) for a panel of genes of known functional importance in melanogenesis. Average fold changes in transformed melanocytes from 3 independent experiments were determined by comparing gene expression levels to the average of 3 reference genes (ACTB, GAPDH, UBC) then to the expression in normal melanocytes. Asterisk (*) indicates a statistically significant difference between melanocytes transformed to grow in soft agar compared to normal melanocytes, p<0.001.

### DETAILED DESCRIPTION

The technology described herein provides therapeutic treatments of melanoma, and in certain embodiments, provides personalized medicine treatments for melanoma. Different subjects can metabolize a therapeutic drug at different rates and in different manners. This variability can result in varying effects of a drug in different subjects when treating a melanoma. Technology described herein optimizes therapeutic methods for treating melanoma by allowing a clinician to track a biomarker linked to a melanoma, and determine whether a subsequent dose of a drug for administration to a subject should be maintained, reduced or increased.

Melanoma

Melanoma, which in some forms is “malignant melanoma,” is a serious form of skin cancer, and can spread to lymph nodes and internal organs. Melanoma presently accounts for 77% of all deaths from skin cancer. Melanoma is a malignant tumor of melanocytes which are found predominantly in skin but also in the bowel and the eye. Melanocytes are normally present in skin, being responsible for the production of the dark pigment melanin. Although Melanoma is one of the less common types of skin cancer it causes the majority of skin cancer related deaths. Around 60,000 new cases of invasive melanoma are diagnosed in the United States each year, more frequently in males and in Caucasians. It is more common in Caucasian populations living in sunny climates or in those who use tanning salons, than in other groups. The World Health Organization reports about 48,000 melanoma related deaths occur worldwide per year.

Early signs of melanoma include changes to the shape or color of existing moles. The mole may itch, ulcerate or bleed. Metastatic melanoma may cause general symptoms like loss of appetite, nausea, vomiting and fatigue. Metastasis as the first symptom of melanoma is possible, however, less than a fifth of melanomas diagnosed early become metastatic. Treatment sometimes is by surgery, chemotherapy and/or radiation therapy.

Superficial Spreading Melanoma

Superficial spreading melanoma (SSM) is the most common type of melanoma in the United States, accounting for about 70% of all diagnosed melanoma cases. This type of melanoma can strike at any age and occurs slightly more often in females than males. SSM is a leading cause of death from cancer in young adults. When SSM occurs in females, it most commonly appears on the legs. In males, it is more likely to develop between the neck and pelvis. This form of melanoma, however, can occur anywhere on the skin's surface.

A typical SSM lesion has irregular borders and is various shades of black, brown, gray, blue, pink, red, or white. Within the lesion there can be a variation in color involving white, pink, brown, and black. In the early stages, SSM usually appears as a flat spot that looks like a freckle spreading sideways on the skin. Over time, the pigmentation in the lesion may darken, and the lesion may grow, develop increasingly irregular borders, and have areas of inflammation within the lesion. The area around the lesion may begin to itch. Occasionally, a SSM may become less pigmented as the subject's immune responses attempt to destroy it. However, this does not indicate that the lesion no longer requires treatment.

Nodular Melanoma

Nodular melanoma (NM) is an aggressive type of melanoma and accounts for about 15% of all melanomas diagnosed in the United States. It can appear anywhere on the body and occurs more often in males than females. It can develop at any age, although it is most often seen in people aged 60 and older. NM often is darkly pigmented, however, some NM lesions can be light brown or even colorless (non-pigmented). A light-colored or non-pigmented NM lesion may escape detection because the appearance is not alarming. An ulcerated and bleeding lesion is common.

NM tends to grow more rapidly in thickness (penetrate the skin) than in diameter and may not have a readily visible phase of development. Instead of arising from a pre-existing mole, NM may appear in a spot where a lesion did not previously exist. Prognosis can be poor because NM tends to deepen more quickly than it widens and can occur in a spot that did not have a previous lesion, decreasing the likelihood of early detection.

Lentigo Maligna Melanoma

Lentigo maligna melanoma (LMM) often occurs on sun-damaged skin in the middle-aged and elderly, especially on the face. This melanoma may be mistaken in its early, and most treatable, stages for a benign “age spot” or “sun spot.” LMM accounts for about 10% of the melanomas diagnosed in the United States. Since LMM is easily mistaken for a benign condition, it can go undetected for years.

LMM often begins as a spreading, flat, patch with irregular borders and variable colors of brown. This lesion is called “lentigo maligna.” This spreading brownish patch may grow slowly for years and is often mistaken for lentigo simplex, which is a benign (non cancerous) brownish patch that can develop in the elderly after years of sun exposure. As the lesion grows and evolves, both the pigmentation and borders tend to become more irregular. This condition often occurs slowly over a period of 10 to 15 years. It also can progress rapidly in a matter of weeks or months. As the lesion grows deeper into the skin, it may become various shades of black and brown. Dark nodules may appear within the irregular borders. These nodules are the invasive tumor, and if large enough to be felt by touch, may feel lumpy.

Acral Lentiginous Melanoma

In the United States, acral lentiginous melanoma (ALM) accounts for about 5% of all diagnosed melanomas. It also is a most common form of melanoma in Asians and people with dark skin, accounting for 50% of melanomas that occur in people with these skin types. ALM sometimes is referred to as a “hidden melanoma” because these lesions occur on parts of the body not easily examined or not thought necessary to examine. ALM develops on the palms, soles, mucous membrane, and underneath or near fingernails and toenails.

In early stages ALM often looks like a bruise or nail streak and is often overlooked until it is well advanced. On the palm or sole melanoma usually begins as an irregularly shaped tan, brown, or black spot. It often is mistakenly attributed to some recent injury, especially when the patient recalls a relatively recent bruise or blow in the general area of the pigmented spot. When melanoma develops on a mucus membrane, it is most likely to develop inside the nose or mouth. Early symptoms include nosebleeds and nasal stuffiness and a pigmented mass inside the mouth. Melanomas also can develop on the mucous membranes of the anus, urinary tract, and female genitalia.

The first sign of melanoma under a nail may be a “nail streak,” which can present as a narrow, dark stripe under the nail. ALM often develops on the thumb or big toe, although it can occur under any fingernail or toenail. Many individuals, especially dark-skinned people, can have fixed nail streaks that are completely benign. A new nail streak not associated with recent trauma, an enlarging nail streak, a wide or very darkly pigmented streak, or a nail that is separating or lifting up from the nail bed may indicate ALM. Another possible indication of advanced ALM is a nail streak with associated pigmentation in the nail fold skin or destruction of the nail plate.

ALM of the fingers or toes also can develop without an obvious nail streak, particularly the non-pigmented variety. ALM may, for example, look very much like a chronic infection of the nail bed. As an ALM tumor increases in size, it often becomes more irregular in shape and color. Some ALM lesions, however, can be lightly colored or colorless. The surface of the ALM lesion may remain flat, even as the tumor invades deeply into the skin. Thickening ALM on the sole of the foot can make walking painful and be mistaken for a plantar wart.

Stages of Melanoma

Stage 0 melanoma is a very early stage disease known as melanoma in situ (Latin for “in place”). Patients with melanoma in situ are classified as TisNOM (tumor in situ). The tumor is limited to the epidermis with no invasion of surrounding tissues, lymph nodes, or distant sites. Melanoma in situ is considered to be very low risk for disease recurrence or spread to lymph nodes or distant sites.

Stage I melanoma is characterized by tumor thickness, presence and number of mitoses, and ulceration status. There is no evidence of regional lymph node or distant metastasis. Stage I melanomas are considered to be low-risk for recurrence and metastasis. There are two subclasses of Stage I melanoma: (i) Stage IA (T1aN0M0), where a tumor is less than or equal to 1 mm, no ulceration, and no mitoses; and (ii) Stage IB (T1bN0M0 or T2aN0M0), where a tumor is less than or equal to 1 mm, with ulceration or mitoses.

Sentinel lymph node biopsy is recommended for Stage I tumors thicker than 1.0 mm and for any ulcerated tumors of any thickness. The purpose is to determine whether any cancer cells have spread to the sentinel node, the first lymph node to receive drainage from the primary tumor. The results of the biopsy may help guide the course of treatment. Sentinel node biopsy often is most accurate when it is performed before surgery that removes the tumor and the surrounding skin.

Surgery is a common treatment for Stage I melanoma. The goal of surgery is to remove any cancer remaining after the biopsy. The procedure is referred to as wide local excision. The surgeon removes the tumor, including the biopsy site, as well as a surgical margin, a surrounding area of normal-appearing skin and underlying subcutaneous tissue. The width of the margin taken depends upon the thickness of the primary tumor. Recent advances in surgery allow surgeons to take narrower margins than before, so a greater amount of normal skin is preserved.

Stage II melanomas also are localized tumors characterized by tumor thickness and ulceration status. There generally is no evidence of regional lymph node or distant metastasis. With treatment, Stage II disease is considered to be intermediate-risk for local recurrence or distant metastasis. There are three subclasses of Stage II melanoma: (a) Stage IIA (T2bN0M0 or T3aN0M0), which includes (i) 2b, where the tumor is 1.01-2.0 mm thick, with ulceration; (ii) T3a, where the tumor is 2.01-4.0 mm thick, with no ulceration; (iii) N0, where the tumor has not spread to nearby lymph nodes; and (iv) M0, where the tumor has not spread to sites distant from the primary tumor; (b) Stage IIB (T3bN0M0 or T4aN0M0Stage IIB, T3bN0M0 or T4aN0M0), which includes (i) T3b, where the tumor is 2.01-4.0 mm thick, with ulceration; (ii) T4a, where the tumor is greater than 4.0 mm thick, with no ulceration; (iii) N0, where the tumor has not spread to nearby lymph nodes; and (iv) M0, where the tumor has not spread to sites distant from the primary tumor; and (c) Stage IIC (T4bN0M0), which includes (i) T4b, where the tumor is greater than 4.0 mm thick, with ulceration; (ii) N0, where the tumor has not spread to nearby lymph nodes; and (iii) M0, where the tumor has not spread to sites distant from the primary tumor.

In addition to biopsy and surgery as described for Stage I, Stage II treatment may include adjuvant therapy, which is a treatment given in addition to a primary cancer treatment, following surgery. Systemic therapies use substances that travel through the bloodstream to reach and affect cancer cells throughout the body. Treatments include interferons, natural proteins produced by the normal cells of most body tissues in response to viral infections and disease. Interferon therapies have been shown to help the body's immune system fight disease more effectively. Studies indicate that low-dose interferon alfa-2a, a manufactured form of interferon, consistently delays relapse in patients with Stage II melanoma and higher-risk Stage IIB disease, but does not extend overall survival. High-dose interferon alfa-2b has been shown to significantly prolong disease-free and overall survival in patients with high-risk Stage IIB and Stage III melanoma. Vaccines, like interferons, may help boost the immune system to fight the return of melanoma. Vaccine therapy has been investigated as a therapy for patients who cannot tolerate the side effects of immunotherapies, such as interferon.

Stage III melanomas are tumors that have spread to regional lymph nodes, or have developed in transit metastasis or satellites. There often is no evidence of distant metastasis. With treatment, Stage III disease is considered to be intermediate-to high-risk for local recurrence or distant metastasis.

Stage III melanomas generally are defined by the number of lymph nodes to which the tumor has spread, whether tumor spread to the lymph nodes is microscopic or macroscopic, the presence of in transit or satellite tumor, and whether the primary tumor that is the source of lymph node spread shows evidence of ulceration. The epidermis that covers a portion of the primary melanoma often is not intact. Ulceration is determined by microscopic evaluation of the tissue by a pathologist, not by what can be seen with the naked eye. Micrometastases are tiny tumors not visible to the naked eye. They can be detected by microscopic evaluation after sentinel lymph node biopsy or elective lymph node dissection. Macrometastases often can be felt during physical examination or seen with the naked eye when inspected by a surgeon or pathologist. Presence often is confirmed by lymph node dissection or when the tumor is seen to extend beyond the lymph node capsule.

Subclasses of Stage III Melanoma include (a) Stage IIIA (T1-T4a N1aM0 or T1-T4aN2aM0), which include (i) T1-T4a, where the tumor is not ulcerated and ranges in size from less than 1.0 mm to more than 4.0 mm thick; (ii) N1a, where micrometastasis is diagnosed in 1 nearby lymph node; (iii) N2a, where micrometastasis is diagnosed in 2-3 nearby lymph nodes; and (iii) M0, where the tumor has not spread to sites distant from the primary tumor; (b) Stage IIIB (T1-T4bN1aM0, T1-T4bN2aM0, T1-T4aN1bM0, T1-T4aN2bM0, or T1-T4a/bN2cM0), which includes (i) T1-T4a, where the tumor is not ulcerated and ranges in size from less than 1.0 mm to more than 4.0 mm thick; (ii) T1-4-b, where the tumor is ulcerated and ranges in size from less than 1.0 mm to more than 4.0 mm thick; (iii) N1b, where macrometastasis is diagnosed in 1 nearby lymph node; (iv) N2b, where macrometastasis is diagnosed in 2-3 nearby lymph nodes; (v) N2c, where presence of in-transit metastases or satellite metastases; and (vi) M0, where the tumor has not spread to sites distant from the primary tumor; and (c) Stage IIIC (T1-4-bN1bN0, T1-4-bN2bM0, T1-4-aN3M0 or T1-4-bN3M0), which includes (i) T1-T4a, where the tumor is not ulcerated and ranges in size from less than 1.0 mm to more than 4.0 mm thick; (ii) T1-4-b, where the tumor is ulcerated and ranges in size from less than 1.0 mm to more than 4.0 mm thick; (iii) N1b, where macrometastasis is diagnosed in 1 nearby lymph node; (iv) N2b, where macrometastasis is diagnosed in 2-3 nearby lymph nodes; (v) N3, where metastasis in 4 or more lymph nodes, the presence of matted lymph nodes, or the combination of in-transit/satellite metastases and metastatic lymph nodes; and (vi) M0, where the tumor has not spread to sites distant from the primary tumor.

In addition to surgery and adjuvant therapy as described above, Stage III melanoma treatment often includes therapeutic lymph node dissection (TLND), which is surgery to remove regional lymph nodes from the area where cancerous lymph nodes were found. Such surgery is highly recommended for patients with macrometastases. The goal of the surgery is to prevent further spread of the disease through the lymphatic system. TLND also plays an important role in controlling the pain often caused by untreated lymph node disease. Lymphatic mapping and sentinel node biopsy generally are not recommended for patients with clinically diagnosed Stage III disease. These procedures may be recommended, however, for patients with certain subgroups of Stage III disease. Adjuvant radiation therapy has not been proven to be of benefit in randomized, controlled studies but is sometimes recommended when the tumor has grown outside the lymph nodes into the surrounding tissue (extracapsular spread). The goal is to control the further spread of the disease.

Stage IV melanomas often are associated with metastasis beyond the regional lymph nodes to distant sites in the body. Common sites of metastasis are to vital organs (lungs, abdominal organs, brain, and bone) and soft tissues (skin, subcutaneous tissues, and distant lymph nodes). Stage IV melanoma may be characterized by the location of the distant metastases; the number and size of tumors; and the serum lactate dehydrogenase (LDH) level. LDH is an enzyme found in the blood and many body tissues. Elevated LDH levels usually indicate that the tumor has spread to internal organs.

Stage IV melanomas generally do not include T or N classification, and include: (a) M1a, where the tumor has metastasized to distant skin, the subcutaneous layer or to distant lymph nodes and serum LDH is normal; (b) M1b, where the tumor has metastasized to the lungs and serum LDH is normal; and (c) M1c, where the tumor has metastasized to vital organs other than the lungs and serum LDH is normal, and there are any distant metastases with elevated LDH.

No treatment so far has definitively shown to prolong survival or cure disease in Stage IV melanoma. Treatments instead focus on relieving uncomfortable symptoms caused by the disease.

Treatments include: surgery to remove cancerous tumors or lymph nodes that have metastasized to other areas of the body, if they are few in number and are causing symptoms; established and experimental systemic therapies; and radiation therapy. Radian therapy generally is reserved for advanced cases where surgery is not possible or may be complicated, and for relieving symptoms of metastatic disease to the brain or bone.

Melanoma Invasion

The basement membrane is a thin extracellular matrix that underlies epithelial and endothelial cells and separates these tissues from stroma. Tumor cells cross the vessel basement membrane and penetrate the underlying stroma when invading tissue to form distant metastases. Tumor cells can produce proteases that degrade the extracellular matrix in the invasion process. In vivo and in vitro assays can be used to test melanoma cell motility and invasion. Assays of subject LDH level can provide an indication of organ invasion by melanoma, as LDS is released by organ disruption.

In vitro invasion assays can be performed using melanoma cell samples gathered as described elsewhere herein.

Several in vitro invasion assays have been developed using various extracellular matrix barriers including amnion, type I collagen gels, and a reconstituted basement membrane termed Matrigel. Porous filters, in some assay embodiments, are coated with a thin layer of Matrigel and placed in a Boyden migration chamber with a chemoattractant in the lower well and tumor cells in the upper well. The entire chamber is then incubated for about 3 to 10 hours, depending on the tumor cells used. After incubation, the filter is removed, fixed, and stained, and the cells on the lower surface of the filter are quantified. Molecules that promote or inhibit invasion can be assayed. At the end of the assay, the invasive cells can be recovered and used for further study. An invasion assay can be used to screen for a variety of compounds in 48-well chambers, in which smaller amounts of test material and fewer cells are needed, in some embodiments. Commercial kits for conducting invasion assays are available. Results obtained using such assays, for example Matrigel-based invasion assays, can show a correlation between the ability of tumor cells to invade in vitro and their invasive behavior in vivo.

Melanoma Cell Lines

Melanoma cells may be obtained from cell cultures using techniques known in the art. As described herein, a “melanoma cell line” comprises cells that initially were derived from a melanoma, and can exist in primary culture or secondary culture (e.g., cells may be passaged one or more times since they were derived from a melanoma). A melanoma cell line can be derived from any melanoma. Such cells sometimes have undergone a change such that they can undergo superior proliferation, growth and passaging in culture relative to primary cells or non-cancerous cells (e.g., non-cancerous cells often can be cultured only for a finite period of time).

A melanoma cell line can be obtained by any suitable procedure. In some embodiments, a method comprises (a) obtaining a melanoma sample from a mammalian host, (b) forming a single cell suspension from the melanoma sample, (c) pelleting the melanoma cells, (d) transferring the melanoma cells into tissue culture using standard sterile culture technique, and (e) maintaining the melanoma cells in tissue culture under conditions that allow the growth of the melanoma cells.

A melanoma sample may be obtained at any suitable time (e.g., time of surgery). A melanoma sample often is handled and manipulated using sterile techniques, and in such a fashion so as to minimize tissue damage. A melanoma sample may be placed on ice in a sterile container and moved to a laboratory laminar flow hood. A portion of a melanoma sample identified for isolation of a melanoma cell line can be excised, and the remainder of the melanoma sample may be stored at a suitable temperature (e.g., −70 degrees Celsius).

A cell suspension can be formed by enzymatically digesting cells, sometimes overnight. For instance, a sample can be suspended in a solution that contains collagenase in some embodiments. A solution also can contain DNase and/or hyaluronidase in certain embodiments, and a cell culture medium can be employed to carry out digestion. A resultant single cell suspension often is pelleted, and pellets can be resuspended in a small volume of tissue culture medium. Resuspended cells can be inoculated into tissue culture medium appropriate for the growth of the cells in culture at a suitable density (e.g., about 5×10⁵ tumor cells/ml).

A fresh tumor sample sometimes is minced into small pieces, which can be placed into culture directly. This method of isolating a melanoma cell line can include (a) obtaining a sample of melanoma from a mammalian host, (b) mincing the sample to obtain fragments thereof, (c) transferring the fragments of fresh tumor into tissue culture, and (d) maintaining the melanoma cells in tissue culture under conditions that allow growth and/or proliferation of the cells.

Regardless of the method used to transfer melanoma cells into tissue culture, once transferred, the cultures can be maintained at about 35 to about 40 degrees Celsius in the presence of about 5-8% CO₂. A suitable medium known in the art for cell proliferation and/or growth may be used, e.g., a medium that utilizes a bicarbonate buffering system and various amino acids and vitamins. A medium utilized sometimes is RPMI 1640 medium, which may be supplemented with bovine serum (e.g., fetal bovine serum), sometimes at a concentration of from about 5 to about 20%. The medium can contain various additional factors as necessary, e.g., when required for the growth of the melanoma cells, or for maintenance of the melanoma cells in an undifferentiated state. Medium and medium components often are readily available, and can be obtained, for instance, from commercial suppliers. Cell cultures can be fed and recultured as necessary, e.g., typically every 1 to 10 days. The tumor cells also can be subjected to differential trypsinization to remove other cells (e.g., stromal cells) that can overgrow the primary tumor cultures. Also, suppression of fibroblast overgrowth can be achieved by supplementing the culture medium with cholera toxin (e.g., 10 ng/ml).

When it appears that a substantially purified culture of the melanoma cells has been obtained (e.g., as judged by the appearance or growth behavior of the cultures), various tests can be carried out to confirm the purity of the cultures. For instance, culture purity can be confirmed by flow cytometry or immunocytology to validate expression of melanoma-associated proteins or gangliosides. Such analysis can be performed using antibodies that are readily available, and as known in the art.

Melanoma cell lines are commercially available (e.g., Wistar Institute (Philadelphia), Trenzyme Biotechnology (Germany)). Non-limiting examples of melanoma cell lines include MALME-3M [HTB-64], SK-MEL-5 [HTB-70], SK-MEL-2 [HTB-68], A375 [CRL-1619], and RPMI-7951 [HTB-66].

Nucleic Acids

A “nucleic acid” as used herein generally refers to a molecule (one, two or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.” Nucleic acids as provided herein include without limitation microRNA, siNA, and antisense RNA. Nucleic acids may be, be at least, be at most, or be about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed microRNA or siNA, microRNA or siNA molecules, precursor microRNA or siNA, microRNA or siNA containing vectors, control nucleic acids, and other molecules, probes and primers. In many embodiments, microRNA are 19-24 nucleotides in length, while microRNA precursors are generally between 62 and 110 nucleotides in humans.

Nucleic acids herein provided may have regions of identity or complementarity to another nucleic acid. It is contemplated that the region of complementarity or identity can be at least 5 contiguous residues, though it is specifically contemplated that the region is, is at least, is at most, or is about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides. It is further understood that such lengths of complementarity are within a precursor microRNA or between a microRNA drug and that a target molecule or a microRNA gene are such lengths.

A nucleic acid may also comprise a vector, including without limitation a plasmid or virus. The vector may code for a pre-processed nucleic acid molecule, or for the mature post-processed molecule (e.g., pre-processed or pos-processed microRNA or siRNA).

As provided herein a “synthetic nucleic acid” means that the nucleic acid does not have a chemical structure or sequence of a naturally occurring nucleic acid. Consequently, it is understood that the term “synthetic microRNA” refers to a “synthetic nucleic acid” that is not isolated from a cell and is artificially manufactured, but which may sometimes function in a cell or under physiological conditions.

While some embodiments may involve synthetic microRNAs or other synthetic nucleic acids, in certain embodiments, the nucleic acid molecule(s) need not be “synthetic.” In such embodiments, a non-synthetic microRNA employed in methods and compositions may have the entire sequence and structure of a naturally occurring microRNA precursor or the mature microRNA. For example, non-synthetic microRNAs used in methods and compositions as herein provided may not have one or more modified nucleotides or nucleotide analogs. In these embodiments, the non-synthetic microRNA may or may not be recombinantly produced. In particular embodiments, the nucleic acid in methods and/or compositions provided herein is specifically a synthetic microRNA and not a non-synthetic microRNA (that is, not a microRNA that qualifies as “synthetic”). In some embodiments a non-synthetic microRNA and not a synthetic microRNA may be utilized. Any embodiments discussed with respect to the use of synthetic microRNAs can be applied with respect to non-synthetic microRNAs, and vice versa.

As used herein the term “naturally occurring” refers to something found in an organism without any intervention by a person; it could refer to a naturally-occurring wild-type or mutant molecule. In some embodiments a synthetic microRNA molecule does not have the sequence of a naturally occurring microRNA molecule. In other embodiments, a synthetic microRNA molecule may have the sequence of a naturally occurring microRNA molecule, but the chemical structure of the molecule, particularly in the part unrelated specifically to the precise sequence (non-sequence chemical structure) differs from chemical structure of the naturally occurring microRNA molecule with that sequence. In some cases, the synthetic microRNA has a sequence and non-sequence chemical structure that are not found in a naturally-occurring microRNA. Moreover, the sequence of the synthetic molecules can identify which microRNA is effectively being provided or inhibited. The endogenous microRNA is referred to herein as the “corresponding microRNA.” Corresponding microRNA sequences that can be used in as herein provided include, but are not limited to, all or a portion of those sequences previously listed herein, as well as any other microRNA sequence, microRNA precursor sequence, or any sequence complementary thereof.

As used herein, “hybridization”, “hybridizes” or “capable of hybridizing” is understood to mean forming a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are known, and are appropriate for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42 degrees C. to about 70 degrees C. It is understood that the temperature and ionic strength of a desired stringency are determined in part by the length of the particular nucleic acid(s), the length and nucleobase content of the target sequence(s), the charge composition of the nucleic acid(s), and the presence or concentration of formamide, tetramethylammonium chloride or other solvent(s) in a hybridization mixture.

It is understood that these ranges, compositions and conditions for hybridization are mentioned by way of non-limiting examples only, and that the desired stringency for a particular hybridization reaction is often determined empirically by comparison to one or more positive or negative controls. Depending on the application envisioned varying conditions of hybridization may be employed to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20.degree. C. to about 50.degree. C. The low or high stringency conditions may be further modified to suit a particular application.

A nucleic acid sometimes includes a nucleotide sequence identical to or substantially identical to a microRNA nucleotide sequence described herein. In some embodiments, a nucleic acid includes a nucleotide sequence that (a) is about 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to a microRNA nucleotide sequence provided herein; (b) results from adding, or removing, 1, 2, 3, 4, 5, 6, 7, 8, 9 or 10 bases to or from a microRNA sequence provided herein; (c) includes 1, 2, 3, 4, 5, 6, 7, 8, 9 or 10 base substitutions relative to a microRNA sequence provided herein; and (d) is complementary to a nucleotide sequence of (a), (b) or (c). Nucleotide sequence identity, and nucleotide substitutions, deletions or additions, can be determined by alignment processes and tools known in the art. Sequence alignments can be implemented using available software, for example.

MicroRNA

A nucleic acid sometimes is a microRNA (miRNA). MicroRNAs (also referred to herein as “miRNAs”) are a class of non-coding regulatory RNAs of approximately 15 to 30 nucleotides in length. The term “microRNA” generally refers to a single-stranded molecule, but in specific embodiments, may also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, microRNA may encompass a single-stranded, double-stranded or partially single-stranded molecule. For example, precursor microRNA may have a self-complementary region, which is up to 100% complementary.

MicroRNAs are highly conserved across a number of species. They regulate gene expression post-transcriptionally, primarily by associating with the 3′ untranslated region (UTR) of their regulatory target mRNAs. MicroRNAs are implicated in cell proliferation, differentiation, and apoptosis. It is understood that some microRNA is derived from genomic sequences or a gene. In this respect, the term “gene” is used for simplicity to refer to the genomic sequence encoding the precursor microRNA for a given microRNA. However, some embodiments may involve genomic sequences of a microRNA that are involved in its expression, such as a promoter or other regulatory sequences. The term “recombinant” may be used and this generally refers to a molecule that has been manipulated in vitro or that is a replicated or expressed product of such a molecule.

Native microRNAs are regulatory RNAs that act as the recognition component of the complex RNA-induced Silencing Complex (RISC) riboprotein complex. The genes encoding microRNAs are longer than the processed mature microRNA molecule. Genomic microRNAs exist in many different forms, including individual genes, genetic clusters of multiple microRNAs, or encoded within the introns of protein coding genes. MicroRNAs are first transcribed as primary transcripts or pri-miRs consisting of RNA transcripts averaging about 1.2 Kb, or within the introns of long protein coding transcripts. Pri-miRs are processed by Drosha enzymes to short, roughly 70 to 120-nucleotide stem-loop structures, known as pre-miRNA in the cell nucleus. These pre-miRNAs then are processed to mature functional microRNAs in the cytoplasm by interaction with the endonucleases Argonaut, Dicer, and others to produce the RISC complex.

MicroRNAs generally inhibit translation or promote mRNA degradation by base-pairing to complementary sequences within the 3′ untranslated regions (UTRs) of regulatory target mRNAs. Individual messenger RNAs (mRNAs) can be targeted by several microRNAs, and a single microRNA can regulate multiple target mRNAs. MicroRNAs can coordinately regulate a set of genes encoding proteins with related functions, providing enormous complexity and the potential of gene regulation.

A microRNA may be inhibited in certain embodiments. A microRNA may be inhibited at a particular stage of microRNA development, including by a molecule that interferes with transcription of the microRNA gene or intron segment, for example. Such a molecule may promote degeneration of, interfere with proper cutting of, or otherwise inactivate pri-microRNA, or otherwise prevent maturation to functional microRNA. A microRNA inhibitor molecule, in some embodiments, may interact with (e.g., bind to, cleave) a gene, intron segment, transcript, pri-microRNA and/or mature microRNA. A microRNA inhibitor molecule may interact with (e.g., bind to) a Drosha, Argonaut, Dicer, or other microRNA processing enzyme in some embodiments. In certain embodiments, a microRNA inhibitory molecule is a single-stranded nucleic acid (e.g., DNA, RNA or derivative or combination thereof) or a siNA (e.g., siRNA). In some embodiments, the microRNA inhibitory molecule corresponds to an anti-sense DNA or RNA sequence of a corresponding mature microRNA, or an antisense oligonucleotide molecule that is complementary to a fragment of the mature microRNA. In other embodiments, a microRNA inhibitory molecule that is an antisense DNA, antisense RNA or antisense olignucleotide can contain additional olignonucleotide sequences that enhance suppression or can contain modifications to the phosphate backbone or modified nucleotide bases that enhance antisense binding and/or confer resistance to degradation.

MicroRNAs can be labeled, used in array analysis, or employed in diagnostic, therapeutic, or prognostic applications, particularly those related to pathological conditions such as melanoma. The microRNA may have been endogenously produced by a cell, or synthesized or produced chemically or by recombinant technology. MicroRNA may be isolated and/or purified. Human microRNA molecules often are referenced herein with the prefix “hsa-miR-”. Unless otherwise indicated, microRNAs referred to in the application are human sequences, and non-human microRNA sequences can be determined and prepared from these (e.g., for applications in non-human subjects).

In some embodiments, a microRNA may used that does not correspond to a known human microRNA. These non-human microRNAs may be used in certain embodiments or there may exist a human microRNA that is homologous to a non-human microRNA. In various embodiments, a mammalian cell, biological sample, or preparation thereof may be employed.

siNA

Certain nucleic acids can be short interference nucleic acids (siNA). siNA refers to a class of nucleic acid molecules capable of mediating sequence specific RNA inhibition (RNAi), for example short interfering RNA (siRNA), double-stranded RNA (dsRNA), microRNA (microRNA) or (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, or epigenetics. For example, siNA molecules can be used to epigenetically silence genes at either or both of the post-transcriptional level and the pre-transcriptional level. In a non-limiting example, epigenetic regulation of gene expression by siNA molecules of the technology can result from siNA mediated modification of chromatin structure to alter gene expression. Thus, an siNA may be used therapeutically to mediate the level of a polypeptide or protein. In some embodiments siNA (e.g., siRNA) are utilized as inhibitors of miRNA (miRNA is described in greater detail hereafter), and methods are known in the art for designing, selecting and making such siRNA.

A siNA may be a double-stranded polynucleotide molecule comprising self-complementary sense and antisense regions, where the antisense region comprises nucleotide sequence that is complementary to nucleotide sequence in a target nucleic acid molecule or a portion thereof and the sense region having nucleotide sequence corresponding to the target nucleic acid sequence or a portion thereof. A siNA can be assembled from two separate oligonucleotides, where one strand is the sense strand and the other is the antisense strand, where the antisense and sense strands are self-complementary. In some embodiments, each strand comprises nucleotide sequence that is complementary to nucleotide sequence in the other strand; such as where the antisense strand and sense strand form a duplex or double stranded structure, for example where the double stranded region is about 1, 2, 3, 4, 5, 6 7, 8, 9, 10, 11, 12, 13, 14, 15, 16, 17, 18, 19, 20, 21, 22, 23, 24, or 25 or more base pairs.

The antisense strand can comprise a nucleotide sequence that is complementary to nucleotide sequence in a target nucleic acid molecule or a portion thereof and the sense strand comprises nucleotide sequence corresponding to the target nucleic acid sequence or a portion thereof. In some embodiments, a siNA can be assembled from a single oligonucleotide, where the self-complementary sense and antisense regions of the siNA are linked by means of a nucleic acid based or non-nucleic acid-based linker(s). A siNA can be a polynucleotide with a hairpin secondary structure, having self-complementary sense and antisense regions, where the antisense region comprises nucleotide sequence that is complementary to nucleotide sequence in a separate target nucleic acid molecule or a portion thereof and the sense region having nucleotide sequence corresponding to the target nucleic acid sequence or a portion thereof. A siNA can be a circular single-stranded polynucleotide having two or more loop structures and a stem comprising self-complementary sense and antisense regions, where the antisense region comprises nucleotide sequence that is complementary to nucleotide sequence in a target nucleic acid molecule or a portion thereof and the sense region having nucleotide sequence corresponding to the target nucleic acid sequence or a portion thereof, and where the circular polynucleotide can be processed either in vivo or in vitro to generate an active siNA molecule capable of mediating RNAi.

In some embodiments a siNA comprises two strands of RNA. In certain embodiments an siNA comprises two strands of DNA. A siNA may sometimes be a hybrid, comprising one strand of RNA and one strand of DNA. One or both strands may also comprise mixed RNA and DNA. In some embodiments a strand of a siNA (e.g., a strand of a siRNA) may be about 5 to about 60 nucleotides in length (e.g., about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44 45 46 47 48, 49, 50, 51, 52, 53, 54, 55, 56, 57, 58 or 59 nucleotides). A siNA strand sometimes may exceed 60 nucleotides.

A siNA may also comprise a single-stranded polynucleotide having a nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), where the single stranded polynucleotide can further comprise a terminal phosphate group, such as a 5′-phosphate or 5′,3′-diphosphate.

In certain embodiments, a siNA molecule may comprise separate sense and antisense sequences or regions, where the sense and antisense regions are covalently linked by nucleotide or non-nucleotide linkers molecules as is known in the art, or are alternately non-covalently linked by ionic interactions, hydrogen bonding, van der Weals interactions, hydrophobic interactions, and/or stacking interactions. In certain embodiments, a siNA molecule comprises a nucleotide sequence that is complementary to nucleotide sequence of a target gene. In some embodiments, the siNA molecule interacts with nucleotide sequence of a target gene in a manner that causes inhibition of expression of the target gene. siNA may sometimes disrupt or interfere with microRNA (miRNA).

Nucleic Acid Modification

Any of the modifications described herein may be applied to a nucleic acid (e.g., microRNA and siRNA) as appropriate. Examples of modifications include alterations to the RNA backbone, sugar or base, and various combinations thereof. Any suitable number of backbone linkages, sugars and/or bases in a microRNA or other nucleic acid can be modified (e.g., independently about 5%, 10%, 15%, 20%, 25%, 30%, 35%, 40%, 45%, 50%, 55%, 60%, 65%, 70%, 75%, 80%, 85%, 90%, 95%, up to 100%). An unmodified microRNA nucleoside is any one of the bases adenine, cytosine, guanine, thymine, or uracil joined to the 1′ carbon of beta-D-ribo-furanose.

A modified base is a nucleotide base other than adenine, guanine, cytosine and uracil at a 1′ position. Non-limiting examples of modified bases include inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and the like. Other non-limiting examples of modified bases include nitropyrrolyl (e.g., 3-nitropyrrolyl), nitroindolyl (e.g., 4-, 5-, 6-nitroindolyl), hypoxanthinyl, isoinosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, difluorotolyl, 4-fluoro-6-methylbenzimidazole, 4-methylbenzimidazole, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenzyl, tetracenyl, pentacenyl and the like.

In some embodiments, for example, a nucleic acid may comprise modified nucleic acid molecules, with phosphate backbone modifications. Non-limiting examples of backbone modifications include phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl modifications. In certain instances, a ribose sugar moiety that naturally occurs in a nucleoside is replaced with a hexose sugar, polycyclic heteroalkyl ring, or cyclohexenyl group. In certain instances, the hexose sugar is an allose, altrose, glucose, mannose, gulose, idose, galactose, talose, or a derivative thereof. The hexose may be a D-hexose, glucose, or mannose. In certain instances, the polycyclic heteroalkyl group may be a bicyclic ring containing one oxygen atom in the ring. In certain instances, the polycyclic heteroalkyl group is a bicyclo[2.2.1]heptane, a bicyclo[3.2.1]octane, or a bicyclo[3.3.1]nonane.

Nitropyrrolyl and nitroindolyl nucleobases are members of a class of compounds known as universal bases. Universal bases are those compounds that can replace any of the four naturally occurring bases without substantially affecting the melting behavior or activity of the oligonucleotide duplex. In contrast to the stabilizing, hydrogen-bonding interactions associated with naturally occurring nucleobases, oligonucleotide duplexes containing 3-nitropyrrolyl nucleobases may be stabilized solely by stacking interactions. The absence of significant hydrogen-bonding interactions with nitropyrrolyl nucleobases obviates the specificity for a specific complementary base. In addition, 4-, 5- and 6-nitroindolyl display very little specificity for the four natural bases. Other universal bases include hypoxanthinyl, isoinosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, and structural derivatives thereof.

Difluorotolyl is a non-natural nucleobase that functions as a universal base. Difluorotolyl is an isostere of the natural nucleobase thymine. But unlike thymine, difluorotolyl shows no appreciable selectivity for any of the natural bases. Other aromatic compounds that function as universal bases are 4-fluoro-6-methylbenzimidazole and 4-methylbenzimidazole. In addition, the relatively hydrophobic isocarbostyrilyl derivatives 3-methyl isocarbostyrilyl, 5-methyl isocarbostyrilyl, and 3-methyl-7-propynyl isocarbostyrilyl are universal bases which cause only slight destabilization of oligonucleotide duplexes compared to the oligonucleotide sequence containing only natural bases. Other non-natural nucleobases include 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenzyl, tetracenyl, pentacenyl, and structural derivates thereof. For a more detailed discussion, including synthetic procedures, of difluorotolyl, 4-fluoro-6-methylbenzimidazole, 4-methylbenzimidazole, and other non-natural bases mentioned above.

In addition, chemical substituents, for example cross-linking agents, may be used to add further stability or irreversibility to the reaction. Non-limiting examples of cross-linking agents include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

A nucleotide analog may also include a “locked” nucleic acid. Certain compositions can be used to essentially “anchor” or “lock” an endogenous nucleic acid into a particular structure. Anchoring sequences serve to prevent disassociation of a nucleic acid siNA complex, and thus not only can prevent copying but may also enable labeling, modification, and/or cloning of the endogeneous sequence. The locked structure may regulate gene expression (i.e. inhibit or enhance transcription or replication), or can be used as a stable structure that can be used to label or otherwise modify the endogenous nucleic acid sequence, or can be used to isolate the endogenous sequence, i.e. for cloning.

Nucleic acid molecules need not be limited to those molecules containing only RNA or DNA, but further encompass chemically-modified nucleotides and non-nucleotides. The percent of non-nucleotides or modified nucleotides may be from 1% to 100% (e.g., about 5, 10, 15, 20, 25, 30, 35, 40, 45, 50, 55, 60, 65, 70, 75, 80, 85, 90 or 95%). In certain embodiments, siNA lack 2′-hydroxy (2′-OH) containing nucleotides. In certain embodiments siNA do not require the presence of nucleotides having a 2′-hydroxy group for mediating RNAi and as such, siNA may include no ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Sometimes siNA molecules can comprise ribonucleotides at about 5, 10, 20, 30, 40, or 50% of the nucleotide positions.

Biomarkers

Provided herein are microRNA (also referred to herein as “miRNA”) biomarkers associated with a melanoma. MicroRNAs can have a nucleotide sequence corresponding to, or derived from, any suitable source, including without limitation, cells from a mammal (e.g., human). In certain embodiments, provided herein are human forms of microRNA biomarkers set forth in Table 1, and in Tables A, B, C, D, and E. A biomarker, according to the invention, can represent a biomarker family, where a biomarker family includes several microRNAs that are members of the biomarker family. For example, microRNA-513 represents a family of microRNAs that includes miR-5,3-a-3p, miR-5,3-a-5p, miR-5,3-b, miR-5,3-c. Thus, methods according to the present invention that apply to particular biomarkers, can also be applied to individual family members of a biomarker family.

In some embodiments, microRNA biomarkers are under-represented in melanoma cells, in which case the microRNA biomarkers are referred to as “miRNA tumor suppressors” herein. In some embodiments, microRNA biomarkers are over-represented in melanoma cells, in which case the microRNA biomarkers are referred to as “miRNA oncogenes” herein. MicroRNA tumor suppressors and mRNA oncogenes are discussed in greater detail hereafter.

In certain embodiments, analysis of a biomarker can allow a clinician to determine whether a subsequent dose of the drug should be increased, decreased or maintained. For example, determining that an over-represented biomarker level is significantly reduced and/or that an under-represented biomarker level is significantly increased after drug treatment provides an indication to a clinician that an administered drug is exerting a therapeutic effect. Based on such a biomarker determination, a clinician can make a decision to maintain a subsequent dose of the drug or lower the subsequent dose. In another example, determining that an over-represented biomarker level is not significantly reduced and/or that an under-represented biomarker level is not significantly increased provides an indication to a clinician that an administered drug is not significantly exerting a therapeutic effect. Based on such a biomarker determination, a clinician could make a decision to increase a subsequent dose of the drug. Given that drugs can be toxic to a subject and exert side effects, methods provided herein optimize therapeutic approaches as they provide the clinician with the ability to “dial in” an efficacious dosage of a drug and minimize side effects. In specific examples, methods provided herein allow a clinician to “dial-up” the dose of a drug to a therapeutically efficacious level, where the dialed-up dosage is below a toxic threshold level. Accordingly, treatment methods described herein can enhance efficacy and reduce the likelihood of toxic side effects.

In some embodiments, analysis of a biomarker can allow a clinician to identify subjects who are more likely to respond to a drug and subjects who are less likely to respond to a drug. In certain embodiments, analysis of a biomarker can allow a clinician to identify subjects at risk of a more aggressive form of melanoma, or more advanced clinical stage of melanoma. A clinician can make such a determination based on whether the presence, absence or amount of a biomarker is below, above or about the same as a biomarker threshold, respectively, in certain embodiments.

Sources of Biomarkers

A fluid or tissue sample often is obtained from a subject for determining presence, absence or amount ex vivo. Non-limiting parts of the body from which a tissue sample may be obtained include leg, arm, abdomen, upper back, lower back, chest, hand, finger, fingernail, foot, toe, toenail, neck, rectum, nose, throat, mouth, scalp, face, spine, throat, heart, lung, breast, kidney, liver, intestine, colon, pancreas, bladder, cervix, testes, muscle, skin, hair, region of inflammation, tumor, region of diffuse cancer cells, and the like, in some embodiments.

A tissue sample can be obtained by any suitable method known in the art, including, without limitation, biopsy (e.g., shave, punch, incisional, excisional, curettage, fine needle aspirate, scoop, scallop, core needle, vacuum assisted, open surgical biopsies) and the like, in certain embodiments. Examples of a fluid that can be obtained from a subject includes, without limitation, blood, cerbrospinal fluid, spinal fluid, lavage fluid (e.g., bronchoalveolar, gastric, peritoneal, ductal, ear, athroscopic), urine, interstitial fluid, feces, sputum, saliva, nasal mucous, prostate fluid, lavage, semen, lymphatic fluid, bile, tears, sweat, breast milk, breast fluid, fluid from region of inflammation, fluid from a tumor region, a diffuse cell overgrowth region and the like, in some embodiments.

A sample from a subject may be processed prior to determining presence, absence or amount of a biomarker. For example, a blood sample from a subject may be processed to yield a certain fraction, including without limitation, plasma, serum, buffy coat, red blood cell layer and the like, and biomarker presence, absence or amount can be determined in the fraction. In certain embodiments, a tissue sample (e.g., tumor biopsy sample) can be processed by slicing the tissue sample and observing the sample under a microscope before and/or after the sliced sample is contacted with an agent that visualizes a biomarker (e.g., antibody). In some embodiments, a tissue sample can be exposed to one or more of the following non-limiting conditions: washing, exposure to high salt or low salt solution (e.g., hypertonic, hypotonic, isotonic solution), exposure to shearing conditions (e.g., sonication, press (e.g., French press)), mincing, centrifugation, separation of cells, separation of tissue and the like. In certain embodiments, a biomarker can be separated from tissue and the presence, absence or amount determined in vitro. A sample also may be stored for a period of time prior to determining the presence, absence or amount of a biomarker (e.g., a sample may be frozen, cryopreserved, maintained in a preservation medium (e.g., formaldehyde)).

A sample can be obtained from a subject at any suitable time of collection after a drug is delivered to the subject. For example, a sample may be collected within about one hour after a drug is delivered to a subject (e.g., within about 5, 10, 15, 20, 25, 30, 35, 40, 45, 55 or 60 minutes of delivering a drug), within about one day after a drug is delivered to a subject (e.g., within about 2, 3, 4, 5, 6, 7, 8, 9, 10, 11, 12, 13, 14, 15, 16, 17, 18, 19, 20, 21, 22, 23 or 24 hours of delivering a drug) or within about two weeks after a drug is delivered to a subject (e.g., within about 2, 3, 4, 5, 6, 7, 8, 9, 10, 11, 12, 13 or 14 days of delivering the drug). A collection may be made on a specified schedule including hourly, daily, semi-weekly, weekly, bi-weekly, monthly, bi-monthly, quarterly, and yearly, and the like, for example. If a drug is administered continuously over a time period (e.g., infusion), the delay may be determined from the first moment of drug is introduced to the subject, from the time the drug administration ceases, or a point in-between (e.g., administration time frame midpoint or other point).

Biomarker Detection

The presence, absence or amount of one or more biomarkers may be determined by any suitable method known in the art, and non-limiting determination methods are described herein. Determining the presence, absence or amount of a biomarker sometimes comprises use of a biological assay. In a biological assay, one or more signals detected in the assay can be converted to the presence, absence or amount of a biomarker. Converting a signal detected in the assay can comprise, for example, use of a standard curve, one or more standards (e.g., internal, external), a chart, a computer program that converts a signal to a presence, absence or amount of biomarker, and the like, and combinations of the foregoing.

The presence, absence or amount of a biomarker can be determined within a subject (e.g., in situ) or outside a subject (e.g., ex vivo). In some embodiments, presence, absence or amount of a biomarker can be determined in cells (e.g., differentiated cells, stem cells), and in certain embodiments, presence, absence or amount of a biomarker can be determined in a substantially cell-free medium (e.g., in vitro). The term “identifying the presence, absence or amount of a biomarker in a subject” as used herein refers to any method known in the art for assessing the biomarker and inferring the presence, absence or amount in the subject (e.g., in situ, ex vivo or in vitro methods).

Biomarker detected in an assay can be full-length biomarker, a biomarker fragment, an altered or modified biomarker (e.g., biomarker derivative, biomarker metabolite), or sum of two or more of the foregoing, for example. Modified biomarkers often have substantial sequence identity to a biomarker described herein. For example, percent identity between a modified biomarker and a biomarker described herein may be in the range of 15-20%, 20-30%, 31-40%, 41-50%, 51-60%, 61-70%, 71-80%, 81-90% and 91-100%, (e.g. 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 percent identity). A modified biomarker often has a sequence (e.g., amino acid sequence or nucleotide sequence) that is 90% or more identical to a sequence of a biomarker described herein. Percent sequence identity can be determined using alignment methods known in the art.

Detection of biomarkers may be performed using any suitable method known in the art, including, without limitation, mass spectrometry, antibody assay (e.g., ELISA), nucleic acid affinity, microarray hybridization, Northern blot, reverse PCR and RT-PCR. For example, RNA purity and concentration may be determined spectrophotometrically (260/280>1.9) on a Nanodrop 1000. RNA quality may be assessed using methods known in the art (e.g., Agilent 2100 Bioanalyzer; RNA 6000 Nano LabChip® and the like).

MicroRNA can be isolated and/or synthesized for use in determination and therapeutic methods described herein. MicroRNAs may be isolated using known molecular biology techniques including nucleic acid amplification (e.g., PCR), transfection, and transduction. In some embodiments microRNAs may be synthesized using synthetic methods known in the art.

MicroRNA may be detected using an array. After an array or a set of probes is prepared and/or the nucleic acid in the sample or probe is labeled, the population of target nucleic acids is contacted with the array or probes under hybridization conditions, where such conditions can be adjusted, as desired, to provide for an optimum level of specificity in view of the particular assay being performed. Suitable hybridization conditions are known in the art.

A single array or set of probes may be contacted with multiple samples. The samples may be labeled with different labels to distinguish the samples. For example, a single array can be contacted with a tumor tissue sample, and a normal tissue sample. Differences between the samples for particular microRNAs corresponding to probes on the array can be readily ascertained and quantified.

The small surface area of the array permits uniform hybridization conditions, such as temperature regulation and salt content. Moreover, because of the small area occupied by the high density arrays, hybridization may be carried out in extremely small fluid volumes (e.g., about 250 μl or less, including volumes of about or less than about 5, 10, 25, 50, 60, 70, 80, 90, 100 μl, or any range derivable therein). In small volumes, hybridization may proceed very rapidly.

Indication for Adjusting or Maintaining Subsequent Drug Dose

An indication for adjusting or maintaining a subsequent drug dose can be based on the presence or absence of a biomarker. For example, when (i) low sensitivity determinations of biomarker levels are available, (ii) biomarker levels shift in response to a drug, (iii) detectable levels of biomarker are present, and/or (iv) a drug is not appreciably toxic at levels of administration, presence or absence of a biomarker can be sufficient for generating an indication of adjusting or maintaining a subsequent drug dose.

An indication for adjusting or maintaining a subsequent drug dose often is based on the amount or level of a biomarker. An amount of a biomarker can be a mean, median, nominal, range, interval, maximum, minimum, or relative amount, in some embodiments. An amount of a biomarker can be expressed with or without a measurement error window in certain embodiments. An amount of a biomarker in some embodiments can be expressed as a biomarker concentration, biomarker weight per unit weight, biomarker weight per unit volume, biomarker moles, biomarker moles per unit volume, biomarker moles per unit weight, biomarker weight per unit cells, biomarker volume per unit cells, biomarker moles per unit cells and the like. Weight can be expressed as femtograms, picograms, nanograms, micrograms, milligrams and grams, for example. Volume can be expressed as femtoliters, picoliters, nanoliters, microliters, milliliters and liters, for example.

Moles can be expressed in picomoles, nanomoles, micromoles, millimoles and moles, for example. In some embodiments, unit weight can be weight of subject or weight of sample from subject, unit volume can be volume of sample from the subject (e.g., blood sample volume) and unit cells can be per one cell or per a certain number of cells (e.g., micrograms of biomarker per 1000 cells). In some embodiments, an amount of biomarker determined from one tissue or fluid can be correlated to an amount of biomarker in another fluid or tissue, as known in the art. For example, if the amount of a biomarker is determined in circulating blood, the amount of the biomarker can be extrapolated to the amount in melanoma cells, in certain embodiments.

An indication for adjusting or maintaining a subsequent drug dose often is generated by comparing a determined level of biomarker in a subject to a predetermined level of biomarker. A predetermined level of biomarker sometimes is linked to a therapeutic or efficacious amount of drug in a subject (e.g., melanoma cells of a subject), sometimes is linked to a toxic level of a drug, sometimes is linked to presence of a condition, sometimes is linked to a treatment midpoint and sometimes is linked to a treatment endpoint, in certain embodiments. A predetermined level of a biomarker sometimes includes time as an element, and in some embodiments, a threshold is a time-dependent signature.

Some treatment methods comprise (i) administering a drug to a subject in one or more administrations (e.g., 1, 2, 3, 4, 5, 6, 7, 8, 9 or 10 doses), (ii) determining the presence, absence or amount of a biomarker in or from the subject after (i), (iii) providing an indication of increasing, decreasing or maintaining a subsequent dose of the drug for administration to the subject, and (iv) optionally administering the subsequent dose to the subject, where the subsequent dose is increased, decreased or maintained relative to the earlier dose(s) in (i). In some embodiments, presence, absence or amount of a biomarker is determined after each dose of drug has been administered to the subject, and sometimes presence, absence or amount of a biomarker is not determined after each dose of the drug has been administered (e.g., a biomarker is assessed after one or more of the first, second, third, fourth, fifth, sixth, seventh, eighth, ninth or tenth dose, but not assessed every time after each dose is administered).

An indication for adjusting a subsequent drug dose can be considered a need to increase or a need to decrease a subsequent drug dose. An indication for adjusting or maintaining a subsequent drug dose can be considered by a clinician, and the clinician may act on the indication in certain embodiments. In some embodiments, a clinician may opt not to act on an indication.

Thus, a clinician can opt to adjust or not adjust a subsequent drug dose based on the indication provided.

An indication of adjusting or maintaining a subsequent drug dose, and/or the subsequent drug dosage, can be provided in any convenient manner. An indication may be provided in tabular form (e.g., in a physical or electronic medium) in some embodiments. For example, a biomarker threshold may be provided in a table, and a clinician may compare the presence, absence or amount of the biomarker determined for a subject to the threshold. The clinician then can identify from the table an indication for subsequent drug dose. In certain embodiments, an indication can be presented (e.g., displayed) by a computer after the presence, absence or amount of a biomarker is provided to computer (e.g., entered into memory on the computer). For example, presence, absence or amount of a biomarker determined for a subject can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount. A subsequent dose can be determined based on certain factors other than biomarker presence, absence or amount, such as weight of the subject, one or more metabolite levels for the subject (e.g., metabolite levels pertaining to liver function) and the like, for example.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

A subject can be prescreened to determine whether or not the presence, absence or amount of a particular biomarker should be determined. Non-limiting examples of prescreens include identifying the presence or absence of a genetic marker (e.g., polymorphism, particular nucleotide sequence); identifying the presence, absence or amount of a particular metabolite (e.g., a metabolite indicative of tumor activity, tissue integrity, tissue invasion, organ invasion, liver activity, kidney activity). A prescreen result can be used by a clinician in combination with the presence, absence or amount of a biomarker to determine whether a subsequent drug dose should be adjusted or maintained.

Tables E, F and G hereafter show the fold change in levels of particular miRNAs that are under-represented, or over-represented, in melanoma samples relative to non-melanoma samples. Such fold change values can be utilized as threshold values upon which adjusting or maintaining a subsequent drug dose can be based, in certain embodiments.

Roles of Particular miRNA Biomarkers in Melanoma

It has been determined that certain miRNA are correlated with certain aspects of melanoma. Certain miRNA have been correlated with melanoma cell apoptosis, melanoma cell proliferation, melanoma metastasis and drug (e.g., DTIC) resistance of melanoma, for example. Tables A, B, C and D specify representative miRNA associated with such functions. The tables also show whether each miRNA acts as (i) a tumor suppressor (miRNA-TS), as it is under-represented in melanoma cells relative to non-melanoma cells, or (ii) an oncogene (miRNA-onco), as it is over-represented in melanoma cells relative to non-melanoma cells. Table 1 hereafter shows the nucleotide sequences of such miRNA (“mature sequence”) and precursor nucleotide sequences for the miRNA (“pri-miR Sequence”).

TABLE A
Representative miRNA Correlated with Apoptosis

| miRNA- | Effect |
|---|---|
| 10 (e.g., 10a) | miRNA-TS |
| 21 | miRNA-onco |
| 126 | miRNA-TS |
| 146 (e.g., 146a) | miRNA-onco |
| 193 (e.g., 193b) | miRNA-TS |
| 203 | miRNA-TS |
| 506-514 members and | miRNA-onco |
| clusters | |

TABLE B
Representative miRNA Correlated with Cell Proliferation

| miRNA- | Effect |
|---|---|
| 21 | miRNA-onco |
| 126 | miRNA-TS |
| 146 (e.g., 146a) | miRNA-onco |
| 155 | miRNA-onco |
| 193 (e.g., 193b) | miRNA-TS |
| 206 | miRNA-TS |
| 506-514 members and | miRNA-onco |
| clusters | |

TABLE C
Representative miRNA Correlated with Metastasis

| miRNA- | Effect |
|---|---|
| 7 | miRNA-TS |
| 21 | miRNA-onco |
| 146 (e.g., 146a) | miRNA-onco |
| 193 (e.g., 193b) | miRNA-TS |
| 206 | miRNA-TS |
| 506-514 members and | miRNA-onco |
| clusters | |

miRNA Tumor Suppressors (miRNA-TS)

As shown in the Tables A, B, and C, certain miRNA function as tumor suppressors of melanoma, in that they are under-represented in melanoma cells. Table D shows the fold-reduction in the level of miRNA in a melanoma sample (e.g., melanoma cells, a blood sample from a subject having melanoma) relative to a normal sample (e.g., non-melanoma cells, a blood sample from a subject not having melanoma) for miRNA correlated with decreased apoptosis, increased cell proliferation and/or increased metastasis.

TABLE D
Tumor Suppressor miRNA Correlated with Apoptosis,
Cell Proliferation and/or Metastasis

| miRNA- | Fold-reduction relative to level in normal cells |
|---|---|
| 1 | about 52 to about 79 (e.g., miRNA-1-1 and -1-2) |
| Let7 | about 4.1 to about 6.2 (e.g., miRNA-let7b) |
| | about 12 to about 18 (e.g., miRNA-let7c) |
| 10 | about 2.5 to about 3.8 (e.g., miRNA-10a) |
| 26 | about 3.0 to about 4.6 (e.g., miRNA-26b) |
| 126 | about 5.1 to about 7.7 |
| 127 | about 3.9 to about 5.9 |
| 193 | about 20 to about 31 (e.g., miRNA-193b) |
| 195 | about 7.2 to about 10.8 |
| 199 | about 5.5 to about 8.3 (e.g., miRNA-199a-1 and -199a-2) |
| 200 | about 42 to about 64 (e.g., miRNA-200a) |
| | about 32 to about 48 (e.g., miRNA-200c) |
| 203 | about 96 to about 144 |
| 206 | about 33 to about 51 |
| 375 | about 16 to about 25 |
| 411 | about 6.1 to about 9.2 |
| 551 | about 0.5 to about 0.8 (e.g., miRNA-551b) |

In some embodiments, an indication to maintain or reduce a subsequent drug dose is provided when the amount of a miRNA biomarker in Table D determined for a treated subject is greater than the reduced amount shown in the table. For example, a subsequent dose may be maintained or reduced when the level of a miRNA-206 is greater than the level that is 33-fold to 51-fold reduced relative to the level in a non-melanoma sample.

In certain embodiments, an indication to maintain or reduce a subsequent dose is provided when the level of a miRNA biomarker is greater than a certain percentage of the fold-reduced level shown in the Table D. The certain percentage in some embodiments is about 10%, 20%, 30%, 40%, 50%, 60%, 70% or 80%. For example, a subsequent dose may be maintained or reduced when the level of a miRNA-206 is greater than a level that is 13.2-fold reduced (33-fold decreased ×40%) relative to the level in a normal sample.

In some embodiments, an indication to increase a subsequent drug dose is provided when the amount of a miRNA biomarker in Table D determined for a treated subject is about the same as, or less than, the reduced amount shown in the table. For example, a subsequent dose may be increased when the level of a miRNA-206 is about the same as, or less than, the level that is 33-fold to 51-fold reduced relative to the level in a non-melanoma sample.

In certain embodiments, an indication to increase a subsequent dose is provided when the level of a miRNA biomarker is less than a certain percentage of the fold-reduced level. The certain percentage in some embodiments is about 60%, 70%, 80% or 90%. For example, a subsequent dose may be increased when the level of a miRNA-206 is less than a level that is 26.4-fold reduced (33-fold decreased×80%) relative to the level in a normal sample.

An indication to maintain, decrease, or increase a subsequent drug dose can be provided based upon determining the presence, absence or amount of one or more of the miRNA biomarkers shown in Table D. For example, 1, 2, 3, 4, 5, 6, 7, 8, 9, 10, 11, 12, 13, 14 or 15 of the biomarkers shown in Table D, in any suitable combination, can be utilized to provide an indication to maintain, decrease, or increase a subsequent drug dose. An indication to maintain, decrease, or increase a subsequent drug dose may be determined using one or more other biomarkers in conjunction with the one or more biomarkers shown in Table D, in some embodiments.

miRNA Oncogenes (miRNA-Onco)

As shown in Tables A, B, and C, some miRNA function as oncogenes for melanoma, in that they are over-represented in melanoma cells. Table E shows the fold-increase in the level of miRNA in a melanoma sample (e.g., melanoma cells, a blood sample from a subject having melanoma) relative to a normal sample (e.g., non-melanoma cells, a blood sample from a subject not having melanoma) for miRNA correlated with decreased apoptosis, increased cell proliferation and/or increased metastasis. “506-514 members and clusters” include microRNA-506, microRNA-507, microRNA-508, microRNA-509, microRNA-510, microRNA-513, microRNA-514, the microRNA-506-514 cluster and the microRNA-506-513 cluster.

TABLE E
Oncogene miRNA

| miRNA- | Fold-increase relative to level in normal cells |
|---|---|
| 21 | about 2.8 to about 4.3 |
| 31 | about 3.5 to about 5.3 |
| 146 | about 6.6 to about 10.0 (e.g., miRNA-146a) |
| 155 | about 2.8 to about 4.3 |
| 211 | about 8.8 to about 13.2 |
| 506-514 | about 18.5 to about 28.0 (e.g., miRNA-509-1, 2 and 3) |
| members and | |
| clusters | |

In some embodiments, an indication to maintain or reduce a subsequent drug dose is provided when the amount of a miRNA biomarker in Table E determined for a treated subject is less than the increased amount shown in the table. For example, a subsequent dose may be maintained or reduced when the level of a miRNA-146 is less than the level that is 6.6-fold to 10.0-fold increased relative to the level in a non-melanoma sample.

In certain embodiments, an indication to maintain or reduce a subsequent dose is provided when the level of a miRNA biomarker is less than a certain percentage of the fold-increased level shown in the Table E. The certain percentage in some embodiments is about 10%, 20%, 30%, 40%, 50%, 60%, 70% or 80%. For example, a subsequent dose may be maintained or reduced when the level of a miRNA-146 is less than a level that is 2.6-fold increased (6.6-fold increased×40%) relative to the level in a normal sample.

In some embodiments, an indication to increase a subsequent drug dose is provided when the amount of a miRNA biomarker in Table E determined for a treated subject is about the same as, or greater than, the reduced amount shown in the table. For example, a subsequent dose may be increased when the level of a miRNA-146 is about the same as, or greater than, the level that is 6.6-fold to 10.0-fold increased relative to the level in a non-melanoma sample.

In certain embodiments, an indication to increase a subsequent dose is provided when the level of a miRNA biomarker is greater than a certain percentage of the fold-reduced level. The certain percentage in some embodiments is about 60%, 70%, 80% or 90%. For example, a subsequent dose may be increased when the level of a miRNA-146 is greater than the level that is 5.3-fold increased (6.6-fold increased×80%) relative to the level in a normal sample.

An indication to maintain, decrease, or increase a subsequent drug dose can be provided based upon determining the presence, absence or amount of one or more of the miRNA biomarkers shown in Table E. For example, 1, 2, 3, 4, 5, 6, 7, 8, 9, 10, 11, 12, 13, 14 or 15 of the biomarkers shown in Table E, in any suitable combination, can be utilized to provide an indication to maintain, decrease, or increase a subsequent drug dose. An indication to maintain, decrease, or increase a subsequent drug dose may be determined using one or more other biomarkers in conjunction with the one or more biomarkers shown in Table E, in some embodiments.

Preparation of Molecules

Certain molecules can be prepared for use in methods described herein. Molecules can be used as a control or standard in an assay or as an active ingredient in a therapeutic, in some embodiments.

Nucleic Acid Preparation

In some embodiments, a nucleic acid is provided for use as a control or standard in an assay, or therapeutic, for example. A nucleic acid may be made by any technique known in the art, such as for example, chemical synthesis, enzymatic production or biological production. Nucleic acids may be recovered or isolated from a biological sample. The nucleic acid may be recombinant or it may be natural or endogenous to the cell (produced from the cell's genome). It is contemplated that a biological sample may be treated in a way so as to enhance the recovery of small nucleic acid molecules such as microRNA. Generally, methods may involve lysing cells with a solution having guanidinium and a detergent.

Nucleic acid synthesis may also be performed according to standard methods. Non-limiting examples of a synthetic nucleic acid (e.g., a synthetic oligonucleotide), include a nucleic acid made by in vitro chemical synthesis using phosphotriester, phosphite, or phosphoramidite chemistry and solid phase techniques or via deoxynucleoside H-phosphonate intermediates. Various different mechanisms of oligonucleotide synthesis have been disclosed elsewhere.

Nucleic acids may be isolated using known techniques. In particular embodiments, methods for isolating small nucleic acid molecules, and/or isolating RNA molecules can be employed. Chromatography is a process used to separate or isolate nucleic acids from protein or from other nucleic acids. Such methods can involve electrophoresis with a gel matrix, filter columns, alcohol precipitation, and/or other chromatography. If a nucleic acid, for example microRNA, from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating, for example, a microRNA from other nucleic acids, a gel matrix may be prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly microRNA used in methods and compositions herein provided. Generally, small RNA molecules may be isolated from cells by methods comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column is effective for such isolation procedures.

A nucleic acid isolation processes may sometimes include: a) lysing cells in the sample with a lysing solution comprising guanidinium, where a lysate with a concentration of at least about 1 M guanidinium is produced; b) extracting nucleic acid molecules from the lysate with an extraction solution comprising phenol; c) adding to the lysate an alcohol solution for form a lysate/alcohol mixture, wherein the concentration of alcohol in the mixture is between about 35% to about 70%; d) applying the lysate/alcohol mixture to a solid support; e) eluting the nucleic acid molecules from the solid support with an ionic solution; and, f) capturing the nucleic acid molecules. The sample may be dried down and resuspended in a liquid and volume appropriate for subsequent manipulation.

Antibodies and Small Molecules

In some embodiments, an antibody or small molecule is provided for use as a control or standard in an assay, or a therapeutic, for example. In some embodiments, an antibody or other small molecule configured to bind to a melanoma cell. An antibody or small molecules may sometimes bind to an mRNA structure encoding for an over-expressed protein.

The term small molecule as used herein means an organic molecule of approximately 1000, 800 or fewer Daltons. In certain embodiments small molecules may diffuse across cell membranes to reach intercellular sites of action. In some embodiments a small molecule binds with high affinity to a biopolymer such as protein, nucleic acid, or polysaccharide and may sometimes alter the activity or function of the biopolymer. In various embodiments small molecules may be natural (such as secondary metabolites) or artificial (such as antiviral drugs); they may have a beneficial effect against a disease (such as drugs) or may be detrimental (such as teratogens and carcinogens).

By way of non-limiting example, small molecules may include ribo- or deoxyribonucleotides, amino acids, monosaccharides and small oligomers such as dinucleotides, peptides such as the antioxidant glutathione, and disaccharides such as sucrose.

The term antibody as used herein is to be understood as meaning a gamma globulin protein found in blood or other bodily fluids of vertebrates, and used by the immune system to identify and neutralize foreign objects, such as bacteria and viruses. Antibodies typically include basic structural units of two large heavy chains and two small light chains.

Specific binding to an antibody requires an antibody that is selected for its affinity for a particular protein. For example, polyclonal antibodies raised to a particular protein, polymorphic variants, alleles, orthologs, and conservatively modified variants, or splice variants, or portions thereof, can be selected to obtain only those polyclonal antibodies that are specifically immunoreactive with melanoma marker proteins or over-expressed proteins and not with other proteins. This selection may be achieved by subtracting out antibodies that cross-react with other molecules.

A drug may be an antibody or a fragment thereof. Antibodies sometimes are IgG, IgM, IgA, IgE, or an isotype thereof (e.g., IgG1, IgG2a, IgG2b or IgG3), sometimes are polyclonal or monoclonal, and sometimes are chimeric, humanized or bispecific versions of such antibodies. Polyclonal and monoclonal antibodies that bind specific antigens are commercially available, and methods for generating such antibodies are known. In general, polyclonal antibodies are produced by injecting an isolated antigen into a suitable animal (e.g., a goat or rabbit); collecting blood and/or other tissues from the animal containing antibodies specific for the antigen and purifying the antibody. As used herein, the term “monoclonal” is to be understood as designating an antibody (or its corresponding fragment) arising from a single clone of an antibody-producing cell such as a B cell, and recognizing a single epitope on the antigen bound. Methods for generating monoclonal antibodies, in general, include injecting an animal with an isolated antigen (e.g., often a mouse or a rat); isolating splenocytes from the animal; fusing the splenocytes with myeloma cells to form hybridomas; isolating the hybridomas and selecting hybridomas that produce monoclonal antibodies which specifically bind the antigen. Examples of monoclonal antibodies are anti MDM 2 antibodies, anti-p53 antibodies (pAB421, DO 1, and an antibody that binds phosphoryl-ser15), anti-dsDNA antibodies and anti-BrdU antibodies, are described hereafter.

Methods for generating chimeric and humanized antibodies also are known and sometimes involve transplanting an antibody variable region from one species (e.g., mouse) into an antibody constant domain of another species (e.g., human). Antigen-binding regions of antibodies (e.g., Fab regions) include a light chain and a heavy chain, and the variable region is composed of regions from the light chain and the heavy chain. Given that the variable region of an antibody is formed from six complementarity-determining regions (CDRs) in the heavy and light chain variable regions, one or more CDRs from one antibody can be substituted (i.e., grafted) with a CDR of another antibody to generate chimeric antibodies. Also, humanized antibodies are generated by introducing amino acid substitutions that render the resulting antibody less immunogenic when administered to humans.

The drug sometimes is an antibody fragment, such as a Fab, Fab′, F(ab)′2, Dab, Fv or single-chain Fv (ScFv) fragment, and methods for generating antibody fragments are known. In some embodiments, a binding partner in one or more hybrids is a single-chain antibody fragment, which sometimes are constructed by joining a heavy chain variable region with a light chain variable region by a polypeptide linker (e.g., the linker is attached at the C-terminus or N-terminus of each chain) by recombinant molecular biology processes. Such fragments often exhibit specificities and affinities for an antigen similar to the original monoclonal antibodies. Bifunctional antibodies sometimes are constructed by engineering two different binding specificities into a single antibody chain and sometimes are constructed by joining two Fab′ regions together, where each Fab′ region is from a different antibody. Antibody fragments may comprise engineered regions such as CDR-grafted or humanized fragments. In certain embodiments the drug is an intact immunoglobulin, and in some embodiments the drug may be a Fab monomer or a Fab dimer.

In various embodiments, the antibody or fragment thereof specifically binds to an epitope, including in some embodiments to a discontinuous epitope, of a melanoma marker or ever-expressed protein. In some embodiments antibodies may be configured to recognize such a protein highly specifically, that is to say that from a mixture of the target molecule and other molecules. This means that, for example, a monoclonal antibody or fragment thereof according to these embodiments, when administered to a subject, may be expected to specifically bind to and neutralize only the desired target, whereas other undesired targets are neither bound nor neutralized. In certain embodiments an antibody drug may bind to a melanoma marker protein or over-expressed protein with extremely high affinity, meaning that that once the complex between a monoclonal antibody or fragment thereof on the one hand and the target molecule on the other hand is formed, it does not readily, or at least does not quickly separate.

Pharmaceutical Formulations

A molecule described herein can be prepared in a pharmaceutically acceptable formulation. The phrase “pharmaceutically acceptable” refers to molecular entities and compositions that do not produce an allergic or other untoward reaction when administered to a human. Solutions of active pharmaceutical agents described herein can be prepared as free base or pharmacologically acceptable salts. Such agents also may be prepared in water suitably mixed with a surfactant, such as hydroxypropylcellulose, in some embodiments. Dispersions may also be prepared in glycerol, liquid polyethylene glycols, mixtures thereof, and in oils. Under ordinary conditions of storage and use, these preparations can contain a preservative to prevent the growth of microorganisms. The pharmaceutical forms suitable for injectable use include sterile aqueous solutions or dispersions and sterile powders for the extemporaneous preparation of sterile injectable solutions or dispersions. The form is often sterile and fluid to the extent that easy syringability exists. It may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be achieved by various antibacterial and antifungal agents, for example, parabens, chlorobutanol, phenol, sorbic acid, thimerosal, and the like. In many cases isotonic agents, for example sugars or sodium chloride, may be included. Prolonged absorption of the injectable compositions can be augmented by the use in the compositions of agents delaying absorption, for example, aluminum monostearate and gelatin.

In certain formulations, a water-based formulation is employed while in others, it may be lipid-based. In particular embodiments, a composition comprising an active pharmaceutical agent or a nucleic acid encoding the same is in a water-based formulation. In other embodiments, the formulation is lipid based.

For parenteral administration in an aqueous solution, for example, the solution is often suitably buffered if necessary and the liquid diluent first rendered isotonic with sufficient saline or glucose. These particular aqueous solutions are suitable for intravenous, intramuscular, subcutaneous, intralesional, and intraperitoneal administration. In this connection, sterile aqueous media which can be employed are known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage may necessarily occur depending on the condition of the subject being treated. The person responsible for administration may, in any event, determine the appropriate dose for the individual subject. Moreover, for human administration, preparations should meet sterility, pyrogenicity, general safety and purity standards as required by FDA Office of Biologics standards.

As used herein, a “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Drug Administration

A drug can be administered to any appropriate subject having a biomarker or needing treatment for a condition described herein. Non-limiting examples of a subject include mammal, human, ape, monkey, ungulate (e.g., equine, bovine, caprine, ovine, porcine, buffalo, camel and the like), canine, feline, rodent (e.g., murine, mouse, rat) and the like. A subject may be male or female, and a drug can be administered to a subject in a particular age group, including, for example, juvenile, pediatric, adolescent, adult and the like.

Any suitable drug can be administered for treating a melanoma. In certain embodiments a drug exerts anti-proliferation effects. In some embodiments, a drug may exert immunosuppressive effects. A drug, in certain embodiments, comprises as an active ingredient an antibody, antibody fragment, single-chain antibody, small molecule, a nucleic acid, nucleic acid derivative, microRNA, (including, without limitation, a microRNA-1, a microRNA-10a, a microRNA-21, a microRNA 27a, a microRNA-31, a microRNA-126, a microRNA 146, a microRNA-155, a microRNA-193, a microRNA-193b, a microRNA-203, a microRNA-211, a microRNA-506, microRNA-507, microRNA-508, a microRNA-509, a microRNA-510, a microRNA-513, microRNA-514, microRNA-506-514 cluster, microRNA-506-513 cluster, and an associated subtype, or combination of the foregoing), microRNA inhibitor, siNA, peptide, polypeptide, protein antibody, antibody fragment, single-chain antibody, small molecule, and the like. Various forms of microRNA or siRNA may be delivered, including post-processed microRNA or siRNA, pre-processed microRNA (e.g., “pri-miRNA”) or siRNA or vector that encodes pre-processed or post-processed microRNA or siRNA. In certain embodiments a drug active ingredient sometimes interacts with a biomarker described herein, sometimes is capable of specifically binding to the biomarker, sometimes modulates the expression, persistence or level of the biomarker, and sometimes is capable of modifying the structure of the biomarker, in certain embodiments.

Methods as presented herein include without limitation the delivery of an effective amount of a microRNA or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease. In some embodiments there may be a step of monitoring the biomarkers to evaluate the effectiveness of treatment and to control toxicity.

Drugs are administered in a manner compatible with the dosage formulation, and in such amount as may be therapeutically effective. Injection of nucleic acids may be delivered by syringe or any other method used for injection of a solution, as long as the nucleic acid and any associated components can pass through the particular gauge of needle required for injection. A syringe system for use in gene therapy that permits multiple injections of predetermined quantities of a solution precisely at any depth is known in the art.

The quantity to be administered depends on the subject to be treated, including, e.g., the aggressiveness of the disease, the size of the affected area, and the previous or other courses of treatment. Precise amounts of active ingredient required to be administered depend on the judgment of the practitioner. Suitable regimes for initial administration and subsequent administration are also variable, but are typified by an initial administration followed by other administrations. Moreover, administration may be through a time release or sustained release mechanism, implemented by formulation and/or mode of administration.

The routes of administration may vary with the location and nature of the site to be targeted, and include, e.g., intratumoral, intramuscular, intradermal, subcutaneous, regional, parenteral, intravenous, intranasal, systemic, and oral administration and formulation. Injection may be direct injection, intratumoral injection, or injection into vasculature of the affected melanoma cell or tumor region. Local, regional, or systemic administration also may be appropriate. Drugs may be administered in multiple injections to a targeted site.

Treatment regimens may vary as well and often depend on melanoma type, location, immune condition, target site, disease progression, and health and age of the patient. Certain melanoma types may require more aggressive treatment. The clinician may be best suited to make such decisions based on the known efficacy and toxicity (if any) of the therapeutic formulations.

Treatments may include various “unit doses.” A unit dose is defined as containing a predetermined quantity of a drug. The quantity to be administered, and the particular route and formulation, are within the skill of those in the clinical arts. A unit dose need not be administered as a single injection but may comprise continuous infusion over a set period of time. With respect to a viral component as presented herein, a unit dose may conveniently be described in terms of mg of nucleic acid or nucleic acid mimetic. Alternatively, the amount specified may be the amount administered as the average daily, average weekly, or average monthly dose.

Some embodiments involve drug dose escalation, where (i) a relatively low dose of a drug is administered to a subject, (ii) presence, absence or amount of a biomarker is assessed, and if the assessment indicates there is no significant efficacious effect of the drug, then (iii) administering a subsequent higher dose of the drug to a subject, where (ii) and (iii) are repeated until a therapeutic effect is observed (e.g., the therapeutic effect may be observed based on the biomarker assessment). Such an approach can allow a clinician to “dial-in” an efficacious amount of a drug for a subject and minimize toxic side effects associated with higher amounts of the drug. A clinician, in some embodiments, may have information pertaining to the amount of drug administered that is likely to result in a significant toxic side effect for subjects and cease administration of drug if a subsequent dose is increased and is expected to have significant toxic side effects (e.g., a clinician may cease administration at or near a specified toxic threshold dose of the drug).

Administration can be in vivo, ex vivo or in vitro. A drug may be prepared as a pharmaceutically acceptable salt in some embodiments. A drug may be prepared as a pharmaceutically acceptable formulation, in certain embodiments, that comprises, for example, one or more of a liposome or other polymatrix, a penetration enhancer, surfactant, fatty acid, bile salt, carrier, excipient, adjuvant and the like.

For in vivo administration, a drug may be formulated for any convenient route of administration, including, without limitation, nasal, topical, oral, pulmonary, parenteral, intrathecal, and intranutrical administration. A drug can be prepared in a unit dosage form for systemic administration, in some embodiments, and may be incorporated into a hard or soft shell gelatin capsule, may be compressed into a tablet, or may be incorporated directly in food of subject's diet, for example. For oral therapeutic administration, a drug may be combined with one or more excipients and used in the form of ingestible tablets, buccal tablets, troches, capsules, elixirs, suspensions, syrups, wafers and the like, in certain embodiments. Such preparations sometimes contain at least 0.001% to 0.1% of active drug by weight and sometimes between about 0.1% to about 60% of the weight of the given unit dosage form.

For in vitro administration a drug may be delivered to cells by any convenient method. For example, a drug may be formulated as described herein, and in some embodiments, a pharmaceutical formulation (e.g., nucleic acid drug) may be exposed to calcium phosphate or calcium chloride co-precipitation, transduction/infection, DEAE-dextran-mediated transfection, lipofection, electroporation, and iontophoresis.

In some embodiments, in vitro administration may be applicable to methods pertaining to drug screening or selection. In certain embodiments, such methods comprise (i) contacting melanoma cells with a drug in vitro, (ii) determining the presence, absence or amount of a biomarker associated with a melanoma, and (iii) selecting a drug for further screening or administration to a subject having a melanoma based on the presence, absence or amount of the biomarker. In some embodiments, a drug is selected for further screening or administration if the amount of an over-represented biomarker is reduced or is absent. In certain embodiments, a drug is selected for further screening if an under-represented biomarker is increased or present.

Melanoma cells may be obtained from primary tissue culture, where the affected tissue is from any suitable source of the body (e.g., skin, blood, organs, bone, muscle and the like) in certain embodiments.

Cancer Treatments

Methods and compositions herein may be useful for the treatment or prevention of a variety of cancers or other abnormal proliferative diseases. Cancers and related disorders that can be treated, prevented, or managed by methods and compositions provided herein include, but are not limited to, cancers of an epithelial cell origin.

The terms “treat” and “treating” as used herein refer to (i) preventing a pathologic condition from occurring (e.g. prophylaxis); (ii) inhibiting the pathologic condition or arresting its development; (iii) relieving the pathologic condition; and/or (iv) ameliorating, alleviating, lessening, and removing symptoms of a disease or condition. A candidate molecule or compound described herein may be in a therapeutically effective amount in a formulation or medicament, which is an amount that can lead to a biological effect (e.g., inhibiting inflammation), or lead to ameliorating, alleviating, lessening, relieving, diminishing or removing symptoms of a disease or condition, for example. The terms also can refer to reducing or stopping a cell proliferation rate (e.g., slowing or halting tumor growth) or reducing the number of proliferating cancer cells (e.g., removing part or all of a tumor). A molecule described herein can be administered to a subject in need thereof to potentially treat a melanoma. In such treatments, the terms “treating,” “treatment” and “therapeutic effect” can refer to reducing or stopping a cell proliferation rate (e.g., slowing or halting tumor growth), reducing the number of proliferating cancer cells (e.g., ablating part or all of a tumor) and alleviating, completely or in part, a melanoma condition.

A drug, which can be a prophylactic or therapeutic agent, can be administered to any appropriate subject having a melanoma as described herein. Non-limiting examples of a subject include mammal, human, ape, monkey, ungulate (e.g., equine, bovine, caprine, ovine, porcine, buffalo, camel and the like), canine, feline, rodent (e.g., murine, mouse, rat) and the like. A subject may be male or female, and a drug can be administered to a subject in a particular age group, including, for example, juvenile, pediatric, adolescent, adult and the like.

Non-limiting examples of drugs include proteinaceous molecules (e.g., peptides, polypeptides, proteins, post-translationally modified proteins, antibodies and the like); small molecules (e.g., less than 1000 Daltons); inorganic or organic compounds; or nucleic acid molecules (e.g., double-stranded or single-stranded DNA, double-stranded or single-stranded RNA, triple helix nucleic acid molecules). In some embodiments, a drug comprises a nucleic acid that includes a nucleotide sequence of a microRNA molecule described herein. A drug can be derived from any known organism (including, but not limited to, animals, plants, bacteria, fungi, and protista, or viruses) or can be a synthetic molecule.

In some embodiments, a drug is a cancer therapeutic used for chemotherapy. Non-limiting examples of cancer therapeutics include an alkylating agent, a protein kinase modulator, a tumor suppressor protein modulator, and/or an angiogenesis inhibitor, in some embodiments. An example of an alkylating agent is dimethyl-triazen imidazole carboxmide (DTIC). In addition to DTIC, polyfunctional alkylating drugs include Procarbazine (Matulane), a Methylhydrazine derivative, Altretamine (Hexylen) and Cisplatin (Platinol). Alkylating agents in general effect an alkyl group transfer, with the major interaction being alkylation of DNA. A primary DNA alkylation site may be the N7 position of guanine but there may be other sites as well. The interaction may involve single or double DNA strands, with cross linking due to bifunctional (2 reactive center) characteristics. Alkylating drugs may also react with carboxyl, sulfhydryl, amino, hydroxyl, and phosphate groups of other cellular constituents. Such drugs may form ethyleneimonium ion as a reactive intermediate. Patients with advanced disease, such as lymph node involvement and distant metastases, have 5-year survival rates of 50% and 10%, respectively. This poor prognosis often results from resistance to cytotoxic drug therapy (e.g., administration of DTIC).

Combination Therapies

A therapy sometimes includes administration of two or more therapeutic agents. In some embodiments, therapy by administration of one or more microRNAs is combined with the administration of one or more therapies such as, but not limited to, chemotherapy, radiation therapy, hormone therapy, and/or biological therapy (e.g., immunotherapy). In specific embodiments, methods herein encompass administration of a microRNA described herein in combination with administration of one or more prophylactic/therapeutic agents (e.g., a cancer therapeutic). A drug suitable for treating a melanoma sometimes is administered in combination with one or more other drugs or inactive ingredients. One or more of the other drugs in a therapy may treat a melanoma in some embodiments, and sometimes, one or more of the other drugs may not specifically treat a melanoma. One or more inactive ingredients may treat a side effect of an active agent that treats the melanoma (e.g., anti-diuretic, anti-nausea, anti-diarrhea, depressant, stimulant and the like), for example. An additional drug may also enhance the curative effect of the primary drug.

In certain embodiments, a therapeutic composition can include a microRNA, complement thereof, or microRNA inhibitor molecule, or expression construct encoding the foregoing. Such compositions can be used in combination with additional therapies to enhance the effect of the therapy employed. These compositions can be provided in a combined amount effective to achieve a desired effect, such as the elimination or amelioration melanoma. This process may involve administering a microRNA (or complement or inhibitor thereof, or expression vector that encodes the foregoing) or additional therapy at the same or different time. Such a therapeutic approach may be performed by administering one or more compositions or pharmacological formulation that includes or more agents, or by administering two or more distinct compositions or formulations, where one composition provides (1) a microRNA, complement or inhibitor thereof, or expression construct that encodes the foregoing; and/or (2) another second therapy.

In certain embodiments an additional therapy, such as in non-limiting example, an immunosuppressive drug, is employed in combination with the drug therapy provided herein. Immunosuppressive drugs typically inhibit or prevent activity of the immune system. In general, such drugs can be categorized as glucocorticoids, cytostatics, antibodies, drugs acting on immunophilins, among others. In some embodiments an additional therapy is an anti-inflammatory drug. In general, such drugs can be classified as steroids, non-steroid anti-inflammatory (NSAID), cyclooxygenase (COX) inhibitor, COX-2 inhibitor, COX-1 inhibitor, non-selective COX inhibitor and others. An additional therapy may sometimes be an antibiotic drug. In general, such drugs may be classified as aminoclycosides, Ansamycins, carbacephem, carapenems, cephalosporins, glycopeptides, macrolides, monobactams, penicillins, polypeptides, quinolones, sulfonamides and tetracyclines, among others. In some embodiments an additional therapy is an anti-viral drug. In general, such drugs can be classified as non-nucleoside reverse transcriptase inhibitors, nucleoside reverse transcriptase inhibitors, protease inhibitors and nucleotide analog reverse transcriptase inhibitors, among others. In certain embodiments an additional therapy is a steroid drug. In general, such drugs may be classified as corticosteroids and anabolic steroids, among others. In some embodiments an additional therapy is a chemotherapy drug. In general, such drugs may be classified as alkylating agents, antimetabolites, anti-tumor antibiotics, topoisomerase inhibitors, mitotic inhibitors and corticosteroids, among others. An additional therapy may sometimes be a hormone therapy drug. In general, such drugs may be classified as anti-estrogens, aromatase inhibitors, progestins, estrogens, anti-androgens and LHRH agonists, among others.

### EXAMPLES

The examples set forth below illustrate certain embodiments and do not limit the technology.

### Example 1

### Methods

The function of microRNAs, that are differentially expressed in melanoma lesions compared to normal donor skin, was examined to identify microRNAs putatively involved in the oncogenesis of malignant melanoma.

Patients and Controls

Thirty-six (36) malignant melanoma skin biopsies and 16 normal skin biopsies were obtained from ILSBio (Chestertown, Md.). Melanoma samples were from both female and male caucasian patients, ages 26-81, and stages IIB to IV. 30 of 36 melanoma patients had documented metastases to various areas of the body. Normal skin samples were obtained from healthy donors.

A375 (CRL-1619), MALME-3 (HTB-102), MALME-3M (HTB-64), RPMI7951 (HTB-66), SK-MEL-2 (HTB-68), and SK-MEL-5 (HTB-70) melanoma cell lines were purchased from the American Type Culture Collection (ATCC, Manassas, Va.; ATCC.org) and cultured in DMEM (Invitrogen. Carlsbad, Calif., Cat. No: 12430-104) containing 10% Fetal Bovine serum (FBS; Invitrogen, Cat No: 16000-044). LOX, M14, M19, M21, UACC.62, and UACC.257 melanoma cell lines were developed internally (e.g., MedImmune cell line collection) and cultured in DMEM containing 10% FBS. Normal human neonatal primary epidermal melanocytes (PCS-200-012), purchased from the ATCC, were cultured in Dermal Cell Basil Media (ATCC PCS-200-030) supplemented with the Melanocyte Growth Kit (ATCC PCS-200-041). An additional normal melanocyte line, NHEM-Ad-Adult Normal Human Epidermal Melanocytes (NHEM-ad-adult) purchased from Lonza (Allendale, N.J.; Lonza.com), was cultured in Dermal Cell Basil Media supplemented with the Melanocyte Growth Kit. All cell lines were cultured in accordance with supplier protocols and suggested media. Cell culture for assays related to miRNAs in the 506-514 cluster was carried out as follows. miRNA expression patterns of a panel of melanoma cell lines were evaluated and the 5 chosen for in vitro functional assays were those that clustered most closely with the melanoma patient samples. Included in this panel were: A375, SK-MEL-2, SK-MEL-5, MALME-3M, and RPMI-7951. All cell lines were purchased from American Type Culture Collection (ATCC, Manassas, Va.) and cultivated in recommended media at 37° C. in a humidified atmosphere with 5% CO₂. Primary epidermal melanocytes also were purchased from ATCC and maintained using suggested media and growth conditions.

Total RNA Extraction and Real Time Quantitative RT-PCR Processing

Total RNA (i.e. both large and small RNA containing mRNA, miRNA, snoRNA, etc), from skin biopsies and cultured cells, were purified with the mirVana miRNA Isolation kit according to the manufacturer's protocol for total RNA (Applied Biosystems/Ambion, Austin, Tex.). RNA quality was assessed on an Agilent 2100 Bioanalyzer using RNA 6000 Nano LabChips. Biotin-labeled, amplified cRNA was generated from 2 micrograms of total RNA using the Affymetrix GeneChip One-Cycle cDNA Synthesis kit and the Affymetrix GeneChip IVT Labeling kit (Affymetrix, Santa Clara, Calif.). Twenty micrograms of each biotin-labeled cRNA was fragmented for hybridization on Affymetrix Human Genome U133 Plus 2.0 GeneChip arrays. All GeneChip washing, staining, and scanning procedures were performed with Affymetrix standard equipment. Data capture and initial array quality assessments were performed with the GeneChip Operating Software (GCOS) tool. MicroRNAs were prepared for expression profiling using the TaqMan MicroRNA Reverse Transcription kit (ABI 4366597) and Multiplex RT for TaqMan MicroRNA Assays, Human Pool Set (ABI 4384791). MicroRNA expression was quantified with the TaqMan Low-Density Human MicroRNA Array v1.0 (ABI 4384792) using standard protocols.

Real Time Quantitative RT-PCR

Real time quantitative RT-PCR for miRNAs in the microRNA-506-514 cluster was performed as follows. Relative fold change for each individual member of the miR-506-514 cluster was determined using TaqMan Low-Density Array (TLDA) microRNA Cards v3.0 (Applied Biosystems, Foster City, Calif.). Single-stranded cDNA was synthesized from 500 ng of total RNA from skin biopsies and 500 ng of total RNA from cell cultures using the Applied Biosystems TaqMan MicroRNA Reverse Transcription Kit and Megaplex RT Primers. Subsequent pre-amplification of specific cDNA targets was performed using the TaqMan Pre-Amp Master Mix kit and Megaplex Pre-Amp primers following the manufacturer's protocol. The microRNA cards were loaded and run on an Applied Biosystems 7900HT Real-Time PCR system using the following cycling parameters: 94.5° C./10 min followed by 40 cycles of 97° C./30 sec, 59.7° C./1 min. Data analysis of the resulting Ct values from each real-time PCR method was conducted with SDS v2.2.2 software (Applied Biosystems). All samples were normalized to the mean Ct value of several calibrator genes (TaqMan calibrators: U6 snRNA, RNU44, RNU45) then a pooled delta Ct value from normal skin or a delta Ct value in normal melanocytes was used to calculate relative fold change levels for each miRNA in tissue and in cell lines, respectively. Statistical analyses of relative expression ratios for both methods were conducted using the Welch's 2-sample t-test; p<0.05 was considered significant.

Samples with inconsistent expression profiles of the endogenous controls (i.e., RNU44, RNU48 and RNU6B) were excluded from further analysis. The significantly differentially expressed miRNAs were identified by t-test with a Bonferroni adjusted p-value <0.05. MicroRNA probes with an absolute fold change 2 and p-value <0.05 were considered to be differentially regulated. An absolute fold change 2 and p-value <0.01 was considered differentially regulated for miRNAs in the 500 cluster (see below for further experimental methods used for analysis of miRNAs in the 500 cluster). MicroRNAs found to be significantly over-expressed by more than 2-fold in melanoma versus normal donor samples were considered potential oncogene miRs (Onco-miRs) for further study; while those under-expressed by more than 2-fold were considered potential tumor suppressor-miRs (TS-miRs).

Functional Analysis of Differentially Expressed microRNAs

Differentially expressed microRNAs were then analyzed for predicted function in cancer phenotypes by the method previously described by Georgantas et al (PNAS 104:4344). The method was used to identify those differentially expressed microRNAs that were associated with oncology-related keywords in PubMed. Additionally, the predicted mRNA targets of microRNAs of interest were compiled from TargetScan (http://www.targetscan.org/), miRBase Targets (http://www.mirbase.org/), and PicTar (pictar.mdc-berlin.de). These mRNA targets also were interrogated against the Pubmed, OMIM, and KEGG databases to identify mRNAs associated with cancer phenotypes. Finally, mRNA targets were analyzed using Ingenuity Pathway Analysis (Ingenuity Systems, Inc, Redwood City, Calif.) and DAVID Bioinformatics Resource (http://david.abcc.ncifcrf.gov/summary.jsp) pathway analysis tools to identify cancer associated molecular networks/pathways most highly predicted to be associated with the mRNA targets, and therefore possibly affected by differentially expressed microRNAs. Those microRNAs identified using these analyses, were considered “cancer-associated” and further investigated as putative Oncogene-microRNAs (Onco-miRs) if over-expressed or Tumor Suppressor-microRNAs (TS-miRs) in under-expressed.

Transfection Reagents and Conditions

MicroRNA mimics and inhibitors. miRIDIAN microRNA hairpin inhibitors, hairpin inhibitor negative controls, miRIDIAN microRNA mimics and mimic negative controls were purchased from Dharmacon Thermo-Fisher (Lafayette, Colo.). MicroRNA inhibitors are synthetic miRNA-specific molecules designed to block miRNA expression and were used to transfect melanoma cell lines. MicroRNA mimics are double-stranded RNA oligonucleotides designed to mimic endogenous mature miRNAs and were used to transfect primary epidermal melanocytes. PrimeFect siRNA Transfection Reagent (Lonza, Walkersville, Md.) was used to transfect all mimics or inhibitors into cells according to the manufacturer's guidelines. Transfection efficiency for all cell lines was determined by utilizing labeled miRNA mimic and inhibitor controls (Dharmacon).

Transfection conditions were optimized as follows. Optimal transfection conditions with PrimeFect siRNA (Lonza, Walkersville, Md.) were determined by transfection of ALEXA 547 labeled non-targeting control miRidian microRNA mimic (250 nm) or miRidian Hairpin Inhibitor Control (10 nm). The optimization protocol included with the PrimeFect reagent was followed in 6 well plates. 24 hrs after transfection, transfection level was measured by FlowCytometry on a FACScaliber (BD). Data was analyzed with FlowJo to determine % cells transfected and mean florescent intensity. The best condition was chosen for further studies, and was scaled based on culture surface area for 384 or 96 well plates as needed.

Melanoma and melanocyte cell lines were transfected as follows. Melanoma cells were seeded in dishes appropriate to the endpoint assay the day before transfection. After approximately 24 hours, the standard culture media was replaced with Opti-MEM reduced serum media (Invitrogen). To inhibit putative onco-miRs, cells were transfected with either 10 nM of each appropriate miRNA hairpin inhibitor or inhibitor negative control. To “replace” putative TS-miRs, cells were transfected with either 100 nM of each appropriate miRNA mimic or mimic negative controls.

For assays involving miRNAs in the microRNA-506-514 cluster in melanoma and melanocyte cell lines, the following conditions and inhibitor concentrations were used. After approximately 24 hours, the standard culture media was replaced with Opti-MEM reduced serum media (Invitrogen) and cells were transfected with either 10 nM of each appropriate miRNA hairpin inhibitor or a final combined concentration of 70 nM (divided equally among the number of inhibitors examined) or 70 nM of the inhibitor negative control. Melanoma cells transfected with the following hairpin inhibitors will be referred to as follows. Inhibitors to the full cluster (e.g., 7 inhibitors in total) include miR-506, miR-507, miR-508, miR-509, miR-510, miR-513, and miR-514; inhibitors to sub-cluster A (e.g., 4 inhibitors in total) include miR-506, miR-507, miR-508, and miR-513; inhibitors; inhibitors to sub-cluster B (e.g., 3 inhibitors in total) include miR-509, miR-510, and miR-514.

Primary melanocyte cells were transfected as follows. Melanocytes were seeded in dishes appropriate to the endpoint assay the day before transfection. After approximately 24 hours, cells were transfected with either 10 nM of each appropriate miRNA mimic or 120 nM of the mimic negative control. Some members of the miR-506-514 cluster have multiple isoforms and/or multiple mature miRNAs. In order to insure appropriate over-expression of all components of the cluster, each isoform identified for a particular miRNA was included in the transfection. Melanocytes transfected with the following miRNA mimics are referred to as follows; mimics to the full cluster (e.g., 12 mimics in total) include miR-506, miR-507, miR-508-3p, miR-508-5p, miR-509-3p, miR-509-5p, miR-510, miR-5,3-a-3p, miR-5,3-a-5p, miR-5,3-b, miR-5,3-c, and miR-514; mimics to sub-cluster A (e.g., 8 mimics total) include miR-506, miR-507, miR-508-3p, miR-508-5p, miR-5,3-a-3p, miR-5,3-a-5p, miR-5,3-b, and miR-5,3-c; mimics to sub-cluster B (e.g., 4 mimics total) include miR-509-3p, miR-509-5p, miR-510, and miR-514.

Cell Growth Assay

Growth inhibition was assessed as follows. Melanoma cell lines and primary melanocytes were seeded in 384-well plates and transfected as indicated with inhibitors to the full cluster, sub-cluster A, sub-cluster B, or negative control. Cell growth was measured 3 and 5 days post-transfection using the Cell Titer-Glo Luminescent Cell Viability Assay (CTG; Promega, Madison, Wis.) according to the manufacturer's protocol. Luminescent data was collected on the EnVision Plate Reader (Perkin Elmer, Waltham, Mass.). Statistical comparisons were conducted using the Welch's 2-sample t-test; p<0.05 was considered significant.

Apoptosis Assay

Apoptosis was assayed as follows. Melanoma cell lines and primary melanocytes for were seeded in 384-well plates and transfected as indicated with inhibitors to over-expressed miRs, or microRNA mimics of under-expressed miRs. Caspase activation was measured 2 days post-transfection using the Caspase-Glo 3/7 Assay (Promega) according to the manufacturer's protocol. Luminescent data was collected on the EnVision Plate Reader (Perkin Elmer). Statistical comparisons were conducted using the Welch's 2-sample t-test; p<0.05 was considered significant. The proportion of cells undergoing apoptosis was measured using the Vybrant Apoptosis Assay Kit #2 with Alexa Fluor 488 annexin V/propidium iodide (PI; Invitrogen). Briefly, melanoma cell lines were seeded in 6-well plates at 500,000 cells per well and transfected as described above. Cells were removed from the dishes using TrypLE Express stable trypsin-like enzyme (Invitrogen), washed with cold PBS, centrifuged, and resuspended in annexin-binding buffer at 1×10⁶ cells/mL. Cells were incubated with Annexin V and PI in a 96-well round-bottom plate at room temperature according to the manufacturer's protocol. As a positive control, non-transfected cells were treated with 250 nM staurosporine (Sigma) overnight prior to annexin V/PI staining. Samples were analyzed by flow cytometry using a BD LSR II flow cytometer (BD Biosciences, San Jose, Calif.) and results were evaluated using FlowJo analysis software (Ashland, Oreg.). Percentages of Annexin V positive/PI negative and Annexin V positive/PI positive cells were quantified and compared to untreated and non-targeting miRNA controls.

Invasion and Migration Assay

Cell invasion and migration assays were performed using Cultrex 96-well BME Cell Invasion or Cell Migration Boyden chambers (Trevigen Inc., Gaithersburg, Md.). Malme-3M cells were transfected as indicated with inhibitors to over-expressed miRs, or microRNA mimics of under-expressed miRs. 48 hours after transfection approximately 50,000 cells were seeded into the upper chamber of the Cultrex dish in serum-free media. For cell invasion assays, chambers were coated with 0.25× basement membrane extract. For migration assays, chambers were uncoated. The lower chamber was filled with 150 μL RPMI media +10% FBS. Non-transfected Malme-3M cells were used as a positive control and non-transfected primary melanocytes were used as a negative control. After 24 hours, cells that invaded or migrated were dissociated and stained with Calcein-AM according to the manufacturer's protocol. Fluorescence was measured at 485 nm excitation/520 nm emission using the SpectraMax M5 (Molecular Devices) to quantify invading/migrating cell numbers. Statistical comparisons were conducted using the Welch's 2-sample t-test; p<0.05 was considered significant.

Soft Agar Colony Formation

Melanocytes were seeded and transfected with 120 nM of miRNA mimics of the desired cluster or sub-clusters, as described. After 36 hrs, cells were trypsinized and approximately 25,000 cells were combined with 0.3% agar. The 0.3% agar/cell mixture was seeded on top of a layer of 0.5% agar, previously added to each well of a 6-well plate and allowed to solidify for 30 min. After 3 weeks of growth, colonies were photographed using a Nikon TE200 microscope, then either counted in 3 fields per well in quadruplicate wells or quantified using the CytoSelect™ Cell Transformation Assay kit (Cell Biolabs, San Diego, Calif.) according to manufacturer's instructions. Briefly, matrix solubilization solution was added to solubilize the agar, then cells were incubated with CyQuant® GR Dye. Viable cells were measured fluorescently in quadruplicate wells for each condition. Statistical comparisons were conducted using the Welch's 2-sample t-test; p<0.05 was considered significant.

### Example 2

### Comparison of miRNA Expression in Patient Malignant Melanoma Biopsies and Normal Donor Skin Biopsies

Patient malignant melanoma biopsies showed notable differences in microRNA expression compared to normal donor skin biopsies. 52 total skin samples (36 from melanoma patients and 16 from healthy patients) were initially selected. Of the 52 samples, six (including five melanoma samples and one healthy sample) were excluded due to atypical expression levels of endogenous controls. 98 miRNAs were identified as being differentially expressed in melanoma skin biopsies compared the healthy controls using t-tests as described herein. The distribution of differentially expressed miRNAs included 83 down-regulated miRNAs, including hsa-miR-203, hsa-miR-26a and miR-200 family; and 15 up-regulated miRNAs, including miR146a and miR-155. A putative X-chromosome miRNA cluster also was identified, referred to herein as the “miR-506-514 cluster”.

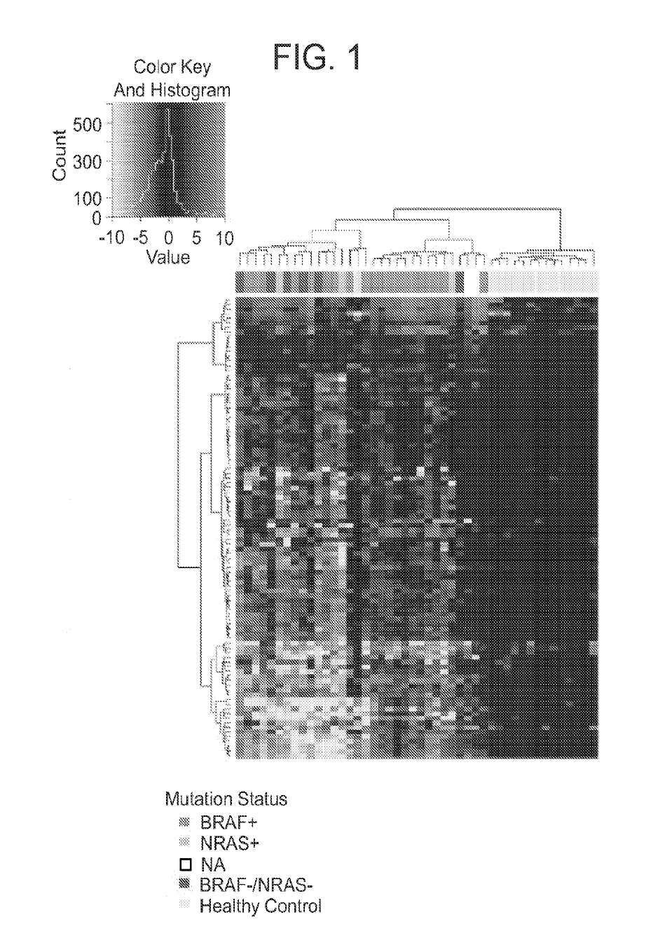

A heatmap of the 98 differentially expressed miRNAs is displayed in FIG. 1. The unsupervised hierarchical clustering of the samples (e.g., along the X-axis) roughly classified the subjects into three groups (e.g., labeled as groups 1, 2 and 3 in FIG. 1). In addition to the cluster filled with the normal controls (e.g., labeled as grouping 3 in FIG. 1), the melanoma patients were further clustered into two groups (e.g., groups 1 and 2 in FIG. 1), one of which is dominated by melanoma patients with a B-Raf mutation (e.g., labeled as group 2 in FIG. 1). The hierarchical clustering in the other dimension (e.g., Y-axis) reveals the co-expression pattern of the miRNAs. Three of the miRNA clusters were highlighted with vertical bars (FIG. 1): the two miR-200 family clusters, highlighted by the gray (e.g., lower) bar in FIG. 1 and the miR-506-514 cluster, highlighted by the black (e.g., upper) bar in FIG. 1.

### Example 3

### Bioinformatics Investigation of miRNAs Identified as being Differentially Expressed in Melanoma and Normal Biopsies

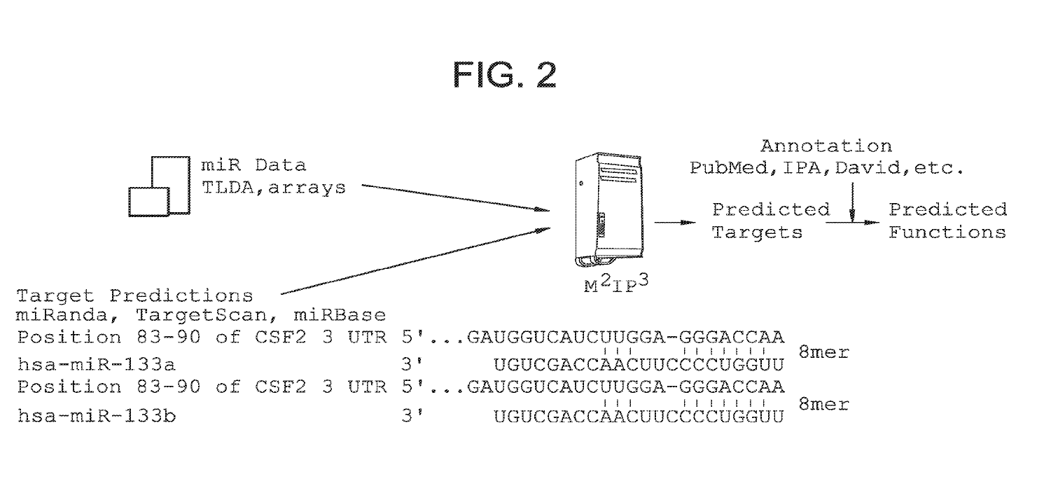

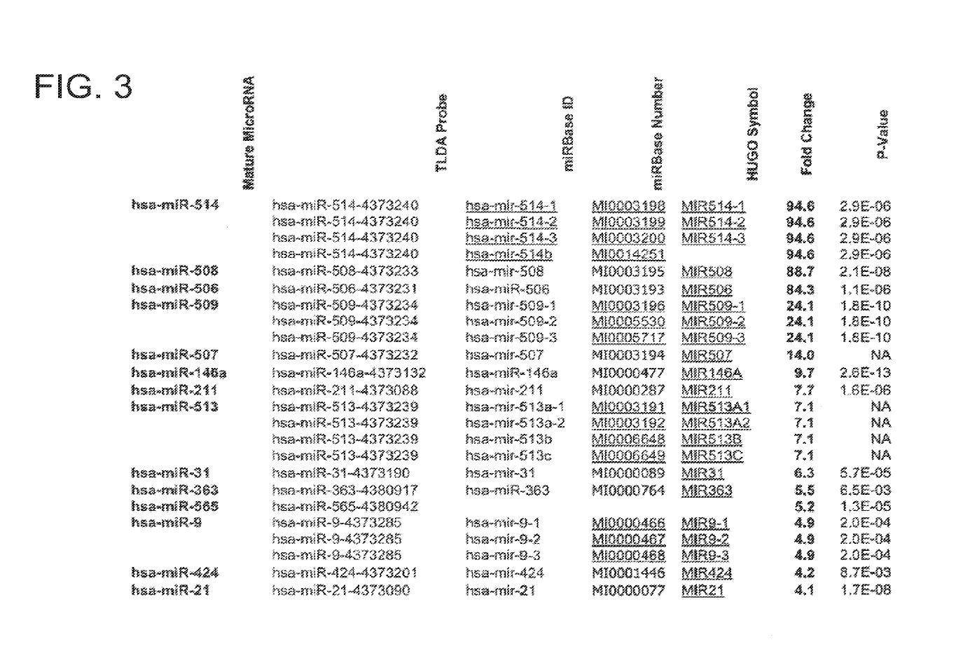

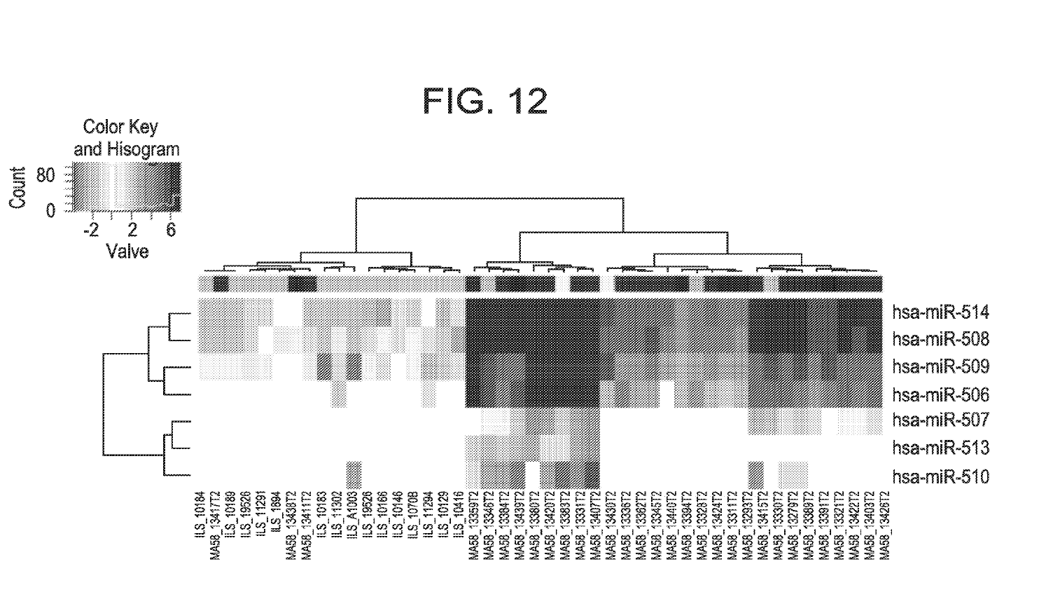

miRNA/mRNA informatics was used to determine which of the microRNAs that were differentially expressed in melanoma and normal biopsies, were putatively involved in melanoma oncogenesis. The chosen miRNAs (e.g., those found to be differentially expressed in melanoma and normal biopsies, and highlighted by the vertical bars in FIG. 1 and FIG. 12 were further investigated using the informatics method of Georgantas et al (PNAS 104:4344), illustrated in FIG. 2, to identify putative microRNA oncogenes and tumor suppressors (e.g., onco-miRs and TS-miRs, respectively). We used three possible criteria to call a microRNA a possible cancer-miR in melanoma. First, we selected miRs that were found in PubMed to be highly associated as onco- or TS-miRs in other cancers. Examples include miR-21 and miR-155 as possible melanoma oncogenes because they were over-expressed in melanoma (FIG. 3) and because there is extensive literature showing them to be oncogenes in other cancers. Second we focused miRs that had both been previously suggested to be involved in other cancers, and that had predicted mRNA targets that are known onco- or TS-genes. MiR-Let-7c is a good example, since it had both been previously published as a tumor suppressor miR, and because informatics identified CCNF, E2F2, E2F6, RAS, etc as some of its mRNA targets that are associated with cancer. Finally, a number of miRs were chosen purely based on being predicted to control known oncogene and tumor-suppressor gene mRNAs. Examples include miR-27a and miR-335, which both targeted a number of mRNAs associated with cellular drug resistance. These mRNAs include CTCF, involved in stress induced apoptosis; NFE2L2, which is a master transcription factor for many proteins involved in cell detoxification; and PLK2, an inhibitor of apoptosis. Using these criteria, five individual over-expressed microRNAs and one putative cluster from the X chromosome (miRNA-506-514 cluster consisting of 14 miR pre-miR5/10 unique mature miRs) were identified as possible melanoma oncogenes. Fourteen individual microRNAs and two microRNA clusters (miR-200c-141 and mir-200a-200b-429) were identified as possible melanoma tumor-suppressors. A non-limiting example of miRs altered in melanoma cells is shown in FIG. 3.

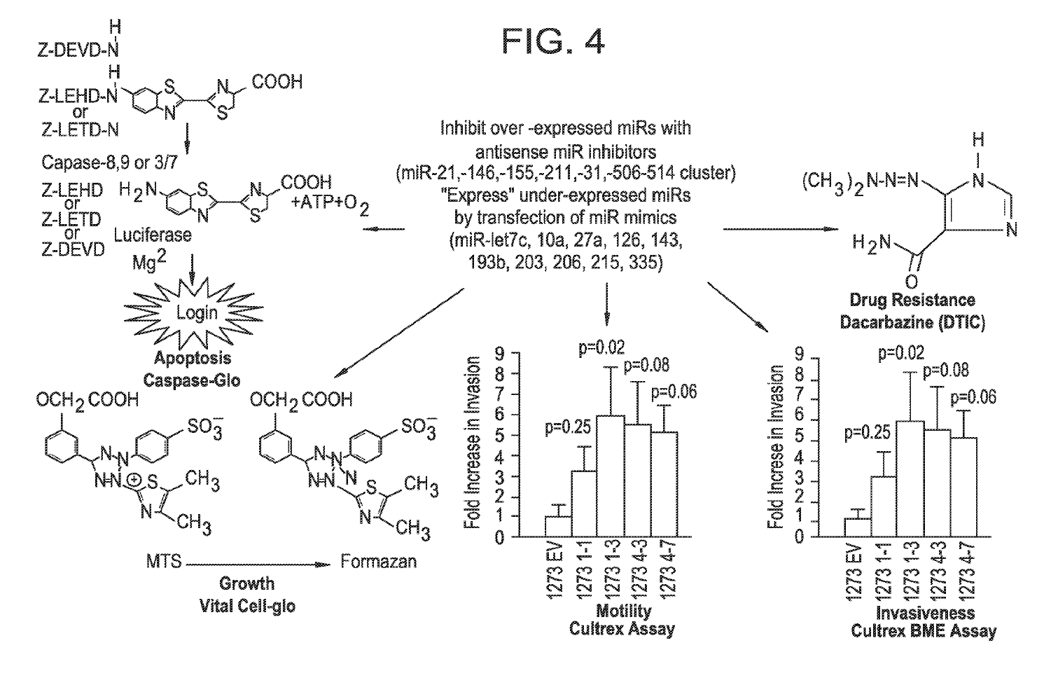

The roles of the chosen microRNAs in the cancerous characteristics of melanoma were further investigated. Melanoma cell growth, apoptosis, migration, and invasion were the most easily investigated and clinically relevant hallmarks of cancer that could be functionally tested, shown schematically in FIG. 4. FIG. 4 shows the high throughput screening (HTS) methods used to determine the effectiveness of the miRs. The five over-expressed miRs and over-expressed miR-506-514 cluster, as well as ten of the under-expressed miRs, were tested for alterations of the cancer characteristics listed above (e.g., melanoma cell growth, apoptosis, migration and invasion).

### Example 4

### Evaluation of microRNAs that Inhibit Growth of Melanoma Cell Lines

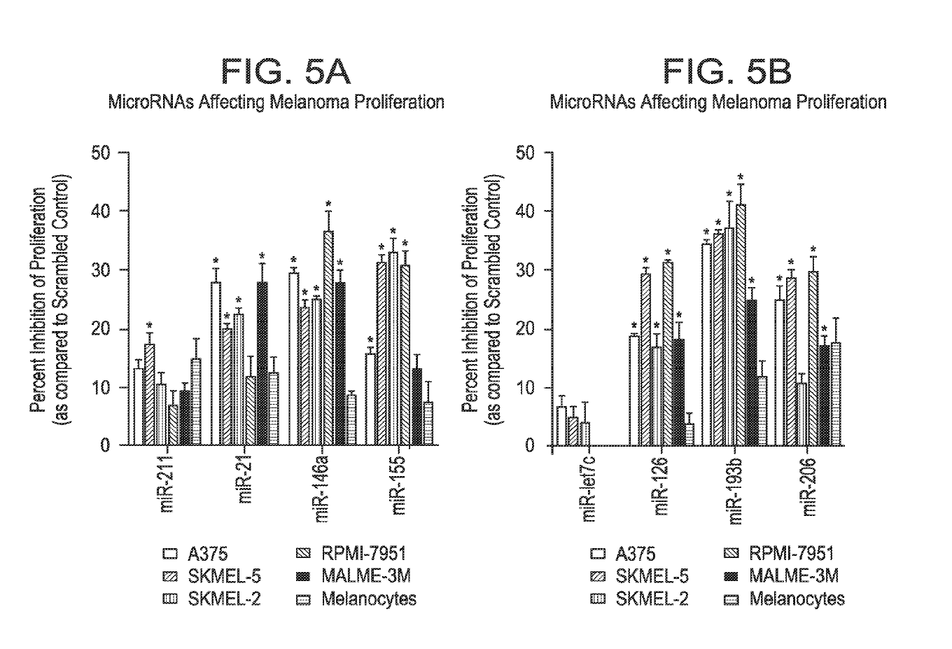

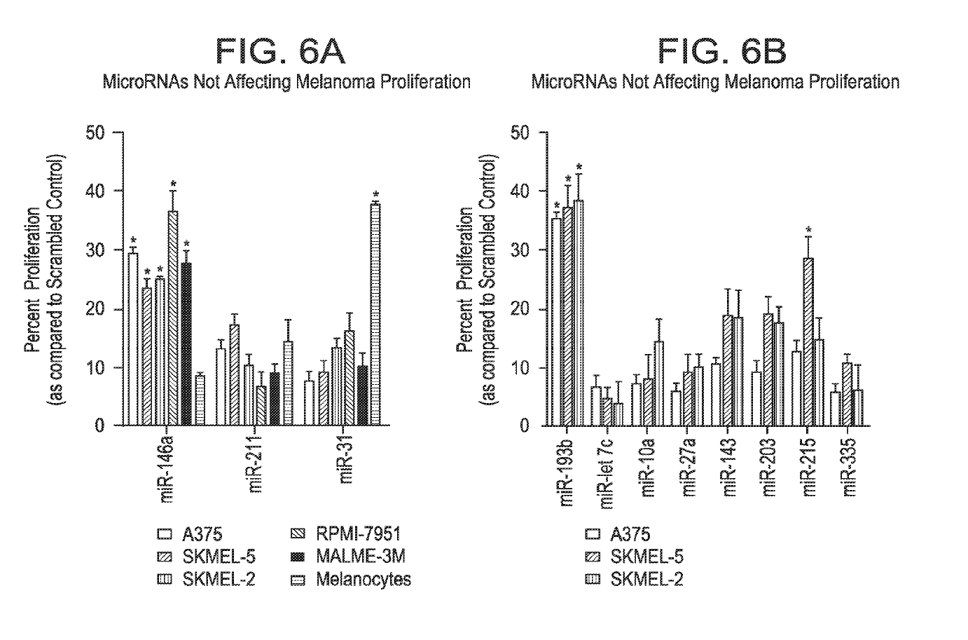

The effects of putative onco- and TS-miRs on cell growth in five different melanoma cell lines and normal melanocytes were measured by luminescent assay at three and five days post transfection (FIGS. 5 and 6). Within this assay, only those microRNA modifications that resulted in a statistically significant decrease in melanoma cell growth in at least 3 different melanoma cell lines, as compared to normal melanocytes, were scored as positive.

Transfection of inhibitors of miR-21, miR-146a, miR-155 significantly decreased cell growth in 4/5 melanoma cell lines for miR-21 and miR-155, and 5/5 melanoma cell lines for miR-146a (FIG. 5A). No synergistic effects by simultaneously inhibition of all five putative onco-miRs were observed (data not shown). Inhibition of miR-31 or miR-211 did not show significant results in more than two cell lines (FIG. 6A).

Transfection of miR mimics to replace the action of putative TS-miRs found that miR-126a or miR-193b significantly decreased melanoma cell growth in 5/5 melanoma cell lines, while miR-206 did so in 3/5 lines (FIG. 5B). As with the onco-miRs, replacing all three of these miRs to assay for synergistic effects did not show any greater inhibition than that of individual miRs alone (data not shown). All of the other putative TS-miRs tested did not show significant effects on cell line growth, or showed effects in only one or two cell lines as compared to their effects in normal melanocytes (FIG. 6B).

### Example 5

### Evaluation of microRNAs that Increase Apoptosis in Melanoma Cell Lines

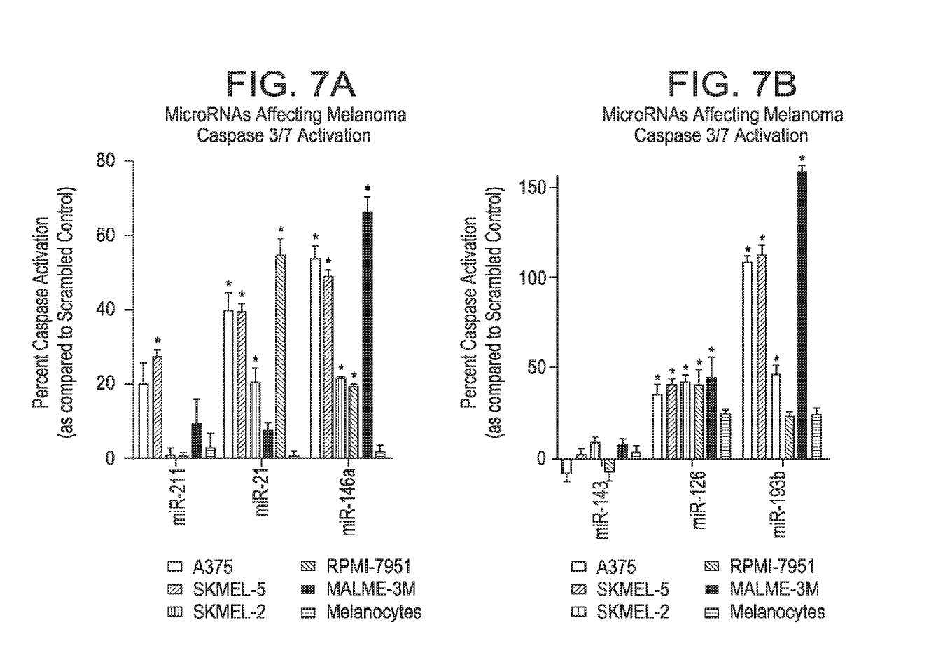

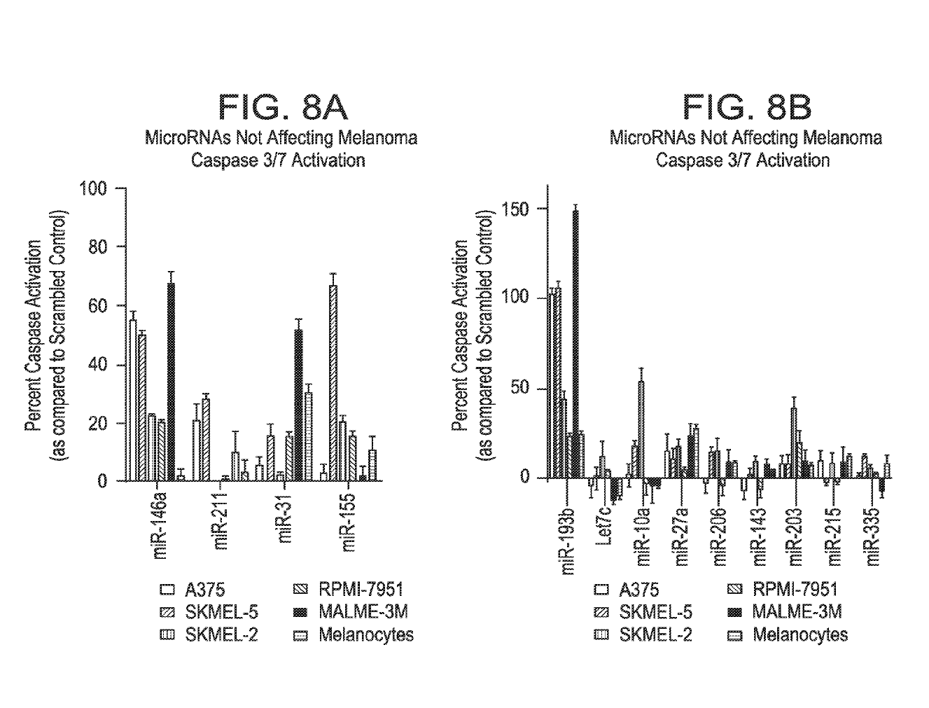

Effects of putative onco- and TS-miRs on apoptosis, specifically activation of Caspase 3 & 7, in five different melanoma cell lines and normal melanocytes were measured by luminescent assay at days 2 and 3 post transfection. To identify the broadest and most durable effects of miR modifications on melanoma, we scored as positive only those modifications yielding statistically significant increases in caspase activation in at least 4/5 melanoma cell lines compared to normal melanocytes. Transfection of an antisense inhibitors of miR-21 or miR-146a significantly increased Caspase 3/7 activation in 4/5 melanoma cell lines (FIG. 7A). We also looked for synergistic effects by simultaneously inhibiting all five putative onco-miRs. As observed when assaying cell growth, the combination of inhibitors to all five putative onco-miRs resulted in caspase activation very similar to that of miR-146a alone (data not shown). Inhibitors of miR-31, miR-155, or miR-211 showed significant effects in only one to two cell lines and were scored as negative for affecting melanoma apoptosis (FIG. 8A).

Transfection of miR mimics for miR-126a or miR-193b significantly increased melanoma apoptosis in 5/5 and 4/5 melanoma cell lines, respectively (FIG. 7B). All of the other putative TS-miRs tested did not show significant effects on cell line caspase activation, or showed effects in only one or two cell lines (FIG. 8B).

### Example 6

### Evaluation of Cell Lines and microRNAs for Migration and Invasion Inhibition Studies

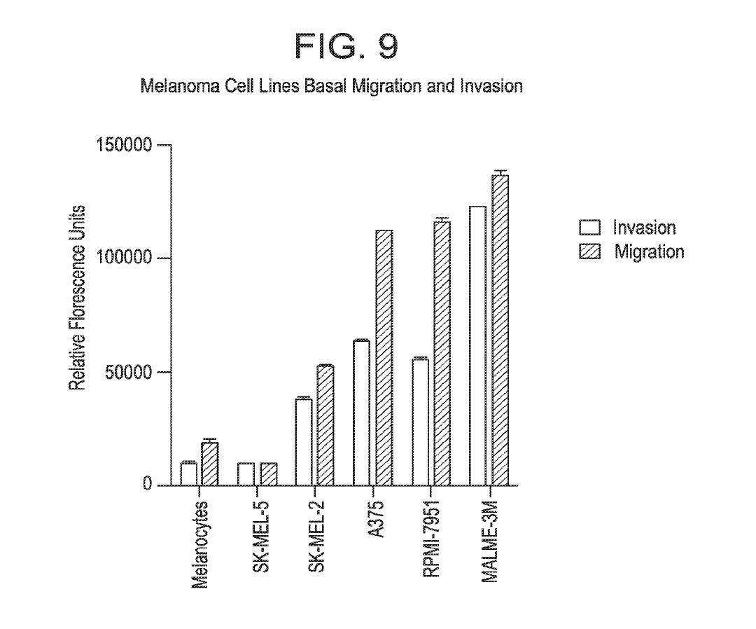

Ninety-six well Boyden chambers plates were used to determine the effects of putative onco- and TS-miRs on migration across a membrane and invasion through Basement Membrane Extract substrate. We began by examining the basal migration and invasion capabilities of the melanoma cell lines and normal melanocytes (FIG. 9). Normal melanocytes and SK-MEL-5 cells showed very little migration or invasion. SK-MEL-2 cells roughly 4× the migration and 2.5× the invasion of normal melanocytes. A375 and RPMI cells showed a high level of about 6× the migration and invasion of normal melanocytes. By far, MALME-3M cells displayed the most vigorous migration (˜12×) and invasion (˜7×) compared to melanocytes. A375 and MALME-3M cells were used for further study of microRNA effects on migration and invasion.

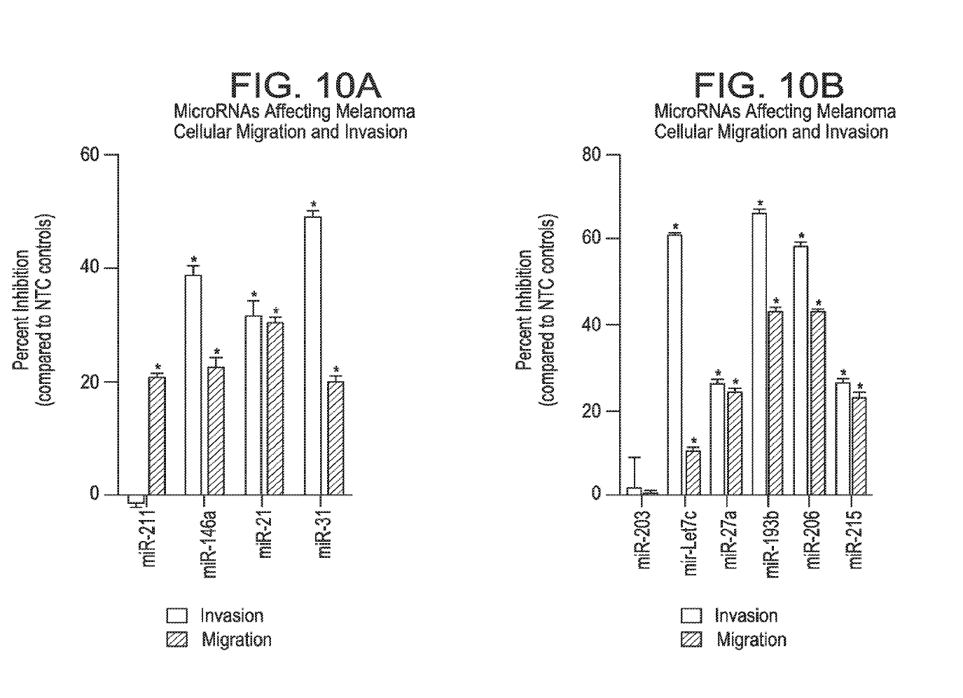

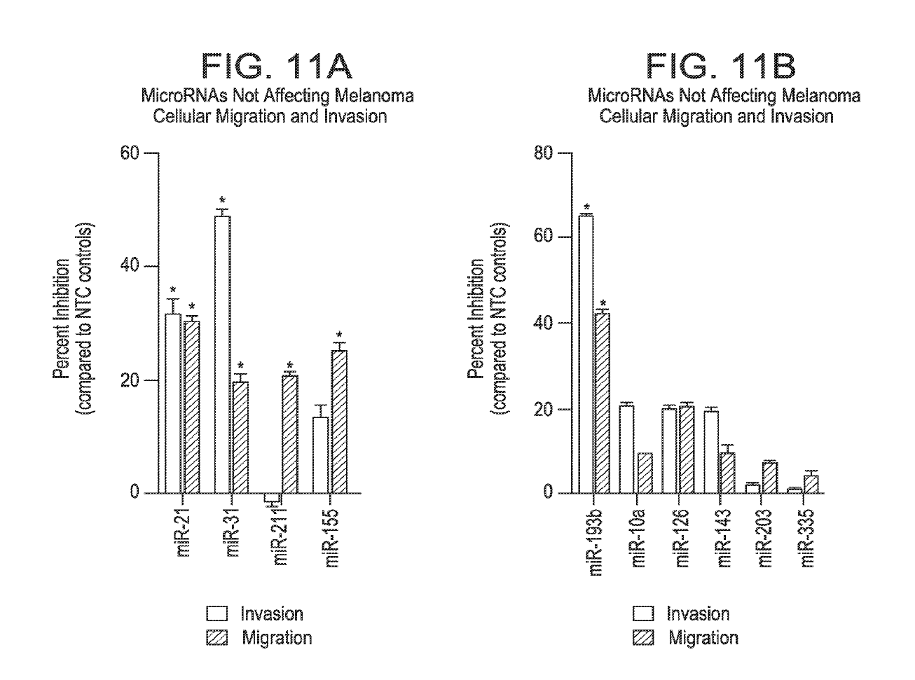

Inhibition of miR-21, miR-31, or miR-146a led to significant decreases in both migration and invasion when compared to melanoma cells transfected with scrambled control inhibitor (FIG. 10A). Inhibition of miR-211 did not show any effect on invasion, but did result in small decrease in migration (FIG. 11A). Finally, inhibition of miR-155 showed only small decreases in migration and invasion (FIG. 11A). Replacement of miR-let7c, miR-27a, miR-193b, miR-206, or miR-215 resulted in significant decreases in both melanoma cell migration and invasion (FIG. 10B). MiR-193b and miR-206 showed the most potent effects. MiR-let7c showed only a small effect on migration, but potently decreased invasion. All other microRNA mimic transfections did not appreciably affect either migration or invasion (FIG. 11B).

### Example 7

### Certain miRNAs in the miRNA-506-514 Cluster are Up-Regulated in Melanoma Patient Biopsies and Cell Lines

Deregulation of miRNA expression has been linked in general to tumor development and progression, but relatively few miRNAs directly involved in melanoma tumorgenesis have been analyzed in detail. New therapeutic targets and biomarkers could prove beneficial for use in melanoma research and treatment. To this end, the miRNA expression profiles of melanoma skin punches and adjacent normal skin were analyzed to identify miRNAs potentially involved in the development and progression of melanomas. Skin biopsies from normal and melanoma tissues were obtained and prepared for analysis as described herein.

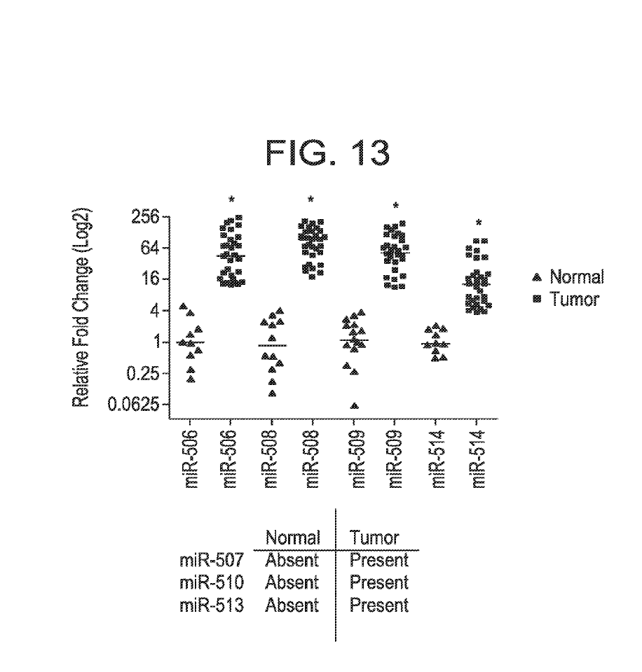

All samples were profiled by miRNA Taqman Low-density Array (TLDA) to determine changes in expression evident in tumor versus normal samples. The results are presented in FIGS. 12-13. A substantial over-expression of all mature microRNAs of the miR-506-514 cluster in melanoma tumor biopsies relative to normal skin was seen, as shown in FIG. 12. miRNAs-506, 508, 509, and 514 were over-expressed an average of between about 30-fold to about 100-fold in melanoma samples, as shown in FIG. 13. miRNAs-507, 510, and 513 were not detectable in normal skin using the TLDA platform, but were present in tumor samples (see FIG. 13). A pooled delta CT value from normal skin was used to calculate relative fold change levels. Lines represent median fold change values for miR-506, miR-508, miR-509, and miR-514. miR-507, miR-510, and miR-513 were not detectable in normal skin (by TLDA), so their differences are shown qualitatively. The asterisk (*) in FIG. 13 indicates a statistically significant difference between tumor and normal, at p<0.001.

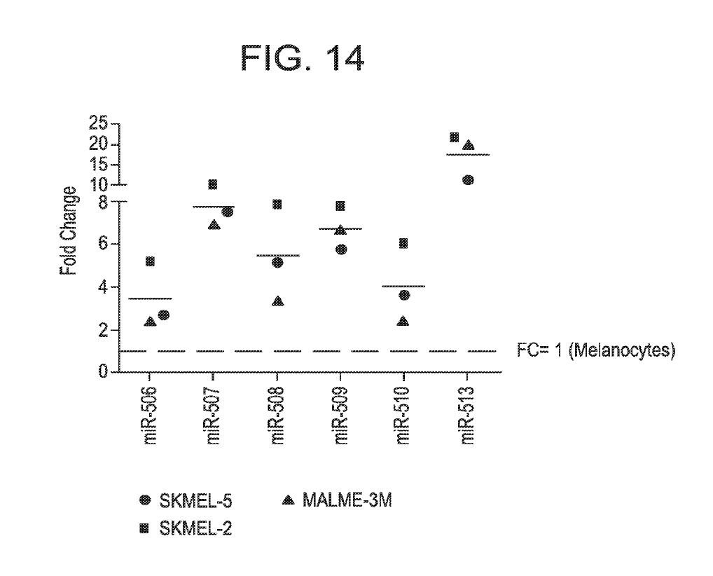

The miRNAs in the miRNA-506-514 cluster have not previously been described in melanoma or assigned any functional relevance in any other cancer type. A panel of melanoma cell lines was assembled and evaluated for use in further studies to investigate the roles of miRNA-506-514 cluster. A group of melanoma cell lines was selected whose miRNA expression profiles clustered most closely with the melanoma skin samples, particularly in terms of the over-expression of the miRNA-506-514 cluster. Using miRNA TLDA, mature miRNAs of the miRNA-506-514 cluster were found to be over-expressed in melanoma cell lines when compared to normal melanocytes, although the magnitude of over-expression was lower than observed in tissues, as shown in FIG. 14. A delta CT value from primary melanocytes was used to calculate relative fold change levels of the miRNA-506-514 cluster in melanoma cell lines. Lines represent mean fold change values of the 3 cell lines indicated in the graph. The differences in magnitude shown herein have been observed in other models comparing cell lines and tissue. The similarity in expression patterns suggested these cell lines could be utilized as a relevant in vitro model.

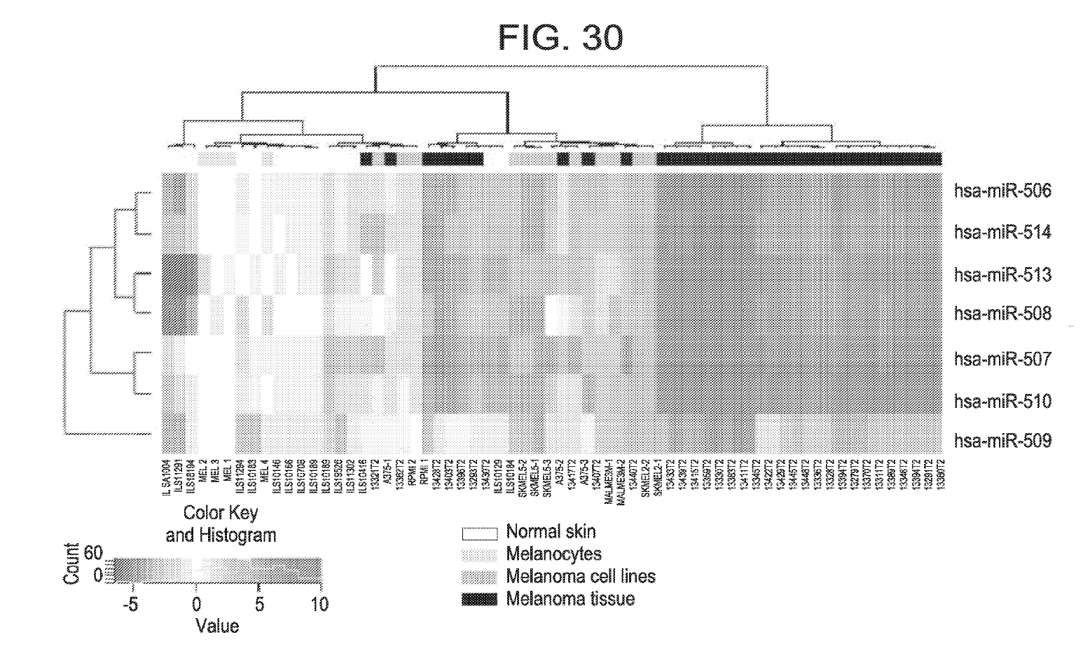

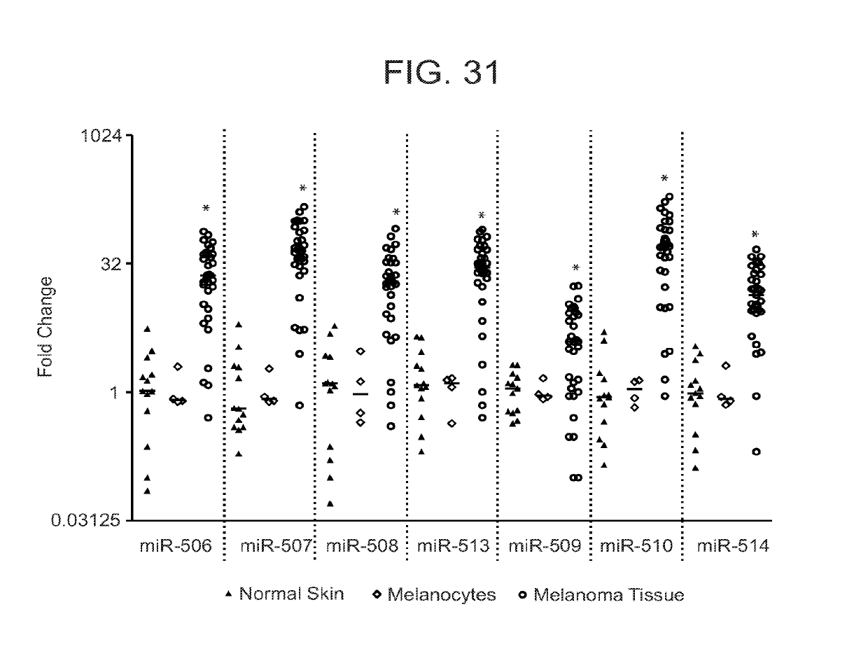

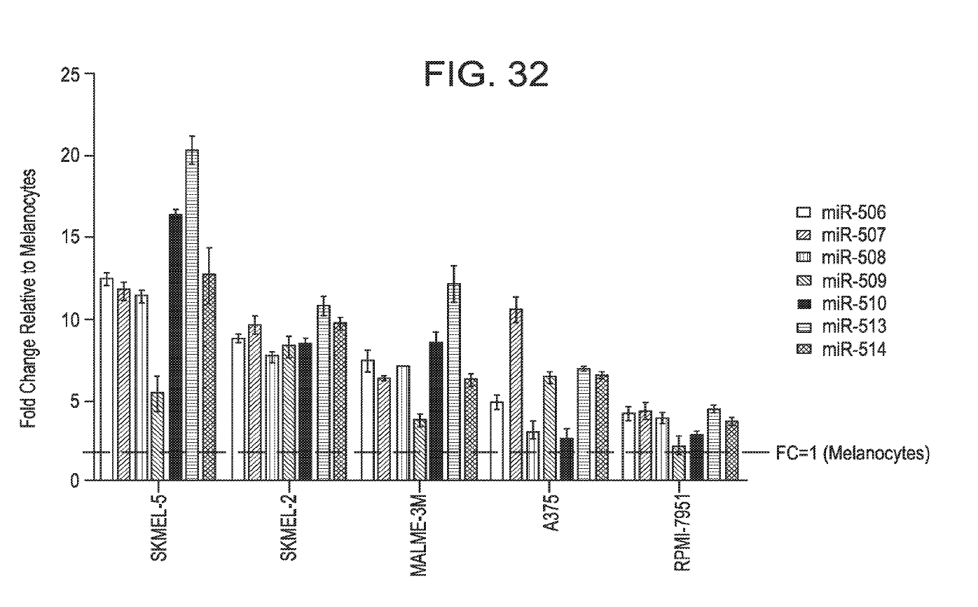

In addition to the TLDA gene expression profiling analysis, the BioMark™ Dynamic Array microfluidics system (Fluidigm Corporation) was also utilized to compare the expression of the miRNA-506-514 cluster in melanocytes, normal skin, melanoma cell lines, and melanoma tissue on the same platform. As shown in FIG. 30, the majority of melanoma samples (23/33) clustered together and substantially overexpressed all mature miRNAs of the miR-506-514 cluster. A second set of melanoma samples (10/33) had some overexpression of these miRs and clustered together with melanoma cell lines consistent with results shown in FIG. 14. In contrast, normal skin and melanocytes with significant underexpression of this set of miRNAs formed a separate and distinct cluster. Because the additional profiling analysis focused on the miRNA-506-514 cluster specifically with the baseline adjusted for expression levels within the cluster, levels of the majority of miRNAs in the cluster were detectable. The miR-506-514 cluster was significantly overexpressed in melanoma tissue and five melanoma cell lines compared to melanocytes and normal skin by an average of between about 30-fold to about 60-fold, as shown in FIG. 31, and between about 4-20 fold, as shown in FIG. 32, which graphically represents the fold changes of the expression level of the miR-506-514 cluster in melanoma cell lines relative to primary melanocytes. Lines represent median fold change values for each member of the miR-506-514 cluster. The asterisk (*) in FIG. 31 indicates a statistically significant difference between tumor and normal, at p<0.001.

### Example 8

### Inhibition of the miRNA-506-miR514 Cluster Substantially Inhibits Melanoma Cell Growth and Invasion/Migration of Melanoma Cells

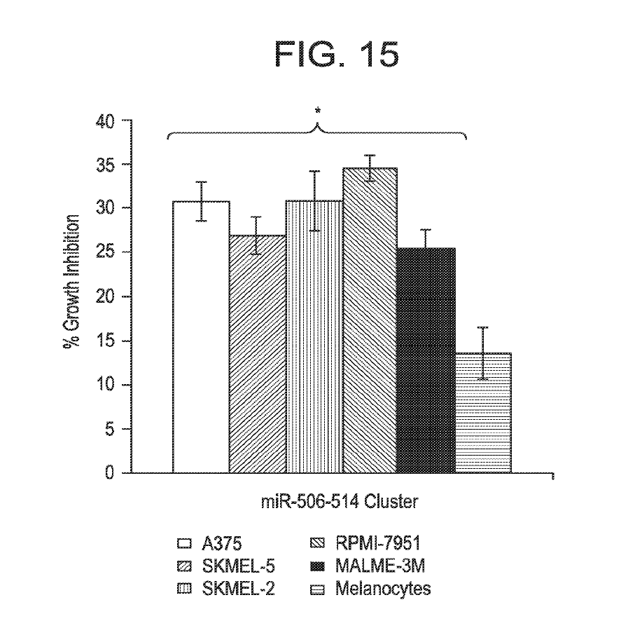

To determine if over-expression of the miRNA-506-514 cluster is involved in melanoma progression, all members of the miRNA-506-514 cluster were simultaneously inhibited in 5 melanoma cell lines (SKMEL-2, SKMEL-5, A375, MALME-3M, and RPMI-7951) using commercially available hairpin inhibitors. 75-85% transfection efficiency was achieved for all cell lines, as measured with a fluorescently labeled miRNA inhibitor (Dharmacon). Cell growth was measured 3 days and 5 days post transfection and compared to cells transfected with miRNA non-targeting inhibitor. Growth of the 5 melanoma cell lines was reduced 25-35% following inhibition of the miRNA-506-514 cluster (see FIG. 15). The results shown in FIG. 15 are from day 3 post transfection and are presented as % inhibition of growth relative to non-targeting control (set to 0%) across 4 independent experiments. An asterisk (*) indicates a statistically significant difference between melanoma cell lines and normal melanocytes, p<0.01. The degree of growth inhibition was significantly different (p<0.001) in melanoma cell lines when compared to normal melanocytes, suggesting that the over-expression of the miRNA-506-514 cluster is important for maintaining melanoma cell growth.

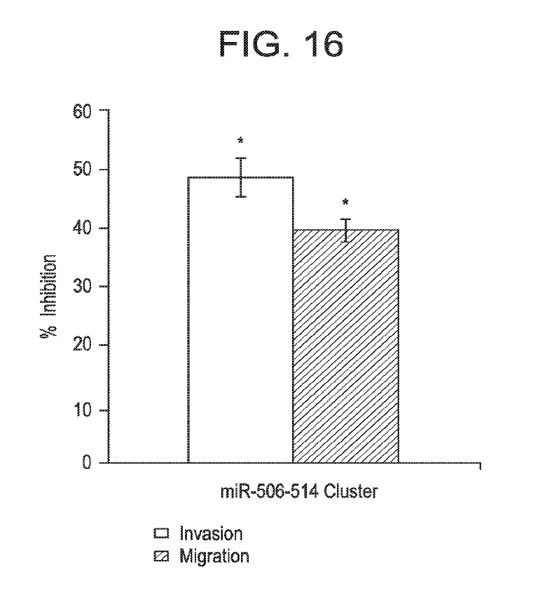

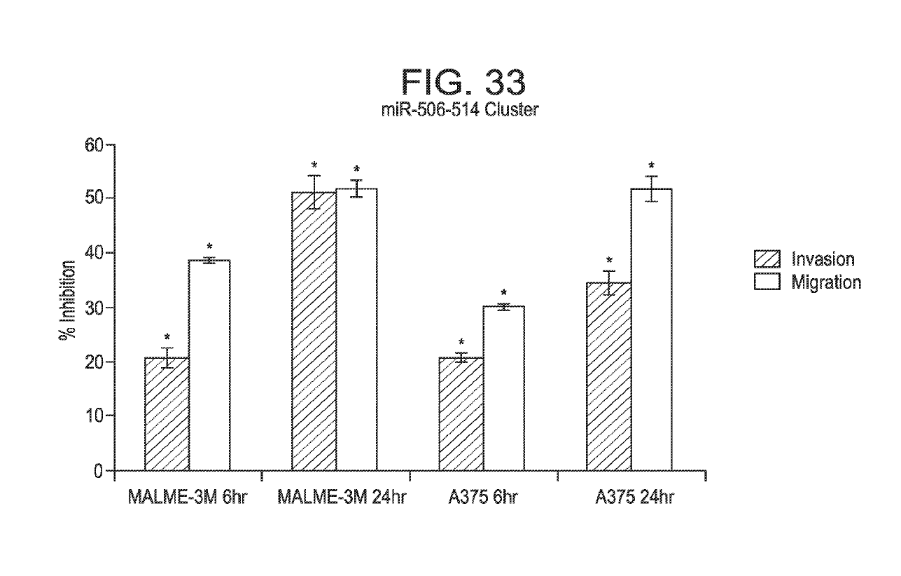

In addition to uncontrolled growth, cancer progression and metastasis is dependent on the ability of cells to migrate and/or invade new tissue. Of the 5 cell lines used to examine growth, the MALME-3M cell line invaded and migrated to the highest level and were therefore utilized for migration/invasion inhibition assays. MALME-3M cells were seeded and transfected as described herein. 48 hrs after transfection, 50,000 MALME-3M cells were seeded into each chamber of a 96-well Cultrex dish, coated with basement membrane extract for invasion or uncoated for migration. 24 hrs after seeding in the assay plates, migrated or invaded cells were stained with Calcein-AM and quantified by measuring fluorescence. Results are shown as % inhibition of invasion or migration relative to non-targeting control (set to 0%). * indicates a statistically significant difference between cells transfected with miRNA-506-514 inhibitors versus the non-targeting control (NTC), p<0.001. Following inhibition of the miRNA-506-514 cluster in MALME-3M cells, migration and invasion were decreased approximately 40% and 52%, respectively, when compared to cell transfected with a control non-targeting inhibitor (see FIG. 16). In additional experiments, inhibition of the miRNA-506-514 cluster was performed in MALME-3M cells and A375 cells, which also invade and migrate at high levels. As shown in FIG. 33, following inhibition of the miR-506-514 cluster in MALME-3M cells, migration and invasion were both decreased approximately 50% at 24 hours when compared to cells transfected with a control non-targeting inhibitor. Similar results were observed in A375 cells, with a 50% and 35% reduction in migration and invasion, respectively. As shown in FIG. 33, this reduction following inhibition of the miRNA cluster was evident even at 6 hours post-transfection at which time there was little apoptosis observed, indicating that the effect on migration and inhibition was not merely due to apoptosis. Melanoma is a highly metastatic cancer, which contributes to the high mortality rate associated with this disease. The involvement of this novel cluster in the invasive and migratory capabilities of melanoma suggests that its over-expression could either be used as a biomarker for metastasis or a target that could prevent the growth of primary tumor, as well as subsequent metastases.

### Example 9

### Melanoma Cell Apoptosis is Substantially Increased Following Inhibition of miRNA-506-514 Cluster

Inhibition of the miRNA-506-514 cluster decreased cell growth, highlighting an important functional role for this cluster in melanoma. Decreased cell numbers sometimes can be a result of growth arrest, sometimes can be due to increased apoptosis and sometimes can be a result of growth arrest and increased apoptosis. Given the ability of cancer cells to acquire mutations and overcome blocks in proliferation, increasing melanoma cell death could prove beneficial to the success of any potential treatment.

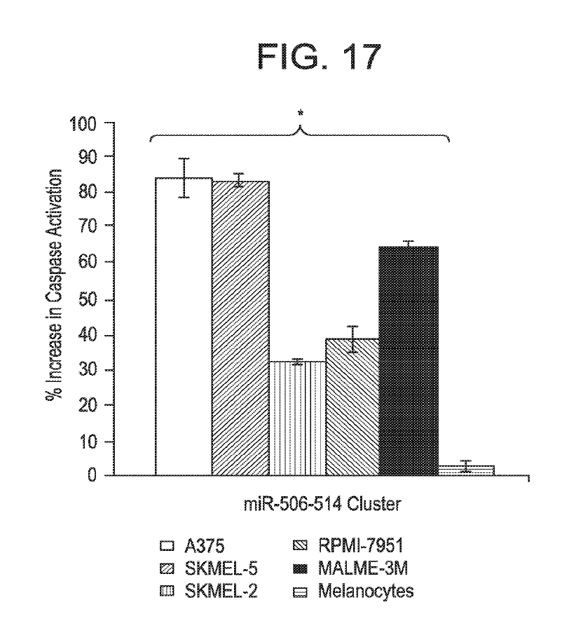

Apoptosis was measured in multiple melanoma cell lines following inhibition of the full miRNA-506-514 cluster by transfecting the appropriate hairpin inhibitors at 10 nM each. The apoptosis assays utilized were (1) caspase 3/7 activation (e.g., caspase is an indicator of apoptosis), and (2) Annexin V flow cytometry. Melanoma cell lines and primary melanocytes were transfected with 10 nM of each anti-miRNA, as described in Example 9. Caspase 3/7 activation was measured by Caspase-Glo 3/7 (Promega) 2 days post transfection. The results shown in FIG. 17 are presented as % increase in caspase 3/7 activity relative to non-targeting control (NTC; set to 0%). An asterisk (*) in the graph indicates a statistically significant difference between melanoma cell line and normal melanocytes, p<0.01. The results indicate that activation of caspase 3/7, an early marker for apoptosis, was increased 50-75% in transfected cell lines at 48 hrs post-transfection, compared to non-targeting miRNA control cells (see FIG. 17).

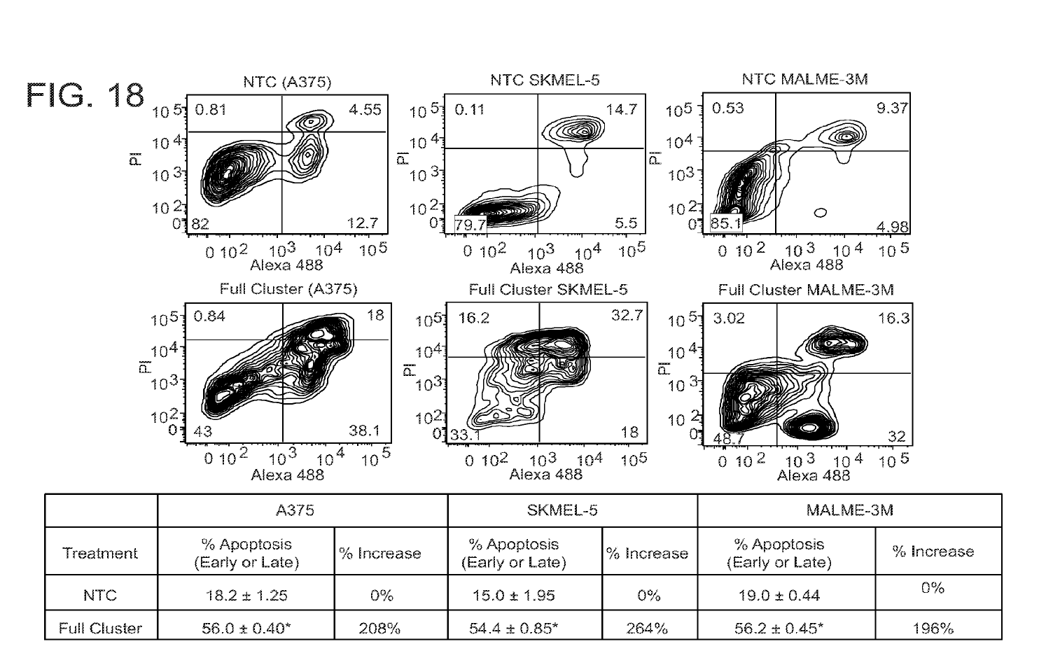

Annexin V/Propidium iodide (PI) I flow cytometry assays were used to measure the levels of phosphatidylserine (PS) present on the cell surface, which is an indicator that cells have begun the apoptotic process. The complete loss of membrane integrity is seen in the final stages of cell death, so the addition of the vital dye PI provided the ability to distinguish between early apoptotic cells (Annexin V positive/PI negative) and late apoptotic cells (Annexin V positive/PI positive) and allowed confirmation of the caspase activation results. Representative flow cytometry results in A375, SKMEL-5, and MALME-3M cells following transfections are shown in FIG. 18. Early apoptotic cells are Annexin+/PI− and appear in the bottom right quadrant. Late apoptotic cells are Annexin+/PI+ and appear in the top right quadrant. The summary table shows total % apoptosis and % increase relative to control. An asterisk (*) indicates a statistically significant difference between cells transfected with miR-506-514 inhibitors versus NTC, p<0.01. Inhibition of the miRNA-506-514 cluster in A375, SKMEL-5, and MALME-3M cells increased the percentage of total apoptotic cells (both early and late) to 56%, 54% and 56%, respectively, compared to 18%, 15% and 19% in non-targeting control (NTC), as shown in FIG. 18. Taken together with the proliferation and invasion results, these data confirmed that the miRNA-506-514 cluster regulates multiple pathways critical for maintaining the cancer phenotype.

### Example 10

### Melanoma Cell Growth in Soft Agar is Significantly Reduced Following Inhibition of miRNA-506-514 Cluster

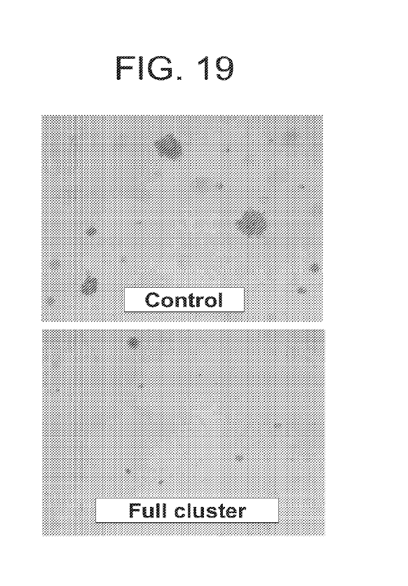

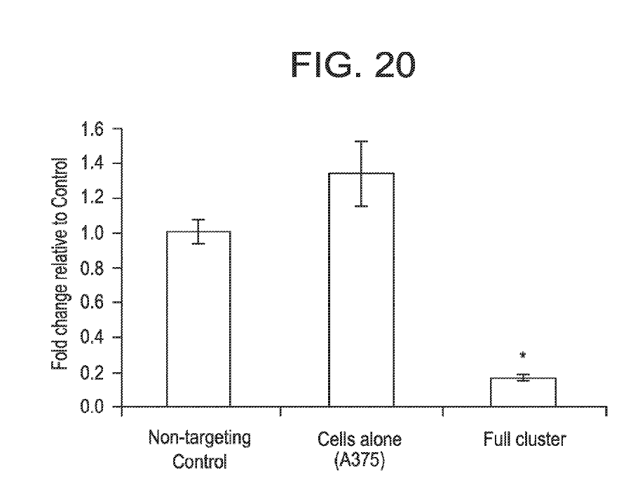

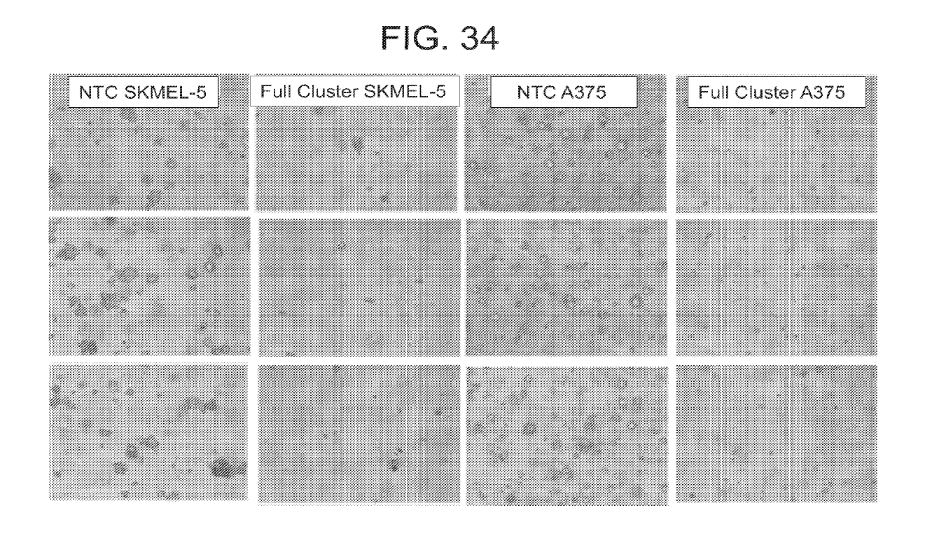

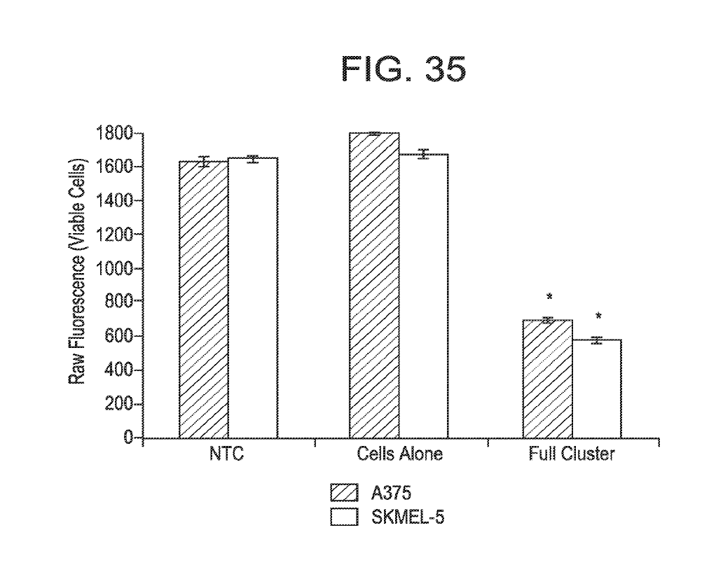

In-vitro-transformed cells and cancer-derived cells are able to survive and grow in the absence of anchorage to an extracellular matrix. The ability to regulate this process would have a profound on tumor progression. Accordingly, we examined the effect of inhibiting the miRNA-506-514 cluster on the ability of melanoma cells to form colonies in soft agar. Melanoma cells were seeded and transfected as described herein. After 48 hrs, cells were trypsinized and 25,000 cells were combined with 0.3% agar. The 0.3% agar/cell mixture was seeded on top of a layer of 0.5% agar, previously added to each well of a 6-well plate and allowed to solidify for 30 min. The seeded cells were maintained for 3 weeks prior to staining with 0.01% crystal violet. FIG. 19 shows representative photographs of colonies photographed at 10× magnification. FIG. 20 graphically represents the increase in number of colonies counted in 2 fields per well in triplicate using a Nikon TE2000 microscope. Results indicate that inhibition of the miRNA-506-514 cluster reduces the number and size of colonies in soft agar by 80% compared to non-targeting miRNA control. Similar experiments performed in both SKMEL-5 and A375 cell lines reduced colony formation by 50% compared to non-targeting control (NTC), as demonstrated by representative photographs of colonies in FIG. 34 and graphically in FIG. 35. The asterisk (*) in the graph in FIGS. 20 and 35 indicates a statistically significant difference between cells transfected with the full cluster compared to cells transfected with non-targeting miRNA control (NTC), at a confidence level of p<0.01. As the capacity for anchorage independent growth represents one of the most important oncogenic properties of cancer cells, these data confirmed a functional role for the miRNA-506-514 cluster that is critical in maintaining the melanoma phenotype.

### Example 11

### Subclusters of the miRNA-506-514 Cluster Showed Equal or Greater Effect on Altering Multiple Cancer Functions than Use of the Full miRNA-506-514 Cluster

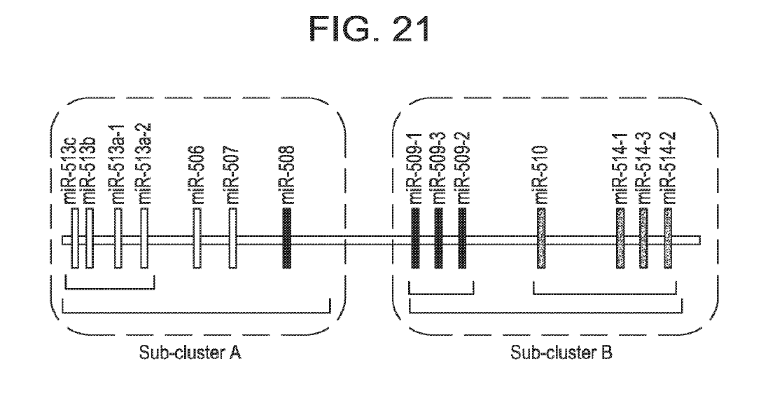

Physical distance mapping and analysis of the phylogenetic relationships of the miRNA-506-514 cluster members suggested a clear separation of the 14 miRs within this cluster into 2 smaller putative sub-clusters; sub-cluster A, consisting of miR-506, miR-507, miR-508-3p, miR-508-5p, miR-5,3-a-3p, miR-5,3-a-5p, miR-5,3-b, and miR-5,3-c, and sub-cluster B, consisting of miR-509-3p, miR-509-5p, miR-510, and miR-514. It is possible that these clusters could be further divided into smaller sub-clusters, where the groupings (i) miR-5,3-a-3p, miR-5,3-a-5p, miR-5,3-b, and miR-5,3-c; (ii) miR-509-3p, and miR-509-5p; and (iii) miR-510, and miR-514, would independently comprise 3 potential sub-clusters. All members of the miRNA-506-514 cluster lie within 100 kb on the X chromosome. A schematic representation of the genomic organization of the miRNA-506-514 cluster coding regions is presented in FIG. 21. FIG. 21 illustrates the physical distances (blocks) and phylogenetic clusters (dotted rectangles) present in this cluster. Individual members were segregated into various sub-clusters for further examination (brackets), based on physical distance and phylogenetic clustering. FIG. 21 is adapted from Zhang et al, Genome Research, 17:612 (2007).

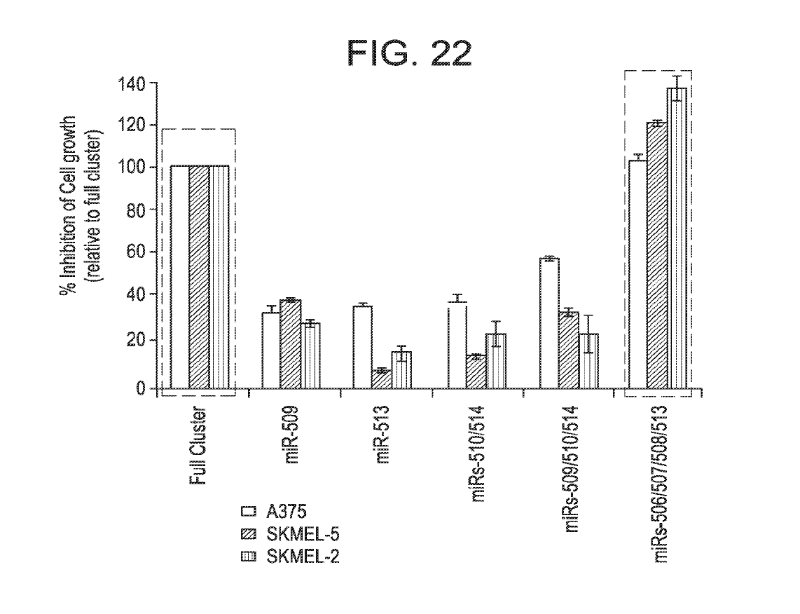

In order to investigate the effect of these putative sub-clusters on the growth of melanoma cell lines, members of each sub-cluster were inhibited in multiple melanoma cell lines, at a final concentration of 70 nM divided equally between all members of a sub-cluster included in the assay. Cells were seeded and transfected as described herein. Cell growth was measured 3 and 5 days post-transfection. The results of cell growth inhibition 5 days post transfection are shown in FIG. 22. The results are presented as % change from the effects observed after inhibiting the full miRNA-506-514 cluster, which allowed identification of any sub-cluster with activity equal to or better than the full miRNA-506-514 cluster. The results revealed that the miRNA-506-507-508-513 sub-cluster (Sub-cluster A) had an equal or greater effect on inhibiting cell growth than the full cluster. Multiple concentrations of these putative sub-clusters were examined, as was each miRNA individually or in smaller combinations of 2 or 3. None of the individual miRNAs or smaller combinations of miRNAs evaluated was able to produce the same effect as the full cluster or sub-cluster A (see FIG. 22 and data not shown).

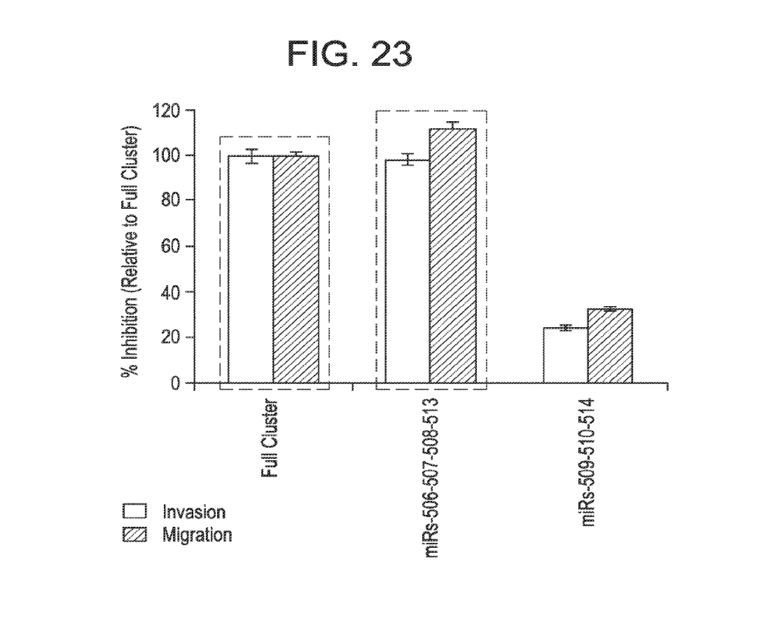

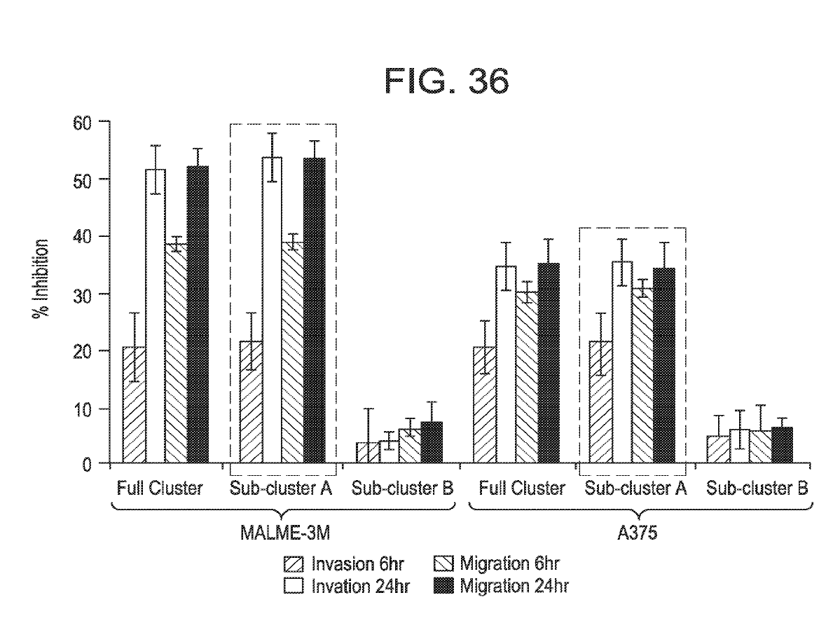

The effect of inhibition of all combinations of putative sub-clusters on melanoma cell invasion and migration was examined using MALME-3M cells. The migration/invasion studies were performed as described herein. The results, presented as % change from the effects observed after inhibiting the full miRNA-506-514 cluster, are shown in FIG. 23. Boxed results in FIG. 23 indicate effects equal to or greater than the effects observed using the full miRNA-506-514 cluster. The results indicate that inhibition of sub-cluster A reduced invasion and migration an amount equal to or greater than the level observed following inhibition of the full cluster. This effect was not observed by inhibiting sub-cluster B (see FIG. 23) or by inhibition of individual miRNAs or smaller combinations of 2 or 3 (data not shown). Similar experiments to study the effect of inhibition at 6 and 24 hours of putative sub-clusters A and B on melanoma cell invasion and migration were additionally examined in both MALME-3M and A375 cells. As shown in FIG. 36, inhibition of sub-cluster A reduced invasion and migration at both 6 hours and 24 hours, but this effect was not seen by inhibiting sub-cluster B.

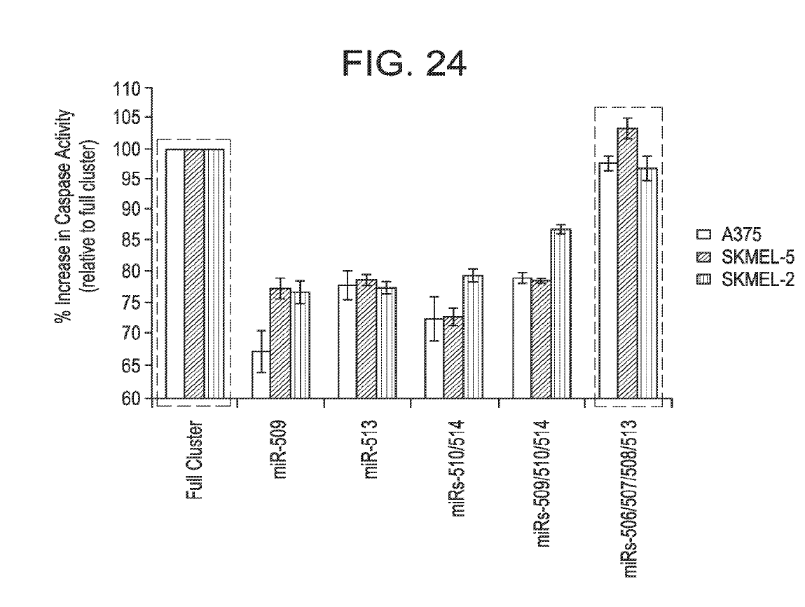

Apoptosis in multiple melanoma cell lines was measured following inhibition of the full miRNA-506-514 cluster or identified putative sub-clusters. Caspase 3/7 activation and Annexin V flow cytometry assays were utilized to assess altered apoptosis, as described herein. Activation of caspase 3/7 (Caspase-Glo) was evaluated 2 days post transfection. The results for caspase activation are shown in FIG. 24 and are presented as % change relative to the full miR-506-514 cluster. Boxed results indicate equal or greater effects compared to full cluster. The results indicate that caspase activation at 48 hrs post-transfection by sub-cluster A was increased to a level equal to or greater than level seen with the full cluster (see FIG. 24).

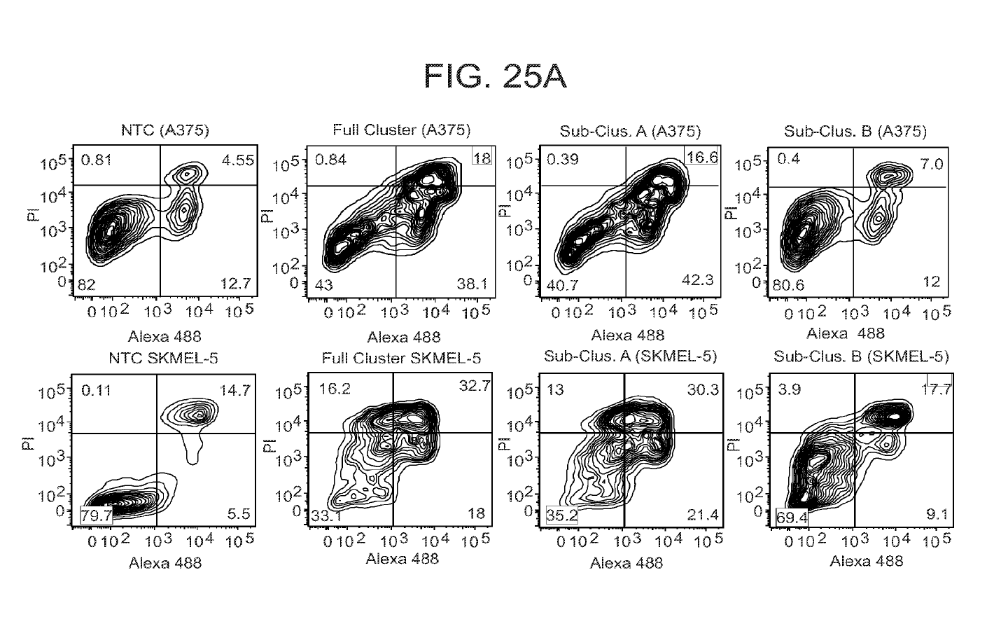

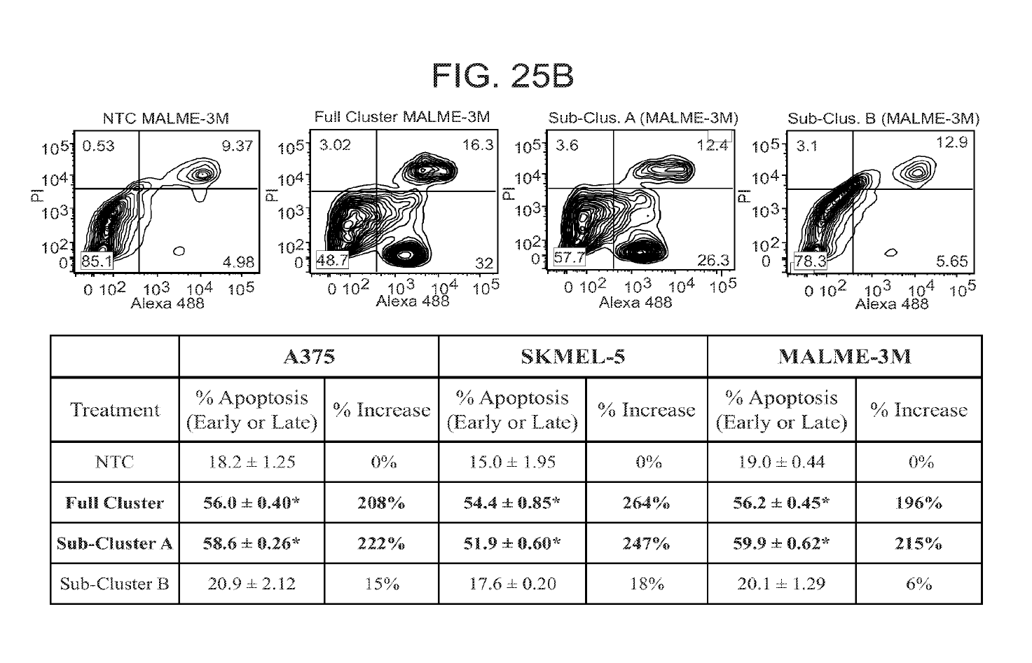

The Annexin V/PI flow cytometry assay confirmed the caspase activation data. Early apoptotic cells (Alexa+/PI−) appear in the bottom right quadrant. Late apoptotic cells (Alexa+/PI+) appear in the top right quadrant. The summary table indicates total % apoptosis and % increase relative to control. The asterisk (*) indicates a statistically significant difference between cells transfected with miRNA inhibitors and NTC, p<0.001. The results from experiments with 3 melanoma cell lines shown in FIG. 25 indicate that sub-cluster A increased the percentage of early and late apoptotic cells (e.g., Annexin V positive/PI negative and Annexin V positive/PI positive, respectively) an amount equal to or greater than the level seen with the full cluster. This result was not observed with sub-cluster B (see FIG. 25) or any of the other sub-cluster combinations tested (data not shown).

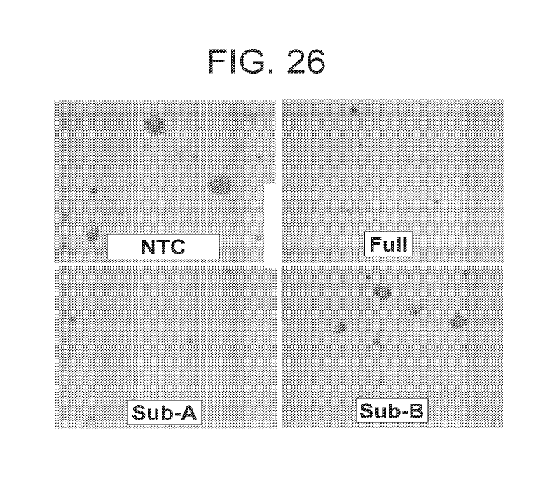

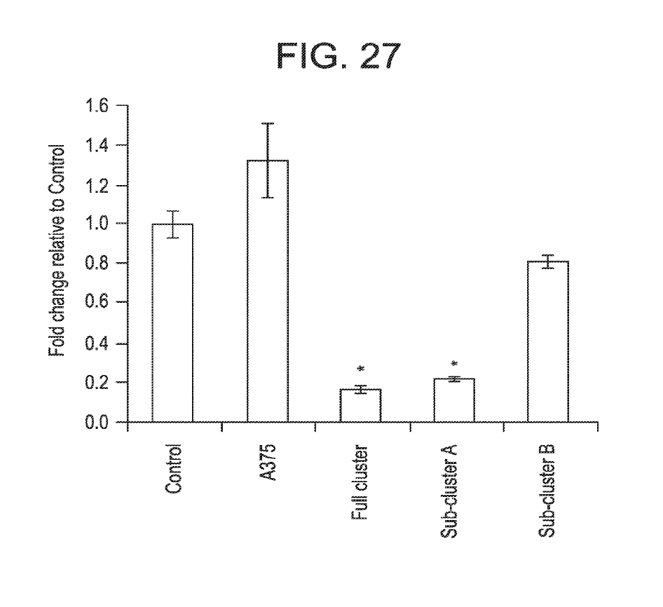

The ability of the inhibition of sub-cluster A to significantly reduce colony formation in soft agar to levels seen following inhibition of the full miRNA-506-514 cluster is shown in FIGS. 26 and 27. FIG. 26 shows representative photographs of colonies photographed at 10× magnification. FIG. 27 graphically represents the increase in number of colonies counted in 2 fields per well in triplicate. The asterisk (*) in FIG. 27 indicates statistically significant differences between cells transfected with the full cluster or sub-cluster A compared to cells transfected with non-targeting miRNA control (NTC), at a confidence level of p<0.01. Given that the phenotypic effects regulated by the full miRNA-506-514 cluster and sub-cluster A are both statistically significant and nearly identical, we can conclude that sub-cluster A (miRNAs-506, -507, -508, and -513) plays an essential role in modulating the pathways important for melanoma growth and progression.

### Example 12

### Over Expression of the Full miRNA-506-514 Cluster miRNAs Transformed Normal Melanocytes

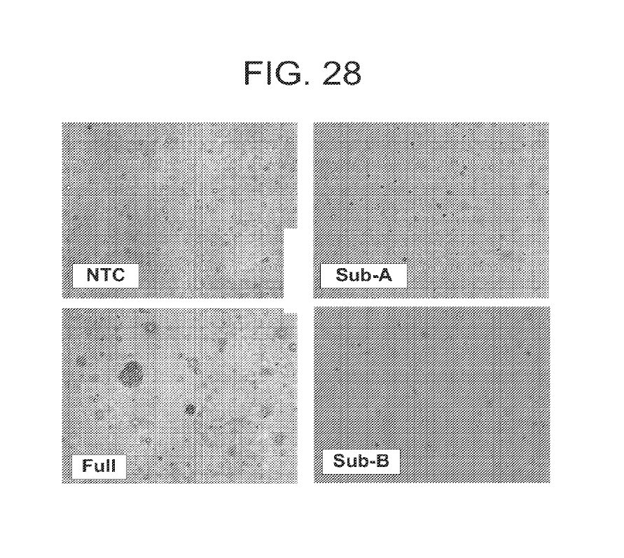

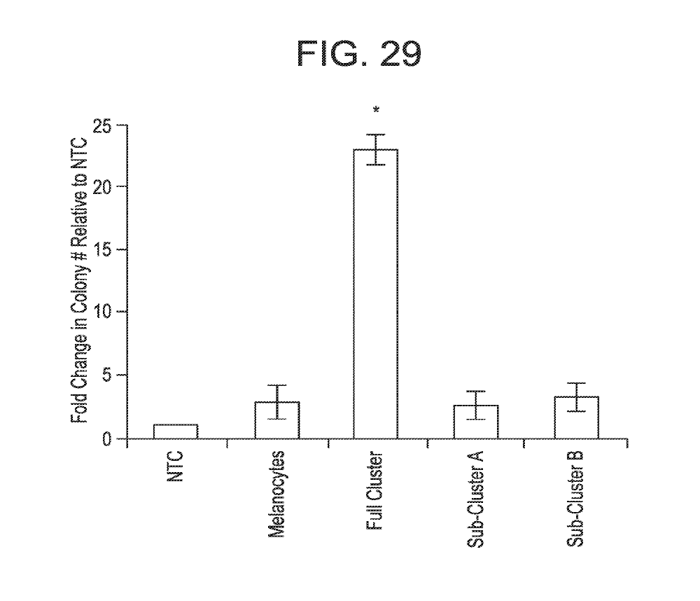

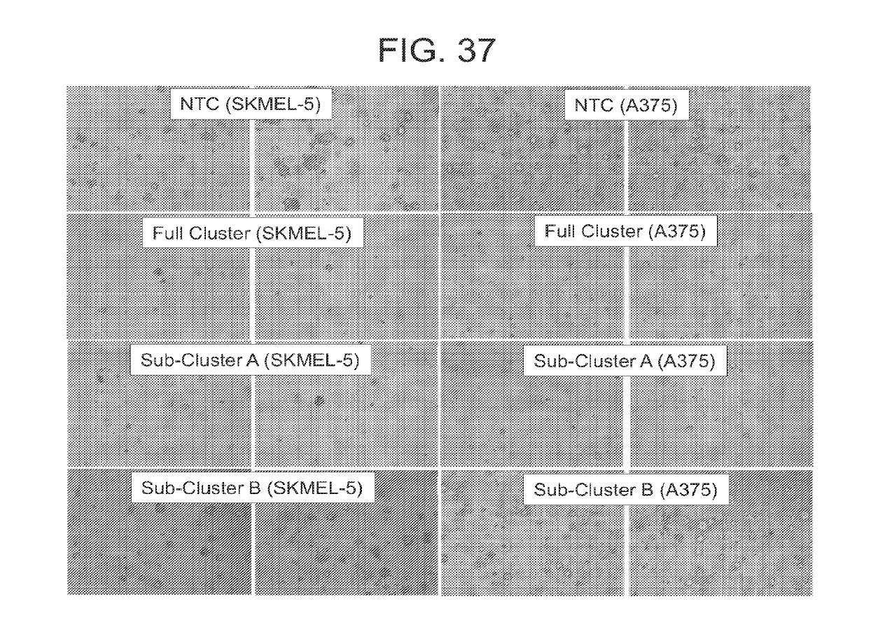

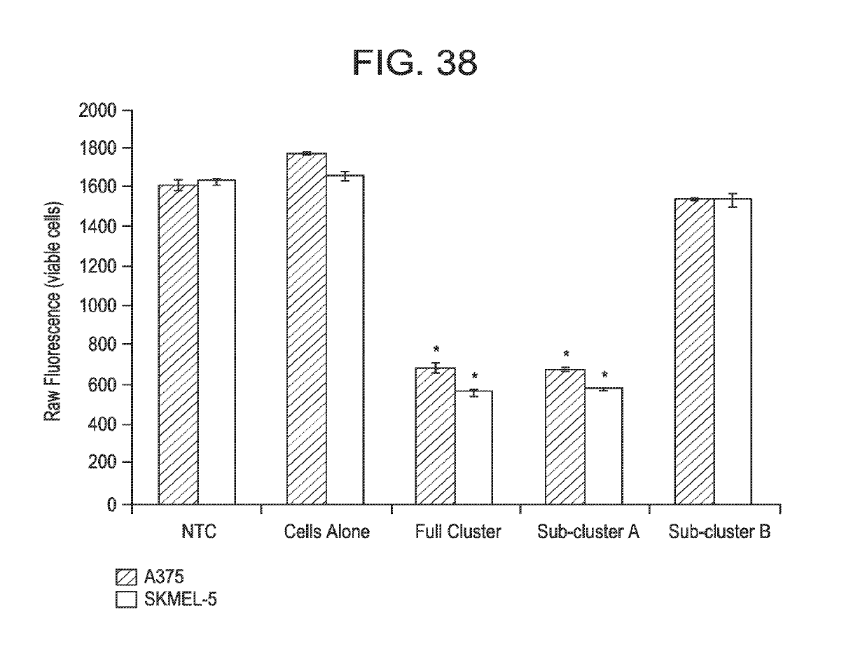

The functional role of the miRNAs in the 506-514 cluster and various sub-clusters in controlling melanoma cell growth, apoptosis, migration/invasion, and anchorage-independent growth has been investigated and described herein. The oncogenic capabilities of the full miRNA-506-514 cluster, or various sub-clusters, also could be of potential interest. A characteristic of oncogenic potential is anchorage independent growth, therefore the effects of over-expressing 506-514 cluster miRNAs was measured on transforming melanocytes to form colonies in soft agar. Normal melanocytes were seeded and transfected with 120 nM of miRNA mimics for the miRNA-506-514 cluster or indicated sub-clusters, as described. Cells were seeded into soft agar as described in example 10. The results of over expression of the miRNA-506-514 cluster or various sub-clusters are presented in FIGS. 28 and 29. FIG. 28 shows representative photographs at 10× magnification. FIG. 29 graphically represents the increase in number of colonies counted in 2 fields per well in triplicate. Results of similar experiments performed in both SKMEL-5 and A375 cells are shown photographically in FIG. 37 and graphically in FIG. 38. The asterisk (*) in FIGS. 29 and 38 indicates a statistically significant difference between cells transfected with the full or sub-clusters compared to cells transfected with non-targeting miRNA control (NTC), at a confidence level of p<0.01.

Over-expression of the full cluster, but not sub-clusters A or B, was able to induce melanocyte transformation, as indicated by a 23-fold increase in colony number relative to melanocytes transfected with NTC (see FIG. 29). Sub-cluster A (miRNAs-506, -507, -508-3p, -508-5p, -5,3-a-3p, -5,3-a-5p, -5,3-b, and -5,3-c) seemed to play a functional role in maintaining melanoma growth, apoptosis, invasion/migration, and anchorage-independent growth, but its over-expression was unable to induce melanocyte transformation to a similar degree as observed with the full cluster. The ability of miRNAs to target multiple pathways supports a model where all members of the miRNA-506-514 cluster are required to initiate cancer formation, but the pathways needed to maintain cancer growth and prevent apoptosis may be distinctly regulated by only a subset of miRNAs (sub-cluster A). Comparing predicted targets of the full cluster versus sub-cluster A may reveal differential genes/pathways that could explain the multi-functional role of this novel cluster in contributing to both the initiation and maintenance of melanoma.

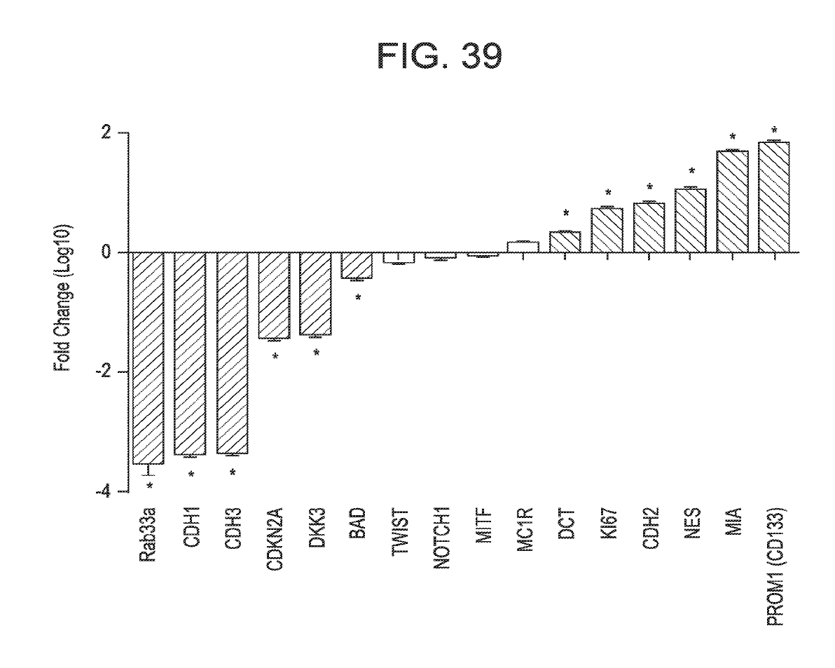

To assess changes in gene expression associated with melanocyte transformation due to over-expression of the miR-506-514 cluster, RNA was isolated from the melanocyte colonies that grew in soft agar and a panel of melanoma and cancer-specific markers were examined by TaqMan RT-PCR. As shown in FIG. 39, miR-506-514 overexpression and growth in soft agar was associated with substantial downregulation of E-cadherin, P-cadherin, p16/CDKN2A, DKK3, RAB33A, TYRP1 and BAD, as well as greatly increased expression of Ki67, MIA, DCT and the stem cell markers PROM1/CD133 and Nestin.

TABLE 1
MicroRNA Sequences and Fold Change of Measured
Characteristic with Respect to Control

| | | | Mature | Mature | |
|---|---|---|---|---|---|
| TLDA | miR- | | Sequence | Sequence | miRBase |
| MicroRNA- | Base | | (or -5p | 2 (or -3p | Accession |
| Probe | ID | pri-miR Sequence | microRNA) | microRNA) | Number |
| hsa-miR- | hsa- | AACAUGUUGUCUG | AUUGACA | | MI0003198 |
| 514- | mir- | UGGUACCCUACUC | CUUCUGU | | |
| 4373240 | 514-1 | UGGAGAGUGACAA | GAGUAGA | | |
| | | UCAUGUAUAAUUAA | | | |
| | | AUUUGAUUGACAC | | | |
| | | UUCUGUGAGUAGA | | | |
| | | GUAACGCAUGACA | | | |
| | | CGUACG | | | |
| hsa-miR- | hsa- | GUUGUCUGUGGUA | AUUGACA | | MI0003199 |
| 514- | mir- | CCCUACUCUGGAG | CUUCUGU | | |
| 4373240 | 514-2 | AGUGACAAUCAUG | GAGUAGA | | |
| | | UAUAACUAAAUUUG | | | |
| | | AUUGACACUUCUG | | | |
| | | UGAGUAGAGUAAC | | | |
| | | GCAUGACAC | | | |
| hsa-miR- | hsa- | GUUGUCUGUGGUA | AUUGACA | | MI0003200 |
| 514- | mir- | CCCUACUCUGGAG | CUUCUGU | | |
| 4373240 | 514-3 | AGUGACAAUCAUG | GAGUAGA | | |
| | | UAUAACUAAAUUUG | | | |
| | | AUUGACACUUCUG | | | |
| | | UGAGUAGAGUAAC | | | |
| | | GCAUGACAC | | | |
| hsa-miR- | hsa- | CAUGUGGUACUCU | UUCUCAA | AUUGACA | MI0014251 |
| 514- | mir- | UCUCAAGAGGGAG | GAGGGAG | CCUCUGU | |
| 4373240 | 514b | GCAAUCAUGUGUA | GCAAUCAU | GAGUGGA | |
| | | AUUAGAUAUGAUU | | | |
| | | GACACCUCUGUGA | | | |
| | | GUGGAGUAACACA | | | |
| | | UG | | | |
| hsa-miR- | hsa- | CCACCUUCAGCUG | UACUCCA | UGAUUGU | MI0003195 |
| 508- | mir- | AGUGUAGUGCCCU | GAGGGCG | AGCCUUU | |
| 4373233 | 508 | ACUCCAGAGGGCG | UCACUCA | UGGAGUA | |
| | | UCACUCAUGUAAAC | UG | GA | |
| | | UAAAACAUGAUUGU | | | |
| | | AGCCUUUUGGAGU | | | |
| | | AGAGUAAUACACAU | | | |
| | | CACGUAACGCAUA | | | |
| | | UUUGGUGG | | | |
| hsa-miR- | hsa- | GCCACCACCAUCA | UAAGGCA | | MI0003193 |
| 506- | miR- | GCCAUACUAUGUG | CCCUUCU | | |
| 4373231 | 506 | UAGUGCCUUAUUC | GAGUAGA | | |
| | | AGGAAGGUGUUAC | | | |
| | | UUAAUAGAUUAAUA | | | |
| | | UUUGUAAGGCACC | | | |
| | | CUUCUGAGUAGAG | | | |
| | | UAAUGUGCAACAU | | | |
| | | GGACAACAUUUGU | | | |
| | | GGUGGC | | | |
| hsa-miR- | hsa- | CAUGCUGUGUGUG | UACUGCA | UGAUUGG | MI0003196 |
| 509- | mir- | GUACCCUACUGCA | GACAGUG | UACGUCU | |
| 4373234 | 509-1 | GACAGUGGCAAUC | GCAAUCA | GUGGGUAG | |
| | | AUGUAUAAUUAAAA | | | |
| | | AUGAUUGGUACGU | | | |
| | | CUGUGGGUAGAGU | | | |
| | | ACUGCAUGACACA | | | |
| | | UG | | | |
| hsa-miR- | hsa- | CAUGCUGUGUGUG | UACUGCA | UGAUUGG | MI0005530 |
| 509- | mir- | GUACCCUACUGCA | GACAGUG | UACGUCU | |
| 4373234 | 509-2 | GACAGUGGCAAUC | GCAAUCA | GUGGGUAG | |
| | | AUGUAUAAUUAAAA | | | |
| | | AUGAUUGGUACGU | | | |
| | | CUGUGGGUAGAGU | | | |
| | | ACUGCAUGACAC | | | |
| hsa-miR- | hsa- | GUGGUACCCUACU | UACUGCA | UGAUUGG | MI0005717 |
| 509- | mir- | GCAGACGUGGCAA | GACGUGG | UACGUCU | |
| 4373234 | 509-3 | UCAUGUAUAAUUAA | CAAUCAUG | GUGGGUAG | |
| | | AAAUGAUUGGUAC | | | |
| | | GUCUGUGGGUAGA | | | |
| | | GUACUGCAU | | | |
| hsa-miR- | hsa- | GUGGUGUCCUACU | UACUCAG | | MI0003197 |
| 510- | mir- | CAGGAGAGUGGCA | GAGAGUG | | |
| 4373235 | 510 | AUCACAUGUAAUUA | GCAAUCAC | | |
| | | GGUGUGAUUGAAA | | | |
| | | CCUCUAAGAGUGG | | | |
| | | AGUAACAC | | | |
| hsa-miR- | hsa- | UUUUGCACCUUUU | GUGCUGU | | MI0003194 |
| 507- | mir- | GGAGUGAA | GUGUAGU | | |
| 4373232 | 507 | | GCUUCAC | | |
| | | | UUCAAGA | | |
| | | | AGUGCCA | | |
| | | | UGCAUGU | | |
| | | | GUCUAGA | | |
| | | | AAUAUGU | | |
| | | | UUUGCAC | | |
| | | | CUUUUGG | | |
| | | | AGUGAAA | | |
| | | | UAAUGCA | | |
| | | | CAACAGA | | |
| | | | UAC | | |
| hsa-miR- | hsa- | CCGAUGUGUAUCC | UGAGAAC | CCUCUGA | MI0000477 |
| 146a- | miR- | UCAGCUUUGAGAA | UGAAUUC | AAUUCAG | |
| 4373132 | 146a | CUGAAUUCCAUGG | CAUGGGUU | UUCUUCAG | |
| | | GUUGUGUCAGUGU | | | |
| | | CAGACCUCUGAAA | | | |
| | | UUCAGUUCUUCAG | | | |
| | | CUGGGAUAUCUCU | | | |
| | | GUCAUCGU | | | |
| hsa-miR- | hsa- | CAGAUCUCAGACA | UCGGGGA | UGUGACA | MI0003686 |
| 542-5p- | mir- | UCUCGGGGAUCAU | UCAUCAU | GAUUGAU | |
| 4378105 | 542- | CAUGUCACGAGAU | GUCACGA | AACUGAAA | |
| | 5p | ACCAGUGUGCACU | GA | | |
| | | UGUGACAGAUUGA | | | |
| | | UAACUGAAAGGUC | | | |
| | | UGGGAGCCACUCA | | | |
| | | UCUUCA | | | |
| hsa-miR- | hsa- | UCACCUGGCCAUG | UUCCCUU | | MI0000287 |
| 211- | mir- | UGACUUGUGGGCU | UGUCAUC | | |
| 4373088 | 211 | UCCCUUUGUCAUC | CUUCGCCU | | |
| | | CUUCGCCUAGGGC | | | |
| | | UCUGAGCAGGGCA | | | |
| | | GGGACAGCAAAGG | | | |
| | | GGUGCUCAGUUGU | | | |
| | | CACUUCCCACAGC | | | |
| | | ACGGAG | | | |
| hsa-miR- | hsa- | GGGAUGCCACAUU | UUCACAG | UAAAUUU | MI0003191 |
| 513- | mir- | CAGCCAUUCAGCG | GGAGGUG | CACCUUU | |
| 4373239 | 513a-1 | UACAGUGCCUUUC | UCAU | CUGAGAA | |
| | | ACAGGGAGGUGUC | | GG | |
| | | AUUUAUGUGAACU | | | |
| | | AAAAUAUAAAUUUC | | | |
| | | ACCUUUCUGAGAA | | | |
| | | GGGUAAUGUACAG | | | |
| | | CAUGCACUGCAUA | | | |
| | | UGUGGUGUCCC | | | |
| hsa-miR- | hsa- | GGAUGCCACAUUC | UUCACAG | UAAAUUU | MI0003192 |
| 513- | mir- | AGCCAUUCAGUGU | GGAGGUG | CACCUUU | |
| 4373239 | 513a-2 | GCAGUGCCUUUCA | UCAU | CUGAGAA | |
| | | CAGGGAGGUGUCA | | GG | |
| | | UUUAUGUGAACUA | | | |
| | | AAAUAUAAAUUUCA | | | |
| | | CCUUUCUGAGAAG | | | |
| | | GGUAAUGUACAGC | | | |
| | | AUGCACUGCAUAU | | | |
| | | GUGGUGUCC | | | |
| hsa-miR- | hsa- | GUGUACAGUGCCU | UUCACAA | | MI0006648 |
| 513- | mir- | UUCACAAGGAGGU | GGAGGUG | | |
| 4373239 | 513b | GUCAUUUAUGUGA | UCAUUUAU | | |
| | | ACUAAAAUAUAAAU | | | |
| | | GUCACCUUUUUGA | | | |
| | | GAGGAGUAAUGUA | | | |
| | | CAGCA | | | |
| hsa-miR- | hsa- | GCGUACAGUGCCU | UUCUCAA | | MI0006649 |
| 513- | mir- | UUCUCAAGGAGGU | GGAGGUG | | |
| 4373239 | 513c | GUCGUUUAUGUGA | UCGUUUAU | | |
| | | ACUAAAAUAUAAAU | | | |
| | | UUCACCUUUCUGA | | | |
| | | GAAGAGUAAUGUA | | | |
| | | CAGCA | | | |
| hsa-miR- | hsa- | GGAGAGGAGGCAA | AGGCAAG | UGCUAUG | MI0000089 |
| 31- | mir-31 | GAUGCUGGCAUAG | AUGCUGG | CCAACAU | |
| 4373190 | | CUGUUGAACUGGG | CAUAGCU | AUUGCCAU | |
| | | AACCUGCUAUGCC | | | |
| | | AACAUAUUGCCAUC | | | |
| | | UUUCC | | | |
| hsa-miR- | hsa- | UGUUGUCGGGUGG | AAUUGCA | CGGGUGG | MI0000764 |
| 363- | miR- | AUCACGAUGCAAU | CGGUAUC | AUCACGA | |
| 4380917 | 363 | UUUGAUGAGUAUC | CAUCUGUA | UGCAAUUU | |
| | | AUAGGAGAAAAAUU | | | |
| | | GCACGGUAUCCAU | | | |
| | | CUGUAAACC | | | |
| hsa-miR- | | | | | |
| 565- | | | | | |
| 4380942 | | | | | |
| hsa-miR-9- | hsa- | CGGGGUUGGUUGU | UCUUUGG | AUAAAGC | MI0000466 |
| 4373285 | mir-9-1 | UAUCUUUGGUUAU | UUAUCUA | UAGAUAA | |
| | | CUAGCUGUAUGAG | GCUGUAU | CCGAAAGU | |
| | | UGGUGUGGAGUCU | GA | | |
| | | UCAUAAAGCUAGAU | | | |
| | | AACCGAAAGUAAAA | | | |
| | | AUAACCCCA | | | |
| hsa-miR-9- | hsa- | GGAAGCGAGUUGU | UCUUUGG | AUAAAGC | MI0000467 |
| 4373285 | mir-9-2 | UAUCUUUGGUUAU | UUAUCUA | UAGAUAA | |
| | | CUAGCUGUAUGAG | GCUGUAU | CCGAAAGU | |
| | | UGUAUUGGUCUUC | GA | | |
| | | AUAAAGCUAGAUAA | | | |
| | | CCGAAAGUAAAAAC | | | |
| | | UCCUUCA | | | |
| hsa-miR-9- | hsa- | GGAGGCCCGUUUC | UCUUUGG | AUAAAGC | MI0000468 |
| 4373285 | mir-9-3 | UCUCUUUGGUUAU | UUAUCUA | UAGAUAA | |
| | | CUAGCUGUAUGAG | GCUGUAU | CCGAAAGU | |
| | | UGCCACAGAGCCG | GA | | |
| | | UCAUAAAGCUAGAU | | | |
| | | AACCGAAAGUAGAA | | | |
| | | AUGAUUCUCA | | | |
| hsa-miR- | hsa- | GUGCUCGGUUUGU | CAAUCAC | UAGGCAG | MI0000742 |
| 34b- | miR- | AGGCAGUGUCAUU | UAACUCC | UGUCAUU | |
| 4373037 | 34b | AGCUGAUUGUACU | ACUGCCAU | AGCUGAU | |
| | | GUGGUGGUUACAA | | UG | |
| | | UCACUAACUCCACU | | | |
| | | GCCAUCAAAACAAG | | | |
| | | GCAC | | | |
| hsa-miR- | hsa- | CGAGGGGAUACAG | CAGCAGC | CAAAACG | MI0001446 |
| 424- | mir- | CAGCAAUUCAUGU | AAUUCAU | UGAGGCG | |
| 4373201 | 424 | UUUGAAGUGUUCU | GUUUUGAA | CUGCUAU | |
| | | AAAUGGUUCAAAAC | | | |
| | | GUGAGGCGCUGCU | | | |
| | | AUACCCCCUCGUG | | | |
| | | GGGAAGGUAGAAG | | | |
| | | GUGGGG | | | |
| hsa-miR- | hsa- | UGUCGGGUAGCUU | UAGCUUA | CAACACC | MI0000077 |
| 21- | mir-21 | AUCAGACUGAUGU | UCAGACU | AGUCGAU | |
| 4373090 | | UGACUGUUGAAUC | GAUGUUGA | GGGCUGU | |
| | | UCAUGGCAACACC | | | |
| | | AGUCGAUGGGCUG | | | |
| | | UCUGACA | | | |
| hsa-miR- | hsa- | CUGUUAAUGCUAA | UUAAUGC | CUCCUAC | MI0000681 |
| 155- | mir- | UCGUGAUAGGGGU | UAAUCGU | AUAUUAG | |
| 4373124 | 155 | UUUUGCCUCCAAC | GAUAGGG | CAUUAACA | |
| | | UGACUCCUACAUA | GU | | |
| | | UUAGCAUUAACAG | | | |
| hsa-miR- | hsa- | CACUCUGCUGUGG | UAUGGCU | AUGUAGG | MI0000810 |
| 135b- | mir- | CCUAUGGCUUUUC | UUUCAUU | GCUAAAA | |
| 4373139 | 135b | AUUCCUAUGUGAU | CCUAUGU | GCCAUGGG | |
| | | UGCUGUCCCAAAC | GA | | |
| | | UCAUGUAGGGCUA | | | |
| | | AAAGCCAUGGGCU | | | |
| | | ACAGUGAGGGGCG | | | |
| | | AGCUCC | | | |
| hsa-miR-7- | hsa- | UUGGAUGUUGGCC | UGGAAGA | CAACAAAU | MI0000263 |
| 4373014 | mir-7-1 | UAGUUCUGUGUGG | CUAGUGA | CACAGUC | |
| | | AAGACUAGUGAUU | UUUUGUU | UGCCAUA | |
| | | UUGUUGUUUUUAG | GU | | |
| | | AUAACUAAAUCGAC | | | |
| | | AACAAAUCACAGUC | | | |
| | | UGCCAUAUGGCAC | | | |
| | | AGGCCAUGCCUCU | | | |
| | | ACAG | | | |
| hsa-miR-7- | hsa- | CUGGAUACAGAGU | UGGAAGA | CAACAAAU | MI0000264 |
| 4373014 | mir-7-2 | GGACCGGCUGGCC | CUAGUGA | CCCAGUC | |
| | | CCAUCUGGAAGAC | UUUUGUU | UACCUAA | |
| | | UAGUGAUUUUGUU | GU | | |
| | | GUUGUCUUACUGC | | | |
| | | GCUCAACAACAAAU | | | |
| | | CCCAGUCUACCUA | | | |
| | | AUGGUGCCAGCCA | | | |
| | | UCGCA | | | |
| hsa-miR-7- | hsa- | AGAUUAGAGUGGC | UGGAAGA | | MI0000265 |
| 4373014 | mir-7-3 | UGUGGUCUAGUGC | CUAGUGA | | |
| | | UGUGUGGAAGACU | UUUUGUU | | |
| | | AGUGAUUUUGUUG | GU | | |
| | | UUCUGAUGUACUA | | | |
| | | CGACAACAAGUCAC | | | |
| | | AGCCGGCCUCAUA | | | |
| | | GCGCAGACUCCCU | | | |
| | | UCGAC | | | |
| hsa-miR- | hsa- | CUGUGUGUGAUGA | UGGCAGU | | MI0001648 |
| 449- | mir- | GCUGGCAGUGUAU | GUAUUGU | | |
| 4373207 | 449a | UGUUAGCUGGUUG | UAGCUGGU | | |
| | | AAUAUGUGAAUGG | | | |
| | | CAUCGGCUAACAU | | | |
| | | GCAACUGCUGUCU | | | |
| | | UAUUGCAUAUACA | | | |
| hsa-miR- | hsa- | UGACCUGAAUCAG | AGGCAGU | CAGCCAC | MI0003673 |
| 449- | mir- | GUAGGCAGUGUAU | GUAUUGU | AACUACC | |
| 4373207 | 449b | UGUUAGCUGGCUG | UAGCUGGC | CUGCCACU | |
| | | CUUGGGUCAAGUC | | | |
| | | AGCAGCCACAACUA | | | |
| | | CCCUGCCACUUGC | | | |
| | | UUCUGGAUAAAUU | | | |
| | | CUUCU | | | |
| hsa-miR- | hsa- | GCUGGGAUGUGUC | UAGGCAG | UUGCUAG | MI0003823 |
| 449- | mir- | AGGUAGGCAGUGU | UGUAUUG | UUGCACU | |
| 4373207 | 449c | AUUGCUAGCGGCU | CUAGCGG | CCUCUCU | |
| | | GUUAAUGAUUUUA | CUGU | GU | |
| | | ACAGUUGCUAGUU | | | |
| | | GCACUCCUCUCUG | | | |
| | | UUGCAUUCAGAAGC | | | |
| hsa-miR- | hsa- | GGCCAGCUGUGAG | UGGCAGU | CAAUCAG | MI0000268 |
| 34a- | mir- | UGUUUCUUUGGCA | GUCUUAG | CAAGUAU | |
| 4373278 | 34a | GUGUCUUAGCUGG | CUGGUUGU | ACUGCCCU | |
| | | UUGUUGUGAGCAA | | | |
| | | UAGUAAGGAAGCA | | | |
| | | AUCAGCAAGUAUAC | | | |
| | | UGCCCUAGAAGUG | | | |
| | | CUGCACGUUGUGG | | | |
| | | GGCCC | | | |
| hsa-miR- | hsa- | UGCCCUAGCAGCG | UAGCAGC | | MI0003188 |
| 503- | mir- | GGAACAGUUCUGC | GGGAACA | | |
| 4373228 | 503 | AGUGAGCGAUCGG | GUUCUGC | | |
| | | UGCUCUGGGGUAU | AG | | |
| | | UGUUUCCGCUGCC | | | |
| | | AGGGUA | | | |
| hsa-miR- | hsa- | AGUCUAGUUACUA | AGGCAGU | AAUCACU | MI0000743 |
| 34c- | miR- | GGCAGUGUAGUUA | GUAGUUA | AACCACA | |
| 4373036 | 34c | GCUGAUUGCUAAU | GCUGAUU | CGGCCAGG | |
| | | AGUACCAAUCACUA | GC | | |
| | | ACCACACGGCCAG | | | |
| | | GUAAAAAGAUU | | | |
| hsa-miR- | hsa- | AGUACCAAAGUGC | CAAAGUG | ACUGUAG | MI0001519 |
| 20b- | mir- | UCAUAGUGCAGGU | CUCAUAG | UAUGGGC | |
| 4373263 | 20b | AGUUUUGGCAUGA | UGCAGGU | ACUUCCAG | |
| | | CUCUACUGUAGUA | AG | | |
| | | UGGGCACUUCCAG | | | |
| | | UACU | | | |
| hsa-miR- | hsa- | UGACCUGAAUCAG | AGGCAGU | CAGCCAC | MI0003673 |
| 449b- | mir- | GUAGGCAGUGUAU | GUAUUGU | AACUACC | |
| 4381011 | 449b | UGUUAGCUGGCUG | UAGCUGGC | CUGCCACU | |
| | | CUUGGGUCAAGUC | | | |
| | | AGCAGCCACAACUA | | | |
| | | CCCUGCCACUUGC | | | |
| | | UUCUGGAUAAAUU | | | |
| | | CUUCU | | | |
| hsa-miR- | hsa- | CCAGUCACGUCCC | UGGACGG | | MI0000481 |
| 184- | mir- | CUUAUCACUUUUC | AGAACUG | | |
| 4373113 | 184 | CAGCCCAGCUUUG | AUAAGGGU | | |
| | | UGACUGUAAGUGU | | | |
| | | UGGACGGAGAACU | | | |
| | | GAUAAGGGUAGGU | | | |
| | | GAUUGA | | | |
| hsa-miR- | hsa- | ACUGCUAACGAAU | CAGUGCA | | MI0000745 |
| 301- | mir- | GCUCUGACUUUAU | AUAGUAU | | |
| 4373064 | 301a | UGCACUACUGUAC | UGUCAAA | | |
| | | UUUACAGCUAGCA | GC | | |
| | | GUGCAAUAGUAUU | | | |
| | | GUCAAAGCAUCUG | | | |
| | | AAAGCAGG | | | |
| hsa-miR- | hsa- | GCCGCAGGUGCUC | CAGUGCA | | MI0005568 |
| 301- | mir- | UGACGAGGUUGCA | AUGAUAU | | |
| 4373064 | 301b | CUACUGUGCUCUG | UGUCAAA | | |
| | | AGAAGCAGUGCAA | GC | | |
| | | UGAUAUUGUCAAA | | | |
| | | GCAUCUGGGACCA | | | |
| hsa-miR- | hsa- | CUGGCCUCCAGGG | UGAAGGU | UUGUACA | MI0003132 |
| 493- | mir- | CUUUGUACAUGGU | CUACUGU | UGGUAGG | |
| 4373218 | 493 | AGGCUUUCAUUCA | GUGCCAGG | CUUUCAUU | |
| | | UUCGUUUGCACAU | | | |
| | | UCGGUGAAGGUCU | | | |
| | | ACUGUGUGCCAGG | | | |
| | | CCCUGUGCCAG | | | |
| hsa-miR- | hsa- | UGUGCAUCGUGGU | UCAAAUG | ACGGAUG | MI0000111 |
| 105- | mir- | CAAAUGCUCAGAC | CUCAGAC | UUUGAGC | |
| 4373157 | 105-1 | UCCUGUGGUGGCU | UCCUGUG | AUGUGCUA | |
| | | GCUCAUGCACCAC | GU | | |
| | | GGAUGUUUGAGCA | | | |
| | | UGUGCUACGGUGU | | | |
| | | CUA | | | |
| hsa-miR- | hsa- | UGUGCAUCGUGGU | UCAAAUG | ACGGAUG | MI0000112 |
| 105- | mir- | CAAAUGCUCAGAC | CUCAGAC | UUUGAGC | |
| 4373157 | 105-2 | UCCUGUGGUGGCU | UCCUGUG | AUGUGCUA | |
| | | GCUUAUGCACCAC | GU | | |
| | | GGAUGUUUGAGCA | | | |
| | | UGUGCUAUGGUGU | | | |
| | | CUA | | | |
| hsa-miR- | hsa- | GUCCCCUCCCCUA | AACAUUC | | MI0003139 |
| 181d- | mir- | GGCCACAGCCGAG | AUUGUUG | | |
| 4373180 | 181d | GUCACAAUCAACAU | UCGGUGG | | |
| | | UCAUUGUUGUCGG | GU | | |
| | | UGGGUUGUGAGGA | | | |
| | | CUGAGGCCAGACC | | | |
| | | CACCGGGGGAUGA | | | |
| | | AUGUCACUGUGGC | | | |
| | | UGGGCCAGACACG | | | |
| | | GCUUAAGGGGAAU | | | |
| | | GGGGAC | | | |
| hsa-miR- | hsa- | UGUUCUAAGGUGC | UAAGGUG | ACUGCCC | MI0000072 |
| 18a- | mir- | AUCUAGUGCAGAU | CAUCUAG | UAAGUGC | |
| 4373118 | 18a | AGUGAAGUAGAUU | UGCAGAU | UCCUUCU | |
| | | AGCAUCUACUGCC | AG | GG | |
| | | CUAAGUGCUCCUU | | | |
| | | CUGGCA | | | |
| hsa-miR-9- | hsa- | CGGGGUUGGUUGU | UCUUUGG | AUAAAGC | MI0000466 |
| 4378074 | mir-9-1 | UAUCUUUGGUUAU | UUAUCUA | UAGAUAA | |
| | | CUAGCUGUAUGAG | GCUGUAU | CCGAAAGU | |
| | | UGGUGUGGAGUCU | GA | | |
| | | UCAUAAAGCUAGAU | | | |
| | | AACCGAAAGUAAAA | | | |
| | | AUAACCCCA | | | |
| hsa-miR-9- | hsa- | GGAAGCGAGUUGU | UCUUUGG | AUAAAGC | MI0000467 |
| 4378074 | mir-9-2 | UAUCUUUGGUUAU | UUAUCUA | UAGAUAA | |
| | | CUAGCUGUAUGAG | GCUGUAU | CCGAAAGU | |
| | | UGUAUUGGUCUUC | GA | | |
| | | AUAAAGCUAGAUAA | | | |
| | | CCGAAAGUAAAAAC | | | |
| | | UCCUUCA | | | |
| hsa-miR-9- | hsa- | GGAGGCCCGUUUC | UCUUUGG | AUAAAGC | MI0000468 |
| 4378074 | mir-9-3 | UCUCUUUGGUUAU | UUAUCUA | UAGAUAA | |
| | | CUAGCUGUAUGAG | GCUGUAU | CCGAAAGU | |
| | | UGCCACAGAGCCG | GA | | |
| | | UCAUAAAGCUAGAU | | | |
| | | AACCGAAAGUAGAA | | | |
| | | AUGAUUCUCA | | | |
| hsa-miR- | hsa- | UGUGUUAAGGUGC | UAAGGUG | UGCCCUA | MI0001518 |
| 18b- | mir- | AUCUAGUGCAGUU | CAUCUAG | AAUGCCC | |
| 4373184 | 18b | AGUGAAGCAGCUU | UGCAGUU | CUUCUGGC | |
| | | AGAAUCUACUGCC | AG | | |
| | | CUAAAUGCCCCUU | | | |
| | | CUGGCA | | | |
| hsa-miR- | hsa- | CUUUGGCGAUCAC | UCUCUGG | GCAAAGC | MI0000803 |
| 330- | mir- | UGCCUCUCUGGGC | GCCUGUG | ACACGGC | |
| 4373047 | 330 | CUGUGUCUUAGGC | UCUUAGGC | CUGCAGA | |
| | | UCUGCAAGAUCAA | | GA | |
| | | CCGAGCAAAGCAC | | | |
| | | ACGGCCUGCAGAG | | | |
| | | AGGCAGCGCUCUG | | | |
| | | CCC | | | |
| hsa-miR- | hsa- | CCUGUGCAGAGAU | AACAUUC | | MI0000270 |
| 181b- | mir- | UAUUUUUUAAAAG | AUUGCUG | | |
| 4373116 | 181b-1 | GUCACAAUCAACAU | UCGGUGG | | |
| | | UCAUUGCUGUCGG | GU | | |
| | | UGGGUUGAACUGU | | | |
| | | GUGGACAAGCUCA | | | |
| | | CUGAACAAUGAAU | | | |
| | | GCAACUGUGGCCC | | | |
| | | CGCUU | | | |
| hsa-miR- | hsa- | CUGAUGGCUGCAC | AACAUUC | | MI0000683 |
| 181b- | mir- | UCAACAUUCAUUG | AUUGCUG | | |
| 4373116 | 181b-2 | CUGUCGGUGGGUU | UCGGUGG | | |
| | | UGAGUCUGAAUCA | GU | | |
| | | ACUCACUGAUCAAU | | | |
| | | GAAUGCAAACUGC | | | |
| | | GGACCAAACA | | | |
| hsa-miR- | hsa- | CUGUGGUGCAUUG | GUGCAUU | CAAUGUU | MI0000091 |
| 33- | mir- | UAGUUGCAUUGCA | GUAGUUG | UCCACAG | |
| 4373048 | 33a | UGUUCUGGUGGUA | CAUUGCA | UGCAUCAC | |
| | | CCCAUGCAAUGUU | | | |
| | | UCCACAGUGCAUC | | | |
| | | ACAG | | | |
| hsa-miR- | hsa- | GCGGGCGGCCCCG | GUGCAUU | CAGUGCC | MI0003646 |
| 33- | mir- | CGGUGCAUUGCUG | GCUGUUG | UCGGCAG | |
| 4373048 | 33b | UUGCAUUGCACGU | CAUUGC | UGCAGCCC | |
| | | GUGUGAGGCGGGU | | | |
| | | GCAGUGCCUCGGC | | | |
| | | AGUGCAGCCCGGA | | | |
| | | GCCGGCCCCUGGC | | | |
| | | ACCAC | | | |
| hsa-miR- | hsa- | GGUUGCUUCAGUG | AACAUUC | ACCAUCG | MI0000697 |
| 213- | mir- | AACAUUCAACGCU | AACGCUG | ACCGUUG | |
| 4373086 | 181a | GUCGGUGAGUUUG | UCGGUGA | AUUGUACC | |
| | | GAAUUCAAAUAAAA | GU | | |
| | | ACCAUCGACCGUU | | | |
| | | GAUUGUACCCUAU | | | |
| | | AGCUAACC | | | |
| hsa-miR- | hsa- | UCUCAGCCUGUGA | CUCUAGA | AAAGUGC | MI0003148 |
| 519c- | mir- | CCCUCUAGAGGGA | GGGAAGC | AUCUUUU | |
| 4373251 | 519c | AGCGCUUUCUGUU | GCUUUCUG | UAGAGGAU | |
| | | GUCUGAAAGAAAA | | | |
| | | GAAAGUGCAUCUU | | | |
| | | UUUAGAGGAUUAC | | | |
| | | AGUUUGAGA | | | |
| hsa-let-7g- | hsa- | AGGCUGAGGUAGU | UGAGGUA | CUGUACA | MI0000433 |
| 4373163 | mir- | AGUUUGUACAGUU | GUAGUUU | GGCCACU | |
| | let-7g | UGAGGGUCUAUGA | GUACAGUU | GCCUUGC | |
| | | UACCACCCGGUAC | | | |
| | | AGGAGAUAACUGU | | | |
| | | ACAGGCCACUGCC | | | |
| | | UUGCCA | | | |
| hsa-miR- | hsa- | UUGAAGGGAGAUC | AGAUCGA | AAUAAUAC | MI0000777 |
| 369-3p- | mir- | GACCGUGUUAUAU | CCGUGUU | AUGGUUG | |
| 4373032 | 369 | UCGCUUUAUUGAC | AUAUUCGC | AUCUUU | |
| | | UUCGAAUAAUACAU | | | |
| | | GGUUGAUCUUUUC | | | |
| | | UCAG | | | |
| hsa-miR- | hsa- | AAGAAAUGGUUUA | UGGUUUA | UAUGUGG | MI0000744 |
| 299-3p- | mir- | CCGUCCCACAUAC | CCGUCCC | GAUGGUA | |
| 4373189 | 299 | AUUUUGAAUAUGU | ACAUACAU | AACCGCUU | |
| | | AUGUGGGAUGGUA | | | |
| | | AACCGCUUCUU | | | |
| hsa-miR- | hsa- | UGCAUGAGUUCGU | UGGUCUA | | MI0003614 |
| 601- | mir- | CUUGGUCUAGGAU | GGAUUGU | | |
| 4380965 | 601 | UGUUGGAGGAGUC | UGGAGGAG | | |
| | | AGAAAAACUACCCC | | | |
| | | AGGGAUCCUGAAG | | | |
| | | UCCUUUGGGUGGA | | | |
| hsa-miR- | hsa- | UCAUGCUGUGGCC | CAAAGCG | | MI0003156 |
| 518b- | mir- | CUCCAGAGGGAAG | CUCCCCU | | |
| 4373246 | 518b | CGCUUUCUGUUGU | UUAGAGGU | | |
| | | CUGAAAGAAAACAA | | | |
| | | AGCGCUCCCCUUU | | | |
| | | AGAGGUUUACGGU | | | |
| | | UUGA | | | |
| hsa-miR- | hsa- | UGGUACUCGGGGA | AGGUUAC | GAAUGUU | MI0001735 |
| 409-5p- | mir- | GAGGUUACCCGAG | CCGAGCA | GCUCGGU | |
| 4373197 | 409 | CAACUUUGCAUCU | ACUUUGC | GAACCCCU | |
| | | GGACGACGAAUGU | AU | | |
| | | UGCUCGGUGAACC | | | |
| | | CCUUUUCGGUAUCA | | | |
| hsa-miR- | hsa- | CCCAAGUCAGGUA | UGAGUAU | | MI0003136 |
| 496- | mir- | CUCGAAUGGAGGU | UACAUGG | | |
| 4373221 | 496 | UGUCCAUGGUGUG | CCAAUCUC | | |
| | | UUCAUUUUAUUUA | | | |
| | | UGAUGAGUAUUAC | | | |
| | | AUGGCCAAUCUCC | | | |
| | | UUUCGGUACUCAA | | | |
| | | UUCUUCUUGGG | | | |
| hsa-miR- | hsa- | GCCGAGACCGAGU | CUGACCU | CUGCCAA | MI0000234 |
| 192- | mir- | GCACAGGGCUCUG | AUGAAUU | UUCCAUA | |
| 4373108 | 192 | ACCUAUGAAUUGA | GACAGCC | GGUCACAG | |
| | | CAGCCAGUGCUCU | | | |
| | | CGUCUCCCCUCUG | | | |
| | | GCUGCCAAUUCCA | | | |
| | | UAGGUCACAGGUA | | | |
| | | UGUUCGCCUCAAU | | | |
| | | GCCAGC | | | |
| hsa-miR- | hsa- | GGCUGAGCCGCAG | AAGCUGC | AGUUCUU | MI0000078 |
| 22- | mir-22 | UAGUUCUUCAGUG | CAGUUGA | CAGUGGC | |
| 4373079 | | GCAAGCUUUAUGU | AGAACUGU | AAGCUUUA | |
| | | CCUGACCCAGCUA | | | |
| | | AAGCUGCCAGUUG | | | |
| | | AAGAACUGUUGCC | | | |
| | | CUCUGCC | | | |
| hsa-let-7f- | hsa- | UCAGAGUGAGGUA | UGAGGUA | CUAUACA | MI0000067 |
| 4373164 | let-7f-1 | GUAGAUUGUAUAG | GUAGAUU | AUCUAUU | |
| | | UUGUGGGGUAGUG | GUAUAGUU | GCCUUCCC | |
| | | AUUUUACCCUGUU | | | |
| | | CAGGAGAUAACUA | | | |
| | | UACAAUCUAUUGC | | | |
| | | CUUCCCUGA | | | |
| hsa-let-7f- | hsa- | UGUGGGAUGAGGU | UGAGGUA | CUAUACA | MI0000068 |
| 4373164 | let-7f-2 | AGUAGAUUGUAUA | GUAGAUU | GUCUACU | |
| | | GUUUUAGGGUCAU | GUAUAGUU | GUCUUUCC | |
| | | ACCCCAUCUUGGA | | | |
| | | GAUAACUAUACAGU | | | |
| | | CUACUGUCUUUCC | | | |
| | | CACG | | | |
| hsa-miR- | hsa- | AUGGUGUUAUCAA | UGUAACA | | MI0000488 |
| 194- | mir- | GUGUAACAGCAAC | GCAACUC | | |
| 4373106 | 194-1 | UCCAUGUGGACUG | CAUGUGGA | | |
| | | UGUACCAAUUUCC | | | |
| | | AGUGGAGAUGCUG | | | |
| | | UUACUUUUGAUGG | | | |
| | | UUACCAA | | | |
| hsa-miR- | hsa- | UGGUUCCCGCCCC | UGUAACA | CCAGUGG | MI0000732 |
| 194- | mir- | CUGUAACAGCAAC | GCAACUC | GGCUGCU | |
| 4373106 | 194-2 | UCCAUGUGGAAGU | CAUGUGGA | GUUAUCUG | |
| | | GCCCACUGGUUCC | | | |
| | | AGUGGGGCUGCUG | | | |
| | | UUAUCUGGGGCGA | | | |
| | | GGGCCAG | | | |
| hsa-miR- | hsa- | GUCGAGGCCGUGG | GUCCGCU | | MI0003579 |
| 572- | mir- | CCCGGAAGUGGUC | CGGCGGU | | |
| 4381017 | 572 | GGGGCCGCUGCGG | GGCCCA | | |
| | | GCGGAAGGGCGCC | | | |
| | | UGUGCUUCGUCCG | | | |
| | | CUCGGCGGUGGCC | | | |
| | | CAGCCAGGCCCGC | | | |
| | | GGGA | | | |
| hsa-miR- | msa- | GAGUUUGGUUUUG | CUAGGUA | GCCCCUG | MI0000812 |
| 331- | mir- | UUUGGGUUUGUUC | UGGUCCC | GGCCUAU | |
| 4373046 | 331 | UAGGUAUGGUCCC | AGGGAUCC | CCUAGAA | |
| | | AGGGAUCCCAGAU | | | |
| | | CAAACCAGGCCCC | | | |
| | | UGGGCCUAUCCUA | | | |
| | | GAACCAACCUAAGC | | | |
| | | UC | | | |
| hsa-miR- | hsa- | GUGGCAGCUUGGU | GUGACAU | | MI0003124 |
| 489- | mir- | GGUCGUAUGUGUG | CACAUAU | | |
| 4373214 | 489 | ACGCCAUUUACUU | ACGGCAGC | | |
| | | GAACCUUUAGGAG | | | |
| | | UGACAUCACAUAUA | | | |
| | | CGGCAGCUAAACU | | | |
| | | GCUAC | | | |
| hsa-miR- | hsa- | UGACUCCUCCAGG | UCUUGGA | CUGGAUG | MI0003133 |
| 432- | mir- | UCUUGGAGUAGGU | GUAGGUC | GCUCCUC | |
| 4373280 | 432 | CAUUGGGUGGAUC | AUUGGGU | CAUGUCU | |
| | | CUCUAUUUCCUUA | GG | | |
| | | CGUGGGCCACUGG | | | |
| | | AUGGCUCCUCCAU | | | |
| | | GUCUUGGAGUAGA | | | |
| | | UCA | | | |
| hsa-miR- | hsa- | UCCCUUUCCCAGG | UGGGUUU | GUUCUCC | MI0003643 |
| 629- | mir- | GGAGGGGCUGGGU | ACGUUGG | CAACGUA | |
| 4380969 | 629 | UUACGUUGGGAGA | GAGAACU | AGCCCAGC | |
| | | ACUUUUACGGUGA | | | |
| | | ACCAGGAGGUUCU | | | |
| | | CCCAACGUAAGCC | | | |
| | | CAGCCCCUCCCCU | | | |
| | | CUGCCU | | | |
| hsa-miR- | hsa- | CGCCUCAGAGCCG | AGAGGUA | UUCCUAU | MI0003130 |
| 202- | mir- | CCCGCCGUUCCUU | UAGGGCA | GCAUAUA | |
| 4378075 | 202 | UUUCCUAUGCAUA | UGGGAA | CUUCUUUG | |
| | | UACUUCUUUGAGG | | | |
| | | AUCUGGCCUAAAG | | | |
| | | AGGUAUAGGGCAU | | | |
| | | GGGAAAACGGGGC | | | |
| | | GGUCGGGUCCUCC | | | |
| | | CCAGCG | | | |
| hsa-miR- | hsa- | GAGGCAAAGUUCU | UCAGUGC | AAAGUUC | MI0000253 |
| 148a- | mir- | GAGACACUCCGAC | ACUACAG | UGAGACA | |
| 4373130 | 148a | UCUGAGUAUGAUA | AACUUUGU | CUCCGACU | |
| | | GAAGUCAGUGCAC | | | |
| | | UACAGAACUUUGU | | | |
| | | CUC | | | |
| hsa-miR- | hsa- | UACUUAUUACUGG | GUGAGUC | | MI0003641 |
| 627- | mir- | UAGUGAGUCUCUA | UCUAAGA | | |
| 4380967 | 627 | AGAAAAGAGGAGG | AAAGAGGA | | |
| | | UGGUUGUUUUCCU | | | |
| | | CCUCUUUUCUUUG | | | |
| | | AGACUCACUACCAA | | | |
| | | UAAUAAGAAAUACU | | | |
| | | ACUA | | | |
| hsa-miR- | hsa- | CAAUAGACACCCAU | GUGUCUU | | MI0003127 |
| 511- | mir- | CGUGUCUUUUGCU | UUGCUCU | | |
| 4373236 | 511-1 | CUGCAGUCAGUAA | GCAGUCA | | |
| | | AUAUUUUUUUGUG | | | |
| | | AAUGUGUAGCAAAA | | | |
| | | GACAGAAUGGUGG | | | |
| | | UCCAUUG | | | |
| hsa-miR- | hsa- | CAAUAGACACCCAU | GUGUCUU | | MI0003128 |
| 511- | mir- | CGUGUCUUUUGCU | UUGCUCU | | |
| 4373236 | 511-2 | CUGCAGUCAGUAA | GCAGUCA | | |
| | | AUAUUUUUUUGUG | | | |
| | | AAUGUGUAGCAAAA | | | |
| | | GACAGAAUGGUGG | | | |
| | | UCCAUUG | | | |
| hsa-miR- | hsa- | CUCCGGUGCCUAC | UGGCUCA | UGCCUAC | MI0000080 |
| 24- | mir- | UGAGCUGAUAUCA | GUUCAGC | UGAGCUG | |
| 4373072 | 24-1 | GUUCUCAUUUUAC | AGGAACAG | AUAUCAGU | |
| | | ACACUGGCUCAGU | | | |
| | | UCAGCAGGAACAG | | | |
| | | GAG | | | |
| hsa-miR- | hsa- | CUCUGCCUCCCGU | UGGCUCA | UGCCUAC | MI0000081 |
| 24- | mir- | GCCUACUGAGCUG | GUUCAGC | UGAGCUG | |
| 4373072 | 24-2 | AAACACAGUUGGU | AGGAACAG | AAACACAG | |
| | | UUGUGUACACUGG | | | |
| | | CUCAGUUCAGCAG | | | |
| | | GAACAGGG | | | |
| hsa-let-7a- | hsa- | UGGGAUGAGGUAG | UGAGGUA | CUAUACA | MI0000060 |
| 4373169 | let-7a-1 | UAGGUUGUAUAGU | GUAGGUU | AUCUACU | |
| | | UUUAGGGUCACAC | GUAUAGUU | GUCUUUC | |
| | | CCACCACUGGGAG | | | |
| | | AUAACUAUACAAUC | | | |
| | | UACUGUCUUUCCUA | | | |
| hsa-let-7a- | hsa- | AGGUUGAGGUAGU | UGAGGUA | CUGUACA | MI0000061 |
| 4373169 | let-7a-2 | AGGUUGUAUAGUU | GUAGGUU | GCCUCCU | |
| | | UAGAAUUACAUCAA | GUAUAGUU | AGCUUUCC | |
| | | GGGAGAUAACUGU | | | |
| | | ACAGCCUCCUAGC | | | |
| | | UUUCCU | | | |
| hsa-let-7a- | hsa- | GGGUGAGGUAGUA | UGAGGUA | CUAUACA | MI0000062 |
| 4373169 | let-7a-3 | GGUUGUAUAGUUU | GUAGGUU | AUCUACU | |
| | | GGGGCUCUGCCCU | GUAUAGUU | GUCUUUC | |
| | | GCUAUGGGAUAAC | | | |
| | | UAUACAAUCUACUG | | | |
| | | UCUUUCCU | | | |
| hsa-miR- | hsa- | GAUCUGUCUGUCU | UACCCUG | CAAAUUC | MI0000266 |
| 10a- | mir- | UCUGUAUAUACCC | UAGAUCC | GUAUCUA | |
| 4373153 | 10a | UGUAGAUCCGAAU | GAAUUUG | GGGGAAUA | |
| | | UUGUGUAAGGAAU | UG | | |
| | | UUUGUGGUCACAA | | | |
| | | AUUCGUAUCUAGG | | | |
| | | GGAAUAUGUAGUU | | | |
| | | GACAUAAACACUCC | | | |
| | | GCUCU | | | |
| hsa-miR- | hsa- | ACCCGGCAGUGCC | CUGUGCG | | MI0000286 |
| 210- | mir- | UCCAGGCGCAGGG | UGUGACA | | |
| 4373089 | 210 | CAGCCCCUGCCCA | GCGGCUGA | | |
| | | CCGCACACUGCGC | | | |
| | | UGCCCCAGACCCA | | | |
| | | CUGUGCGUGUGAC | | | |
| | | AGCGGCUGAUCUG | | | |
| | | UGCCUGGGCAGCG | | | |
| | | CGACCC | | | |
| hsa-miR- | hsa- | CUGAGGAGCAGGG | UUCACAG | AGGGCUU | MI0000085 |
| 27a- | mir- | CUUAGCUGCUUGU | UGGCUAA | AGCUGCU | |
| 4373287 | 27a | GAGCAGGGUCCAC | GUUCCGC | UGUGAGCA | |
| | | ACCAAGUCGUGUU | | | |
| | | CACAGUGGCUAAG | | | |
| | | UUCCGCCCCCCAG | | | |
| hsa-miR- | hsa- | ACUGGUCGGUGAU | UAGGUAG | UCGACAG | MI0001150 |
| 196b- | mir- | UUAGGUAGUUUCC | UUUCCUG | CACGACA | |
| 4373103 | 196b | UGUUGUUGGGAUC | UUGUUGGG | CUGCCUUC | |
| | | CACCUUUCUCUCG | | | |
| | | ACAGCACGACACU | | | |
| | | GCCUUCAUUACUU | | | |
| | | CAGUUG | | | |
| hsa-miR- | hsa- | CCGGGACCCAGUU | UUCAAGU | CCUGUUC | MI0000084 |
| 26b- | mir- | CAAGUAAUUCAGG | AAUUCAG | UCCAUUA | |
| 4373069 | 26b | AUAGGUUGUGUGC | GAUAGGU | CUUGGCUC | |
| | | UGUCCAGCCUGUU | | | |
| | | CUCCAUUACUUGG | | | |
| | | CUCGGGGACCGG | | | |
| hsa-miR- | hsa- | GGGCAGUCUUUGC | UGUAAAC | CUUUCAG | MI0000749 |
| 30e-3p- | mir- | UACUGUAAACAUCC | AUCCUUG | UCGGAUG | |
| 4373057 | 30e | UUGACUGGAAGCU | ACUGGAAG | UUUACAGC | |
| | | GUAAGGUGUUCAG | | | |
| | | AGGAGCUUUCAGU | | | |
| | | CGGAUGUUUACAG | | | |
| | | CGGCAGGCUGCCA | | | |
| hsa-miR- | hsa- | AGGGCUCCUGACU | ACUGGAC | CUCCUGA | MI0000786 |
| 422b- | mir- | CCAGGUCCUGUGU | UUGGAGU | CUCCAGG | |
| 4373016 | 378 | GUUACCUAGAAAUA | CAGAAGG | UCCUGUGU | |
| | | GCACUGGACUUGG | | | |
| | | AGUCAGAAGGCCU | | | |
| hsa-miR- | hsa- | CUCGGGAGGGGCG | GGGGGUC | UCCGAGC | MI0003628 |
| 615- | miR- | GGAGGGGGGUCCC | CCCGGUG | CUGGGUC | |
| 4380991 | 615 | CGGUGCUCGGAUC | CUCGGAUC | UCCCUCUU | |
| | | UCGAGGGUGCUUA | | | |
| | | UUGUUCGGUCCGA | | | |
| | | GCCUGGGUCUCCC | | | |
| | | UCUUCCCCCCAAC | | | |
| | | CCCCC | | | |
| hsa-miR- | hsa- | UGUCCCCCCCGGC | UCAGUGC | | MI0000462 |
| 152- | miR- | CCAGGUUCUGUGA | AUGACAG | | |
| 4373126 | 152 | UACACUCCGACUC | AACUUGG | | |
| | | GGGCUCUGGAGCA | | | |
| | | GUCAGUGCAUGAC | | | |
| | | AGAACUUGGGCCC | | | |
| | | GGAAGGACC | | | |
| hsa-miR- | hsa- | GGCCGGCUGGGGU | AUCACAU | GGGGUUC | MI0000079 |
| 23a- | miR- | UCCUGGGGAUGGG | UGCCAGG | CUGGGGA | |
| 4373074 | 23a | AUUUGCUUCCUGU | GAUUUCC | UGGGAUUU | |
| | | CACAAAUCACAUUG | | | |
| | | CCAGGGAUUUCCA | | | |
| | | ACCGACC | | | |
| hsa-let-7e- | hsa- | CCCGGGCUGAGGU | UGAGGUA | CUAUACG | MI0000066 |
| 4373165 | let-7e | AGGAGGUUGUAUA | GGAGGUU | GCCUCCU | |
| | | GUUGAGGAGGACA | GUAUAGUU | AGCUUUCC | |
| | | CCCAAGGAGAUCA | | | |
| | | CUAUACGGCCUCC | | | |
| | | UAGCUUUCCCCAGG | | | |
| hsa-miR- | hsa- | UGCAGGCCUCUGU | UGAUAUG | | MI0000486 |
| 190- | mir- | GUGAUAUGUUUGA | UUUGAUA | | |
| 4373110 | 190 | UAUAUUAGGUUGU | UAUUAGGU | | |
| | | UAUUUAAUCCAACU | | | |
| | | AUAUAUCAAACAUA | | | |
| | | UUCCUACAGUGUC | | | |
| | | UUGCC | | | |
| hsa-miR- | hsa- | UUGAAGGGAGAUC | AGAUCGA | AAUAAUAC | MI0000777 |
| 369-5p- | miR- | GACCGUGUUAUAU | CCGUGUU | AUGGUUG | |
| 4373195 | 369 | UCGCUUUAUUGAC | AUAUUCGC | AUCUUU | |
| | | UUCGAAUAAUACAU | | | |
| | | GGUUGAUCUUUUC | | | |
| | | UCAG | | | |
| hsa-miR- | hsa- | GGGCAGUCUUUGC | UGUAAAC | CUUUCAG | MI0000749 |
| 30e-5p- | mir- | UACUGUAAACAUCC | AUCCUUG | UCGGAUG | |
| 4373058 | 30e | UUGACUGGAAGCU | ACUGGAAG | UUUACAGC | |
| | | GUAAGGUGUUCAG | | | |
| | | AGGAGCUUUCAGU | | | |
| | | CGGAUGUUUACAG | | | |
| | | CGGCAGGCUGCCA | | | |
| hsa-miR- | hsa- | GCUGCUGUUGGGA | AGACCCU | | MI0003189 |
| 504- | mir- | GACCCUGGUCUGC | GGUCUGC | | |
| 4373229 | 504 | ACUCUAUCUGUAU | ACUCUAUC | | |
| | | UCUUACUGAAGGG | | | |
| | | AGUGCAGGGCAGG | | | |
| | | GUUUCCCAUACAG | | | |
| | | AGGGC | | | |
| hsa-miR- | hsa- | AUCUCUUACACAG | UAGCACC | UGACCGA | MI0000735 |
| 29c- | miR- | GCUGACCGAUUUC | AUUUGAA | UUUCUCC | |
| 4373289 | 29c | UCCUGGUGUUCAG | AUCGGUUA | UGGUGUUC | |
| | | AGUCUGUUUUUGU | | | |
| | | CUAGCACCAUUUG | | | |
| | | AAAUCGGUUAUGA | | | |
| | | UGUAGGGGGA | | | |
| hsa-miR- | hsa- | GUGGCCUCGUUCA | UUCAAGU | CCUAUUC | MI0000083 |
| 26a- | miR- | AGUAAUCCAGGAU | AAUCCAG | UUGGUUA | |
| 4373070 | 26a-1 | AGGCUGUGCAGGU | GAUAGGCU | CUUGCACG | |
| | | CCCAAUGGGCCUA | | | |
| | | UUCUUGGUUACUU | | | |
| | | GCACGGGGACGC | | | |
| hsa-miR- | hsa- | GGCUGUGGCUGGA | UUCAAGU | CCUAUUC | MI0000750 |
| 26a- | miR- | UUCAAGUAAUCCA | AAUCCAG | UUGAUUA | |
| 4373070 | 26a-2 | GGAUAGGCUGUUU | GAUAGGCU | CUUGUUUC | |
| | | CCAUCUGUGAGGC | | | |
| | | CUAUUCUUGAUUA | | | |
| | | CUUGUUUCUGGAG | | | |
| | | GCAGCU | | | |
| hsa-miR- | hsa- | UGCCCUGGCUCAG | UACAGUA | CAGUUAU | MI0000103 |
| 101- | miR- | UUAUCACAGUGCU | CUGUGAU | CACAGUG | |
| 4373159 | 101-1 | GAUGCUGUCUAUU | AACUGAA | CUGAUGCU | |
| | | CUAAAGGUACAGU | | | |
| | | ACUGUGAUAACUG | | | |
| | | AAGGAUGGCA | | | |
| hsa-miR- | hsa- | ACUGUCCUUUUUC | UACAGUA | | MI0000739 |
| 101- | miR- | GGUUAUCAUGGUA | CUGUGAU | | |
| 4373159 | 101-2 | CCGAUGCUGUAUA | AACUGAA | | |
| | | UCUGAAAGGUACA | | | |
| | | GUACUGUGAUAAC | | | |
| | | UGAAGAAUGGUGGU | | | |
| hsa-miR- | hsa- | UACUUGAAGAGAA | GAAGUUG | | MI0000790 |
| 382- | mir- | GUUGUUCGUGGUG | UUCGUGG | | |
| 4373019 | 382 | GAUUCGCUUUACU | UGGAUUCG | | |
| | | UAUGACGAAUCAU | | | |
| | | UCACGGACAACAC | | | |
| | | UUUUUUCAGUA | | | |
| hsa-miR- | hsa- | UGGAGUGGGGGGG | CUGGCCC | | MI0000804 |
| 328- | mir- | CAGGAGGGGCUCA | UCUCUGC | | |
| 4373049 | 328 | GGGAGAAAGUGCA | CCUUCCGU | | |
| | | UACAGCCCCUGGC | | | |
| | | CCUCUCUGCCCUU | | | |
| | | CCGUCCCCUG | | | |
| hsa-miR- | hsa- | GGCUACAGUCUUU | UUCCCUU | | MI0000284 |
| 204- | miR- | CUUCAUGUGACUC | UGUCAUC | | |
| 4373094 | 204 | GUGGACUUCCCUU | CUAUGCCU | | |
| | | UGUCAUCCUAUGC | | | |
| | | CUGAGAAUAUAUG | | | |
| | | AAGGAGGCUGGGA | | | |
| | | AGGCAAAGGGACG | | | |
| | | UUCAAUUGUCAUC | | | |
| | | ACUGGC | | | |
| hsa-miR- | hsa- | UGUGAUCACUGUC | CUGAAGC | UCGGAUC | MI0000472 |
| 127- | mir- | UCCAGCCUGCUGA | UCAGAGG | CGUCUGA | |
| 4373147 | 127 | AGCUCAGAGGGCU | GCUCUGAU | GCUUGGCU | |
| | | CUGAUUCAGAAAG | | | |
| | | AUCAUCGGAUCCG | | | |
| | | UCUGAGCUUGGCU | | | |
| | | GGUCGGAAGUCUC | | | |
| | | AUCAUC | | | |
| hsa-miR- | hsa- | GCGACUGUAAACA | UGUAAAC | CUUUCAG | MI0000088 |
| 30a-3p- | miR- | UCCUCGACUGGAA | AUCCUCG | UCGGAUG | |
| 4373062 | 30a | GCUGUGAAGCCAC | ACUGGAAG | UUUGCAGC | |
| | | AGAUGGGCUUUCA | | | |
| | | GUCGGAUGUUUGC | | | |
| | | AGCUGC | | | |
| hsa-miR- | hsa- | AGAUGUGCUCUCC | GCGACCC | GAAAUCA | MI0003575 |
| 551b- | miR- | UGGCCCAUGAAAU | AUACUUG | AGCGUGG | |
| 4380945 | 551b | CAAGCGUGGGUGA | GUUUCAG | GUGAGACC | |
| | | GACCUGGUGCAGA | | | |
| | | ACGGGAAGGCGAC | | | |
| | | CCAUACUUGGUUU | | | |
| | | CAGAGGCUGUGAG | | | |
| | | AAUAA | | | |
| hsa-miR- | hsa- | CUCCGGUGCCUAC | UGGCUCA | UGCCUAC | MI0000080 |
| 189- | mir- | UGAGCUGAUAUCA | GUUCAGC | UGAGCUG | |
| 4378067 | 24-1 | GUUCUCAUUUUAC | AGGAACAG | AUAUCAGU | |
| | | ACACUGGCUCAGU | | | |
| | | UCAGCAGGAACAG | | | |
| | | GAG | | | |
| hsa-miR- | hsa- | CGAGGAUGGGAGC | UGGGUCU | AACUGGC | MI0000487 |
| 193a- | mir- | UGAGGGCUGGGUC | UUGCGGG | CUACAAA | |
| 4373107 | 193a | UUUGCGGGCGAGA | CGAGAUGA | GUCCCAGU | |
| | | UGAGGGUGUCGGA | | | |
| | | UCAACUGGCCUAC | | | |
| | | AAAGUCCCAGUUC | | | |
| | | UCGGCCCCCG | | | |
| hsa-miR- | hsa- | AGGGCUCCUGACU | ACUGGAC | CUCCUGA | MI0000786 |
| 378- | mir- | CCAGGUCCUGUGU | UUGGAGU | CUCCAGG | |
| 4373024 | 378 | GUUACCUAGAAAUA | CAGAAGG | UCCUGUGU | |
| | | GCACUGGACUUGG | | | |
| | | AGUCAGAAGGCCU | | | |
| hsa-miR- | hsa- | CCAGAGGUUGUAA | UACCCUG | ACAGAUU | MI0000267 |
| 10b- | miR- | CGUUGUCUAUAUA | UAGAACC | CGAUUCU | |
| 4373152 | 10b | UACCCUGUAGAAC | GAAUUUG | AGGGGAAU | |
| | | CGAAUUUGUGUGG | UG | | |
| | | UAUCCGUAUAGUC | | | |
| | | ACAGAUUCGAUUC | | | |
| | | UAGGGGAAUAUAU | | | |
| | | GGUCGAUGCAAAA | | | |
| | | ACUUCA | | | |
| hsa-miR- | hsa- | ACUUGGAGAGAGG | AGAGGCU | GUCAUAC | MI0002469 |
| 485-3p- | mir- | CUGGCCGUGAUGA | GGCCGUG | ACGGCUC | |
| 4378095 | 485 | AUUCGAUUCAUCAA | AUGAAUUC | UCCUCUCU | |
| | | AGCGAGUCAUACA | | | |
| | | CGGCUCUCCUCUC | | | |
| | | UUUUAGU | | | |
| hsa-miR- | hsa- | GGUACCUGAGAAG | AAUAUAAC | | MI0002465 |
| 410- | mir- | AGGUUGUCUGUGA | ACAGAUG | | |
| 4378093 | 410 | UGAGUUCGCUUUU | GCCUGU | | |
| | | AUUAAUGACGAAUA | | | |
| | | UAACACAGAUGGC | | | |
| | | CUGUUUUCAGUACC | | | |
| hsa-miR- | hsa- | GUGAAUUAGGUAG | UAGGUAG | | MI0000238 |
| 196a- | miR- | UUUCAUGUUGUUG | UUUCAUG | | |
| 4373104 | 196a-1 | GGCCUGGGUUUCU | UUGUUGGG | | |
| | | GAACACAACAACAU | | | |
| | | UAAACCACCCGAUU | | | |
| | | CAC | | | |
| hsa-miR- | hsa- | UGCUCGCUCAGCU | UAGGUAG | CGGCAAC | MI0000279 |
| 196a- | miR- | GAUCUGUGGCUUA | UUUCAUG | AAGAAAC | |
| 4373104 | 196a-1 | GGUAGUUUCAUGU | UUGUUGGG | UGCCUGAG | |
| | | UGUUGGGAUUGAG | | | |
| | | UUUUGAACUCGGC | | | |
| | | AACAAGAAACUGCC | | | |
| | | UGAGUUACAUCAG | | | |
| | | UCGGUUUUCGUCG | | | |
| | | AGGGC | | | |
| hsa-let-7b- | hsa- | CGGGGUGAGGUAG | UGAGGUA | CUAUACA | MI0000063 |
| 4373168 | let-7b | UAGGUUGUGUGGU | GUAGGUU | ACCUACU | |
| | | UUCAGGGCAGUGA | GUGUGGUU | GCCUUCCC | |
| | | UGUUGCCCCUCGG | | | |
| | | AAGAUAACUAUACA | | | |
| | | ACCUACUGCCUUC | | | |
| | | CCUG | | | |
| hsa-miR- | hsa- | UUGGUACUUGGAG | AAUCGUA | | MI0003530 |
| 487b- | mir- | AGUGGUUAUCCCU | CAGGGUC | | |
| 4378102 | 487b | GUCCUGUUCGUUU | AUCCACUU | | |
| | | UGCUCAUGUCGAA | | | |
| | | UCGUACAGGGUCA | | | |
| | | UCCACUUUUUCAG | | | |
| | | UAUCAA | | | |
| hsa-miR- | hsa- | ACCGCAGGGAAAA | UAAUGCC | | MI0000767 |
| 365- | mir- | UGAGGGACUUUUG | CCUAAAAA | | |
| 4373194 | 365-1 | GGGGCAGAUGUGU | UCCUUAU | | |
| | | UUCCAUUCCACUA | | | |
| | | UCAUAAUGCCCCU | | | |
| | | AAAAAUCCUUAUUG | | | |
| | | CUCUUGCA | | | |
| hsa-miR- | hsa- | AGAGUGUUCAAGG | UAAUGCC | AGGGACU | MI0000769 |
| 365- | mir- | ACAGCAAGAAAAAU | CCUAAAAA | UUCAGGG | |
| 4373194 | 365-2 | GAGGGACUUUCAG | UCCUUAU | GCAGCUGU | |
| | | GGGCAGCUGUGUU | | | |
| | | UUCUGACUCAGUC | | | |
| | | AUAAUGCCCCUAAA | | | |
| | | AAUCCUUAUUGUU | | | |
| | | CUUGCAGUGUGCA | | | |
| | | UCGGG | | | |
| hsa-miR- | hsa- | CGCUGGCGACGGG | UCGUACC | CAUUAUU | MI0000471 |
| 126- | mir- | ACAUUAUUACUUUU | GUGAGUA | ACUUUUG | |
| 4373269 | 126 | GGUACGCGCUGUG | AUAAUGCG | GUACGCG | |
| | | ACACUUCAAACUCG | | | |
| | | UACCGUGAGUAAU | | | |
| | | AAUGCGCCGUCCA | | | |
| | | CGGCA | | | |
| hsa-miR- | hsa- | CAGGGUGUGUGAC | UGUGACU | | MI0000474 |
| 134- | mir- | UGGUUGACCAGAG | GGUUGAC | | |
| 4373141 | 134 | GGGCAUGCACUGU | CAGAGGGG | | |
| | | GUUCACCCUGUGG | | | |
| | | GCCACCUAGUCAC | | | |
| | | CAACCCUC | | | |
| hsa-miR- | hsa- | CGCUGGCGACGGG | UCGUACC | CAUUAUU | MI0000471 |
| 126- | mir- | ACAUUAUUACUUUU | GUGAGUA | ACUUUUG | |
| 4378064 | 126 | GGUACGCGCUGUG | AUAAUGCG | GUACGCG | |
| | | ACACUUCAAACUCG | | | |
| | | UACCGUGAGUAAU | | | |
| | | AAUGCGCCGUCCA | | | |
| | | CGGCA | | | |
| hsa-miR- | hsa- | ACUUGGAGAGAGG | AGAGGCU | GUCAUAC | MI0002469 |
| 485-5p- | mir- | CUGGCCGUGAUGA | GGCCGUG | ACGGCUC | |
| 4373212 | 485 | AUUCGAUUCAUCAA | AUGAAUUC | UCCUCUCU | |
| | | AGCGAGUCAUACA | | | |
| | | CGGCUCUCCUCUC | | | |
| | | UUUUAGU | | | |
| hsa-miR- | hsa- | ACCUCUCUAACAAG | UUCACAG | AGAGCUU | MI0000440 |
| 27b- | mir- | GUGCAGAGCUUAG | UGGCUAA | AGCUGAU | |
| 4373068 | 27b | CUGAUUGGUGAAC | GUUCUGC | UGGUGAAC | |
| | | AGUGAUUGGUUUC | | | |
| | | CGCUUUGUUCACA | | | |
| | | GUGGCUAAGUUCU | | | |
| | | GCACCUGAAGAGA | | | |
| | | AGGUG | | | |
| hsa-miR- | hsa- | CCGGGGAGAAGUA | AUCAUGA | | MI0001723 |
| 433- | miR- | CGGUGAGCCUGUC | UGGGCUC | | |
| 4373205 | 433 | AUUAUUCAGAGAG | CUCGGUGU | | |
| | | GCUAGAUCCUCUG | | | |
| | | UGUUGAGAAGGAU | | | |
| | | CAUGAUGGGCUCC | | | |
| | | UCGGUGUUCUCCA | | | |
| | | GG | | | |
| hsa-miR- | hsa- | UGGUACUUGGAGA | UAGUAGA | UAUGUAA | MI0003675 |
| 411- | mir- | GAUAGUAGACCGU | CCGUAUA | CACGGUC | |
| 4381013 | 411 | AUAGCGUACGCUU | GCGUACG | CACUAACC | |
| | | UAUCUGUGACGUA | | | |
| | | UGUAACACGGUCC | | | |
| | | ACUAACCCUCAGUA | | | |
| | | UCAAAUCCAUCCCC | | | |
| | | GAG | | | |
| hsa-miR- | hsa- | UAAAAGGUAGAUU | AUCAUAG | GUAGAUU | MI0000784 |
| 376a- | mir- | CUCCUUCUAUGAG | AGGAAAA | CUCCUUC | |
| 4378104 | 376a-1 | UACAUUAUUUAUGA | UCCACGU | UAUGAGUA | |
| | | UUAAUCAUAGAGG | | | |
| | | AAAAUCCACGUUUUC | | | |
| hsa-miR- | hsa- | GGUAUUUAAAAGG | AUCAUAG | | MI0003529 |
| 376a- | mir- | UAGAUUUUCCUUC | AGGAAAA | | |
| 4378104 | 376a-2 | UAUGGUUACGUGU | UCCACGU | | |
| | | UUGAUGGUUAAUC | | | |
| | | AUAGAGGAAAAUCC | | | |
| | | ACGUUUUCAGUAUC | | | |
| hsa-miR- | hsa- | GCCAACCCAGUGU | CCCAGUG | ACAGUAG | MI0000242 |
| 199a- | mir- | UCAGACUACCUGU | UUCAGAC | UCUGCAC | |
| 4378068 | 199a-1 | UCAGGAGGCUCUC | UACCUGU | AUUGGUUA | |
| | | AAUGUGUACAGUA | UC | | |
| | | GUCUGCACAUUGG | | | |
| | | UUAGGC | | | |
| hsa-miR- | hsa- | AGGAAGCUUCUGG | CCCAGUG | ACAGUAG | MI0000281 |
| 199a- | mir- | AGAUCCUGCUCCG | UUCAGAC | UCUGCAC | |
| 4378068 | 199a-2 | UCGCCCCAGUGUU | UACCUGU | AUUGGUUA | |
| | | CAGACUACCUGUU | UC | | |
| | | CAGGACAAUGCCG | | | |
| | | UUGUACAGUAGUC | | | |
| | | UGCACAUUGGUUA | | | |
| | | GACUGGGCAAGGG | | | |
| | | AGAGCA | | | |
| hsa-miR- | hsa- | UGCGCUCCUCUCA | UCCCUGA | ACGGGUU | MI0000446 |
| 125b- | miR- | GUCCCUGAGACCC | GACCCUA | AGGCUCU | |
| 4373148 | 125b-1 | UAACUUGUGAUGU | ACUUGUGA | UGGGAGCU | |
| | | UUACCGUUUAAAU | | | |
| | | CCACGGGUUAGGC | | | |
| | | UCUUGGGAGCUGC | | | |
| | | GAGUCGUGCU | | | |
| hsa-miR- | hsa- | ACCAGACUUUUCC | UCCCUGA | UCACAAG | MI0000470 |
| 125b- | miR- | UAGUCCCUGAGAC | GACCCUA | UCAGGCU | |
| 4373148 | 125b-2 | CCUAACUUGUGAG | ACUUGUGA | CUUGGGAC | |
| | | GUAUUUUAGUAAC | | | |
| | | AUCACAAGUCAGG | | | |
| | | CUCUUGGGACCUA | | | |
| | | GGCGGAGGGGA | | | |
| hsa-miR- | hsa- | AGAGAUGGUAGAC | UGGUAGA | UAUGUAA | MI0000787 |
| 379- | miR- | UAUGGAACGUAGG | CUAUGGA | CAUGGUC | |
| 4373023 | 379 | CGUUAUGAUUUCU | ACGUAGG | CACUAACU | |
| | | GACCUAUGUAACA | | | |
| | | UGGUCCACUAACU | | | |
| | | CU | | | |
| hsa-miR- | hsa- | CCUGUUGCCACAA | AACCCGU | CAAGCUU | MI0000102 |
| 100- | mir- | ACCCGUAGAUCCG | AGAUCCG | GUAUCUA | |
| 4373160 | 100 | AACUUGUGGUAUU | AACUUGUG | UAGGUAUG | |
| | | AGUCCGCACAAGC | | | |
| | | UUGUAUCUAUAGG | | | |
| | | UAUGUGUCUGUUA | | | |
| | | GG | | | |
| hsa-miR- | hsa- | AUCAUUCAGAAAUG | AUGACCU | | MI0000291 |
| 215- | mir- | GUAUACAGGAAAAU | AUGAAUU | | |
| 4373084 | 215 | GACCUAUGAAUUG | GACAGAC | | |
| | | ACAGACAAUAUAGC | | | |
| | | UGAGUUUGUCUGU | | | |
| | | CAUUUCUUUAGGC | | | |
| | | CAAUAUUCUGUAU | | | |
| | | GACUGUGCUACUU | | | |
| | | CAA | | | |
| hsa-miR- | hsa- | GUGAUAAUGUAGC | UUGUGCU | AUGGUUC | MI0000294 |
| 218- | mir- | GAGAUUUUCUGUU | UGAUCUA | CGUCAAG | |
| 4373081 | 218-1 | GUGCUUGAUCUAA | ACCAUGU | CACCAUGG | |
| | | CCAUGUGGUUGCG | | | |
| | | AGGUAUGAGUAAA | | | |
| | | ACAUGGUUCCGUC | | | |
| | | AAGCACCAUGGAA | | | |
| | | CGUCACGCAGCUU | | | |
| | | UCUACA | | | |
| hsa-miR- | hsa- | GACCAGUCGCUGC | UUGUGCU | CAUGGUU | MI0000295 |
| 218- | mir- | GGGGCUUUCCUUU | UGAUCUA | CUGUCAA | |
| 4373081 | 218-2 | GUGCUUGAUCUAA | ACCAUGU | GCACCGCG | |
| | | CCAUGUGGUGGAA | | | |
| | | CGAUGGAAACGGA | | | |
| | | ACAUGGUUCUGUC | | | |
| | | AAGCACCGCGGAA | | | |
| | | AGCACCGUGCUCU | | | |
| | | CCUGCA | | | |
| hsa-miR- | hsa- | AUCUGAGUUGGGA | GUCCCUC | | MI0003657 |
| 642- | mir- | GGGUCCCUCUCCA | UCCAAAU | | |
| 4380995 | 642 | AAUGUGUCUUGGG | GUGUCUUG | | |
| | | GUGGGGGAUCAAG | | | |
| | | ACACAUUUGGAGA | | | |
| | | GGGAACCUCCCAA | | | |
| | | CUCGGCCUCUGCC | | | |
| | | AUCAUU | | | |
| hsa-miR- | hsa- | GAGAGAAGCACUG | ACUGGAC | | MI0001444 |
| 422a- | mir- | GACUUAGGGUCAG | UUAGGGU | | |
| 4373200 | 422a | AAGGCCUGAGUCU | CAGAAGGC | | |
| | | CUCUGCUGCAGAU | | | |
| | | GGGCUCUCUGUCC | | | |
| | | CUGAGCCAAGCUU | | | |
| | | UGUCCUCCCUGG | | | |
| hsa-miR- | hsa- | UAAAAGGUAGAUU | AUCAUAG | GUAGAUU | MI0000784 |
| 376a- | mir- | CUCCUUCUAUGAG | AGGAAAA | CUCCUUC | |
| 4373026 | 376a-1 | UACAUUAUUUAUGA | UCCACGU | UAUGAGUA | |
| | | UUAAUCAUAGAGG | | | |
| | | AAAAUCCACGUUUUC | | | |
| hsa-miR- | hsa- | GGUAUUUAAAAGG | AUCAUAG | | MI0003529 |
| 376a- | mir- | UAGAUUUUCCUUC | AGGAAAA | | |
| 4373026 | 376a-2 | UAUGGUUACGUGU | UCCACGU | | |
| | | UUGAUGGUUAAUC | | | |
| | | AUAGAGGAAAAUCC | | | |
| | | ACGUUUUCAGUAUC | | | |
| hsa-miR- | hsa- | GGCCUGGCUGGAC | ACAGCAG | UGCCUGU | MI0000290 |
| 214- | mir- | AGAGUUGUCAUGU | GCACAGA | CUACACU | |
| 4373085 | 214 | GUCUGCCUGUCUA | CAGGCAGU | UGCUGUGC | |
| | | CACUUGCUGUGCA | | | |
| | | GAACAUCCGCUCA | | | |
| | | CCUGUACAGCAGG | | | |
| | | CACAGACAGGCAG | | | |
| | | UCACAUGACAACCC | | | |
| | | AGCCU | | | |
| hsa-miR- | hsa- | GGGCUUUCAAGUC | CAAGUCA | AAAAUGG | MI0000301 |
| 224- | mir- | ACUAGUGGUUCCG | CUAGUGG | UGCCCUA | |
| 4373187 | 225 | UUUAGUAGAUGAU | UUCCGUU | GUGACUA | |
| | | UGUGCAUUGUUUC | | CA | |
| | | AAAAUGGUGCCCU | | | |
| | | AGUGACUACAAAG | | | |
| | | CCC | | | |
| hsa-miR- | hsa- | AGCUUCCCUGGCU | UAGCAGC | CCAAUAU | MI0000489 |
| 195- | mir- | CUAGCAGCACAGA | ACAGAAA | UGGCUGU | |
| 4373105 | 195 | AAUAUUGGCACAG | UAUUGGC | GCUGCUCC | |
| | | GGAAGCGAGUCUG | | | |
| | | CCAAUAUUGGCUG | | | |
| | | UGCUGCUCCAGGC | | | |
| | | AGGGUGGUG | | | |
| hsa-miR- | hsa- | UGGCCGAUUUUGG | UUUGGCA | AAUCAUG | MI0000098 |
| 96- | mir-96 | CACUAGCACAUUU | CUAGCAC | UGCAGUG | |
| 4373010 | | UUGCUUGUGUCUC | AUUUUUG | CCAAUAUG | |
| | | UCCGCUCUGAGCA | CU | | |
| | | AUCAUGUGCAGUG | | | |
| | | CCAAUAUGGGAAA | | | |
| hsa-miR- | hsa- | AACACAGUGGGCA | UUCAACG | | MI0000097 |
| 95- | miR- | CUCAAUAAAUGUCU | GGUAUUU | | |
| 4373011 | 95 | GUUGAAUUGAAAU | AUUGAGCA | | |
| | | GCGUUACAUUCAA | | | |
| | | CGGGUAUUUAUUG | | | |
| | | AGCACCCACUCUG | | | |
| | | UG | | | |
| hsa-miR- | hsa- | CUCAGGUGCUCUG | AUCACAU | UGGGUUC | MI0000439 |
| 23b- | mir- | GCUGCUUGGGUUC | UGCCAGG | CUGGCAU | |
| 4373073 | 23b | CUGGCAUGCUGAU | GAUUACC | GCUGAUUU | |
| | | UUGUGACUUAAGA | | | |
| | | UUAAAAUCACAUUG | | | |
| | | CCAGGGAUUACCA | | | |
| | | CGCAACCACGACC | | | |
| | | UUGGC | | | |
| hsa-miR- | hsa- | CUGAAAUAGGUUG | AAUAUUA | | MI0003678 |
| 656- | miR- | CCUGUGAGGUGUU | UACAGUC | | |
| 4380920 | 656 | CACUUUCUAUAUG | AACCUCU | | |
| | | AUGAAUAUUAUACA | | | |
| | | GUCAACCUCUUUC | | | |
| | | CGAUAUCGAAUC | | | |
| hsa-miR- | hsa- | CUUGGGAAUGGCA | AAACCGU | | MI0001729 |
| 451- | mir- | AGGAAACCGUUAC | UACCAUU | | |
| 4373209 | 451 | CAUUACUGAGUUU | ACUGAGUU | | |
| | | AGUAAUGGUAAUG | | | |
| | | GUUCUCUUGCUAU | | | |
| | | ACCCAGA | | | |
| hsa-miR- | hsa- | GAGCUGCUUGCCU | UUUGGCA | UGGUUCU | MI0000272 |
| 182- | mir- | CCCCCCGUUUUUG | AUGGUAG | AGACUUG | |
| 4373271 | 182 | GCAAUGGUAGAAC | AACUCAC | CCAACUA | |
| | | UCACACUGGUGAG | ACU | | |
| | | GUAACAGGAUCCG | | | |
| | | GUGGUUCUAGACU | | | |
| | | UGCCAACUAUGGG | | | |
| | | GCGAGGACUCAGC | | | |
| | | CGGCAC | | | |
| hsa-miR- | hsa- | CCAGAGGACACCU | CCCAGUG | ACAGUAG | MI0000282 |
| 199b- | mir- | CCACUCCGUCUAC | UUUAGAC | UCUGCAC | |
| 4373100 | 199b | CCAGUGUUUAGAC | UAUCUGU | AUUGGUUA | |
| | | UAUCUGUUCAGGA | UC | | |
| | | CUCCCAAAUUGUA | | | |
| | | CAGUAGUCUGCAC | | | |
| | | AUUGGUUAGGCUG | | | |
| | | GGCUGGGUUAGAC | | | |
| | | CCUCGG | | | |
| hsa-miR- | hsa- | GCCAACCCAGUGU | CCCAGUG | ACAGUAG | MI0000242 |
| 199a- | mir- | UCAGACUACCUGU | UUCAGAC | UCUGCAC | |
| 4373272 | 199a-1 | UCAGGAGGCUCUC | UACCUGU | AUUGGUUA | |
| | | AAUGUGUACAGUA | UC | | |
| | | GUCUGCACAUUGG | | | |
| | | UUAGGC | | | |
| hsa-miR- | hsa- | AGGAAGCUUCUGG | CCCAGUG | ACAGUAG | MI0000281 |
| 199a- | mir- | AGAUCCUGCUCCG | UUCAGAC | UCUGCAC | |
| 4373272 | 199a-2 | UCGCCCCAGUGUU | UACCUGU | AUUGGUUA | |
| | | CAGACUACCUGUU | UC | | |
| | | CAGGACAAUGCCG | | | |
| | | UUGUACAGUAGUC | | | |
| | | UGCACAUUGGUUA | | | |
| | | GACUGGGCAAGGG | | | |
| | | AGAGCA | | | |
| hsa-miR- | hsa- | CCCAUUGGCAUAA | AACCCGU | CAAGCUC | MI0000101 |
| 99a- | mir- | ACCCGUAGAUCCG | AGAUCCG | GCUUCUA | |
| 4373008 | 99a | AUCUUGUGGUGAA | AUCUUGUG | UGGGUCUG | |
| | | GUGGACCGCACAA | | | |
| | | GCUCGCUUCUAUG | | | |
| | | GGUCUGUGUCAGU | | | |
| | | GUG | | | |
| hsa-miR- | hsa- | CCACCCCGGUCCU | CAGCAGC | CAAACCA | MI0003138 |
| 497- | mir- | GCUCCCGCCCCAG | ACACUGU | CACUGUG | |
| 4373222 | 497 | CAGCACACUGUGG | GGUUUGU | GUGUUAGA | |
| | | UUUGUACGGCACU | | | |
| | | GUGGCCACGUCCA | | | |
| | | AACCACACUGUGG | | | |
| | | UGUUAGAGCGAGG | | | |
| | | GUGGGGGAGGCAC | | | |
| | | CGCCGAGG | | | |
| hsa-miR- | hsa- | GCAUCCUGUACUG | UCCUGUA | CGGGGCA | MI0002470 |
| 486- | mir- | AGCUGCCCCGAGG | CUGAGCU | GCUCAGU | |
| 4378096 | 486 | CCCUUCAUGCUGC | GCCCCGAG | ACAGGAU | |
| | | CCAGCUCGGGGCA | | | |
| | | GCUCAGUACAGGA | | | |
| | | UAC | | | |
| hsa-miR- | hsa- | AAGAAAUGGUUUA | UGGUUUA | UAUGUGG | MI0000744 |
| 299-5p- | mir- | CCGUCCCACAUAC | CCGUCCC | GAUGGUA | |
| 4373188 | 299 | AUUUUGAAUAUGU | ACAUACAU | AACCGCUU | |
| | | AUGUGGGAUGGUA | | | |
| | | AACCGCUUCUU | | | |
| hsa-miR- | hsa- | CACCUUGUCCUCA | GUCCAGU | GGAUUCC | MI0000461 |
| 145- | mir- | CGGUCCAGUUUUC | UUUCCCA | UGGAAAU | |
| 4373133 | 145 | CCAGGAAUCCCUU | GGAAUCC | ACUGUUCU | |
| | | AGAUGCUAAGAUG | CU | | |
| | | GGGAUUCCUGGAA | | | |
| | | AUACUGUUCUUGA | | | |
| | | GGUCAUGGUU | | | |
| hsa-miR- | hsa- | GCUAAGCACUUAC | AACUGUU | CUCAUCU | MI0001733 |
| 452- | mir- | AACUGUUUGCAGA | UGCAGAG | GCAAAGA | |
| 4373281 | 452 | GGAAACUGAGACU | GAAACUGA | AGUAAGUG | |
| | | UUGUAACUAUGUC | | | |
| | | UCAGUCUCAUCUG | | | |
| | | CAAAGAAGUAAGU | | | |
| | | GCUUUGC | | | |
| hsa-miR- | hsa- | CCGCAGAGUGUGA | UAUGGCA | GUGAAUU | MI0000273 |
| 183- | mir- | CUCCUGUUCUGUG | CUGGUAG | ACCGAAG | |
| 4373114 | 183 | UAUGGCACUGGUA | AAUUCACU | GGCCAUAA | |
| | | GAAUUCACUGUGA | | | |
| | | ACAGUCUCAGUCA | | | |
| | | GUGAAUUACCGAA | | | |
| | | GGGCCAUAAACAG | | | |
| | | AGCAGAGACAGAU | | | |
| | | CCACGA | | | |
| hsa-let-7c- | hsa- | GCAUCCGGGUUGA | UGAGGUA | | MI0000064 |
| 4373167 | let-7c | GGUAGUAGGUUGU | GUAGGUU | | |
| | | AUGGUUUAGAGUU | GUAUGGUU | | |
| | | ACACCCUGGGAGU | | | |
| | | UAACUGUACAACCU | | | |
| | | UCUAGCUUUCCUU | | | |
| | | GGAGC | | | |
| hsa-miR- | hsa- | GCGCAGCGCCCUG | UGAGAUG | GGUGCAG | MI0000459 |
| 143- | mir- | UCUCCCAGCCUGA | AAGCACU | UGCUGCA | |
| 4373134 | 143 | GGUGCAGUGCUGC | GUAGCUC | UCUCUGGU | |
| | | AUCUCUGGUCAGU | | | |
| | | UGGGAGUCUGAGA | | | |
| | | UGAAGCACUGUAG | | | |
| | | CUCAGGAAGAGAG | | | |
| | | AAGUUGUUCUGCA | | | |
| | | GC | | | |
| hsa-miR- | hsa- | UGUUUUGAGCGGG | UCAAGAG | UUUUUCA | MI0000816 |
| 335- | mir- | GGUCAAGAGCAAU | CAAUAAC | UUAUUGC | |
| 4373045 | 335 | AACGAAAAAUGUUU | GAAAAAU | UCCUGACC | |
| | | GUCAUAAACCGUU | GU | | |
| | | UUUCAUUAUUGCU | | | |
| | | CCUGACCUCCUCU | | | |
| | | CAUUUGCUAUAUU | | | |
| | | CA | | | |
| hsa-miR- | hsa- | UCAUUGGUCCAGA | GGUCCAG | | MI0000240 |
| 198- | mir- | GGGGAGAUAGGUU | AGGGGAG | | |
| 4373101 | 198 | CCUGUGAUUUUUC | AUAGGUUC | | |
| | | CUUCUUCUCUAUA | | | |
| | | GAAUAAAUGA | | | |
| hsa-miR- | hsa- | GUGGUCUCAGAAU | AACUGGC | CGGGGUU | MI0003137 |
| 193b- | mir- | CGGGGUUUUGAGG | CCUCAAA | UUGAGGG | |
| 4373185 | 193b | GCGAGAUGAGUUU | GUCCCGCU | CGAGAUGA | |
| | | AUGUUUUAUCCAA | | | |
| | | CUGGCCCUCAAAG | | | |
| | | UCCCGCUUUUGGG | | | |
| | | GUCAU | | | |
| hsa-miR- | hsa- | GUGUAUUCUACAG | UCUACAG | GGAGACG | MI0000261 |
| 139- | mir- | UGCACGUGUCUCC | UGCACGU | CGGCCCU | |
| 4373176 | 139 | AGUGUGGCUCGGA | GUCUCCAG | GUUGGAGU | |
| | | GGCUGGAGACGCG | | | |
| | | GCCCUGUUGGAGU | | | |
| | | AAC | | | |
| hsa-miR- | hsa- | GCUAAGCACUUAC | AACUGUU | CUCAUCU | MI0001733 |
| 452- | mir- | AACUGUUUGCAGA | UGCAGAG | GCAAAGA | |
| 4378077 | 452 | GGAAACUGAGACU | GAAACUGA | AGUAAGUG | |
| | | UUGUAACUAUGUC | | | |
| | | UCAGUCUCAUCUG | | | |
| | | CAAAGAAGUAAGU | | | |
| | | GCUUUGC | | | |
| hsa-miR- | hsa- | CCGGGCCCCUGUG | UAACACU | CAUCUUA | MI0000737 |
| 200a- | mir- | AGCAUCUUACCGG | GUCUGGU | CCGGACA | |
| 4373273 | 200a | ACAGUGCUGGAUU | AACGAUGU | GUGCUGGA | |
| | | UCCCAGCUUGACU | | | |
| | | CUAACACUGUCUG | | | |
| | | GUAACGAUGUUCA | | | |
| | | AAGGUGACCCGC | | | |
| hsa-miR- | hsa- | GCCGGCGCCCGAG | UCUGGCU | AGGGAGG | MI0000478 |
| 149- | mir- | CUCUGGCUCCGUG | CCGUGUC | GACGGGG | |
| 4373128 | 149 | UCUUCACUCCCGU | UUCACUC | GCUGUGC | |
| | | GCUUGUCCGAGGA | CC | | |
| | | GGGAGGGAGGGAC | | | |
| | | GGGGGCUGUGCUG | | | |
| | | GGGCAGCUGGA | | | |
| hsa-miR- | hsa- | CCAGCUCGGGCAG | UAAUACU | CAUCUUA | MI0000342 |
| 200b- | mir- | CCGUGGCCAUCUU | GCCUGGU | CUGGGCA | |
| 4381028 | 200b | ACUGGGCAGCAUU | AAUGAUGA | GCAUUGGA | |
| | | GGAUGGAGUCAGG | | | |
| | | UCUCUAAUACUGC | | | |
| | | CUGGUAAUGAUGA | | | |
| | | CGGCGGAGCCCUG | | | |
| | | CACG | | | |
| hsa-miR- | hsa- | CCCUCGUCUUACC | UAAUACU | CGUCUUA | MI0000650 |
| 200c- | mir- | CAGCAGUGUUUGG | GCCGGGU | CCCAGCA | |
| 4373096 | 200c | GUGCGGUUGGGAG | AAUGAUG | GUGUUUGG | |
| | | UCUCUAAUACUGC | GA | | |
| | | CGGGUAAUGAUGG | | | |
| | | AGG | | | |
| hsa-miR- | hsa- | CCCCGCGACGAGC | UUUGUUC | | MI0000783 |
| 375- | mir- | CCCUCGCACAAAC | GUUCGGC | | |
| 4373027 | 375 | CGGACCUGAGCGU | UCGCGUGA | | |
| | | UUUGUUCGUUCGG | | | |
| | | CUCGCGUGAGGC | | | |
| hsa-miR- | hsa- | CCGGGCCCCUGUG | UAACACU | CAUCUUA | MI0000737 |
| 200a- | mir- | AGCAUCUUACCGG | GUCUGGU | CCGGACA | |
| 4378069 | 200a | ACAGUGCUGGAUU | AACGAUGU | GUGCUGGA | |
| | | UCCCAGCUUGACU | | | |
| | | CUAACACUGUCUG | | | |
| | | GUAACGAUGUUCA | | | |
| | | AAGGUGACCCGC | | | |
| hsa-miR- | hsa- | CGCCGGCCGAUGG | UAAUACU | | MI0001641 |
| 429- | mir- | GCGUCUUACCAGA | GUCUGGU | | |
| 4373203 | 429 | CAUGGUUAGACCU | AAAACCGU | | |
| | | GGCCCUCUGUCUA | | | |
| | | AUACUGUCUGGUA | | | |
| | | AAACCGUCCAUCC | | | |
| | | GCUGC | | | |
| hsa-miR- | hsa- | CGGCCGGCCCUGG | UAACACU | CAUCUUC | MI0000457 |
| 141- | mir- | GUCCAUCUUCCAG | GUCUGGU | CAGUACA | |
| 4373137 | 141 | UACAGUGUUGGAU | AAAGAUGG | GUGUUGGA | |
| | | GGUCUAAUUGUGA | | | |
| | | AGCUCCUAACACU | | | |
| | | GUCUGGUAAAGAU | | | |
| | | GGCUCCCGGGUGG | | | |
| | | GUUC | | | |
| hsa-miR- | hsa- | CCUCAGAAGAAAGA | UUUGGUC | | MI0000822 |
| 133b- | mir- | UGCCCCCUGCUCU | CCCUUCA | | |
| 4373172 | 133b | GGCUGGUCAAACG | ACCAGCUA | | |
| | | GAACCAAGUCCGU | | | |
| | | CUUCCUGAGAGGU | | | |
| | | UUGGUCCCCUUCA | | | |
| | | ACCAGCUACAGCA | | | |
| | | GGGCUGGCAAUGC | | | |
| | | CCAGUCCUUGGAGA | | | |
| hsa-miR- | hsa- | CUCCUCAGAUCAG | AGAUCAG | | MI0000791 |
| 383- | mir- | AAGGUGAUUGUGG | AAGGUGA | | |
| 4373018 | 383 | CUUUGGGUGGAUA | UUGUGGCU | | |
| | | UUAAUCAGCCACA | | | |
| | | GCACUGCCUGGUC | | | |
| | | AGAAAGAG | | | |
| hsa-miR- | hsa- | UGCUUCCCGAGGC | UGGAAUG | | MI0000490 |
| 206- | mir- | CACAUGCUUCUUU | UAAGGAA | | |
| 4373092 | 206 | AUAUCCCCAUAUG | GUGUGUGG | | |
| | | GAUUACUUUGCUA | | | |
| | | UGGAAUGUAAGGA | | | |
| | | AGUGUGUGGUUUC | | | |
| | | GGCAAGUG | | | |
| hsa-miR-1- | hsa- | UGGGAAACAUACU | UGGAAUG | | MI0000651 |
| 4373161 | mir-1-1 | UCUUUAUAUGCCC | UAAAGAA | | |
| | | AUAUGGACCUGCU | GUAUGUAU | | |
| | | AAGCUAUGGAAUG | | | |
| | | UAAAGAAGUAUGUA | | | |
| | | UCUCA | | | |
| hsa-miR-1- | hsa- | ACCUACUCAGAGU | UGGAAUG | | MI0000437 |
| 4373161 | mir-1-2 | ACAUACUUCUUUAU | UAAAGAA | | |
| | | GUACCCAUAUGAA | GUAUGUAU | | |
| | | CAUACAAUGCUAU | | | |
| | | GGAAUGUAAAGAA | | | |
| | | GUAUGUAUUUUUG | | | |
| | | GUAGGC | | | |
| hsa-miR- | hsa- | ACAAUGCUUUGCU | UUUGGUC | | MI0000450 |
| 133a- | mir- | AGAGCUGGUAAAA | CCCUUCA | | |
| 4373142 | 133a-1 | UGGAACCAAAUCG | ACCAGCUG | | |
| | | CCUCUUCAAUGGA | | | |
| | | UUUGGUCCCCUUC | | | |
| | | AACCAGCUGUAGC | | | |
| | | UAUGCAUUGA | | | |
| hsa-miR- | hsa- | GGGAGCCAAAUGC | UUUGGUC | | MI0000451 |
| 133a- | mir- | UUUGCUAGAGCUG | CCCUUCA | | |
| 4373142 | 133a-2 | GUAAAAUGGAACCA | ACCAGCUG | | |
| | | AAUCGACUGUCCA | | | |
| | | AUGGAUUUGGUCC | | | |
| | | CCUUCAACCAGCU | | | |
| | | GUAGCUGUGCAUU | | | |
| | | GAUGGCGCCG | | | |
| hsa-miR- | hsa- | GUGUUGGGGACUC | GUGAAAU | | MI0000283 |
| 203- | mir- | GCGCGCUGGGUCC | GUUUAGG | | |
| 4373095 | 203 | AGUGGUUCUUAAC | ACCACUAG | | |
| | | AGUUCAACAGUUC | | | |
| | | UGUAGCGCAAUUG | | | |
| | | UGAAAUGUUUAGG | | | |
| | | ACCACUAGACCCG | | | |
| | | GCGGGCGCGGCGA | | | |
| | | CAGCGA | | | |
| hsa-miR- | hsa- | AAAGAUCCUCAGAC | UCCUUCA | GAUUUCA | MI0000285 |
| 205- | mir- | AAUCCAUGUGCUU | UUCCACC | GUGGAGU | |
| 4373093 | 205 | CUCUUGUCCUUCA | GGAGUCUG | GAAGUUC | |
| | | UUCCACCGGAGUC | | | |
| | | UGUCUCAUACCCA | | | |
| | | ACCAGAUUUCAGU | | | |
| | | GGAGUGAAGUUCA | | | |
| | | GGAGGCAUGGAGC | | | |
| | | UGACA | | | |

| | | | | | 95% |
|---|---|---|---|---|---|
| TLDA | HUGO | Study | Diag. | Fold | Conf. |
| MicroRNA- | Gene | Fold | Fold | Chng. P- | Int. Fold- |
| Probe | Symbol | Chng. | Chng. | Value | Chng. |
| hsa-miR- | MIR514-1 | 94.6 | 36.27424 | 2.88E−06 | 10.289772 |
| 514- | | | | | |
| 4373240 | | | | | |
| hsa-miR- | MIR514-2 | 94.6 | 36.27424 | 2.88E−06 | 10.289772 |
| 514- | | | | | |
| 4373240 | | | | | |
| hsa-miR- | MIR514-3 | 94.6 | 36.27424 | 2.88E−06 | 10.289772 |
| 514- | | | | | |
| 4373240 | | | | | |
| hsa-miR- | | 94.6 | 36.27424 | 2.88E−06 | 10.289772 |
| 514- | | | | | |
| 4373240 | | | | | |
| hsa-miR- | MIR508 | 88.7 | 61.84137 | 2.13E−08 | 22.050345 |
| 508- | | | | | |
| 4373233 | | | | | |
| hsa-miR- | MIR506 | 84.3 | 49.37615 | 1.12E−06 | 15.87256 |
| 506- | | | | | |
| 4373231 | | | | | |
| hsa-miR- | MIR509-1 | 24.1 | 23.23591 | 1.82E−10 | 11.361844 |
| 509- | | | | | |
| 4373234 | | | | | |
| hsa-miR- | MIR509-2 | 24.1 | 23.23591 | 1.82E−10 | 11.361844 |
| 509- | | | | | |
| 4373234 | | | | | |
| hsa-miR- | MIR509-3 | 24.1 | 23.23591 | 1.82E−10 | 11.361844 |
| 509- | | | | | |
| 4373234 | | | | | |
| hsa-miR- | MIR510 | 14.3 | 2.066301 | 0.832012 | 7.383E−14 |
| 510- | | | | | |
| 4373235 | | | | | |
| hsa-miR- | MIR507 | 14.0 | 2.29949 | NA | NA |
| 507- | | | | | |
| 4373232 | | | | | |
| hsa-miR- | MIR146A | 9.7 | 8.310579 | 2.61E−13 | 5.4515097 |
| 146a- | | | | | |
| 4373132 | | | | | |
| hsa-miR- | MIR542 | 9.3 | 1.208753 | 0.630428 | 0.5341426 |
| 542-5p- | | | | | |
| 4378105 | | | | | |
| hsa-miR- | MIR211 | 7.7 | 10.9937 | 1.57E−06 | 4.8111974 |
| 211- | | | | | |
| 4373088 | | | | | |
| hsa-miR- | MIR513A1 | 7.1 | NA | NA | NA |
| 513- | | | | | |
| 4373239 | | | | | |
| hsa-miR- | MIR513A2 | 7.1 | NA | NA | NA |
| 513- | | | | | |
| 4373239 | | | | | |
| hsa-miR- | MIR513B | 7.1 | NA | NA | NA |
| 513- | | | | | |
| 4373239 | | | | | |
| hsa-miR- | MIR513C | 7.1 | NA | NA | NA |
| 513- | | | | | |
| 4373239 | | | | | |
| hsa-miR- | MIR31 | 6.3 | 4.39148 | 5.74E−05 | 2.2339275 |
| 31- | | | | | |
| 4373190 | | | | | |
| hsa-miR- | MIR363 | 5.5 | 18.57955 | 0.006502 | 2.9781352 |
| 363- | | | | | |
| 4380917 | | | | | |
| hsa-miR- | | 5.2 | 4.87536 | 1.25E−05 | 2.6056788 |
| 565- | | | | | |
| 4380942 | | | | | |
| hsa-miR-9- | MIR9-1 | 4.9 | 3.982485 | 0.000197 | 1.998928 |
| 4373285 | | | | | |
| hsa-miR-9- | MIR9-2 | 4.9 | 3.982485 | 0.000197 | 1.998928 |
| 4373285 | | | | | |
| hsa-miR-9- | MIR9-3 | 4.9 | 3.982485 | 0.000197 | 1.998928 |
| 4373285 | | | | | |
| hsa-miR- | MIR34B | 4.2 | 2.255301 | 0.134443 | 0.7626607 |
| 34b- | | | | | |
| 4373037 | | | | | |
| hsa-miR- | MIR424 | 4.2 | 2.608973 | 0.008728 | 1.3013931 |
| 424- | | | | | |
| 4373201 | | | | | |
| hsa-miR- | MIR21 | 4.1 | 3.628786 | 1.71E−08 | 2.4670837 |
| 21- | | | | | |
| 4373090 | | | | | |
| hsa-miR- | MIR155 | 3.9 | 3.603945 | 4.2E−06 | 2.1985486 |
| 155- | | | | | |
| 4373124 | | | | | |
| hsa-miR- | MIR135B | 3.7 | 2.926567 | 0.0027 | 1.4884041 |
| 135b- | | | | | |
| 4373139 | | | | | |
| hsa-miR-7- | MIR7-1 | 3.5 | 1.261623 | 0.362518 | 0.7587347 |
| 4373014 | | | | | |
| hsa-miR-7- | MIR7-2 | 3.5 | 1.261623 | 0.362518 | 0.7587347 |
| 4373014 | | | | | |
| hsa-miR-7- | MIR7-3 | 3.5 | 1.261623 | 0.362518 | 0.7587347 |
| 4373014 | | | | | |
| hsa-miR- | MIR449A | 3.4 | 2.029582 | 0.078965 | 0.9163214 |
| 449- | | | | | |
| 4373207 | | | | | |
| hsa-miR- | MIR449B | 3.4 | 2.029582 | 0.078965 | 0.9163214 |
| 449- | | | | | |
| 4373207 | | | | | |
| hsa-miR- | NA | 3.4 | 2.029582 | 0.078965 | 0.9163214 |
| 449- | | | | | |
| 4373207 | | | | | |
| hsa-miR- | MIR34A | 3.0 | 2.766221 | 9.76E−05 | 1.7199658 |
| 34a- | | | | | |
| 4373278 | | | | | |
| hsa-miR- | MIR503 | 3.0 | 1.995789 | 0.116483 | 0.8320872 |
| 503- | | | | | |
| 4373228 | | | | | |
| hsa-miR- | MIR34C | 2.9 | 2.875911 | 0.043799 | 1.0323556 |
| 34c- | | | | | |
| 4373036 | | | | | |
| hsa-miR- | MIR20B | 2.8 | 2.439498 | 0.007199 | 1.2896759 |
| 20b- | | | | | |
| 4373263 | | | | | |
| hsa-miR- | MIR449B | 2.8 | 2.051943 | 0.131115 | 0.7976108 |
| 449b- | | | | | |
| 4381011 | | | | | |
| hsa-miR- | MIR184 | 2.8 | 2.708411 | 0.143378 | 0.678377 |
| 184- | | | | | |
| 4373113 | | | | | |
| hsa-miR- | MIR301A | 2.6 | 2.244631 | 0.000142 | 1.5158881 |
| 301- | | | | | |
| 4373064 | | | | | |
| hsa-miR- | MIR301B | 2.6 | 2.244631 | 0.000142 | 1.5158881 |
| 301- | | | | | |
| 4373064 | | | | | |
| hsa-miR- | MIR493 | 2.5 | 3.72265 | 0.103384 | 0.6198478 |
| 493- | | | | | |
| 4373218 | | | | | |
| hsa-miR- | MIR105-1 | 2.4 | 20.17832 | NA | NA |
| 105- | | | | | |
| 4373157 | | | | | |
| hsa-miR- | MIR105-2 | 2.4 | 20.17832 | NA | NA |
| 105- | | | | | |
| 4373157 | | | | | |
| hsa-miR- | MIR181D | 2.2 | 2.074895 | 0.000643 | 1.3879593 |
| 181d- | | | | | |
| 4373180 | | | | | |
| hsa-miR- | MIR18A | 2.2 | 1.810556 | 0.011685 | 1.1481725 |
| 18a- | | | | | |
| 4373118 | | | | | |
| hsa-miR-9- | MIR9-1 | 2.0 | 1.618821 | 0.211586 | 0.7535053 |
| 4378074 | | | | | |
| hsa-miR-9- | MIR9-2 | 2.0 | 1.618821 | 0.211586 | 0.7535053 |
| 4378074 | | | | | |
| hsa-miR-9- | MIR9-3 | 2.0 | 1.618821 | 0.211586 | 0.7535053 |
| 4378074 | | | | | |
| hsa-miR- | MIR18B | 2.0 | 5.693522 | NA | NA |
| 18b- | | | | | |
| 4373184 | | | | | |
| hsa-miR- | MIR330 | 2.0 | 1.530397 | 0.1015 | 0.9167586 |
| 330- | | | | | |
| 4373047 | | | | | |
| hsa-miR- | MIR181B1 | 2.0 | 1.893247 | 0.000154 | 1.3853619 |
| 181b- | | | | | |
| 4373116 | | | | | |
| hsa-miR- | MIR181B2 | 2.0 | 1.893247 | 0.000154 | 1.3853619 |
| 181b- | | | | | |
| 4373116 | | | | | |
| hsa-miR- | MIR33A | 2.0 | 0.479794 | 0.268767 | 0.1157152 |
| 33- | | | | | |
| 4373048 | | | | | |
| hsa-miR- | MIR33B | 2.0 | 0.479794 | 0.268767 | 0.1157152 |
| 33- | | | | | |
| 4373048 | | | | | |
| hsa-miR- | Mir181a-1 | 1.9 | 2.07388 | 0.003645 | 1.2837157 |
| 213- | | | | | |
| 4373086 | | | | | |
| hsa-miR- | MIR519C | 1.6 | 2.160278 | 0.062363 | 0.9554607 |
| 519c- | | | | | |
| 4373251 | | | | | |
| hsa-let-7g- | MIRLET7G | −2.0 | −2.115722 | 0.000497 | −1.413239 |
| 4373163 | | | | | |
| hsa-miR- | MIR369 | −2.0 | −2.186415 | 0.356392 | −0.324381 |
| 369-3p- | | | | | |
| 4373032 | | | | | |
| hsa-miR- | MIR299 | −2.0 | −0.872859 | 0.791611 | −0.264625 |
| 299-3p- | | | | | |
| 4373189 | | | | | |
| hsa-miR- | MIR601 | −2.0 | −1.891452 | 0.090509 | −0.899664 |
| 601- | | | | | |
| 4380965 | | | | | |
| hsa-miR- | MIR518B | −2.1 | −3.242438 | 0.00535 | −1.449911 |
| 518b- | | | | | |
| 4373246 | | | | | |
| hsa-miR- | MIR409 | −2.1 | −2.257482 | 0.121021 | −0.788539 |
| 409-5p- | | | | | |
| 4373197 | | | | | |
| hsa-miR- | MIR496 | −2.1 | −2.648886 | 0.219127 | −0.514426 |
| 496- | | | | | |
| 4373221 | | | | | |
| hsa-miR- | MIR192 | −2.1 | −2.616647 | 2.59E−05 | −1.724866 |
| 192- | | | | | |
| 4373108 | | | | | |
| hsa-miR- | MIR22 | −2.1 | −2.634333 | 4.57E−05 | −1.704122 |
| 22- | | | | | |
| 4373079 | | | | | |
| hsa-let-7f- | MIRLET7F1 | −2.2 | −2.413059 | 3.26E−05 | −1.638248 |
| 4373164 | | | | | |
| hsa-let-7f- | MIRLET7F2 | −2.2 | −2.413059 | 3.26E−05 | −1.638248 |
| 4373164 | | | | | |
| hsa-miR- | MIR194-1 | −2.2 | −2.550789 | 0.000322 | −1.568458 |
| 194- | | | | | |
| 4373106 | | | | | |
| hsa-miR- | MIR194-2 | −2.2 | −2.550789 | 0.000322 | −1.568458 |
| 194- | | | | | |
| 4373106 | | | | | |
| hsa-miR- | MIR572 | −2.2 | −2.778764 | 0.001657 | −1.518361 |
| 572- | | | | | |
| 4381017 | | | | | |
| hsa-miR- | MIR331 | −2.2 | −2.52479 | 5.86E−08 | −1.889135 |
| 331- | | | | | |
| 4373046 | | | | | |
| hsa-miR- | MIR489 | −2.4 | −2.309346 | 0.017606 | −1.165151 |
| 489- | | | | | |
| 4373214 | | | | | |
| hsa-miR- | MIR432 | −2.4 | −3.61536 | 0.000114 | −1.965293 |
| 432- | | | | | |
| 4373280 | | | | | |
| hsa-miR- | MIR629 | −2.5 | −2.637864 | 0.001695 | −1.468833 |
| 629- | | | | | |
| 4380969 | | | | | |
| hsa-miR- | MIR202 | −2.5 | −3.410693 | 0.140753 | −0.636513 |
| 202- | | | | | |
| 4378075 | | | | | |
| hsa-miR- | MIR148A | −2.6 | −3.331875 | 4.31E−06 | −2.086704 |
| 148a- | | | | | |
| 4373130 | | | | | |
| hsa-miR- | MIR627 | −2.6 | −3.608625 | 0.00018 | −1.917908 |
| 627- | | | | | |
| 4380967 | | | | | |
| hsa-miR- | MIR511-1 | −2.6 | −3.160728 | 1.46E−05 | −1.955368 |
| 511- | | | | | |
| 4373236 | | | | | |
| hsa-miR- | MIR511-2 | −2.6 | −3.160728 | 1.46E−05 | −1.955368 |
| 511- | | | | | |
| 4373236 | | | | | |
| hsa-miR- | MIR24-1 | −2.6 | −2.929054 | 1.34E−07 | −2.06145 |
| 24- | | | | | |
| 4373072 | | | | | |
| hsa-miR- | MIR24-2 | −2.6 | −2.929054 | 1.34E−07 | −2.06145 |
| 24- | | | | | |
| 4373072 | | | | | |
| hsa-let-7a- | MIRLET7A1 | −2.7 | −3.386539 | 0.00114 | −1.701688 |
| 4373169 | | | | | |
| hsa-let-7a- | MIRLET7A2 | −2.7 | −3.386539 | 0.00114 | −1.701688 |
| 4373169 | | | | | |
| hsa-let-7a- | MIRLET7A3 | −2.7 | −3.386539 | 0.00114 | −1.701688 |
| 4373169 | | | | | |
| hsa-miR- | MIR10A | −2.8 | −3.163245 | 2E−06 | −2.062524 |
| 10a- | | | | | |
| 4373153 | | | | | |
| hsa-miR- | MIR210 | −2.8 | −3.314238 | 2.17E−06 | −2.119572 |
| 210- | | | | | |
| 4373089 | | | | | |
| hsa-miR- | MIR27A | −2.8 | −3.238325 | 7.86E−07 | −2.132781 |
| 27a- | | | | | |
| 4373287 | | | | | |
| hsa-miR- | MIR196B | −2.9 | −3.668072 | 0.000473 | −1.843677 |
| 196b- | | | | | |
| 4373103 | | | | | |
| hsa-miR- | MIR26B | −3.0 | −3.784939 | 8.99E−11 | −2.729121 |
| 26b- | | | | | |
| 4373069 | | | | | |
| hsa-miR- | MIR30E | −3.0 | −3.40464 | 8.41E−09 | −2.386588 |
| 30e-3p- | | | | | |
| 4373057 | | | | | |
| hsa-miR- | MIR378 | −3.1 | −2.21983 | 0.058477 | −0.970422 |
| 422b- | | | | | |
| 4373016 | | | | | |
| hsa-miR- | MIR615 | −3.1 | −4.145301 | 1.52E−05 | −2.291168 |
| 615- | | | | | |
| 4380991 | | | | | |
| hsa-miR- | MIR152 | −3.2 | −3.773565 | 1.57E−09 | −2.634857 |
| 152- | | | | | |
| 4373126 | | | | | |
| hsa-miR- | MIR23A | −3.3 | −3.736023 | 9.5E−07 | −2.326541 |
| 23a- | | | | | |
| 4373074 | | | | | |
| hsa-let-7e- | MIRLET7E | −3.3 | −3.631678 | 0.003922 | −1.56211 |
| 4373165 | | | | | |
| hsa-miR- | MIR190 | −3.3 | −3.523254 | 2.77E−06 | −2.189989 |
| 190- | | | | | |
| 4373110 | | | | | |
| hsa-miR- | MIR369 | −3.3 | −2.223348 | 0.085782 | −0.887583 |
| 369-5p- | | | | | |
| 4373195 | | | | | |
| hsa-miR- | MIR30E | −3.3 | −3.687065 | 3.09E−08 | −2.478113 |
| 30e-5p- | | | | | |
| 4373058 | | | | | |
| hsa-miR- | MIR504 | −3.3 | −3.217027 | 0.004991 | −1.471454 |
| 504- | | | | | |
| 4373229 | | | | | |
| hsa-miR- | MIR29C | −3.4 | −4.157279 | 2.77E−08 | −2.702234 |
| 29c- | | | | | |
| 4373289 | | | | | |
| hsa-miR- | MIR26A1 | −3.4 | −4.154968 | 1.12E−10 | −2.921961 |
| 26a- | | | | | |
| 4373070 | | | | | |
| hsa-miR- | MIR26A2 | −3.4 | −4.154968 | 1.12E−10 | −2.921961 |
| 26a- | | | | | |
| 4373070 | | | | | |
| hsa-miR- | MIR101-1 | −3.5 | −4.088785 | 7.6E−10 | −2.816267 |
| 101- | | | | | |
| 4373159 | | | | | |
| hsa-miR- | MIR101-2 | −3.5 | −4.088785 | 7.6E−10 | −2.816267 |
| 101- | | | | | |
| 4373159 | | | | | |
| hsa-miR- | MIR382 | −3.5 | −4.357766 | 3.43E−06 | −2.478523 |
| 382- | | | | | |
| 4373019 | | | | | |
| hsa-miR- | MIR328 | −3.5 | −3.82034 | 1.89E−09 | −2.645469 |
| 328- | | | | | |
| 4373049 | | | | | |
| hsa-miR- | MIR204 | −3.5 | −2.284185 | 0.133167 | −0.768049 |
| 204- | | | | | |
| 4373094 | | | | | |
| hsa-miR- | MIR127 | −3.8 | −4.852117 | 6.97E−08 | −2.942705 |
| 127- | | | | | |
| 4373147 | | | | | |
| hsa-miR- | MIR30A | −4.0 | −4.095968 | 2.31E−08 | −2.673761 |
| 30a-3p- | | | | | |
| 4373062 | | | | | |
| hsa-miR- | MIR551B | −4.0 | −0.70496 | 0.30415 | −0.355645 |
| 551b- | | | | | |
| 4380945 | | | | | |
| hsa-miR- | MIR24-1 | −4.0 | −4.661145 | 1.15E−05 | −2.497524 |
| 189- | | | | | |
| 4378067 | | | | | |
| hsa-miR- | MIR193A | −4.2 | −4.721566 | 4.94E−08 | −2.909501 |
| 193a- | | | | | |
| 4373107 | | | | | |
| hsa-miR- | MIR378 | −4.2 | −3.845659 | 1.65E−05 | −2.210415 |
| 378- | | | | | |
| 4373024 | | | | | |
| hsa-miR- | MIR10B | −4.3 | −4.702263 | 3.63E−07 | −2.783047 |
| 10b- | | | | | |
| 4373152 | | | | | |
| hsa-miR- | MIR485 | −4.3 | −3.99003 | 5.37E−05 | −2.135541 |
| 485-3p- | | | | | |
| 4378095 | | | | | |
| hsa-miR- | MIR410 | −4.4 | −4.952677 | 4.1E−07 | −2.865782 |
| 410- | | | | | |
| 4378093 | | | | | |
| hsa-miR- | MIR196A1 | −4.5 | −5.11188 | 2.06E−09 | −3.263696 |
| 196a- | | | | | |
| 4373104 | | | | | |
| hsa-miR- | MIR196A2 | −4.5 | −5.11188 | 2.06E−09 | −3.263696 |
| 196a- | | | | | |
| 4373104 | | | | | |
| hsa-let-7b- | MIRLET7B | −4.5 | −5.197677 | 7.76E−11 | −3.488737 |
| 4373168 | | | | | |
| hsa-miR- | MIR487B | −5.0 | −5.552066 | 1.63E−08 | −3.343102 |
| 487b- | | | | | |
| 4378102 | | | | | |
| hsa-miR- | MIR365-1 | −5.1 | −6.169501 | 2.34E−10 | −3.915028 |
| 365- | | | | | |
| 4373194 | | | | | |
| hsa-miR- | MIR365-2 | −5.1 | −6.169501 | 2.34E−10 | −3.915028 |
| 365- | | | | | |
| 4373194 | | | | | |
| hsa-miR- | MIR126 | −5.2 | −6.369204 | 4.37E−13 | −4.342754 |
| 126- | | | | | |
| 4373269 | | | | | |
| hsa-miR- | MIR134 | −5.2 | −5.987322 | 4.52E−08 | −3.438969 |
| 134- | | | | | |
| 4373141 | | | | | |
| hsa-miR- | MIR126 | −5.3 | −6.479142 | 1.02E−12 | −4.401254 |
| 126- | | | | | |
| 4378064 | | | | | |
| hsa-miR- | MIR485 | −5.3 | −3.454889 | 0.022973 | −1.203653 |
| 485-5p- | | | | | |
| 4373212 | | | | | |
| hsa-miR- | MIR27B | −5.3 | −6.103759 | 5.53E−09 | −3.638981 |
| 27b- | | | | | |
| 4373068 | | | | | |
| hsa-miR- | MIR433 | −5.5 | −4.226366 | 1.49E−05 | −2.321472 |
| 433- | | | | | |
| 4373205 | | | | | |
| hsa-miR- | MIR411 | −5.5 | −7.695649 | 1.23E−10 | −4.637895 |
| 411- | | | | | |
| 4381013 | | | | | |
| hsa-miR- | MIR376A1 | −5.6 | −2.184801 | 0.072672 | −0.926041 |
| 376a- | | | | | |
| 4378104 | | | | | |
| hsa-miR- | MIR376A2 | −5.6 | −2.184801 | 0.072672 | −0.926041 |
| 376a- | | | | | |
| 4378104 | | | | | |
| hsa-miR- | MIR199A1 | −5.7 | −6.854587 | 4.01E−10 | −4.173382 |
| 199a- | | | | | |
| 4378068 | | | | | |
| hsa-miR- | MIR199A2 | −5.7 | −6.854587 | 4.01E−10 | −4.173382 |
| 199a- | | | | | |
| 4378068 | | | | | |
| hsa-miR- | MIR125B1 | −5.7 | −6.928742 | 4.17E−11 | −4.359948 |
| 125b- | | | | | |
| 4373148 | | | | | |
| hsa-miR- | MIR125B2 | −5.7 | −6.928742 | 4.17E−11 | −4.359948 |
| 125b- | | | | | |
| 4373148 | | | | | |
| hsa-miR- | MIR379 | −5.9 | −5.834828 | 2.36E−08 | −3.434768 |
| 379- | | | | | |
| 4373023 | | | | | |
| hsa-miR- | MIR100 | −6.0 | −7.254331 | 1.95E−08 | −3.998893 |
| 100- | | | | | |
| 4373160 | | | | | |
| hsa-miR- | MIR215 | −6.0 | −5.28082 | 0.000312 | −2.252762 |
| 215- | | | | | |
| 4373084 | | | | | |
| hsa-miR- | MIR218-1 | −6.3 | −7.075135 | 8.61E−10 | −4.239413 |
| 218- | | | | | |
| 4373081 | | | | | |
| hsa-miR- | MIR218-2 | −6.3 | −7.075135 | 8.61E−10 | −4.239413 |
| 218- | | | | | |
| 4373081 | | | | | |
| hsa-miR- | MIR642 | −6.8 | −3.716804 | 3.68E−06 | −2.260962 |
| 642- | | | | | |
| 4380995 | | | | | |
| hsa-miR- | MIR422A | −6.9 | −5.309461 | 8.22E−06 | −2.722392 |
| 422a- | | | | | |
| 4373200 | | | | | |
| hsa-miR- | MIR376A1 | −7.0 | −8.584561 | 8.69E−09 | −4.658695 |
| 376a- | | | | | |
| 4373026 | | | | | |
| hsa-miR- | MIR376A2 | −7.0 | −8.584561 | 8.69E−09 | −4.658695 |
| 376a- | | | | | |
| 4373026 | | | | | |
| hsa-miR- | MIR214 | −7.1 | −8.616543 | 6.3E−12 | −5.407976 |
| 214- | | | | | |
| 4373085 | | | | | |
| hsa-miR- | MIR224 | −7.2 | −8.266594 | 9.75E−12 | −5.294334 |
| 224- | | | | | |
| 4373187 | | | | | |
| hsa-miR- | MIR195 | −7.2 | −8.975379 | 3.99E−05 | −3.676586 |
| 195- | | | | | |
| 4373105 | | | | | |
| hsa-miR- | MIR96 | −7.4 | −3.698506 | 0.029069 | −1.170063 |
| 96- | | | | | |
| 4373010 | | | | | |
| hsa-miR- | MIR95 | −7.4 | −9.307827 | 1.96E−11 | −5.664539 |
| 95- | | | | | |
| 4373011 | | | | | |
| hsa-miR- | MIR23B | −7.6 | −7.538382 | 1.05E−09 | −4.40427 |
| 23b- | | | | | |
| 4373073 | | | | | |
| hsa-miR- | MIR656 | −7.6 | −4.33361 | 0.0007 | −1.948361 |
| 656- | | | | | |
| 4380920 | | | | | |
| hsa-miR- | MIR451 | −8.0 | −10.31393 | 1.43E−07 | −4.851491 |
| 451- | | | | | |
| 4373209 | | | | | |
| hsa-miR- | MIR182 | −8.2 | −9.838357 | 8.72E−13 | −6.088575 |
| 182- | | | | | |
| 4373271 | | | | | |
| hsa-miR- | MIR199B | −8.7 | −8.375536 | 1.73E−10 | −4.932592 |
| 199b- | | | | | |
| 4373100 | | | | | |
| hsa-miR- | MIR199A1 | −9.0 | −8.271019 | 3.47E−09 | −4.639533 |
| 199a- | | | | | |
| 4373272 | | | | | |
| hsa-miR- | MIR199A2 | −9.0 | −8.271019 | 3.47E−09 | −4.639533 |
| 199a- | | | | | |
| 4373272 | | | | | |
| hsa-miR- | MIR99A | −9.2 | −10.7254 | 2.18E−09 | −5.59986 |
| 99a- | | | | | |
| 4373008 | | | | | |
| hsa-miR- | MIR497 | −9.2 | −10.63973 | 6.78E−13 | −6.51092 |
| 497- | | | | | |
| 4373222 | | | | | |
| hsa-miR- | MIR486 | −9.4 | −11.17136 | 3.9E−08 | −5.412317 |
| 486- | | | | | |
| 4378096 | | | | | |
| hsa-miR- | MIR299 | −9.5 | −6.689598 | 2.82E−06 | −3.308525 |
| 299-5p- | | | | | |
| 4373188 | | | | | |
| hsa-miR- | MIR145 | −10.6 | −11.77992 | 2.55E−13 | −7.155997 |
| 145- | | | | | |
| 4373133 | | | | | |
| hsa-miR- | MIR452 | −10.6 | −3.771781 | 2.33E−05 | −2.157941 |
| 452- | | | | | |
| 4373281 | | | | | |
| hsa-miR- | MIR183 | −11.3 | −8.157773 | 1.23E−05 | −3.518427 |
| 183- | | | | | |
| 4373114 | | | | | |
| hsa-let-7c- | MIRLET7C | −12.5 | −15.01269 | 1.57E−15 | −9.496025 |
| 4373167 | | | | | |
| hsa-miR- | MIR143 | −15.2 | −18.20404 | 1.18E−13 | −10.32602 |
| 143- | | | | | |
| 4373134 | | | | | |
| hsa-miR- | MIR335 | −19.4 | −23.53579 | 1.92E−13 | −12.79855 |
| 335- | | | | | |
| 4373045 | | | | | |
| hsa-miR- | MIR198 | −20.8 | −2.933641 | 0.080181 | −0.85724 |
| 198- | | | | | |
| 4373101 | | | | | |
| hsa-miR- | MIR193B | −23.0 | −25.20123 | 1.57E−18 | −16.15343 |
| 193b- | | | | | |
| 4373185 | | | | | |
| hsa-miR- | MIR139 | −23.5 | −11.62359 | 5.87E−10 | −6.518436 |
| 139- | | | | | |
| 4373176 | | | | | |
| hsa-miR- | MIR452 | −27.4 | −15.69486 | 2.02E−11 | −8.776732 |
| 452- | | | | | |
| 4378077 | | | | | |
| hsa-miR- | MIR200A | −30.5 | −9.123886 | 1.27E−06 | −4.505046 |
| 200a- | | | | | |
| 4373273 | | | | | |
| hsa-miR- | MIR149 | −36.1 | −36.15662 | 5.75E−13 | −17.99738 |
| 149- | | | | | |
| 4373128 | | | | | |
| hsa-miR- | MIR200B | −36.8 | −35.61339 | 8.29E−11 | −14.96079 |
| 200b- | | | | | |
| 4381028 | | | | | |
| hsa-miR- | MIR200C | −37.1 | −39.75681 | 1.4E−12 | −18.33635 |
| 200c- | | | | | |
| 4373096 | | | | | |
| hsa-miR- | MIR375 | −43.9 | −20.60739 | 5.69E−06 | −6.695575 |
| 375- | | | | | |
| 4373027 | | | | | |
| hsa-miR- | MIR200A | −49.0 | −53.12863 | 1.07E−14 | −25.73069 |
| 200a- | | | | | |
| 4378069 | | | | | |
| hsa-miR- | MIR429 | −49.2 | −34.95381 | 7.61E−13 | −16.98012 |
| 429- | | | | | |
| 4373203 | | | | | |
| hsa-miR- | MIR141 | −50.6 | −42.89899 | 1.59E−10 | −16.87656 |
| 141- | | | | | |
| 4373137 | | | | | |
| hsa-miR- | MIR133B | −53.0 | −64.35787 | 6.94E−08 | −21.51917 |
| 133b- | | | | | |
| 4373172 | | | | | |
| hsa-miR- | MIR383 | −75.3 | −30.02161 | 4.12E−08 | −11.51144 |
| 383- | | | | | |
| 4373018 | | | | | |
| hsa-miR- | MIR206 | −75.4 | −41.83691 | 0.000165 | −7.632828 |
| 206- | | | | | |
| 4373092 | | | | | |
| hsa-miR-1- | MIR1-1 | −87.8 | −65.7283 | 1.29E−08 | −21.54726 |
| 4373161 | | | | | |
| hsa-miR-1- | MIR1-2 | −87.8 | −65.7283 | 1.29E−08 | −21.54726 |
| 4373161 | | | | | |
| hsa-miR- | MIR133A1 | −117.9 | −58.59496 | 1.07E−06 | −15.54888 |
| 133a- | | | | | |
| 4373142 | | | | | |
| hsa-miR- | MIR133A2 | −117.9 | −58.59496 | 1.07E−06 | −15.54888 |
| 133a- | | | | | |
| 4373142 | | | | | |
| hsa-miR- | MIR203 | −120.5 | −91.97567 | 3.75E−09 | −26.37271 |
| 203- | | | | | |
| 4373095 | | | | | |
| hsa-miR- | MIR205 | −131.6 | −38.99383 | 3.95E−08 | −12.97793 |
| 205- | | | | | |
| 4373093 | | | | | |

### Example 13

### Examples of Embodiments

Provided hereafter are examples of certain embodiments.

A1. A method, comprising:
(a) administering an anti-cancer drug to a subject having melanoma;
(b) identifying or determining the presence, absence or amount of a biomarker in the subject, wherein the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(c) maintaining a subsequent dosage of the drug or adjusting a subsequent dosage of the drug administered to the subject based on the presence, absence or amount of the biomarker identified in the subject.

A2. A method, comprising:
(a) administering an anti-cancer drug to a subject having melanoma;
(b) identifying or determining the presence, absence or amount of a biomarker in the subject, wherein the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(c) determining whether the dosage of the drug subsequently administered to the subject is adjusted based on the presence, absence or amount of the biomarker identified in the subject.

A3. A method, comprising:
(a) identifying or determining the presence, absence or amount of a biomarker in a subject having melanoma to whom an anti-cancer drug has been administered, wherein the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) maintaining a subsequent dosage of the drug or adjusting a subsequent dosage of the drug administered to the subject based on the presence, absence or amount of the biomarker identified in the subject.

A4. A method, comprising:
(a) identifying or determining the presence, absence or amount of a biomarker in a subject having melanoma to whom an anti-cancer drug has been administered, wherein the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) determining whether the dosage of the drug subsequently administered to the subject is adjusted based on the presence, absence or amount of the biomarker identified in the subject.

A5. A method, comprising:
(a) receiving information comprising the presence, absence or amount of a biomarker in a subject having melanoma to whom an anti-cancer drug has been administered, wherein the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) maintaining a subsequent dosage of the drug or adjusting a subsequent dosage of the drug administered to the subject based on the presence, absence or amount of the biomarker identified in the subject.

A6. A method, comprising:
(a) identifying or determining the presence, absence or amount of a biomarker in a subject having melanoma to whom an anti-cancer drug has been administered, wherein the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) transmitting the presence, absence or amount of the biomarker to a decision maker who maintains a subsequent dosage of the drug or adjusts a subsequent dosage of the drug administered to the subject based on the presence, absence or amount of the biomarker identified in the subject.

A7. A method, comprising:
(a) identifying or determining the presence, absence or amount of a biomarker in a subject having melanoma to whom an anti-cancer drug has been administered, wherein the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) transmitting an indication to maintain a subsequent dosage of the drug or adjust a subsequent dosage of the drug administered to the subject based on the presence, absence or amount of the biomarker identified in the subject.

A8. A method for optimizing therapeutic efficacy of a treatment of melanoma in a subject, comprising:
(a) identifying or determining the presence, absence or amount of a biomarker in a subject having melanoma to whom an anti-cancer drug has been administered, wherein the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) maintaining a subsequent dosage of the drug or adjusting a subsequent dosage of the drug administered to the subject based on the presence, absence or amount of the biomarker identified in the subject.

A9. A method for reducing toxicity of a treatment of melanoma in a subject, comprising:
(a) identifying or determining the presence, absence or amount of a biomarker in a subject having melanoma to whom an anti-cancer drug has been administered, wherein the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) maintaining a subsequent dosage of the drug or adjusting a subsequent dosage of the drug administered to the subject based on the presence, absence or amount of the biomarker identified in the subject.

B1. A method for treating melanoma in a subject, comprising:
administering a composition that delivers to a subject in need thereof a microRNA composition in an amount effective to treat the melanoma in the subject,
wherein the microRNA composition comprises a microRNA selected from the group consisting of microRNA selected from the group consisting of microRNA-10, microRNA-126, microRNA-193, microRNA-203, microRNA-206, and combinations thereof.

B2. A method, comprising:
contacting melanoma cells with a microRNA composition in an amount effective to inhibit proliferation of the melanoma cells,
wherein the microRNA composition comprises a microRNA selected from the group consisting of microRNA-126, microRNA-193, microRNA-206, and combinations thereof.

B3. A method, comprising:
contacting melanoma cells with a microRNA composition in an amount effective to induce apoptosis of the melanoma cells,
wherein the microRNA composition comprises a microRNA selected from the group consisting of microRNA-10, microRNA-126, microRNA-193, microRNA-203, and combinations thereof.

B4. A method for treating melanoma in a subject, comprising:
administering a composition that delivers to a subject in need thereof a microRNA inhibitor composition in an amount effective to treat the melanoma in the subject,
wherein the microRNA inhibitor composition comprises an inhibitor of a microRNA-21, microRNA-146, microRNA-155, microRNA-506, microRNA-507, microRNA-508, microRNA-509, microRNA-510, microRNA-513, microRNA-514, microRNA-506-514 cluster, microRNA-506-513 cluster, and combinations thereof.

B5. A method, comprising:
contacting melanoma cells with a microRNA inhibitor composition in an amount effective to inhibit proliferation of the melanoma cells,
wherein the microRNA inhibitor composition comprises an inhibitor of a microRNA, which microRNA is selected from the group consisting of microRNA-21, microRNA-146, microRNA-155, microRNA-506, microRNA-507, microRNA-508, microRNA-509, microRNA-510, microRNA-513, microRNA-514, microRNA-506-514 cluster, microRNA-506-513 cluster, and combinations thereof.

B6. A method, comprising:
contacting melanoma cells with a microRNA inhibitor composition in an amount effective to induce apoptosis of the melanoma cells,
wherein the microRNA inhibitor composition comprises an inhibitor of a microRNA, which microRNA is selected from the group consisting of microRNA-21, microRNA-146, microRNA-506, microRNA-507, microRNA-508, microRNA-509, microRNA-510, microRNA-513, microRNA-514, microRNA-506-514 cluster, microRNA-506-513 cluster, and combinations thereof.

C1. The method of any one of claims A1 to A9, wherein the presence, absence or amount of a microRNA-21, microRNA-146 and microRNA-155 is determined.

C2. The method of any one of claims B4 to B6, wherein a composition comprising one or more microRNA inhibitors of a microRNA selected from the group consisting of microRNA-21, microRNA-146 and microRNA-155 is utilized.

C3. The method of any one of claims A1 to A9, wherein the presence, absence or amount of a microRNA-506, microRNA-507, microRNA-508, microRNA-509, microRNA-510, microRNA-513 and microRNA-514 is determined.

C3.1. The method of any one of claims A1 to A9, wherein the presence, absence or amount of a microRNA-506, microRNA-507, microRNA-508 and microRNA-513 is determined.

C4. The method of any one of claims B4 to B6, wherein a composition comprising one or more microRNA inhibitors of a microRNA selected from the group consisting of microRNA-506, microRNA-507, microRNA-508, microRNA-509, microRNA-510, microRNA-513 and microRNA-514 is utilized.

C4.1. The method of any one of claims B4 to B6, wherein a composition comprising one or more microRNA inhibitors of a microRNA selected from the group consisting of microRNA-506, microRNA-507, microRNA-508 and microRNA-513 is utilized.

C5. The method of any one of claims A1-B3, wherein the microRNA-193 is a microRNA-193b.

C6. The method of any one of claims A1-B3, wherein the microRNA-10 is a microRNA-10a.

C7. The method of any one of claims A1-A9 and B4-B6, wherein the microRNA-146 is a microRNA-146a.

C7.1. The method of any one of claims A1-A9 and B4 to B6, wherein the microRNA-509 is microRNA-509-1, -2 or -3.

C8. The method of any one of claims B2, B3, B5 and B6, wherein the melanoma cells are in a tumor.

D1. A method for treating melanoma in a subject, comprising:
administering a composition to a subject in need thereof in an amount effective to treat melanoma in the subject,
wherein the composition comprises one or more components that deliver to the subject (i) imidazole carboxamide, and (ii) a microRNA composition that increases sensitivity of melanoma cells to imidazole carboxamide anti-cell proliferation activity.

D2. A method, comprising:
contacting melanoma cells with a composition in an amount effective to inhibit proliferation of the melanoma cells,
wherein the composition comprises one or more components that deliver (i) imidazole carboxamide, and (ii) a microRNA composition that increases sensitivity of melanoma cells to imidazole carboxamide anti-cell proliferation activity.

D3. A method, comprising:
contacting melanoma cells with a composition in an amount effective to induce apoptosis of the melanoma cells,
wherein the composition comprises one or more components that deliver (i) imidazole carboxamide, and (ii) a microRNA composition that increases sensitivity of melanoma cells to imidazole carboxamide anti-cell proliferation activity.

E1. A method, comprising:
(a) administering a composition that delivers an imidazole carboxamide drug to a subject having melanoma;
(b) identifying or determining the presence, absence or amount of a biomarker in the subject, wherein the biomarker is selected from the group consisting of a microRNA that increases sensitivity of melanoma cells to imidazole carboxamide anti-cell proliferation activity; and
(c) maintaining a subsequent dosage of the drug or adjusting a subsequent dosage of the drug administered to the subject based on the presence, absence or amount of the biomarker identified in the subject.

E2. A method, comprising:
(a) administering a composition that delivers an imidazole carboxamide drug to a subject having melanoma;
(b) identifying or determining the presence, absence or amount of a biomarker in the subject, wherein the biomarker is selected from the group consisting of a microRNA that increases sensitivity of melanoma cells to imidazole carboxamide anti-cell proliferation activity; and
(c) determining whether the dosage of the drug subsequently administered to the subject is adjusted based on the presence, absence or amount of the biomarker identified in the subject.

E3. A method, comprising:
(a) identifying or determining the presence, absence or amount of a biomarker in a subject having melanoma to whom a composition that delivers an imidazole carboxamide drug has been administered, wherein the biomarker is selected from the group consisting of a microRNA that increases sensitivity of melanoma cells to imidazole carboxamide anti-cell proliferation activity; and
(b) maintaining a subsequent dosage of the drug or adjusting a subsequent dosage of the drug administered to the subject based on the presence, absence or amount of the biomarker identified in the subject.

E4. A method, comprising:
(a) identifying or determining the presence, absence or amount of a biomarker in a subject having melanoma to whom a composition that delivers an imidazole carboxamide drug has been administered, wherein the biomarker is selected from the group consisting of a microRNA that increases sensitivity of melanoma cells to imidazole carboxamide anti-cell proliferation activity; and
(b) determining whether the dosage of the drug subsequently administered to the subject is adjusted based on the presence, absence or amount of the biomarker identified in the subject.

E5. A method, comprising:
(a) receiving information comprising the presence, absence or amount of a biomarker in a subject having melanoma to whom a composition that delivers an imidazole carboxamide drug has been administered, wherein the biomarker is selected from the group consisting of a microRNA that increases sensitivity of melanoma cells to imidazole carboxamide anti-cell proliferation activity; and
(b) maintaining a subsequent dosage of the drug or adjusting a subsequent dosage of the drug administered to the subject based on the presence, absence or amount of the biomarker identified in the subject.

E6. A method, comprising:
(a) identifying or determining the presence, absence or amount of a biomarker in a subject having melanoma to whom a composition that delivers an imidazole carboxamide drug has been administered, wherein the biomarker is selected from the group consisting of a microRNA that increases sensitivity of melanoma cells to imidazole carboxamide anti-cell proliferation activity; and
(b) transmitting the presence, absence or amount of the biomarker to a decision maker who maintains a subsequent dosage of the drug or adjusts a subsequent dosage of the drug administered to the subject based on the presence, absence or amount of the biomarker identified in the subject.

E7. A method, comprising:
(a) identifying or determining the presence, absence or amount of a biomarker in a subject having melanoma to whom a composition that delivers an imidazole carboxamide drug has been administered, wherein the biomarker is selected from the group consisting of a microRNA that increases sensitivity of melanoma cells to imidazole carboxamide anti-cell proliferation activity; and
(b) transmitting an indication to maintain a subsequent dosage of the drug or adjust a subsequent dosage of the drug administered to the subject based on the presence, absence or amount of the biomarker identified in the subject.

E8. A method for optimizing therapeutic efficacy of a treatment of melanoma in a subject, comprising:
(a) identifying or determining the presence, absence or amount of a biomarker in a subject having melanoma to whom a composition that delivers an imidazole carboxamide drug has been administered, wherein the biomarker is selected from the group consisting of a microRNA that increases sensitivity of melanoma cells to imidazole carboxamide anti-cell proliferation activity; and
(b) maintaining a subsequent dosage of the drug or adjusting a subsequent dosage of the drug administered to the subject based on the presence, absence or amount of the biomarker identified in the subject.

E9. A method for reducing toxicity of a treatment of melanoma in a subject, comprising:
(a) identifying or determining the presence, absence or amount of a biomarker in a subject having melanoma to whom a composition that delivers an imidazole carboxamide drug has been administered, wherein the biomarker is selected from the group consisting of a microRNA that increases sensitivity of melanoma cells to imidazole carboxamide anti-cell proliferation activity; and
(b) maintaining a subsequent dosage of the drug or adjusting a subsequent dosage of the drug administered to the subject based on the presence, absence or amount of the biomarker identified in the subject.

E10. A method, comprising:
(a) identifying or determining the presence, absence or amount of a biomarker in a subject having melanoma, wherein the biomarker is selected from the group consisting of a microRNA that increases sensitivity of melanoma cells to imidazole carboxamide anti-cell proliferation activity; and
(b) determining whether a composition that delivers an imidazole carboxamide drug is administered, or not administered, to the subject based on the presence, absence or amount of the biomarker.

E11. A method, comprising:
(a) receiving information comprising the presence, absence or amount of a biomarker in a subject having melanoma, wherein the biomarker is selected from the group consisting of a microRNA that increases sensitivity of melanoma cells to imidazole carboxamide anti-cell proliferation activity; and
(b) determining whether a composition that delivers an imidazole carboxamide drug is administered, or not administered, to the subject based on the presence, absence or amount of the biomarker.

E12. A method, comprising:
(a) identifying or determining the presence, absence or amount of a biomarker in a subject having melanoma, wherein the biomarker is selected from the group consisting of a microRNA that increases sensitivity of melanoma cells to imidazole carboxamide anti-cell proliferation activity; and
(b) transmitting the presence, absence or amount of the biomarker to a decision maker who determines whether a composition that delivers an imidazole carboxamide drug is administered to the subject based on the presence, absence or amount of the biomarker.

E13. A method, comprising:
(a) identifying or determining the presence, absence or amount of a biomarker in a subject having melanoma, wherein the biomarker is selected from the group consisting of a microRNA that increases sensitivity of melanoma cells to imidazole carboxamide anti-cell proliferation activity; and
(b) providing an indication for administering, or not administering, a composition that delivers an imidazole carboxamide drug to the subject based on the presence, absence or amount of the biomarker.

E14. The method of any one of claims E10 to E13, comprising administering, or not administering, the composition.

E15. The method of any one of claims E10 to E13, comprising administering the composition, wherein the composition includes a microRNA composition that increases sensitivity of melanoma cells to imidazole carboxamide anti-cell proliferation activity.

E16. The method of any one of claims E10 to E13, comprising administering the composition and administering a composition that includes a microRNA composition that increases sensitivity of melanoma cells to imidazole carboxamide anti-cell proliferation activity.

E17. A method, comprising:
(a) identifying or determining the presence, absence or amount of a biomarker in a subject having melanoma, wherein the biomarker is selected from the group consisting of a microRNA that increases sensitivity of melanoma cells to imidazole carboxamide anti-cell proliferation activity; and
(b) administering an imidazole carboxamide drug to the subject based on the presence or amount of the biomarker identified.

E18. The method of claim E17, wherein the decision maker administers, or does not administer, the composition based on the presence, absence or amount of the biomarker.

E19. The method of claim E17, comprising administering a composition that includes one or more components that deliver to the subject a microRNA composition that increases sensitivity of melanoma cells to imidazole carboxamide anti-cell proliferation activity.

E20. A method, comprising:
(a) identifying or determining the presence, absence or amount of a biomarker in a subject having melanoma, wherein the biomarker is selected from the group consisting of a microRNA that increases sensitivity of melanoma cells to imidazole carboxamide anti-cell proliferation activity; and
(b) not administering an imidazole carboxamide drug to the subject based on the absence or amount of the biomarker identified.

E21. The method of claim E20, comprising selecting a composition that does not deliver imidazole carboxamide for administration to the subject.

E22. The method of claim E21, wherein the composition does not deliver an alkylating agent.

E23. The method of claim E21 or E22, wherein the composition is administered to the subject.

F1. The method of any one of claims D1-E23, wherein the microRNA composition comprises a microRNA selected from the group consisting of a microRNA-27, a microRNA-143, microRNA-215, microRNA-335, and combinations thereof.

F2. The method of claim F1, wherein the microRNA-10 is a microRNA-10a.

F3. The method of claim F1, wherein the microRNA-27 is a microRNA-27a or -27b.

F4. The method of claim F1, wherein the microRNA-143 is a microRNA-143a.

F5. The method of any one of claims D1-E23, wherein the microRNA is present at decreased levels in melanoma cells relative to non-cancerous quiescent cells.

F6. The method of any one of claims D1-E23, wherein the microRNA modulates expression of IL-6 receptor or a IL-6 receptor pathway member.

F7. The method of claim D2 or D3, wherein the melanoma cells are in a tumor.

G1. A method, comprising:
(a) administering an anti-cancer drug to a subject having metastatic melanoma;
(b) identifying or determining the presence, absence or amount of a biomarker in the subject, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(c) maintaining a subsequent dosage of the drug or adjusting a subsequent dosage of the drug administered to the subject based on the presence, absence or amount of the biomarker identified in the subject.

G2. A method, comprising:
(a) administering an anti-cancer drug to a subject having metastatic melanoma;
(b) identifying or determining the presence, absence or amount of a biomarker in the subject, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(c) determining whether the dosage of the drug subsequently administered to the subject is adjusted based on the presence, absence or amount of the biomarker identified in the subject.

G3. A method, comprising:
(a) identifying or determining the presence, absence or amount of a biomarker in a subject having metastatic melanoma to whom an anti-cancer drug has been administered, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) maintaining a subsequent dosage of the drug or adjusting a subsequent dosage of the drug administered to the subject based on the presence, absence or amount of the biomarker identified in the subject.

G4. A method, comprising:
(a) identifying or determining the presence, absence or amount of a biomarker in a subject having metastatic melanoma to whom an anti-cancer drug has been administered, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) determining whether the dosage of the drug subsequently administered to the subject is adjusted based on the presence, absence or amount of the biomarker identified in the subject.

G5. A method, comprising:
(a) receiving information comprising the presence, absence or amount of a biomarker in a subject having metastatic melanoma to whom an anti-cancer drug has been administered, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) maintaining a subsequent dosage of the drug or adjusting a subsequent dosage of the drug administered to the subject based on the presence, absence or amount of the biomarker identified in the subject.

G6. A method, comprising:
(a) identifying or determining the presence, absence or amount of a biomarker in a subject having metastatic melanoma to whom an anti-cancer drug has been administered, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) transmitting the presence, absence or amount of the biomarker to a decision maker who maintains a subsequent dosage of the drug or adjusts a subsequent dosage of the drug administered to the subject based on the presence, absence or amount of the biomarker identified in the subject.

G7. A method, comprising:
(a) identifying or determining the presence, absence or amount of a biomarker in a subject having metastatic melanoma to whom an anti-cancer drug has been administered, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) transmitting an indication to maintain a subsequent dosage of the drug or adjust a subsequent dosage of the drug administered to the subject based on the presence, absence or amount of the biomarker identified in the subject.

G8. A method for optimizing therapeutic efficacy of a treatment of metastatic melanoma in a subject, comprising:
(a) identifying or determining the presence, absence or amount of a biomarker in a subject having metastatic melanoma to whom an anti-cancer drug has been administered, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) maintaining a subsequent dosage of the drug or adjusting a subsequent dosage of the drug administered to the subject based on the presence, absence or amount of the biomarker identified in the subject.

G9. A method for reducing toxicity of a treatment of metastatic melanoma in a subject, comprising:
(a) identifying or determining the presence, absence or amount of a biomarker in a subject having metastatic melanoma to whom an anti-cancer drug has been administered, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) maintaining a subsequent dosage of the drug or adjusting a subsequent dosage of the drug administered to the subject based on the presence, absence or amount of the biomarker identified in the subject.

G10. A method, comprising:
(a) identifying or determining the presence, absence or amount of a biomarker in a subject, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) determining whether the subject is at risk, or not at risk, of having metastatic melanoma based on the presence, absence or amount of the biomarker.

G11. A method, comprising:
(a) receiving information comprising the presence, absence or amount of a biomarker in a subject, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) determining whether the subject is at risk, or not at risk, of having metastatic melanoma based on the presence, absence or amount of the biomarker.

G12. A method, comprising:
(a) identifying or determining the presence, absence or amount of a biomarker in a subject having melanoma, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) transmitting the presence, absence or amount of the biomarker to a decision maker who determines whether the subject is at risk, or not at risk, of having metastatic melanoma based on the presence, absence or amount of the biomarker.

G13. A method, comprising:
(a) identifying or determining the presence, absence or amount of a biomarker in a subject having melanoma, wherein the biomarker is selected from the group consisting of a microRNA-let7, microRNA-21, microRNA-146, microRNA-193, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) providing an indication that the subject is at risk, or not at risk, of having metastatic melanoma based on the presence, absence or amount of the biomarker.

G14. The method of any one of claims G10 to G13, comprising administering a composition that treats melanoma to a subject at risk of metastatic melanoma.

G15. The method of any one of claims G10 to G13, comprising not administering a composition that treats melanoma to a subject not at risk of metastatic melanoma.

G16. The method of any one of claims G10 to G15, wherein the subject has melanoma.

G17. The method of claim G16, wherein the subject has been diagnosed with melanoma.

H1. A method for treating metastatic melanoma in a subject, comprising:
administering a composition that delivers to a subject in need thereof a microRNA composition in an amount effective to inhibit metastasis of the melanoma cells in the subject,
wherein the microRNA composition comprises a microRNA selected from the group consisting of microRNA-let7, microRNA-193, microRNA-206, and combinations thereof.

H2. A method, comprising:
contacting metastatic melanoma cells with a microRNA composition in an amount effective to inhibit metastasis of the melanoma cells,
wherein the microRNA composition comprises a microRNA selected from the group consisting of microRNA-let7, microRNA-193, microRNA-206, and combinations thereof.

H3. A method, comprising:
contacting metastatic melanoma cells with a microRNA composition in an amount effective to inhibit metastasis of the melanoma cells,
wherein the microRNA composition comprises a microRNA selected from the group consisting of microRNA-let7, microRNA-193, microRNA-206, and combinations thereof.

H4. A method for treating metastatic melanoma in a subject, comprising:
administering a composition that delivers to a subject in need thereof a microRNA inhibitor composition in an amount effective to inhibit metastasis of the melanoma in the subject,
wherein the microRNA inhibitor composition comprises an inhibitor of a microRNA selected form the group consisting of microRNA-21, microRNA-146, microRNA-506, microRNA-507, microRNA-508, microRNA-509, microRNA-510, microRNA-513, microRNA-514, microRNA-506-514 cluster, microRNA-506-513 cluster, and combinations thereof.

H5. A method, comprising:
contacting metastatic melanoma cells with a microRNA inhibitor composition in an amount effective to inhibit metastasis of the melanoma cells,
wherein the microRNA inhibitor composition comprises an inhibitor of a microRNA selected from the group consisting of microRNA-21, microRNA-146, microRNA-506, microRNA-507, microRNA-508, microRNA-509, microRNA-510, microRNA-513, microRNA-514, microRNA-506-514 cluster, microRNA-506-513 cluster, and combinations thereof.

I1. The method of any one of claims G1 to H5, wherein the metastasis is invasion by melanoma cells of non-cancer tissue.

I1.1. The method of claim I1, wherein the tissue is not skin.

I2. The method of any one of claims G1 to H5, wherein the metastasis is migration of melanoma cells.

I3. The method of any one of claims G1 to H3, wherein the microRNA-let7 is a microRNA-let7c.

I4. The method of any one of claims G1 to H3, wherein the microRNA-509 is a microRNA-509-1, -2 or -3.

I5. The method of any one of claims G1 to H3, wherein the microRNA-193 is a microRNA-193b.

I6. The method of any one of claims G1 to G17, H4 and H5, wherein the microRNA-146 is a microRNA-146a.

I7. The method of any one of claims G1 to G17, wherein the presence, absence or amount of microRNA-506, microRNA-507, microRNA-508, microRNA-509, microRNA-510, microRNA-513 and microRNA-514 is determined.

I8. The method of any one of claims G1 to G17, wherein the presence, absence or amount of microRNA-506, microRNA-507, microRNA-508 and microRNA-513 is determined.

I9. The method of claims H4 or H5, wherein a composition comprising a microRNA inhibitor or microRNA inhibitors of a microRNA-506, microRNA-507, microRNA-508, microRNA-509, microRNA-510, microRNA-513 and microRNA-514 is utilized.

I9.1. The method of claims H4 or H5, wherein a composition comprising a microRNA inhibitor or microRNA inhibitors of a microRNA-506, microRNA-507, microRNA-508 and microRNA-513 is utilized.

I10. The method of claim H2, H3 or H5, wherein the metastatic melanoma cells are in a tumor.

J1. The method of any one of claims A1-I10, wherein the melanoma is metastatic melanoma.

J2. The method of any one of claims A1-J1, wherein the microRNA is a human microRNA.

J3. The method of any one of claims A1-J2, wherein the subject is human.

J4. The method of any one of claims A1-A9, C1, E1-E23, F1-F4, G1-G17 and I3-I9, wherein the presence, absence or amount of the biomarker is determined from a biological sample from the subject.

J5. The method of claim J4, wherein sample contains blood or a blood fraction.

J6. The method of claim J4, wherein the sample contains a skin biopsy product.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1-100. (canceled)

101. An anti-cancer drug for use in treating a subject having melanoma, characterized in that the maintenance of a subsequent dosage of the drug or adjustment of a subsequent dosage of the drug administered to the subject is based on the presence, absence or amount of a biomarker selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof.

102. A method for optimizing therapeutic efficacy of a treatment of melanoma in a subject, comprising:
(a) identifying or determining the presence, absence or amount of a biomarker in a subject having melanoma to whom an anti-cancer drug has been administered, wherein the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) maintaining a subsequent dosage of the drug or adjusting a subsequent dosage of the drug administered to the subject based on the presence, absence or amount of the biomarker identified in the subject.

103. A method for reducing toxicity of a treatment of melanoma in a subject, comprising: (a) identifying or determining the presence, absence or amount of a biomarker in a subject having melanoma to whom an anti-cancer drug has been administered, wherein the biomarker is selected from the group consisting of a microRNA-let7, microRNA-10, microRNA-21, microRNA-126, microRNA-146, microRNA-155, microRNA-193, microRNA-203, microRNA-206, microRNA-506, microRNA-507, microRNA-508, microRNA-509, microRNA-510, microRNA-513, microRNA-514, microRNA-506-514 cluster, microRNA-506-513 cluster, and combinations thereof; and
(b) maintaining a subsequent dosage of the drug or adjusting a subsequent dosage of the drug administered to the subject based on the presence, absence or amount of the biomarker identified in the subject.

104. A microRNA composition for use in treating melanoma in a subject, characterized in that the microRNA composition comprises a microRNA selected from the group consisting of microRNA selected from the group consisting of microRNA-10, microRNA-126, microRNA-193, microRNA-203, microRNA-206, and combinations thereof.

105. A microRNA composition of claim 104, wherein the microRNA composition inhibits proliferation of the melanoma cells.

106. A microRNA composition of claim 104, wherein the microRNA composition induces apoptosis of the melanoma cells.

107. A microRNA inhibitor composition for use in treating melanoma in a subject, characterized in that the microRNA inhibitor composition comprises an inhibitor of a microRNA-21, microRNA-146, microRNA-155, microRNA-506, microRNA-507, microRNA-508, microRNA-509, microRNA-510, microRNA-513, microRNA-514, microRNA-506-514 cluster, microRNA-506-513 cluster, and combinations thereof.

108. A microRNA composition of claim 107, wherein the microRNA composition inhibits proliferation of the melanoma cells.

109. A microRNA composition of claim 107, wherein the microRNA composition induces apoptosis of the melanoma cells.
